(19) United States
(12) Patent Application Publication
(10) Pub. No.: US 2023/0346919 A1
(43) Pub. Date: Nov. 2, 2023

(54) SARS COV-2 VACCINES AND HIGH THROUGHPUT SCREENING ASSAYS BASED ON VESICULAR STOMATITIS VIRUS VECTORS

(71) Applicant: Washington University, St. Louis, MO (US)

(72) Inventors: Sean Whelan, St. Louis, MO (US); Paul Rothlauf, St. Louis, MO (US); Michael Diamond, St. Louis, MO (US); Brett Case, St. Louis, MO (US)

(73) Assignee: Washington University, St. Louis, MO (US)

(21) Appl. No.: 17/918,792

(22) PCT Filed: Apr. 14, 2021
(86) PCT No.: PCT/US21/27275

§ 371 Date: Oct. 13, 2022

Related U.S. Application Data
(60) Provisional application No. 63/049,890, filed on Jul. 9, 2020.
(60) Provisional application No. 63/027,615, filed on May 20, 2020.
(60) Provisional application No. 63/009,723, filed on Apr. 14, 2020.

(51) Int. Cl. A61K 39/215; A61P 31/14; C12N 15/86; G01N 33/50; G01N 33/569

(52) U.S. Cl. CPC A61K 39/215; A61P 31/14; C12N 15/86; G01N 33/5091; G01N 33/56983; G01N 2333/165

(57) ABSTRACT

Compositions and methods useful for vaccinating for or preventing a Coronavinus infection, antibody screening, and variant prediction are provided. The present disclosure also provides for compositions and methods useful for sera diagnostics, treatment screening, and methods of making the compositions described herein.

### CROSS-REFERENCE TO RELATED APPLICATIONS

This application claims priority from U.S. Provisional Application Ser. No. 63/009,723 filed on 14 Apr. 2020; 63/027,615 filed on 20 May 2020; and 63/049,890 filed on 9 Jul. 2020, which are incorporated herein by reference in their entireties.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD

The present disclosure generally relates to vaccines, screening tools, and assays for use in SARS CoV-2 applications.

### SUMMARY

Among the various aspects of the present disclosure is the provision of compositions and methods for using and making Coronavirus (SARS CoV-2) vaccines and screening assays. 1. A recombinant vesicular stomatitis virus (rVSV) comprising at least a portion of a coronavirus spike protein (S) protein or a functional fragment or a functional variant thereof. An aspect of the present disclosure provides for a recombinant vesicular stomatitis virus (rVSV) construct or vaccine platform comprising: at least a portion of a vesicular stomatitis virus (VSV); and/or at least a portion of, a functional fragment of, or functional variant of the spike (S) of SARS-CoV-2 (SEQ ID NO: 1) and/or at least about 80% identical to SEQ ID NO: 1. Another aspect of the present disclosure provides for a recombinant vesicular stomatitis virus (rVSV) comprising in its genome a nucleic acid sequence encoding at least a portion of, a functional fragment of, or functional variant of the spike (S) of SARS-CoV-2 (SEQ ID NO: 1) and/or at least about 80% identical to a functional portion or fragment of SEQ ID NO: 1. In some embodiments, the rVSV is replication-competent. In some embodiments, the at least a portion of, a functional fragment, or functional variant of the spike (S) of SARS-CoV-2 comprises one or more mutations or attenuations or truncations. In some embodiments, at least a portion of the rVSV comprises genes encoding a leader region (Le), a reporter gene (e.g., eGFP), nucleocapsid (N), phosphoprotein (P), matrix (M), glycoprotein (G), SARS-CoV-2 (S), large polymerase (L), or trailer region (Tr), or combinations thereof. In some embodiments, the VSV construct displays the S protein in an antigenic form that resembles native infectious SARS-CoV-2. In some embodiments, the coding region of the glycoprotein (G) of the VSV is replaced by the coding region of a spike (S) protein of SARS-CoV-2 or a functional variant, portion, or fragment of SEQ ID NO: 1. In some embodiments, the spike (S) is selected from a functional mutation, insertion, deletion, or substitution thereof and 80% identical to SEQ ID NO: 1. In some embodiments, S is selected from any one of SEQ ID NO: 1 or 4-9 or a functional portion or fragment thereof. In some embodiments, the rVSV comprises a tail mutation capable of allowing for its incorporation into a VSV construct. In some embodiments, the tail mutation is selected from one or more of the following: S_{Δ21} (tail mutant 21 amino acid truncation or deletion of residues 1253-1273) or S_{AA} (tail mutant K1269A/H1271A). In some embodiments, a spike mutation can be selected from one or more of the following: T345A; T345A/L517R; T345A/E484A; T345N; T345S; T345S/F486S; T346G/E484A; R346K/E484K; R346G; R346K; A352D; A372T; A372T/E484K; K378E; K378Q; R408K; K417N; L441R; K444E; K444E/E484A; K444E/E484K; K444N; K444R; V445G; G446D; G446V; N450D; N450K; N450Y; L452R; K458Q; Q474P; G476D; G476S; S4771; S477G; S477N; S477N/S514F; S477R; T478I; T478P; P479L; P479S; V483F; V483G; E484; E484A; E484D; E484G; E484K; F486L; F486S; F486Y; F486V; E488A; F490L; F490S; S494P; P499L; N501Y; G504D; S514F; L517R; K535R; D614G; or combinations thereof or any other substitutions or deletions at the aforementioned amino acid residue site. In some embodiments, the spike (S) has a mutation or deletion selected from S_{AA}, S_{Δ21}, K535R, or a functional variant, mutant, or fragment thereof having at least 80% identity to SEQ ID NO: 1. In some embodiments, the nucleic acid sequence encoding at least a portion of a Coronavirus spike protein (S) protein, or functional fragment or variant thereof, substantially replaces the endogenous VSV viral glycoprotein (G) in the VSV genome. In some embodiments, the nucleic acid encoding the at least a portion or a functional fragment of a Coronavirus spike protein (S) protein or a functional fragment or variant thereof has a sequence 80% identical to a functional portion or fragment of the Coronavirus spike protein portion of SEQ ID NO: 2 or SEQ ID NO: 3; or the nucleic acid encoding the at least a portion of a Coronavirus spike protein (S) protein or functional fragment or variant thereof encodes a polypeptide having a sequence 80% identical to a functional portion or fragment of SEQ ID NO: 1 or SEQ ID NO: 4-9. In some embodiments, at least a portion of a Coronavirus spike protein (S) protein or functional fragment or variant thereof comprises an amino acid sequence that is identical to the at least a portion of the Coronavirus spike protein sequence of SEQ ID NO: 3 or SEQ ID NO: 8-12. Yet another aspect of the present disclosure provides for a pharmaceutical composition comprising the rVSV of any preceding claim, and a pharmaceutically acceptable carrier. In some embodiments, the composition comprises an adjuvant. Yet another aspect of the present disclosure provides for a Coronavirus vaccine comprising the rVSV of any one of the preceding claims. In some embodiments, the adjuvant is K3 CpG. In some embodiments, the vaccine is a replication-competent vaccine against SARS-CoV-2. In some embodiments, the recombinant VSV is a live virus. Yet another aspect of the present disclosure provides for a nucleic acid encoding the rVSV according to any one of the preceding aspects or embodiments. In some embodiments, the nucleic acid has a sequence that is at least 80% identical to the portion encoding the Coronavirus spike protein of a functional portion or fragment of SEQ ID NO: 2 or SEQ ID NO: 3. Yet another aspect of the present disclosure provides for an expression vector comprising the nucleic acid of any one of the preceding claims. Yet another aspect of the present disclosure provides for a cell comprising the nucleic acid according to any one of the preceding aspects or embodiments. Yet another aspect of the present disclosure provides for a method for inducing an immune response against Coronavirus in a subject, the method comprising administering to the subject at least one dose of a composition comprising the rVSV of any one of the preceding aspects or embodiments. In some embodiments, the administration of the composition generates a robust neutralizing antibody response that targets both a SARS-CoV-2 S protein and a receptor binding domain (RBD) subunit. In some embodiments, the administration of the composition stimulates both humoral and cellular immunity. In some embodiments, the administration of the composition results in a response comprising decreases lung or peripheral organ viral loads, pro-inflammatory cytokine responses, and/or consequent lung disease. In some embodiments, the administration of the composition results in protection from alveolar inflammation, lung consolidation, or viral pneumonia. In some embodiments, the administration of the composition results in protection against severe SARS-CoV-2 infection and lung disease. In some embodiments, the subject produces protective antibodies in the sera of the subject. In some embodiments, passive transfer of immune sera from immunized subject decreases viral burden or inflammation in the lung. In some embodiments, a second dose substantially boosts the response. In some embodiments, the route of administration is intramuscular or intranasal. In some embodiments, the subject is a human. In some embodiments, the subject has been exposed to Coronavirus. In some embodiments, the subject does not have, but is at risk of developing a Coronavirus infection. In some embodiments, the subject is traveling to a region where the Coronavirus is prevalent. Yet another aspect of the present disclosure provides for a method for protecting a subject from Coronavirus, comprising administering to the subject at least one dose of the recombinant virus of any one of the preceding aspects or embodiments, wherein, optionally, the rVSV confers protection against SARS-CoV-2-induced lung infection and/or inflammation, such as pneumonia. In some embodiments, the subject is a human. In some embodiments, the subject is exposed to Coronavirus. In some embodiments, the subject does not have, but is at risk of developing a Coronavirus infection. In some embodiments, the subject is traveling to a region where the Coronavirus is prevalent. Yet another aspect of the present disclosure provides for a method of treating a Coronavirus infection in a subject, the method comprising administering to the subject the sera of a subject that received a composition comprising the rVSV of any one of the preceding aspects or embodiments; or administering to the subject the composition comprising the rVSV of any one of the preceding aspects or embodiments. In some embodiments, the subject is a human. Yet another aspect of the present disclosure provides for a method of making virus-like particles (VLP) comprising: transfecting a cell with the expression vector; culturing the cell under conditions such that the cell produces a Coronavirus VLP; and/or collecting the Coronavirus VLP. In some embodiments, the cells are BSRT7 cells, Vero, or MA104. In some embodiments, the cells are infected with Vaccinia VTF7-3 and transfected with an infectious molecular clone encoding S or an S mutant and helper plasmids N, P, L, and G to rescue recombinant virus. In some embodiments, the methods further comprise infecting the cells with a rescue supernatant, viral particles of which contain VSV G in trans. Yet another aspect of the present disclosure provides for a method for screening treatments for Coronavirus comprising: providing a cell infected with the rVSV of any one of the preceding aspects or embodiments comprising a reporter gene; contacting the cell with an experimental therapeutic agent; and/or detecting the reporter gene expression to determine if the experimental therapeutic agent neutralized the rVSV. Yet another aspect of the present disclosure provides for a method for screening a subject for Coronavirus antibodies: providing a biological sample from a subject; contacting the cell with rVSV of any one of the preceding aspects or embodiments comprising a reporter gene; and/or detecting the reporter gene expression to determine if antibodies in the sample neutralized the rVSV, compared to a cell not having Coronavirus antibodies or a biological sample from a subject not having been infected with Coronavirus. In some embodiments, the vesicular stomatitis virus (VSV) encodes a SARS-CoV-2 spike or mutant or truncated or attenuated variant thereof at least 80% identical to a functional portion or fragment of SEQ ID NO: 1. Yet another aspect of the present disclosure provides for a neutralization assay comprising: a rVSV construct expressing a reporter gene and a SARS-CoV-2 spike protein or mutant or truncated or attenuated variant thereof at least 80% identical to a functional portion or fragment of SEQ ID NO: 1. In some embodiments, the SARS-CoV-2 spike protein is S_{Δ21} or S_{AA} or mutant, or truncated or attenuated variant thereof at least 80% identical to a functional portion or fragment of SEQ ID NO: 1. In some embodiments, the assay is a BSL2 assay for evaluating SARS-CoV-2 entry and its inhibition by antibodies. In some embodiments, the assay is a high-throughput-imaging-based neutralization assay at biosafety level 2. In some embodiments, the assay is capable of testing inhibitors of SARS-CoV-2 mediated entry under reduced biosafety containment. In some embodiments, the inhibitors are selected from monoclonal antibodies (e.g., to the spike protein) or an ACE receptor (e.g., soluble ACE2-Fc). In some embodiments, the neutralization assay correlates with a focus-reduction neutralization test with a clinical isolate of SARS-CoV-2 at biosafety level 3. In some embodiments, the spike protein is a functional spike protein having ACE2 receptor binding activity. Yet another aspect of the present disclosure provides for a method of measuring neutralization comprising: infecting a cell with an rVSV-CoV-2-S comprising a reporter gene construct and imaging the reporter gene after contact with a test agent (e.g., serum from subjects having a previous infection or vaccinated) or treatment with an inhibiting agent (e.g., mAb, ACE2-Fc). In some embodiments, samples are then analyzed for neutralization activity. In some embodiments, imaging is performed using a fluorescence microscope with automated counting analysis software. Yet another aspect of the present disclosure provides for a method of identifying escape mutants comprising: selecting a spike protein mutation; generating a VSV-SARS-CoV-2-S having the mutation; and/or contacting the VSV-SARS-CoV-2-S with an inhibitor of spike function; identifying if the mutation is an escape mutation based on reporter gene signal. In some embodiments, the spike mutation represents a nucleotide sequence of a circulating viral strain. In some embodiments, the inhibitor of spike function is a monoclonal antibody, a soluble receptors, or other inhibitors of spike function, such as antibodies, Fc, receptor decoys, vaccine induced antibodies, or molecules. In some embodiments, the method further comprises comparing escape mutation results to sequence data obtained from surveillance of circulating viruses. In some embodiments, if escape mutations correlate with circulating viral mutations, a vaccine can be designed to include the mutation.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. Strategy for construction and recovery of rVSV-SARS-CoV-2 variants. (A) Schematic representation of rVSV-SARS-CoV-2 constructs, their rescue, and autonomous propagation. The genomic RNA of the infectious molecular clones of vesicular stomatitis virus (VSV) are shown in which the coding region of the glycoprotein (G) was replaced by the spike (S) of SARS-CoV-2. A series of variants incorporating mutations in the cytoplasmic tail of SARS-CoV-2 S to enhance expression at the plasma membrane and incorporation into VSV particles are shown. (B) For rescue of virus from cDNA, BSRT7/5 cells were infected with vaccinia virus expressing T7 RNA polymerase as source of transcriptase (vTF7-3) and subsequently transfected with plasmids encoding the VSV N, P, L, and G genes and the indicated molecular clone. Cell culture fluids were harvested at 56-72 hours post-transfection and used to inoculate fresh Vero cells, which were then monitored for infection. (C) Infection and eGFP expression of rVSV-eGFP-SARS-CoV-2-S_{AA} at 44 hours post-infection on Vero cells.

FIG. 2. Forward genetic selection of a variant of rVSV-eGFP-SARS-CoV-2-S_{AA}. (A) Schematic of the forward genetics approach taken to select mutants of rVSV-eGFP-SARS-CoV-2-S_{AA}. The cell culture fluids from the transfection were repeatedly passaged on Vero cells, and monitored by plaque formation. As the kinetics of plaque formation and plaque size increased, we isolated 6 plaques. A representative plaque assay showing viral entry and spread as mediated solely by SARS-CoV-2 S is shown. (B) The viral RNA was extracted and analyzed by next generation sequencing and compared to the infectious molecular clone sequence. All variants acquired a cysteine to stop mutation in the cytoplasmic tail of SARS-CoV-2 spike, which we termed SARS-CoV-2-SΔ21, as it truncates the cytoplasmic tail by 21 amino acid residues and eliminates the plasma membrane targeting sequence. (C) A side-by-side comparison of plaque size of rVSV-eGFP-SARS-CoV-2-S_{AA} and rVSV-eGFP-SARS-CoV-2-SΔ21 on Vero and Vero-Furin cells at 92 hour post infection. Samples were scanned using a biomolecular imager and eGFP expression is shown. (D) Growth and eGFP expression by rVSV-eGFP-SARS-CoV-2-SΔ21. The African Green Monkey kidney cell line, MA104, was infected with rVSV-eGFP-SARS-CoV-2-SΔ21 at an MOI of 0.05 and images were acquired at 18 hours post-infection on MA104, Vero, and Vero E6 cells. Samples were scanned using a biomolecular imager and eGFP expression is shown.

FIG. 3. Neutralization of rVSV-eGFP-SARS-CoV-2-SΔ21 infectivity by soluble, human ACE2-Fc. A high throughput 96-well plate format neutralization assay is shown. The indicated concentrations of purified, recombinant, human ACE2-Fc or murine ACE2-Fc were incubated with equal volumes of rVSV-eGFP-SARS-CoV-2-SΔ21 or rVSV-eGFP at 37° C. for 1 hr. MA104 cells were then added to the virus-ACE2 mixture and eGFP signal per condition was measured using a biomolecular imager after 8 hours. One representative image is displayed.

FIG. 4. Protein composition of rVSV-eGFP-SARS-CoV-2-SΔ21. (A) Coomassie-stained high-bis 8% SDS-PAGE gel under reducing and denaturing conditions. Wild-type VSV and rVSV-eGFP-SARS-CoV-2-SΔ21 were grown on BSRT7/5 cells in the presence of VSV G, sucrose-gradient purified and treated with PNGase F prior to loading on the gel. All of the VSV proteins, as well as SARS-CoV-2 Si and S₂, and PNGase F are labeled. (B) Coomassie-stained, 8% SDS-PAGE gel run under reducing and denaturing conditions. Samples are the same as in (A), with the addition of non-PNGaseF-treated virions. (C) Western blot of sucrose gradient-purified rVSV-eGFP-SARS-CoV-2-SΔ21 grown on BSRT7/5 cells in the presence of VSV G, sucrose gradient-purified wild-type VSV, and rVSV-eGFP-SARS-CoV-2-SΔ21 grown on MA104 cells. S was detected using the SARS-neutralizing antibody CR3022 and an HRP-fused secondary was used to detect the primary.

FIG. 5. Graphical abstract describing the administering an VSV vaccine to a mouse, harvesting immune sera, and measuring the amount of neutralizing Abs in the sera; vaccinating a mouse expressing human ACE2, challenging the vaccinated mouse with SARS-CoV-2, and measuring viral burden; and treating a mouse expressing human ACE2 with immune sera, challenging the mouse with SARS-CoV-2, and measuring viral burden.

FIG. 6. Immunogenicity of VSV-eGFP-SARS-CoV-2. (A) Scheme of vaccination and SARS-CoV-2 challenge. (B-D) Four-week-old female BALB/c mice were immunized with VSV-eGFP or VSV-eGFP-SARS-CoV-2. Some of the immunized mice were boosted with their respective vaccines four weeks after primary vaccination. IgG responses in the sera of vaccinated mice were evaluated three weeks after priming or boosting by ELISA for binding to SARS-CoV-2 S (B) or RBD (C) or two weeks after priming or boosting by focus reduction neutralization test (FRNT) (D) (n=15 per group; one-way ANOVA with Dunnett's post-test: ****p<0.0001). Bars indicate median values. (E and F) Four-week-old K18-hACE2 transgenic mice were immunized with VSV-eGFP or VSV-eGFP-SARS-CoV-2 via an intranasal route. Three weeks later, serum was harvested and levels of anti-SARS-CoV-2 RBD antibodies (IgM, IgA, IgG1, IgG2b, IgG2c, IgG3, and total IgG) were determined by ELISA (n=3-7 per group; Mann-Whitney test: *p<0.05) (E), or neutralizing antibody titers were determined by FRNT (F) (n=7 per group; Mann-Whitney test: ***p<0.001). See FIG. 10.

FIG. 7. VSV-eGFP-SARS-CoV-2 Protects Mice against SARS-CoV-2 Infection. (A-E) Three weeks after priming or boosting with VSV-eGFP or VSV-eGFP-SARS-CoV-2, immunized animals were treated with anti-Ifnar1 mAb and one day later, animals were transduced with 2.5×10⁸ PFU of AdV-hACE2 by intranasal administration. Five days later, animals were challenged with 3×10⁵ PFU of SARS-CoV-2 via intranasal administration. At 4 or 8 dpi tissues were harvested, and viral burden was determined in the lung ([A] and [B]), spleen (C), heart (D), and nasal washes (E) by plaque (A) or RT-qPCR ([B]-[E]) assay (n=7-8 mice per group; Kruskal-Wallis test with Dunn's post-test ([A]-[E]): ns, not significant, *p<0.05, **p<0.01, ***p<0.001, ****p<0.0001). Dotted lines indicate the limit of detection. Bars indicate median values. (F) SARS-CoV-2 RNA in situ hybridization of lungs of mice vaccinated with VSV-eGFP or VSV-eGFP-SARS-CoV-2 and challenged with SARS-CoV-2 at 4 dpi. Images show 40×—(left; scale bars, 100 μm), 200×—(middle; scale bars, 100 μm), and 400×—magnification (right; scale bars, 10 μm; representative images from n=3 lungs per group; 10 fields per slide).

FIG. 8. VSV-eGFP-SARS-CoV-2 Protects Mice from SARS-CoV-2 Lung Inflammation. (A) Lungs of VSV-eGFP- or VSV-eGFP-SARS-CoV-2-immunized mice were evaluated at 4 dpi for cytokine and chemokine expression by RT-qPCR assay. Data are shown as fold change in gene expression compared to fully naive, age-matched animals after normalization to Gapdh (n=7-8 per group, Kruskal-Wallis test with Dunn's post-test: *p<0.05, **p<0.01, ***p<0.001, ****p<0.0001). Bars indicate median values. (B) Hematoxylin and eosin staining of lung sections from immunized mice at 8 dpi with SARS-CoV-2 (3×10⁵ PFU). Images show 40×—(left; scale bars, 250 μm), 200×—(second from left; scale bars, 50 μm), 400×—(third from left; scale bars, 25 μm), and 630×—magnification (right; scale bars, 10 μm). Arrows indicate neutrophils in the alveolar septum (green) and airspace (blue). Representative images are shown from n=3 lungs per group; 10 fields per slide).

FIG. 9. Vaccine-Induced Sera Limits SARS-CoV-2 Infection. (A) Passive transfer of immune sera and SARS-CoV-2 challenge scheme. Ten-week-old female BALB/c mice were treated with anti-Ifnar1 mAb, and one day later animals were transduced with 2.5×10⁸ PFU of AdV-hACE2 by intranasal administration. Four days later, animals were administered 100 μL of pooled immune sera collected from VSV-eGFP or VSV-eGFP-SARS-CoV-2 vaccinated mice after one or two immunizations. One day later, animals were challenged with 3×10⁵ PFU of SARS-CoV-2 via intranasal administration. (B-F) At 4 dpi tissues were harvested, and viral burden was determined in the lung ([B] and [C]), spleen (D), heart (E), and nasal washes (F) by plaque (B) or RT-qPCR ([C]-[F]) assays (n=7 mice per group; Kruskal-Wallis test with Dunn's post-test ([B]-[F]): *p<0.05, **p<0.01, ****p<0.0001). Dotted lines indicate the limit of detection. (G) Lungs of mice treated with immune sera were evaluated at 4 dpi for cytokine expression by RT-qPCR assay. Data are shown as fold change in gene expression compared to naive, age-matched animals after normalization to Gapdh (n=7 per group, Kruskal-Wallis test with Dunn's post-test: *p<0.05, **p<0.01). (B-G) Bars indicate median values.

FIG. 10. Gradient-purified vaccine preparations (related to FIG. 6). VSV-eGFP-SARS-CoV-2 and VSV-eGFP were purified on a 15-45% sucrose-NTE gradient. Purified virions were subsequently treated with PNGase F (+) or mock (−), and proteins were separated on an 8% reducing and denaturing SDS-PAGE gel. Arrows correspond to proteins in VSV particles.

FIG. 11. Schematic of VSV-SARS-CoV-2 vaccines. Codon optimized (*) or codon optimized and wild-type (**) nucleotide sequences of the SARS-CoV-2 spike lacking the final 63 nucleotides were inserted into the VSV genome in place of the native G gene. Viruses were rescued, plaque-purified, and entire genomes were sequenced to validate the vaccine stocks.

FIG. 12. Vaccination of rhesus macaques with non-purified VSV-SARS-CoV-2. Rhesus macaques were vaccinated intramuscularly with a single 10⁷ pfu dose of VSV-SARS-CoV-2 (wild-type nucleotide sequence) or VSV-Ebola. A) Blood, nasal swabs, and throat swabs were tested for levels of VSV RNA at the days indicated post-vaccination. B) Spike-specific IgG levels were measured at the indicated days post-vaccination. C) Neutralizing antibody titers were measured at the days indicated post-vaccination (day 0=day challenged with SARS-CoV-2).

FIG. 13. Neutralizing antibody titers in rhesus macaques vaccinated with purified VSV-SARS-CoV-2. Rhesus macaques (n=4 per group) were vaccinated with a single 10⁷ pfu dose of VSV-SARS-CoV-2 (wild-type nucleotide sequence) and serum neutralizing antibody titers were measured at the indicated days post-vaccination using an established GFP-reduction neutralization test using VSV-eGFP-SARS-CoV-2. IM=intramuscular, IN=intranasal, IM+adj=intramuscular with K3 CpG adjuvant.

FIG. 14. Graphical abstract describing the rescue of replication-competent VSV expressing a functional SARS-CoV-2 spike (which can be a mutant, variant, or truncated version of the S protein) and the measurement of viral neutralization.

FIG. 15. Generation and Characterization of an Infectious VSV-SARS-CoV-2 Chimera. (A) A schematic diagram depicting the genomic organization of the VSV recombinants. Shown 3′ to 5′ are the leader region (Le), eGFP, nucleocapsid (N), phosphoprotein (P), matrix (M), glycoprotein (G) or SARS-CoV-2 S, large polymerase (L), and trailer region (Tr). On right, infection of Vero CCL81 cells with supernatant from cells transfected with the eGFP reporter VSV-SARS-CoV-2-S_{AA}. Images were acquired 44 h post-infection (hpi) using a fluorescence microscope, and GFP and transmitted light images were merged using ImageJ. Shown at bottom, the alignment of the cytoplasmic tail of the VSV-SARS-CoV-2-S_{AA} and the sequence resulting from forward genetic selection of a mutant, which truncated the cytoplasmic tail by 21 amino acids. Mutations deviating from the wild-type spike are indicated in red, and an asterisk signifies a mutation to a stop codon. (B) Plaque assays were performed to compare the spread of VSV-SARS-CoV-2-S_{AA} rescue supernatant and VSV-SARS-CoV-2-S_{Δ21} on Vero CCL81, Vero E6, Vero-furin, and MA104 cells. Plates were scanned on a biomolecular imager and expression of eGFP is shown 92 hpi (representative images are shown; n>3 except for S_{AA} on Vero E6, Vero-furin, and MA104 cells). (C) The indicated cell types were infected with VSV-SARS-CoV-2-S_{Δ21} at an MOI of 0.5. Cells and supernatants were harvested at 24 hpi and titrated on MA104 cells (data are pooled from three or more independent experiments; error bars indicate standard deviation of the mean. (D Top: the indicated cells were infected with VSV-SARS-CoV-2-S_{Δ21} at an MOI of 2. Images were acquired 7.5 hpi using a fluorescence microscope and GFP, and transmitted light images were processed and merged using ImageJ (data are representative of two independent experiments). Bottom: Plaque assays were performed on the indicated cell types using VSV-SARS-CoV-2-S_{Δ21}. Images showing GFP expression were acquired 48 hpi using a biomolecular imager (data are representative of at least three independent experiments; standard deviations of the mean are shown). (E) Western blotting was performed on concentrated VSV-SARS-CoV-2-S_{Δ21} and wild-type VSV particles on an 8% non-reducing SDS-PAGE gel. S1 was detected using a cross-reactive anti-SARS-CoV mAb (CR3022) (data are representative of two independent experiments). (F) BSRT7/5 cells were inoculated at an MOI of 10 with VSV-eGFP, G-complemented VSV-SARS-CoV-2-S_{Δ21}, or mock infected (not shown), and were metabolically labeled with [³⁵S] methionine and cysteine for 20 h starting at 5 hpi in the presence of actinomycin D. Viral supernatants were analyzed by SDS-PAGE. A representative phosphor-image is shown from two independent experiments. An asterisk indicates a band that also was detected in the mock lane (not shown). (G) Purified VSV-WT and VSV-SARS-CoV-2-S_{Δ21} particles were subjected to negative stain electron microscopy; scale bars are equivalent to 100 nm. Prefusion structures of each respective glycoprotein are modeled above each EM image (PDB: 512S and 6VSB). See also FIG. 20 and FIG. 21.

FIG. 16. Development of a SARS-CoV-2 Focus-Forming Assay and a VSV-SARS-CoV-2-S_{Δ21} eGFP-Reduction Assay. (A-C) Representative focus forming assay images (A) of viral stocks generated from each producer cell type (top) were developed on the indicated cell substrates (indicated on the left side). Data are representative of two independent experiments. Foci obtained in (A) were counted (B) and the size was determined (C) using an ImmunoSpot plate reader (*p<0.05, **p<0.01, ***p<0.001 by one-way ANOVA with Tukey's multiple comparisons test; error bars indicate standard error of the mean). (D) Representative serial dilution series of VSV-SARS-CoV-2-S_{Δ21} on Vero E6 cells. The total number of infected cells per well was quantified using an automated microscope. Insets of enhanced magnification are shown in red. Data are representative of two independent experiments.

FIG. 17. Neutralization of VSV-SARS-CoV-2-S_{Δ21} and SARS-CoV-2 by Human Monoclonal Antibodies and hACE2 Decoy Receptors. (A and B) Cross-reactive mAbs isolated from a SARS-CoV survivor were tested for neutralizing activity against SARS-CoV-2 (A) or VSV-SARS-CoV-2-S_{Δ21} (B) (n=2 and 3, respectively). (C and D) SARS-CoV-2 RBD-specific antibodies obtained from a phage library were tested for their capacity to neutralize SARS-CoV-2 (C) or VSV-SARS-CoV-2-S_{Δ21} (D) (n=2 and 2, respectively). (E and F) hACE2-Fc or mACE2-Fc were tested for their neutralization activity against SARS-CoV-2 (E) or VSV-SARS-CoV-2-S_{Δ21} (F) (n=2 and 3, respectively). Error bars in (A)-(F) represent the standard error of the mean. See also FIG. 22.

FIG. 18. Human Immune Serum Neutralization of SARS-CoV-2 and VSV-SARS-CoV-2-S_{Δ21}. (A and B) Representative neutralization curves of serum from SARS-CoV-2-infected donors with low, medium, and high inhibitory activity against SARS-CoV-2 (A) or VSV-SARS-CoV-2-S_{Δ21} (B) (n=2 and 2, respectively). Error bars in (A) and (B) represent the standard error of the mean. (C) EC₅₀ values of all human serum tested for neutralization of SARS-CoV-2 and VSV-SARS-CoV-2-S_{Δ21}. Differences in the geometric mean or median titers were 3.0-fold between FRNT and GRNT assays. See also FIG. 23.

FIG. 19. Correlation Analysis of Neutralization of SARS-CoV-2 and VSV-SARS-CoV-2-S_{Δ21}. EC₅₀ values determined in FIG. 17A-FIG. 17D and FIG. 18A-FIG. 18B were used to determine correlation between neutralization assays. Spearman's correlation rand p values are indicated.

FIG. 20. Rescue of a chimeric VSV expressing the SARS-CoV-2 S protein and forward genetic selection of a gain-of-function mutant, Related to FIG. 15. (A) BSRT7/5 cells were infected with vaccinia virus vTF7-3, transfected with plasmids allowing T7-driven expression of VSV N, P, L, and G, and an infectious molecular cDNA of VSV-SARS-CoV-2-SAA to produce replication-competent VSV-SARS-CoV-2-SAA. (B) Alignment of the membrane proximal region, transmembrane domain, and cytoplasmic tail of various recombinants that were generated. Successful rescue and indication of spread are noted. (C) VSV-SARS-CoV-2-SAA was passaged iteratively on Vero CCL81 cells. Several clones were plaque-purified an amplified on Vero CCL81 cells. RNA from infected cells was extracted and deep sequenced to identify mutants.

FIG. 21. VSV-SARS-CoV-2-SΔ21 can infect human lung adenocarcinoma cells, Related to FIG. 15. Calu-3 cells were inoculated with VSV-SARS-CoV-2-S_{Δ21} or VSV-eGFP at an MA104-calculated MOI of 20. At 7 hpi, cells were stained with the nuclear Hoechst 33342 stain (blue) and images in FITC and DAPI fields (overlaid) were taken using an automated microscope. Representative images from 5 independent experiments are shown.

FIG. 22. Inhibition of VSV-SARS-CoV-2-S_{Δ21} but not VSV with hACE2-Fc receptor decoy proteins, Related to FIG. 17. VSV-SARS-CoV-2-SΔ21 and VSV were incubated with the indicated human or murine ACE2-Fc receptor decoy proteins, and virus-antibody mixtures were used to infect Vero E6 cells in a GRNT assay. Error bars represent the standard error of the mean. Data are representative of three independent experiments.

FIG. 23. Human immune serum neutralization of SARS-CoV-2 and VSV-SARS-CoV-2-S_{Δ21}, Related to FIG. 18. As described in FIG. 18, human serum samples from PCR confirmed SARS-CoV-2-infected patients were tested in FRNT (A-G) and GRNT (H-N) assays with SARS-CoV-2 and VSV-SARS-CoV-2-S_{Δ21}.

FIG. 24. Graphical abstract describing the identification and generation of escape mutants, measured the neutralization capability of antibodies on the escape mutants, and testing of antibody cocktails on escape mutants.

FIG. 25. VSV-SARS-CoV-2 escape mutant isolation. (A) Outline of escape mutant selection experiment. 2B04 and a control anti-influenza virus mAb were tested for neutralizing activity against VSV-SARS-CoV-2. The concentration of 2B04 added in the overlay completely inhibited viral infection (middle panel). Data are representative of two independent experiments. Plaque assays were performed to isolate the VSV-SARS-CoV-2 escape mutant on Vero E6 TMPRSS2 cells (red arrow indicated). Plaque assays with 2B04 in the overlay (bottom plaque in the right panel); plaque assays without Ab in the overlay (top plaque in the right panel). Data are representative of three independent experiments. (B) Schematic of S gene, which underwent Sanger sequencing to identify mutations (left panel). For validation, each VSV-SARS-CoV-2 mutant was tested in plaque assays with or without 2B04 in the overlay on Vero cells (right panel). Representative images of two independent experiments are shown. (C) Neutralizing mAbs. ^{a} The order of immunogens used to immunize the mice, as described in STAR methods. ^{b} Neutralization of SARS-CoV-2 by each mAb was assessed by focus-reduction neutralization test. The half-maximal effective concentration (EC₅₀ value) was determined by nonlinear regression. Results are the geometric mean from three to four independent experiments. ^{c} mAb was identified as mouse IgG1 and expressed as human IgG1.

FIG. 26. Mapping of escape mutations. The surface model of RBD (from PDB 6M0J) is depicted, and contact residues of the SARS-CoV-2 RBD-hACE2 interfaces are colored in brown. Amino acids whose substitution confers resistance to each mAb in plaque assays are indicated for 2B04 (green), 2H04 (lemon), 1B07 (blue), SARS2-01 (yellow), SARS2-02 (teal), SARS2-07 (tangerine), SARS2-16 (violet), SARS2-19 (red), SARS2-32 (fuschia), and SARS2-38 (magenta). See FIG. 32 and FIG. 33.

FIG. 27. Map of cross-neutralizing activity of VSV-SARS-CoV-2 mutants and neutralization potency of hACE2 decoy receptors against each VSV-SARS-CoV-2 mutant. (A) Neutralization of VSV-SARS-CoV-2 mutants was evaluated by plaque assays. Degree of resistance was defined as percentage by expressing the number of plaques formed by each mutant in the presence versus absence of antibody and is represented as a heatmap from white (low degree of resistance) to red (high degree of resistance). Representative images of two independent experiments are shown in FIG. 34. (B) Neutralization assay of VSV-SARS-CoV-2 mutants in the presence of hACE2-Fc. Virus was incubated with mACE2 or hACE2 at concentrations ranging from 9 ng/mL to 20 μg/mL for 1 h a 37° C., and cells were scored for infection at 7.5 h post-inoculation by automated microscopy. IC₅₀ values were calculated for each virus-hACE2 combination from three independent experiments (*p<0.05, **p<0.01, ***p<0.001, ****p<0.0001; one-way ANOVA with Dunnett's post-test; error bars indicate SEM). (C) Representative neutralization curves of wild-type and F486S mutant VSV-SARS-CoV-2 with hACE2-Fc and mACE2-Fc. Error bars represent the SEM. Data are representative of three independent experiments. Neutralization curves are provided in FIG. 35.

FIG. 28. Neutralization potency of human serum against each VSV-SARS-CoV-2 mutant. (A) Neutralization potency of four human sera against VSV-SARS-CoV-2 mutants. IC₅₀ values were calculated from three independent experiments. Neutralization potency is represented as a rainbow color map from red (most potent with low IC₅₀) to violet (less potent with high IC₅₀). LOD indicates limit of detection (1:80). (B) Representative neutralization curves of wild-type, S477N, and E484A mutant with four different human sera. Error bars represent SEM. Data are representative of three independent experiments. (C) Neutralization potency of additional 16 human sera against VSV-SARS-CoV-2 mutants. IC₅₀ values were calculated from one independent experiment each. Neutralization potency is represented as a rainbow color map from red (most potent with low IC₅₀) to violet (less potent with high IC₅₀). Neutralization curves are provided in FIG. 36. (D) Serum samples from 18 individuals were collected at different time points post-onset of COVID-19 symptoms and screened using two ELISA assays (Euroimmun or Epitope). The serum identifier numbers in the first column correspond to those of FIG. 28 and FIG. 36. IgG index values were calculated by dividing the optical density (O.D.) of the serum sample by a reference O.D. control, and ratios were interpreted using the following criteria as recommended by the manufacturer: negative (−)<0.8, indeterminate (+/−) 0.8-1.1, and positive (+)≥1.1.

FIG. 29. Mapping of additional VSV-SARS-CoV-2 escape mutants. The surface model of RBD (from PDB 6M0J) is depicted, and contact residues of the SARS-CoV-2 RBD-hACE2 interfaces are colored in brown. Amino acids whose substitution confers resistance to each mAb in the plaque assays are indicated for SARS2-21 (lime), SARS2-22 (green), SARS2-23 (blue), SARS2-31 (yellow), SARS2-34 (cyan), SARS2-55 (orange), SARS2-58 (magenta), SARS2-66 (red), and SARS2-71 (pink). See FIG. 37 and FIG. 38.

FIG. 30. Position and frequency of RBD amino acid substitutions in SARS-CoV-2. (A) RBD amino acid substitutions in currently circulating SARS-CoV-2 viruses isolated from humans. For each site of escape, we counted the sequences in GISAID with an amino acid change (323,183 total sequences at the time of the analysis). Variant circulating frequency is represented as a rainbow color map from red (less circulating with low frequency) to violet (most circulating with high frequency). A black cell indicates that the variant has not yet been isolated from a patient. A rainbow cell with cross indicates that the variant has been isolated from a patient but does not appear in those 50 escape mutants. (B) Location of natural sequence variation within the RBD. The RBD is modeled as a surface representation. Variant frequency is rainbow colored, as in (A). Black coloration indicates that variation at that residue has not yet been isolated.

FIG. 31. Sequential selection of 2B04 and 2H04 escape mutants. (A) Plaque assays were performed to isolate the VSV-SARS-CoV-2-S wild-type, E484A, E484K, and F486S escape mutant on Vero E6 TMPRSS2 cells in the present of the indicated mAb in the overlay. Representative images of three independent experiments are shown. (B) The surface model of RBD (from PDB 6M0J) is depicted, and contact residues of the SARS-CoV-2 RBD-hACE2 interfaces are colored in brown. 2B04 escape mutants including E484A, E484K, and F486S are indicated in green. Amino acids whose substitution confers resistance to 2H04 in the plaque assays are indicated in lemon. (C) Wild-type and sequentially identified double mutants were tested for neutralizing activity using a plaque assay with the indicated mAb in the overlay. mAb concentrations added were the same as those used to select the escape mutants. Representative images of two independent experiments are shown.

FIG. 32. Isolation of VSV-SARS-CoV-2 escape mutants by plaque assay. Related to FIG. 26. (A) RBD-specific antibodies were tested for neutralizing activity against VSV-SARS-CoV-2. MAbs in the left panel were purified from Expi293F cells transfected with antibody expression vector (pABVec6W) expressing heavy chain V-D-J and light Chain V-J cloned from single B cells. MAbs in the right panel were from hybridomas that bound to SARS-CoV-2-infected Vero CCL81 cells by flow cytometry. Data are representative of two independent experiments. (B) Plaque assays were performed to isolate the VSV-SARS-CoV-2-S escape mutant on Vero E6 TMPRSS2 cells in the present of the indicated mAb in the overlay. Representative images of two independent experiments are shown.

FIG. 33. Validation of selected VSV-SARS-CoV-2 mutants. Related to FIG. 26. Plaque assays were performed to validate the VSV-SARS-CoV-2 mutant on Vero cells in the presence and absence of the mAb in the overlay. MAb concentrations added in the overlay were the same as those used to select the escape mutants. Representative images of two independent experiments are shown.

FIG. 34. Plaque assay validation of cross-neutralization of VSV-SARS-CoV-2 mutants. Related to FIG. 27A. Wild-type and identified VSV-SARS-CoV-2 mutants were tested for neutralizing activity using a plaque assay with the indicated mAb in the overlay. MAb concentrations added were the same as those used to select the escape mutants. Representative images of two independent experiments are shown.

FIG. 35. Neutralization of VSV-SARS-CoV-2 mutants by hACE2 decoy receptors. Related to FIG. 27B and FIG. 27C. hACE2-Fc or mACE2-Fc were tested for neutralizing activity against wild-type and mutant VSV-SARS-CoV-2 (n=3). Error bars represent the SEM. Data are representative of three independent experiments.

FIG. 36. Neutralization of VSV-SARS-CoV-2 mutants by human sera. Related to FIG. 28. (A) Four human sera were tested for neutralization of wild-type and mutant VSV-SARS-CoV-2 (n=3). Error bars represent the SEM. Data are representative of three independent experiments. Related to FIG. 28A and FIG. 28B. (B) Sixteen human sera were tested for neutralization of wild-type and 5 mutant VSV-SARS-CoV-2 (n=1). Related to FIG. 28C.

FIG. 37. A second neutralization escape selection campaign with nine additional mAbs. Related to FIG. 29. (A) Nine additional RBD-specific antibodies were tested for neutralization activity against VSV-SARS-CoV-2. Data are representative of two independent experiments. (B) Plaque assays were performed to isolate the VSV-SARS-CoV-2 escape mutant on Vero E6 TMPRSS2 cells in the presence of the indicated mAb in the overlay. Representative images of six independent experiments are shown.

FIG. 38. Validation of selected VSV-SARS-CoV-2 mutants. Related to FIG. 29. Plaque assays were performed to validate the VSV-SARS-CoV-2 mutant on Vero cells in the presence and absence of mAb in the overlay. MAb concentration added in the overlay were the same as those used to select the escape mutants. Representative images of two independent experiments are shown.

### DETAILED DESCRIPTION

The present disclosure is based, at least in part, on the design and use of a VSV recombinant in which the native glycoprotein (G) has been replaced by the spike gene of SARS-CoV-2 the causal agent of COVID-19. Described herein, is a vaccine candidate and a high throughput screening tool for use under biosafety level 2 conditions to identify inhibitors that work by blocking SARS-CoV-2 spike protein mediated steps in infection.

In one variation, we modified the viral genome to express a marker gene (eGFP) as a reporter of infection. That virus allows for high throughput screening to hunt for inhibitors of SARS-CoV-2 entry, including but not limited to proteins, such as antibodies and soluble receptors, co-receptors, peptides including those that block the refolding steps of the spike that are necessary for fusion, and small molecules that block spike mediated infection.

The reporter gene can be any reporter gene known in the art.

The VSV recombinants generated and disclosed herein can be used as vaccines.

Described herein is the design and production of SARS-COV2 vaccines using VSV vectored spike proteins and mutants that can make replication-competent VSV viruses expressing the SARS-CoV-2 spike. The same viral vectors with eGFP can be used in neutralization assays to screen for potential therapeutics.

Growth properties (for generating enough for immunization) are more desirable for the newly generated vaccines and grow 2-logs higher than previous versions. It was shown herein that rVSV-SΔ21 grows well, is neutralized by soluble ACE2, and it was shown herein that it can be neutralized by antibodies.

The VSV constructs can comprise components as described in U.S. application Ser. Nos. 12/089,353; 13/979,179; 16/343,113; PCT/US2006/045104; PCT/US2015/065388; and PCT/US2017/057361, which are incorporated herein by reference.

Sequence Listings

Provided herein are recombinant vesicular stomatis viruses (rVSV). In some embodiments, the rVSV can comprise in its genome a nucleic acid sequence for encoding an enhanced Green Fluorescent Protein (eGFP). In some embodiments, the recombinant viruses comprise in their genome a nucleic acid sequence encoding a SARS-CoV-2 spike protein or fragment thereof. In some embodiments, the rVSV can comprise variants with about 80% identity to the spike. Exemplary nucleic acid sequence for an rVSV can be a rVSV of SEQ ID NO: 2 and SEQ ID NO: 3. The rVSV S gene is at least about 80% identical to the S gene of SARS-CoV-2 isolate Wuhan-Hu-1 (GenBank MN908947.3) with a cytoplasmic tail truncation that facilitates its incorporation into VSV. This could be 21 residue truncation but many others work. Since 21 residues is a very small portion of S the sequence, the S portion of the rVSV will always easily be at least 80% identical to the sequence provided above.

Amino Acid spike reference sequence for the S gene of SARS-COV-2
isolate Wuhan-Hu-1 (GenBank MN908947.3) (SEQ ID NO: 1):

| | |
|---|---|
| MFVFLVLLPL VSSQCVNLTT RTQLPPAYTN SFTRGVYYPD KVFRSSVLHS TQDLFLPFFS | 60 |
| NVTWFHAIHV SGTNGTKRFD NPVLPFNDGV YFASTEKSNI IRGWIFGTTL DSKTQSLLIV | 120 |
| NNATNVVIKV CEFQFCNDPF LGVYYHKNNK SWMESEFRVY SSANNCTFEY VSQPFLMDLE | 180 |
| GKQGNFKNLR EFVFKNIDGY FKIYSKHTPI NLVRDLPQGF SALEPLVDLP IGINITRFQT | 240 |
| LLALHRSYLT PGDSSSGWTA GAAAYYVGYL QPRTFLLKYN ENGTITDAVD CALDPLSETK | 300 |
| CTLKSFTVEK GIYQTSNFRV QPTESIVRFP NITNLCPFGE VFNATRFASV YAWNRKRISN | 360 |
| CVADYSVLYN SASFSTFKCY GVSPTKLNDL CFTNVYADSF VIRGDEVRQI APGQTGKIAD | 420 |
| YNYKLPDDFT GCVIAWNSNN LDSKVGGNYN YLYRLFRKSN LKPFERDIST EIYQAGSTPC | 480 |
| NGVEGFNCYF PLQSYGFQPT NGVGYQPYRV VVLSFELLHA PATVCGPKKS TNLVKNKCVN | 540 |
| FNFNGLTGTG VLTESNKKFL PFQQFGRDIA DTTDAVRDPQ TLEILDITPC SFGGVSVITP | 600 |
| GTNTSNQVAV LYQDVNCTEV PVAIHADQLT PTWRVYSTGS NVFQTRAGCL IGAEHVNNSY | 660 |
| ECDIPIGAGI CASYQTQTNS PRRARSVASQ SIIAYTMSLG AENSVAYSNN SIAIPTNFTI | 720 |
| SVTTEILPVS MTKTSVDCTM YICGDSTECS NLLLQYGSFC TQLNRALTGI AVEQDKNTQE | 780 |
| VFAQVKQIYK TPPIKDFGGF NFSQILPDPS KPSKRSFIED LLFNKVTLAD AGFIKQYGDC | 840 |
| LGDIAARDLI CAQKFNGLTV LPPLLTDEMI AQYTSALLAG TITSGWTFGA GAALQIPFAM | 900 |
| QMAYRFNGIG VTQNVLYENQ KLIANQFNSA IGKIQDSLSS TASALGKLQD VVNQNAQALN | 960 |
| TLVKQLSSNF GAISSVLNDI LSRLDKVEAE VQIDRLITGR LQSLQTYVTQ QLIRAAEIRA | 1020 |
| SANLAATKMS ECVLGQSKRV DFCGKGYHLM SFPQSAPHGV VFLHVTYVPA QEKNFTTAPA | 1080 |
| ICHDGKAHFP REGVFVSNGT HWFVTQRNFY EPQIITTDNT FVSGNCDVVI GIVNNTVYDP | 1140 |
| LQPELDSFKE ELDKYFKNHT SPDVDLGDIS GINASVVNIQ KEIDRLNEVA KNLNESLIDL | 1200 |
| QELGKYEQYI KWPWYIWLGF IAGLIAIVMV TIMLCCMTSC CSCLKGCCSC GSCQKFDEDD | 1260 |
| SEPVLLGVKL RYT | 1273 |

As an example, the recombinant virus can have a spike protein in place of the VSV G but can also include the S sequence in addition to VSV G. Also disclosed herein are variants that were generated. The variants all have at least 80% identity in the Spike. Spike variants can include amino acid substitutions, as disclosed herein (see e.g., TABLE 2 in Example 5), such as “wild type” “D614G variant background” or “complex variants—e.g. UK, SA, Brazil (E484K)”. As an example, an N501Y spike mutation is found in South Africa (501Y.V2) (e.g., K417N, E484K and N501Y) and the U.K. (B.1.1.7) variant.

Nucleic Acid Sequence of pVSV(1+)-eGFP-SARS-COV-2-S_{AA} plasmid
(SEQ ID NO: 2):
ACGAAGACAAACAAACCATTATTATCATTAAAAGGCTCAGGAGAAACTTTAACAGTAAT
CAGAATTCTCGAGATGGTGAGCAAGGGCGAGGAGCTGTTCACCGGGGTGGTGCCCATCC
TGGTCGAGCTGGACGGCGACGTAAACGGCCACAAGTTCAGCGTGTCCGGCGAGGGCGAG
GGCGATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGTGAA
GTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATCGA
GGACGGCAGCGTGCAGCTCGCCGACCACTACCAGCAGAACACCCCCATCGGCGACGGCC
CCGTGCTGCTGCCCGACAACCACTACCTGAGCACCCAGTCCGCCCTGAGCAAAGACCCC
AACGAGAAGCGCGATCACATGGTCCTGCTGGAGTTCGTGACCGCCGCCGGGATCACTCT
CGGCATGGACGAGCTGTACAAGTGATGGCCATATGAAAAAAACTAACAGTAATCAAAAT
GTCTGTTACAGTCAAGAGAATCATTGACAACACAGTCATAGTTCCAAAACTTCCTGCAA
ATGAGGATCCAGTGGAATACCCGGCAGATTACTTCAGAAAATCAAAGGAGATTCCTCTT
TACATCAATACTACAAAAAGTTTGTCAGATCTAAGAGGATATGTCTACCAAGGCCTCAA
ATCCGGAAATGTATCAATCATACATGTCAACAGCTACTTGTATGGAGCATTAAAGGACA
TCCGGGGTAAGTTGGATAAAGATTGGTCAAGTTTCGGAATAAACATCGGGAAAGCAGGG
GATACAATCGGAATATTTGACCTTGTATCCTTGAAAGCCCTGGACGGCGTACTTCCAGA
TGGAGTATCGGATGCTTCCAGAACCAGCGCAGATGACAAATGGTTGCCTTTGTATCTAC
TTGGCTTATACAGAGTGGGCAGAACACAAATGCCTGAATACAGAAAAAAGCTCATGGAT
GGGCTGACAAATCAATGCAAAATGATCAATGAACAGTTTGAACCTCTTGTGCCAGAAGG
TCGTGACATTTTTGATGTGTGGGGAAATGACAGTAATTACACAAAAATTGTCGCTGCAG
TGGACATGTTCTTCCACATGTTCAAAAAACATGAATGTGCCTCGTTCAGATACGGAACT
ATTGTTTCCAGATTCAAAGATTGTGCTGCATTGGCAACATTTGGACACCTCTGCAAAAT
AACCGGAATGTCTACAGAAGATGTAACGACCTGGATCTTGAACCGAGAAGTTGCAGATG
AAATGGTCCAAATGATGCTTCCAGGCCAAGAAATTGACAAGGCCGATTCATACATGCCT
TATTTGATCGACTTTGGATTGTCTTCTAAGTCTCCATATTCTTCCGTCAAAAACCCTGC
CTTCCACTTCTGGGGGCAATTGACAGCTCTTCTGCTCAGATCCACCAGAGCAAGGAATG
CCCGACAGCCTGATGACATTGAGTATACATCTCTTACTACAGCAGGTTTGTTGTACGCT
TATGCAGTAGGATCCTCTGCCGACTTGGCACAACAGTTTTGTGTTGGAGATAACAAATA
CACTCCAGATGATAGTACCGGAGGATTGACGACTAATGCACCGCCACAAGGCAGAGATG
TGGTCGAATGGCTCGGATGGTTTGAAGATCAAAACAGAAAACCGACTCCTGATATGATG
CAGTATGCGAAAAGAGCAGTCATGTCACTGCAAGGCCTAAGAGAGAAGACAATTGGCAA
GTATGCTAAGTCAGAATTTGACAAATGACCCTATAATTCTCAGATCACCTATTATATAT
TATGCTACATATGAAAAAAACTAACAGATATCATGGATAATCTCACAAAAGTTCGTGAG
TATCTCAAGTCCTACTCTCGTCTAGATCAGGCGGTAGGAGAGATAGATGAGATCGAAGC
ACAACGAGCTGAAAAGTCCAATTATGAGTTGTTCCAAGAGGACGGAGTGGAAGAGCATA
CTAGGCCCTCTTATTTTCAGGCAGCAGATGATTCTGACACAGAATCTGAACCAGAAATT
GAAGACAATCAAGGCTTGTATGTACCAGATCCGGAAGCTGAGCAAGTTGAAGGCTTTAT
ACAGGGGCCTTTAGATGACTATGCAGATGAGGACGTGGATGTTGTATTCACTTCGGACT
GGAAACAGCCTGAGCTTGAATCCGACGAGCATGGAAAGACCTTACGGTTGACATTGCCA
GAGGGTTTAAGTGGAGAGCAGAAATCCCAGTGGCTTTTGACGATTAAAGCAGTCGTTCA
AAGTGCCAAACACTGGAATCTGGCAGAGTGCACATTTGAAGCATCGGGAGAAGGGGTCA
TCATAAAAAAGCGCCAGATAACTCCGGATGTATATAAGGTCACTCCAGTGATGAACACA
CATCCGTACCAATCAGAAGCCGTATCAGATGTTTGGTCTCTCTCAAAGACATCCATGAC
TTTCCAACCCAAGAAAGCAAGTCTTCAGCCTCTCACCATATCCTTGGATGAATTGTTCT
CATCTAGAGGAGAATTCATCTCTGTCGGAGGTAACGGACGAATGTCTCATAAAGAGGCC
ATCCTGCTCGGTCTGAGGTACAAAAAGTTGTACAATCAGGCGAGAGTCAAATATTCTCT
GTAGACTATGAAAAAAAGTAACAGATATCACAATCTAAGTGTTATCCCAATCCATTCAT
CATGAGTTCCTTAAAGAAGATTCTCGGTCTGAAGGGGAAAGGTAAGAAATCTAAGAAAT
TAGGGATCGCACCACCCCCTTATGAAGAGGACACTAGCATGGAGTATGCTCCGAGCGCT
CCAATTGACAAATCCTATTTTGGAGTTGACGAGATGGACACCTATGATCCGAATCAATT
AAGATATGAGAAATTCTTCTTTACAGTGAAAATGACGGTTAGATCTAATCGTCCGTTCA
GAACATACTCAGATGTGGCAGCCGCTGTATCCCATTGGGATCACATGTACATCGGAATG
GCAGGGAAACGTCCCTTCTACAAAATCTTGGCTTTTTTGGGTTCTTCTAATCTAAAGGC
CACTCCAGCGGTATTGGCAGATCAAGGTCAACCAGAGTATCACGCTCACTGCGAAGGCA
GGGCTTATTTGCCACATAGGATGGGGAAGACCCCTCCCATGCTCAATGTACCAGAGCAC
TTCAGAAGACCATTCAATATAGGTCTTTACAAGGGAACGATTGAGCTCACAATGACCAT
CTACGATGATGAGTCACTGGAAGCAGCTCCTATGATCTGGGATCATTTCAATTCTTCCA
AATTTTCTGATTTCAGAGAGAAGGCCTTAATGTTTGGCCTGATTGTCGAGAAAAAGGCA
TCTGGAGCGTGGGTCCTGGATTCTATCAGCCACTTCAAATGAGCTAGTCTAGCTTCCAG
CTTCTGAACAATCCCCGGTTTACTCAGTCTCTCCTAATTCCAGCCTTTCGAACAACTAA
TATCCTGTCTTTTCTATCCCTATGAAAAAAACTAACAGAGATCGATCTGTTTCCTTGAC
ACGCGTACCATGTTTGTTTTTCTTGTTTTATTGCCACTAGTCTCTAGTCAGTGTGTTAA
TCTTACAACCAGAACTCAATTACCCCCTGCATACACTAATTCTTTCACACGTGGTGTTT
ATTACCCTGACAAAGTTTTCAGATCCTCAGTTTTACATTCAACTCAGGACTTGTTCTTA
CCTTTCTTTTCCAATGTTACTTGGTTCCATGCTATACATGTCTCTGGGACCAATGGTAC
TAAGAGGTTTGATAACCCTGTCCTACCATTTAATGATGGTGTTTATTTTGCTTCCACTG
AGAAGTCTAACATAATAAGAGGCTGGATTTTTGGTACTACTTTAGATTCGAAGACCCAG
TCCCTACTTATTGTTAATAACGCTACTAATGTTGTTATTAAAGTCTGTGAATTTCAATT
TTGTAATGATCCATTTTTGGGTGTTTATTACCACAAAAACAACAAAAGTTGGATGGAAA
GTGAGTTCAGAGTTTATTCTAGTGCGAATAATTGCACTTTTGAATATGTCTCTCAGCCT
TTTCTTATGGACCTTGAAGGAAAACAGGGTAATTTCAAAAATCTTAGGGAATTTGTGTT
TAAGAATATTGATGGTTATTTTAAAATATATTCTAAGCACACGCCTATTAATTTAGTGC
GTGATCTCCCTCAGGGTTTTTCGGCTTTAGAACCATTGGTAGATTTGCCAATAGGTATT
AACATCACTAGGTTTCAAACTTTACTTGCTTTACATAGAAGTTATTTGACTCCTGGTGA
TTCTTCTTCAGGTTGGACAGCTGGTGCTGCAGCTTATTATGTGGGTTATCTTCAACCTA
GGACTTTTCTATTAAAATATAATGAAAATGGAACCATTACAGATGCTGTAGACTGTGCA
CTTGACCCTCTCTCAGAAACAAAGTGTACGTTGAAATCCTTCACTGTAGAAAAAGGAAT
CTATCAAACTTCTAACTTTAGAGTCCAACCAACAGAATCTATTGTTAGATTTCCTAATA
TTACAAACTTGTGCCCTTTTGGTGAAGTTTTTAACGCCACCAGATTTGCATCTGTTTAT
GCTTGGAACAGGAAGAGAATCAGCAACTGTGTTGCTGATTATTCTGTCCTATATAATTC
CGCATCATTTTCCACTTTTAAGTGTTATGGAGTGTCTCCTACTAAATTAAATGATCTCT
GCTTTACTAATGTCTATGCAGATTCATTTGTAATTAGAGGTGATGAAGTCAGACAAATC
GCTCCAGGGCAAACTGGAAAGATTGCTGATTATAATTATAAATTACCAGATGATTTTAC
AGGCTGCGTTATAGCTTGGAATTCTAACAATCTTGATTCTAAGGTTGGTGGTAATTATA
ATTACCTGTATAGATTGTTTAGGAAGTCTAATCTCAAACCTTTTGAGAGAGATATTTCA
ACTGAAATCTATCAGGCCGGTAGCACACCTTGTAATGGTGTTGAAGGTTTTAATTGTTA
CTTTCCTTTACAATCATATGGTTTCCAACCCACTAATGGTGTTGGTTACCAACCATACA
GAGTAGTAGTACTTTCTTTTGAACTTCTACATGCACCAGCAACTGTTTGTGGACCTAAA
AAGTCTACTAATTTGGTTAAAAACAAATGTGTCAATTTCAACTTCAATGGTTTAACAGG
CACAGGTGTTCTTACTGAGTCTAACAAAAAGTTTCTGCCTTTCCAACAATTTGGCAGAG
ACATTGCTGACACTACTGATGCTGTCCGTGATCCACAGACACTTGAGATTCTTGACATT
ACACCATGTTCTTTTGGTGGTGTCAGTGTTATAACACCAGGAACAAATACTTCTAACCA
GGTTGCTGTTCTTTATCAGGATGTTAACTGCACAGAAGTCCCTGTTGCTATTCATGCAG
ATCAACTTACTCCTACTTGGCGTGTTTATTCTACAGGTTCTAATGTTTTTCAAACACGT
GCAGGCTGTTTAATAGGGGCTGAACATGTCAACAACTCATATGAGTGTGACATACCCAT
TGGTGCAGGTATATGCGCTAGTTATCAGACTCAGACTAATTCTCCTCGGCGGGCACGTA
GTGTAGCTAGTCAATCCATCATTGCCTACACTATGTCACTTGGTGCAGAAAATTCAGTT
GCTTACTCTAATAACTCTATTGCCATACCCACAAATTTTACTATTAGTGTTACCACAGA
AATTCTACCAGTGTCTATGACCAAGACATCAGTAGATTGTACAATGTACATTTGTGGTG
ATTCAACTGAATGCAGCAATCTTTTGTTGCAATATGGCAGTTTTTGTACACAATTAAAC
CGTGCTTTAACTGGAATAGCTGTTGAACAAGACAAAAACACCCAAGAAGTTTTTGCACA
AGTCAAACAAATTTACAAAACACCACCAATTAAAGATTTTGGTGGTTTTAATTTTTCAC
AAATATTACCAGATCCATCAAAACCAAGCAAGAGGTCATTTATTGAAGATCTACTTTTC
AACAAAGTGACACTTGCAGATGCTGGCTTCATCAAACAATATGGTGATTGCCTTGGTGA
TATTGCTGCTAGAGACCTCATTTGTGCACAAAAGTTTAACGGCCTTACTGTTTTGCCAC
CTTTGCTCACAGATGAAATGATTGCTCAATACACTTCTGCACTGTTAGCGGGTACAATC
ACTTCTGGTTGGACCTTTGGTGCAGGTGCTGCATTACAAATACCATTTGCTATGCAAAT
GGCTTATAGGTTTAATGGTATTGGAGTTACACAGAATGTTCTCTATGAGAACCAAAAAT
TGATTGCCAACCAATTTAATAGTGCTATTGGCAAAATTCAAGACTCACTTTCTTCCACA
GCAAGTGCACTTGGAAAACTTCAAGATGTGGTCAACCAAAATGCACAAGCTTTAAACAC
GCTTGTTAAACAACTTAGCTCCAATTTTGGTGCAATTTCAAGTGTTTTAAATGATATCC
TTTCACGTCTTGACAAAGTTGAGGCTGAAGTGCAAATTGATAGGTTGATCACAGGCAGA
CTTCAAAGTTTGCAGACATATGTGACTCAACAATTAATTAGAGCTGCAGAAATCAGAGC
TTCTGCTAATCTTGCTGCTACTAAAATGTCAGAGTGTGTACTTGGACAATCAAAAAGAG
TTGATTTTTGTGGAAAGGGCTATCATCTTATGTCCTTCCCTCAGTCAGCACCTCATGGT
GTAGTCTTCTTGCATGTGACTTATGTCCCTGCACAAGAAAAGAACTTCACAACTGCTCC
TGCCATTTGTCATGATGGAAAAGCACACTTTCCTCGTGAAGGTGTCTTTGTTTCAAATG
GCACACACTGGTTTGTAACACAAAGGAATTTTTATGAACCACAAATCATTACTACAGAC
AACACATTTGTGTCTGGTAACTGTGATGTTGTAATAGGAATTGTCAACAACACAGTTTA
TGATCCTTTGCAACCTGAATTAGACTCATTCAAGGAGGAGTTAGATAAATATTTTAAGA
ATCATACATCACCAGATGTTGATTTAGGTGACATCTCTGGCATTAATGCTTCAGTTGTA
AACATTCAAAAAGAAATTGACCGCCTCAATGAGGTTGCCAAGAATTTAAATGAATCTCT
CATCGATCTCCAAGAACTTGGAAAGTATGAGCAGTATATAAAATGGCCATGGTACATTT
GGCTAGGTTTTATAGCTGGCTTGATTGCCATAGTAATGGTGACAATTATGCTTTGCTGT
ATGACCAGTTGCTGTAGTTGTCTCAAGGGCTGTTGTTCTTGTGGATCCTGCTGCAAATT
TGATGAAGACGACTCTGAGCCAGTGCTCAAAGGAGTCGCATTAGCTTACACATAAGCGG
CCGCCCTGCACACAGATTCTTCATGTTTGAACCAAATCAACTTGTGATATCATGCTCAA
AGAGGCCTTAATTATATTTTAATTTTTAATTTTTATGAAAAAAACTAACAGCAATCATG
GAAGTCCACGATTTTGAGACCGACGAGTTCAATGATTTCAATGAAGATGACTATGCCAC
AAGAGAATTCCTGAATCCCGATGAGCGCATGACGTACTTGAATCATGCTGATTACAATT
TGAATTCTCCTCTAATTAGTGATGATATTGACAATTTGATCAGGAAATTCAATTCTCTT
CCGATTCCCTCGATGTGGGATAGTAAGAACTGGGATGGAGTTCTTGAGATGTTAACATC
ATGTCAAGCCAATCCCATCTCAACATCTCAGATGCATAAATGGATGGGAAGTTGGTTAA
TGTCTGATAATCATGATGCCAGTCAAGGGTATAGTTTTTTACATGAAGTGGACAAAGAG
GCAGAAATAACATTTGACGTGGTGGAGACCTTCATCCGCGGCTGGGGCAACAAACCAAT
TGAATACATCAAAAAGGAAAGATGGACTGACTCATTCAAAATTCTCGCTTATTTGTGTC
AAAAGTTTTTGGACTTACACAAGTTGACATTAATCTTAAATGCTGTCTCTGAGGTGGAA
TTGCTCAACTTGGCGAGGACTTTCAAAGGCAAAGTCAGAAGAAGTTCTCATGGAACGAA
CATATGCAGGATTAGGGTTCCCAGCTTGGGTCCTACTTTTATTTCAGAAGGATGGGCTT
ACTTCAAGAAACTTGATATTCTAATGGACCGAAACTTTCTGTTAATGGTCAAAGATGTG
ATTATAGGGAGGATGCAAACGGTGCTATCCATGGTATGTAGAATAGACAACCTGTTCTC
AGAGCAAGACATCTTCTCCCTTCTAAATATCTACAGAATTGGAGATAAAATTGTGGAGA
GGCAGGGAAATTTTTCTTATGACTTGATTAAAATGGTGGAACCGATATGCAACTTGAAG
CTGATGAAATTAGCAAGAGAATCAAGGCCTTTAGTCCCACAATTCCCTCATTTTGAAAA
TCATATCAAGACTTCTGTTGATGAAGGGGCAAAAATTGACCGAGGTATAAGATTCCTCC
ATGATCAGATAATGAGTGTGAAAACAGTGGATCTCACACTGGTGATTTATGGATCGTTC
AGACATTGGGGTCATCCTTTTATAGATTATTACACTGGACTAGAAAAATTACATTCCCA
AGTAACCATGAAGAAAGATATTGATGTGTCATATGCAAAAGCACTTGCAAGTGATTTAG
CTCGGATTGTTCTATTTCAACAGTTCAATGATCATAAAAAGTGGTTCGTGAATGGAGAC
TTGCTCCCTCATGATCATCCCTTTAAAAGTCATGTTAAAGAAAATACATGGCCCACAGC
TGCTCAAGTTCAAGATTTTGGAGATAAATGGCATGAACTTCCGCTGATTAAATGTTTTG
AAATACCCGACTTACTAGACCCATCGATAATATACTCTGACAAAAGTCATTCAATGAAT
AGGTCAGAGGTGTTGAAACATGTCCGAATGAATCCGAACACTCCTATCCCTAGTAAAAA
GGTGTTGCAGACTATGTTGGACACAAAGGCTACCAATTGGAAAGAATTTCTTAAAGAGA
TTGATGAGAAGGGCTTAGATGATGATGATCTAATTATTGGTCTTAAAGGAAAGGAGAGG
GAACTGAAGTTGGCAGGTAGATTTTTCTCCCTAATGTCTTGGAAATTGCGAGAATACTT
TGTAATTACCGAATATTTGATAAAGACTCATTTCGTCCCTATGTTTAAAGGCCTGACAA
TGGCGGACGATCTAACTGCAGTCATTAAAAAGATGTTAGATTCCTCATCCGGCCAAGGA
TTGAAGTCATATGAGGCAATTTGCATAGCCAATCACATTGATTACGAAAAATGGAATAA
CCACCAAAGGAAGTTATCAAACGGCCCAGTGTTCCGAGTTATGGGCCAGTTCTTAGGTT
ATCCATCCTTAATCGAGAGAACTCATGAATTTTTTGAGAAAAGTCTTATATACTACAAT
GGAAGACCAGACTTGATGCGTGTTCACAACAACACACTGATCAATTCAACCTCCCAACG
AGTTTGTTGGCAAGGACAAGAGGGTGGACTGGAAGGTCTACGGCAAAAAGGATGGAGTA
TCCTCAATCTACTGGTTATTCAAAGAGAGGCTAAAATCAGAAACACTGCTGTCAAAGTC
TTGGCACAAGGTGATAATCAAGTTATTTGCACACAGTATAAAACGAAGAAATCGAGAAA
CGTTGTAGAATTACAGGGTGCTCTCAATCAAATGGTTTCTAATAATGAGAAAATTATGA
CTGCAATCAAAATAGGGACAGGGAAGTTAGGACTTTTGATAAATGACGATGAGACTATG
CAATCTGCAGATTACTTGAATTATGGAAAAATACCGATTTTCCGTGGAGTGATTAGAGG
GTTAGAGACCAAGAGATGGTCACGAGTGACTTGTGTCACCAATGACCAAATACCCACTT
GTGCTAATATAATGAGCTCAGTTTCCACAAATGCTCTCACCGTAGCTCATTTTGCTGAG
AACCCAATCAATGCCATGATACAGTACAATTATTTTGGGACATTTGCTAGACTCTTGTT
GATGATGCATGATCCTGCTCTTCGTCAATCATTGTATGAAGTTCAAGATAAGATACCGG
GCTTGCACAGTTCTACTTTCAAATACGCCATGTTGTATTTGGACCCTTCCATTGGAGGA
GTGTCGGGCATGTCTTTGTCCAGGTTTTTGATTAGAGCCTTCCCAGATCCCGTAACAGA
AAGTCTCTCATTCTGGAGATTCATCCATGTACATGCTCGAAGTGAGCATCTGAAGGAGA
TGAGTGCAGTATTTGGAAACCCCGAGATAGCCAAGTTTCGAATAACTCACATAGACAAG
CTAGTAGAAGATCCAACCTCTCTGAACATCGCTATGGGAATGAGTCCAGCGAACTTGTT
AAAGACTGAGGTTAAAAAATGCTTAATCGAATCAAGACAAACCATCAGGAACCAGGTGA
TTAAGGATGCAACCATATATTTGTATCATGAAGAGGATCGGCTCAGAAGTTTCTTATGG
TCAATAAATCCTCTGTTCCCTAGATTTTTAAGTGAATTCAAATCAGGCACTTTTTTGGG
AGTCGCAGACGGGCTCATCAGTCTATTTCAAAATTCTCGTACTATTCGGAACTCCTTTA
AGAAAAAGTATCATAGGGAATTGGATGATTTGATTGTGAGGAGTGAGGTATCCTCTTTG
ACACATTTAGGGAAACTTCATTTGAGAAGGGGATCATGTAAAATGTGGACATGTTCAGC
TACTCATGCTGACACATTAAGATACAAATCCTGGGGCCGTACAGTTATTGGGACAACTG
TACCCCATCCATTAGAAATGTTGGGTCCACAACATCGAAAAGAGACTCCTTGTGCACCA
TGTAACACATCAGGGTTCAATTATGTTTCTGTGCATTGTCCAGACGGGATCCATGACGT
CTTTAGTTCACGGGGACCATTGCCTGCTTATCTAGGGTCTAAAACATCTGAATCTACAT
CTATTTTGCAGCCTTGGGAAAGGGAAAGCAAAGTCCCACTGATTAAAAGAGCTACACGT
CTTAGAGATGCTATCTCTTGGTTTGTTGAACCCGACTCTAAACTAGCAATGACTATACT
TTCTAACATCCACTCTTTAACAGGCGAAGAATGGACCAAAAGGCAGCATGGGTTCAAAA
GAACAGGGTCTGCCCTTCATAGGTTTTCGACATCTCGGATGAGCCATGGTGGGTTCGCA
TCTCAGAGCACTGCAGCATTGACCAGGTTGATGGCAACTACAGACACCATGAGGGATCT
GGGAGATCAGAATTTCGACTTTTTATTCCAAGCAACGTTGCTCTATGCTCAAATTACCA
CCACTGTTGCAAGAGACGGATGGATCACCAGTTGTACAGATCATTATCATATTGCCTGT
AAGTCCTGTTTGAGACCCATAGAAGAGATCACCCTGGACTCAAGTATGGACTACACGCC
CCCAGATGTATCCCATGTGCTGAAGACATGGAGGAATGGGGAAGGTTCGTGGGGACAAG
AGATAAAACAGATCTATCCTTTAGAAGGGAATTGGAAGAATTTAGCACCTGCTGAGCAA
TCCTATCAAGTCGGCAGATGTATAGGTTTTCTATATGGAGACTTGGCGTATAGAAAATC
TACTCATGCCGAGGACAGTTCTCTATTTCCTCTATCTATACAAGGTCGTATTAGAGGTC
GAGGTTTCTTAAAAGGGTTGCTAGACGGATTAATGAGAGCAAGTTGCTGCCAAGTAATA
CACCGGAGAAGTCTGGCTCATTTGAAGAGGCCGGCCAACGCAGTGTACGGAGGTTTGAT
TTACTTGATTGATAAATTGAGTGTATCACCTCCATTCCTTTCTCTTACTAGATCAGGAC
CTATTAGAGACGAATTAGAAACGATTCCCCACAAGATCCCAACCTCCTATCCGACAAGC
AACCGTGATATGGGGGTGATTGTCAGAAATTACTTCAAATACCAATGCCGTCTAATTGA
AAAGGGAAAATACAGATCACATTATTCACAATTATGGTTATTCTCAGATGTCTTATCCA
TAGACTTCATTGGACCATTCTCTATTTCCACCACCCTCTTGCAAATCCTATACAAGCCA
TTTTTATCTGGGAAAGATAAGAATGAGTTGAGAGAGCTGGCAAATCTTTCTTCATTGCT
AAGATCAGGAGAGGGGTGGGAAGACATACATGTGAAATTCTTCACCAAGGACATATTAT
TGTGTCCAGAGGAAATCAGACATGCTTGCAAGTTCGGGATTGCTAAGGATAATAATAAA
GACATGAGCTATCCCCCTTGGGGAAGGGAATCCAGAGGGACAATTACAACAATCCCTGT
TTATTATACGACCACCCCTTACCCAAAGATGCTAGAGATGCCTCCAAGAATCCAAAATC
CCCTGCTGTCCGGAATCAGGTTGGGCCAATTACCAACTGGCGCTCATTATAAAATTCGG
AGTATATTACATGGAATGGGAATCCATTACAGGGACTTCTTGAGTTGTGGAGACGGCTC
CGGAGGGATGACTGCTGCATTACTACGAGAAAATGTGCATAGCAGAGGAATATTCAATA
GTCTGTTAGAATTATCAGGGTCAGTCATGCGAGGCGCCTCTCCTGAGCCCCCCAGTGCC
CTAGAAACTTTAGGAGGAGATAAATCGAGATGTGTAAATGGTGAAACATGTTGGGAATA
TCCATCTGACTTATGTGACCCAAGGACTTGGGACTATTTCCTCCGACTCAAAGCAGGCT
TGGGGCTTCAAATTGATTTAATTGTAATGGATATGGAAGTTCGGGATTCTTCTACTAGC
CTGAAAATTGAGACGAATGTTAGAAATTATGTGCACCGGATTTTGGATGAGCAAGGAGT
TTTAATCTACAAGACTTATGGAACATATATTTGTGAGAGCGAAAAGAATGCAGTAACAA
TCCTTGGTCCCATGTTCAAGACGGTCGACTTAGTTCAAACAGAATTTAGTAGTTCTCAA
ACGTCTGAAGTATATATGGTATGTAAAGGTTTGAAGAAATTAATCGATGAACCCAATCC
CGATTGGTCTTCCATCAATGAATCCTGGAAAAACCTGTACGCATTCCAGTCATCAGAAC
AGGAATTTGCCAGAGCAAAGAAGGTTAGTACATACTTTACCTTGACAGGTATTCCCTCC
CAATTCATTCCTGATCCTTTTGTAAACATTGAGACTATGCTACAAATATTCGGAGTACC
CACGGGTGTGTCTCATGCGGCTGCCTTAAAATCATCTGATAGACCTGCAGATTTATTGA
CCATTAGCCTTTTTTATATGGCGATTATATCGTATTATAACATCAATCATATCAGAGTA
GGACCGATACCTCCGAACCCCCCATCAGATGGAATTGCACAAAATGTGGGGATCGCTAT
AACTGGTATAAGCTTTTGGCTGAGTTTGATGGAGAAAGACATTCCACTATATCAACAGT
GTTTGGCAGTTATCCAGCAATCATTTCCGATTAGGTGGGAGGCTATTTCAGTAAAAGGA
GGATACAAGCAGAAGTGGAGTACTAGAGGTGATGGGCTCCCAAAAGATACCCGAATTTC
AGACTCCTTGGCCCCAATCGGGAACTGGATCAGATCTTTGGAATTGGTCCGAAACCAAG
TTCGTCTAAATCCATTCAATAAGATCTTGTTCAATCAGCTATGTCGTACAGTGGATAAT
CATTTGAAGTGGTCAAATTTGCGAAAAAACACAGGAATGATTGAATGGATCAATGGGCG
AATTTCAAAAGAAGACCGGTCTATACTGATGTTGAAGAGTGACCTACATGAGGAAAACT
CTTGGAGAGATTAAAAAATCAGGAGGAGACTCCAAACTTTAAGTATGAAAAAAACTTTG
ATCCTTAAGACCCTCTTGTGGTTTTTATTTTTTTATCTGGTTTTGTGGTCTTCGTGGGT
CGGCATGGCATCTCCACCTCCTCGCGGTCCGACCTGGGCATCCGAAGGAGGACGTCGTC
CACTCGGATGGCTAAGGGAGAGCTCGGATCCGGCTGCTAACAAAGCCCGAAAGGAAGCT
GAGTTGGCTGCTGCCACCGCTGAGCAATAACTAGCATAACCCCTTGGGGCCTCTAAACG
GGTCTTGAGGGGTTTTTTGCTGAAAGGAGGAACTATATCCGGATCGAGATCCTCTAGAG
TCGACCTGCAGGCATGCAAGCTTGTATTCTATAGTGTCACCTAAATCGTATGTGTATGA
TACATAAGGTTATGTATTAATTGTAGCCGCGTTCTAACGACAATATGTACAAGCCTAAT
TGTGTAGCATCTGGCTTACTGAAGCAGACCCTATCATCTCTCTCGTAAACTGCCGTCAG
AGTCGGTTTGGTTGGACGAACCTTCTGAGTTTCTGGTAACGCCGTCCCGCACCCGGAAA
TGGTCAGCGAACCAATCAGCAGGGTCATCGCTAGCCAGATCCTCTACGCCGGACGCATC
GTGGCCGGCATCACCGGCGCCACAGGTGCGGTTGCTGGCGCCTATATCGCCGACATCAC
CGATGGGGAAGATCGGGCTCGCCACTTCGGGCTCATGAGCGCTTGTTTCGGCGTGGGTA
TGGTGGCAGGCCCCGTGGCCGGGGGACTGTTGGGCGCCATCTCCTTGCACCATTCCTTG
CGGCGGCGGTGCTCAACGGCCTCAACCTACTACTGGGCTGCTTCCTAATGCAGGAGTCG
CATAAGGGAGAGCGTCGAATGGTGCACTCTCAGTACAATCTGCTCTGATGCCGCATAGT
TAAGCCAGCCCCGACACCCGCCAACACCCGCTGACGCGCCCTGACGGGCTTGTCTGCTC
CCGGCATCCGCTTACAGACAAGCTGTGACCGTCTCCGGGAGCTGCATGTGTCAGAGGTT
TTCACCGTCATCACCGAAACGCGCGAGACGAAAGGGCCTCGTGATACGCCTATTTTTAT
AGGTTAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCTCAT
GAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCT
CACCCAGAAACGCTGGTGAAAGTAAAAGATGCTGAAGATCAGTTGGGTGCACGAGTGGG
TTACATCGAACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATTATGCA
GTGCTGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCCTTGA
TCGTTGGGAACCGGAGCTGAATGAAGCCATACCAAACGACGAGCGTGACACCACGATGC
CTGTAGCAATGGCAACAACGTTGCGCAAACTATTAACTGGCGAACTACTTACTCTAGCT
TCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTATC
TACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGA
TTGATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTAGA
AAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGGATAACCGTAT
TACCGCCTTTGAGTGAGCTGATACCGCTCGCCGCAGCCGAACGACCGAGCGCAGCGAGT
CAGTGAGCGAGGAAGCGGAAGAGCGCCCAATACGCAAACCGCCTCTCCCCGCGCGTTGG
CCGATTCATTAATGCAGGGGGATCTCGATCCCGCGAAATTAATACGACTCACTATAGG
Nucleic Acid Sequence of rVSV-eGFP-SARS-COV-2-SΔ21 plasmid
(SEQ ID NO: 3):
ACGACAAACAAACCATTATTATCATTAAAAGGCTCAGGAGAAACTTTAACAGTAATCAG
AATTCTCGAGATGGTGAGCAAGGGCGAGGAGCTGTTCACCGGGGTGGTGCCCATCCTGG
TCGAGCTGGACGGCGACGTAAACGGCCACAAGTTCAGCGTGTCCGGCGAGGGCGAGGGC
GATGCCACCTACGGCAAGCTGACCCTGAAGTTCATCTGCACCACCGGCAAGCTGCCCGT
GCCCTGGCCCACCCTCGTGACCACCCTGACCTACGGCGTGCAGTGCTTCAGCCGCTACC
CCGACCACATGAAGCAGCACGACTTCTTCAAGTCCGCCATGCCCGAAGGCTACGTCCAG
GAGCGCACCATCTTCTTCAAGGACGACGGCAACTACAAGACCCGCGCCGAGGTGAAGTT
CGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATG
GCCGACAAGCAGAAGAACGGCATCAAGGTGAACTTCAAGATCCGCCACAACATCGAGGA
CGGCAGCGTGCAGCTCGCCGACCACTACCAGCAGAACACCCCCATCGGCGACGGCCCCG
TGCTGCTGCCCGACAACCACTACCTGAGCACCCAGTCCGCCCTGAGCAAAGACCCCAAC
GAGAAGCGCGATCACATGGTCTTGCTGGAGTTCGTGACCGCCGCCGGGATCACTCTCGG
CATGGACGAGCTGTACAAGTGATGGCCATATGAAAAAAACTAACAGTAATCAAAATGTC
TGTTACAGTCAAGAGAATCATTGACAACACAGTCATAGTTCCAAAACTTCCTGCAAATG
AGGATCCAGTGGAATACCCGGCAGATTACTTCAGAAAATCAAAGGAGATTCCTCTTTAC
ATCAATACTACAAAAAGTTTGTCAGATCTAAGAGGATATGTCTACCAAGGCCTCAAATC
CGGAAATGTATCAATCATACATGTCAACAGCTACTTGTATGGAGCATTAAAGGACATCC
GGGGTAAGTTGGATAAAGATTGGTCAAGTTTCGGAATAAACATCGGGAAAGCAGGGGAT
ACAATCGGAATATTTGACCTTGTATCCTTGAAAGCCCTGGACGGCGTACTTCCAGATGG
AGTATCGGATGCTTCCAGAACCAGCGCAGATGACAAATGGTTGCCTTTGTATCTACTTG
GCTTATACAGAGTGGGCAGAACACAAATGCCTGAATACAGAAAAAAGCTCATGGATGGG
CTGACAAATCAATGCAAAATGATCAATGAACAGTTTGAACCTCTTGTGCCAGAAGGTCG
TGACATTTTTGATGTGTGGGGAAATGACAGTAATTACACAAAAATTGTCGCTGCAGTGG
ACATGTTCTTCCACATGTTCAAAAAACATGAATGTGCCTCGTTCAGATACGGAACTATT
GTTTCCAGATTCAAAGATTGTGCTGCATTGGCAACATTTGGACACCTCTGCAAAATAAC
CGGAATGTCTACAGAAGATGTAACGACCTGGATCTTGAACCGAGAAGTTGCAGATGAAA
TGGTCCAAATGATGCTTCCAGGCCAAGAAATTGACAAGGCCGATTCATACATGCCTTAT
TTGATCGACTTTGGATTGTCTTCTAAGTCTCCATATTCTTCCGTCAAAAACCCTGCCTT
CCACTTCTGGGGGCAATTGACAGCTCTTCTGCTCAGATCCACCAGAGCAAGGAATGCCC
GACAGCCTGATGACATTGAGTATACATCTCTTACTACAGCAGGTTTGTTGTACGCTTAT
GCAGTAGGATCCTCTGCCGACTTGGCACAACAGTTTTGTGTTGGAGATAACAAATACAC
TCCAGATGATAGTACCGGAGGATTGACGACTAATGCACCGCCACAAGGCAGAGATGTGG
TCGAATGGCTCGGATGGTTTGAAGATCAAAACAGAAAACCGACTCCTGATATGATGCAG
TATGCGAAAAGAGCAGTCATGTCACTGCAAGGCCTAAGAGAGAAGACAATTGGCAAGTA
TGCTAAGTCAGAATTTGACAAATGACCCTATAATTCTCAGATCACCTATTATATATTAT
GCTACATATGAAAAAAACTAACAGATATCATGGATAATCTCACAAAAGTTCGTGAGTAT
CTCAAGTCCTACTCTCGTCTAGATCAGGCGGTAGGAGAGATAGATGAGATCGAAGCACA
ACGAGCTGAAAAGTCCAATTATGAGTTGTTCCAAGAGGACGGAGTGGAAGAGCATACTA
GGCCCTCTTATTTTCAGGCAGCAGATGATTCTGACACAGAATCTGAACCAGAAATTGAA
GACAATCAAGGCTTGTATGTACCAGATCCGGAAGCTGAGCAAGTTGAAGGCTTTATACA
GGGGCCTTTAGATGACTATGCAGATGAGGACGTGGATGTTGTATTCACTTCGGACTGGA
AACAGCCTGAGCTTGAATCCGACGAGCATGGAAAGACCTTACGGTTGACATTGCCAGAG
GGTTTAAGTGGAGAGCAGAAATCCCAGTGGCTTTTGACGATTAAAGCAGTCGTTCAAAG
TGCCAAACACTGGAATCTGGCAGAGTGCACATTTGAAGCATCGGGAGAAGGGGTCATCA
TAAAAAAGCGCCAGATAACTCCGGATGTATATAAGGTCACTCCAGTGATGAACACACAT
CCGTACCAATCAGAAGCCGTATCAGATGITTGGTCTCTCTCAAAGACATCCATGACTTT
CCAACCCAAGAAAGCAAGTCTTCAGCCTCTCACCATATCCTTGGATGAATTGTTCTCAT
CTAGAGGAGAATTCATCTCTGTCGGAGGTAACGGACGAATGTCTCATAAAGAGGCCATC
CTGCTCGGTCTGAGGTACAAAAAGTTGTACAATCAGGCGAGAGTCAAATATTCTCTGTA
GACTATGAAAAAAAGTAACAGATATCACAATCTAAGTGTTATCCCAATCCATTCATCAT
GAGTTCCTTAAAGAAGATTCTCGGTCTGAAGGGGAAAGGTAAGAAATCTAAGAAATTAG
GGATCGCACCACCCCCTTATGAAGAGGACACTAGCATGGAGTATGCTCCGAGCGCTCCA
ATTGACAAATCCTATTTTGGAGTTGACGAGATGGACACCTATGATCCGAATCAATTAAG
ATATGAGAAATTCTTCTTTACAGTGAAAATGACGGTTAGATCTAATCGTCCGTTCAGAA
CATACTCAGATGTGGCAGCCGCTGTATCCCATTGGGATCACATGTACATCGGAATGGCA
GGGAAACGTCCCTTCTACAAAATCTTGGCTTTTTTGGGTTCTTCTAATCTAAAGGCCAC
TCCAGCGGTATTGGCAGATCAAGGTCAACCAGAGTATCACGCTCACTGCGAAGGCAGGG
CTTATTTGCCACATAGGATGGGGAAGACCCCTCCCATGCTCAATGTACCAGAGCACTTC
AGAAGACCATTCAATATAGGTCTTTACAAGGGAACGATTGAGCTCACAATGACCATCTA
CGATGATGAGTCACTGGAAGCAGCTCCTATGATCTGGGATCATTTCAATTCTTCCAAAT
TTTCTGATTTCAGAGAGAAGGCCTTAATGTTTGGCCTGATTGTCGAGAAAAAGGCATCT
GGAGCGTGGGTCCTGGATTCTATCAGCCACTTCAAATGAGCTAGTCTAGCTTCCAGCTT
CTGAACAATCCCCGGTTTACTCAGTCTCTCCTAATTCCAGCCTTTCGAACAACTAATAT
CCTGTCTTTTCTATCCCTATGAAAAAAACTAACAGAGATCGATCTGTTTCCTTGACACG
CGTACCATGTTTGTTTTTCTTGTTTTATTGCCACTAGTCTCTAGTCAGTGTGTTAATCT
TACAACCAGAACTCAATTACCCCCTGCATACACTAATTCTTTCACACGTGGTGTTTATT
ACCCTGACAAAGTTTTCAGATCCTCAGTTTTACATTCAACTCAGGACTTGTTCTTACCT
TTCTTTTCCAATGTTACTTGGTTCCATGCTATACATGTCTCTGGGACCAATGGTACTAA
GAGGTTTGATAACCCTGTCCTACCATTTAATGATGGTGTTTATTTTGCTTCCACTGAGA
AGTCTAACATAATAAGAGGCTGGATTTTTGGTACTACTTTAGATTCGAAGACCCAGTCC
CTACTTATTGTTAATAACGCTACTAATGTTGTTATTAAAGTCTGTGAATTTCAATTTTG
TAATGATCCATTTTTGGGTGTTTATTACCACAAAAACAACAAAAGTTGGATGGAAAGTG
AGTTCAGAGTTTATTCTAGTGCGAATAATTGCACTTTTGAATATGTCTCTCAGCCTTTT
CTTATGGACCTTGAAGGAAAACAGGGTAATTTCAAAAATCTTAGGGAATTTGTGTTTAA
GAATATTGATGGTTATTTTAAAATATATTCTAAGCACACGCCTATTAATTTAGTGCGTG
ATCTCCCTCAGGGTTTTTCGGCTTTAGAACCATTGGTAGATTTGCCAATAGGTATTAAC
ATCACTAGGTTTCAAACTTTACTTGCTTTACATAGAAGTTATTTGACTCCTGGTGATTC
TTCTTCAGGTTGGACAGCTGGTGCTGCAGCTTATTATGTGGGTTATCTTCAACCTAGGA
CTTTTCTATTAAAATATAATGAAAATGGAACCATTACAGATGCTGTAGACTGTGCACTT
GACCCTCTCTCAGAAACAAAGTGTACGTTGAAATCCTTCACTGTAGAAAAAGGAATCTA
TCAAACTTCTAACTTTAGAGTCCAACCAACAGAATCTATTGTTAGATTTCCTAATATTA
CAAACTTGTGCCCTTTTGGTGAAGTTTTTAACGCCACCAGATTTGCATCTGTTTATGCT
TGGAACAGGAAGAGAATCAGCAACTGTGTTGCTGATTATTCTGTCCTATATAATTCCGC
ATCATTTTCCACTTTTAAGTGTTATGGAGTGTCTCCTACTAAATTAAATGATCTCTGCT
TTACTAATGTCTATGCAGATTCATTTGTAATTAGAGGTGATGAAGTCAGACAAATCGCT
CCAGGGCAAACTGGAAAGATTGCTGATTATAATTATAAATTACCAGATGATTTTACAGG
CTGCGTTATAGCTTGGAATTCTAACAATCTTGATTCTAAGGTTGGTGGTAATTATAATT
ACCTGTATAGATTGTTTAGGAAGTCTAATCTCAAACCTTTTGAGAGAGATATTICAACT
GAAATCTATCAGGCCGGTAGCACACCTTGTAATGGTGTTGAAGGTTTTAATTGTTACTT
TCCTTTACAATCATATGGTTTCCAACCCACTAATGGTGTTGGTTACCAACCATACAGAG
TAGTAGTACTTTCTTTTGAACTTCTACATGCACCAGCAACTGTTTGTGGACCTAAAAAG
TCTACTAATTTGGTTAAAAACAAATGTGTCAATTTCAACTTCAATGGTTTAACAGGCAC
AGGTGTTCTTACTGAGTCTAACAAAAAGTTTCTGCCTTTCCAACAATTTGGCAGAGACA
TTGCTGACACTACTGATGCTGTCCGTGATCCACAGACACTTGAGATTCTTGACATTACA
CCATGTTCTTTTGGTGGTGTCAGTGTTATAACACCAGGAACAAATACTTCTAACCAGGT
TGCTGTTCTTTATCAGGATGTTAACTGCACAGAAGTCCCTGTTGCTATTCATGCAGATC
AACTTACTCCTACTTGGCGTGTTTATTCTACAGGTTCTAATGTTTTTCAAACACGTGCA
GGCTGTTTAATAGGGGCTGAACATGTCAACAACTCATATGAGTGTGACATACCCATTGG
TGCAGGTATATGCGCTAGTTATCAGACTCAGACTAATTCTCCTCGGCGGGCACGTAGTG
TAGCTAGTCAATCCATCATTGCCTACACTATGTCACTTGGTGCAGAAAATTCAGTTGCT
TACTCTAATAACTCTATTGCCATACCCACAAATTTTACTATTAGTGTTACCACAGAAAT
TCTACCAGTGTCTATGACCAAGACATCAGTAGATTGTACAATGTACATTTGTGGTGATT
CAACTGAATGCAGCAATCTTTTGTTGCAATATGGCAGTTTTTGTACACAATTAAACCGT
GCTTTAACTGGAATAGCTGTTGAACAAGACAAAAACACCCAAGAAGTTTTTGCACAAGT
CAAACAAATTTACAAAACACCACCAATTAAAGATTTTGGTGGTTTTAATTTTTCACAAA
TATTACCAGATCCATCAAAACCAAGCAAGAGGTCATTTATTGAAGATCTACTTTTCAAC
AAAGTGACACTTGCAGATGCTGGCTTCATCAAACAATATGGTGATTGCCTTGGTGATAT
TGCTGCTAGAGACCTCATTTGTGCACAAAAGTTTAACGGCCTTACTGTTTTGCCACCTT
TGCTCACAGATGAAATGATTGCTCAATACACTTCTGCACTGTTAGCGGGTACAATCACT
TCTGGTTGGACCTTTGGTGCAGGTGCTGCATTACAAATACCATTTGCTATGCAAATGGC
TTATAGGTTTAATGGTATTGGAGTTACACAGAATGTTCTCTATGAGAACCAAAAATTGA
TTGCCAACCAATTTAATAGTGCTATTGGCAAAATTCAAGACTCACTTTCTTCCACAGCA
AGTGCACTTGGAAAACTTCAAGATGTGGTCAACCAAAATGCACAAGCTTTAAACACGCT
TGTTAAACAACTTAGCTCCAATTTTGGTGCAATTTCAAGTGTTTTAAATGATATCCTTT
CACGTCTTGACAAAGTTGAGGCTGAAGTGCAAATTGATAGGTTGATCACAGGCAGACTT
CAAAGTTTGCAGACATATGTGACTCAACAATTAATTAGAGCTGCAGAAATCAGAGCTTC
TGCTAATCTTGCTGCTACTAAAATGTCAGAGTGTGTACTTGGACAATCAAAAAGAGTTG
ATTTTTGTGGAAAGGGCTATCATCTTATGTCCTTCCCTCAGTCAGCACCTCATGGTGTA
GTCTTCTTGCATGTGACTTATGTCCCTGCACAAGAAAAGAACTTCACAACTGCTCCTGC
CATTTGTCATGATGGAAAAGCACACTTTCCTCGTGAAGGTGTCTTTGTTTCAAATGGCA
CACACTGGTTTGTAACACAAAGGAATTTTTATGAACCACAAATCATTACTACAGACAAC
ACATTTGTGTCTGGTAACTGTGATGTTGTAATAGGAATTGTCAACAACACAGTTTATGA
TCCTTTGCAACCTGAATTAGACTCATTCAAGGAGGAGTTAGATAAATATTTTAAGAATC
ATACATCACCAGATGTTGATTTAGGTGACATCTCTGGCATTAATGCTTCAGTTGTAAAC
ATTCAAAAAGAAATTGACCGCCTCAATGAGGTTGCCAAGAATTTAAATGAATCTCTCAT
CGATCTCCAAGAACTTGGAAAGTATGAGCAGTATATAAAATGGCCATGGTACATTTGGC
TAGGTTTTATAGCTGGCTTGATTGCCATAGTAATGGTGACAATTATGCTTTGCTGTATG
ACCAGTTGCTGTAGTTGTCTCAAGGGCTGTTGTTCTTGTGGATCCTGATGCAAATTTGA
TGAAGACGACTCTGAGCCAGTGCTCAAAGGAGTCGCATTAGCTTACACATAAGCGGCCG
CCCTGCACACAGATTCTTCATGTTTGAACCAAATCAACTTGTGATATCATGCTCAAAGA
GGCCTTAATTATATTTTAATTTTTAATTTTTATGAAAAAAACTAACAGCAATCATGGAA
GTCCACGATTTTGAGACCGACGAGTTCAATGATTTCAATGAAGATGACTATGCCACAAG
AGAATTCCTGAATCCCGATGAGCGCATGACGTACTTGAATCATGCTGATTACAATTTGA
ATTCTCCTCTAATTAGTGATGATATTGACAATTTGATCAGGAAATTCAATTCTCTTCCG
ATTCCCTCGATGTGGGATAGTAAGAACTGGGATGGAGTTCTTGAGATGTTAACATCATG
TCAAGCCAATCCCATCTCAACATCTCAGATGCATAAATGGATGGGAAGTTGGTTAATGT
CTGATAATCATGATGCCAGTCAAGGGTATAGTTTTTTACATGAAGTGGACAAAGAGGCA
GAAATAACATTTGACGTGGTGGAGACCTTCATCCGCGGCTGGGGCAACAAACCAATTGA
ATACATCAAAAAGGAAAGATGGACTGACTCATTCAAAATTCTCGCTTATTTGTGTCAAA
AGTTTTTTGACTTACACAAGTTGACATTAATCTTAAATGCTGTCTCTGAGGTGGAATTG
CTCAACTTGGCGAGGACTTTCAAAGGCAAAGTCAGAAGAAGTTCTCATGGAACGAACAT
ATGCAGGATTAGGGTTCCCAGCTTGGGTCCTACTTTTATTTCAGAAGGATGGGCTTACT
TCAAGAAACTTGATATTCTAATGGACCGAAACTTTCTGTTAATGGTCAAAGATGTGATT
ATAGGGAGGATGCAAACGGTGCTATCCATGGTATGTAGAATAGACAACCTGTTCTCAGA
GCAAGACATCTTCTCCCTTCTAAATATCTACAGAATTGGAGATAAAATTGTGGAGAGGC
AGGGAAATTTTTCTTATGACTTGATTAAAATGGTGGAACCGATATGCAACTTGAAGCTG
ATGAAATTAGCAAGAGAATCAAGGCCTTTAGTCCCACAATTCCCTCATTTTGAAAATCA
TATCAAGACTTCTGTTGATGAAGGGGCAAAAATTGACCGAGGTATAAGATTCCTCCATG
ATCAGATAATGAGTGTGAAAACAGTGGATCTCACACTGGTGATTTATGGATCGTTCAGA
CATTGGGGTCATCCTTTTATAGATTATTACACTGGACTAGAAAAATTACATTCCCAAGT
AACCATGAAGAAAGATATTGATGTGTCATATGCAAAAGCACTTGCAAGTGATTTAGCTC
GGATTGTTCTATTTCAACAGTTCAATGATCATAAAAAGTGGTTCGTGAATGGAGACTTG
CTCCCTCATGATCATCCCTTTAAAAGTCATGTTAAAGAAAATACATGGCCCACAGCTGC
TCAAGTTCAAGATTTTGGAGATAAATGGCATGAACTTCCGCTGATTAAATGTTTTGAAA
TACCCGACTTACTAGACCCATCGATAATATACTCTGACAAAAGTCATTCAATGAATAGG
TCAGAGGTGTTGAAACATGTCCGAATGAATCCGAACACTCCTATCCCTAGTAAAAAGGT
GTTGCAGACTATGTTGGACACAAAGGCTACCAATTGGAAAGAATTTCTTAAAGAGATTG
ATGAGAAGGGCTTAGATGATGATGATCTAATTATTGGTCTTAAAGGAAAGGAGAGGGAA
CTGAAGTTGGCAGGTAGATTTTTCTCCCTAATGTCTTGGAAATTGCGAGAATACTTTGT
AATTACCGAATATTTGATAAAGACTCATTTCGTCCCTATGTTTAAAGGCCTGACAATGG
CGGACGATCTAACTGCAGTCATTAAAAAGATGTTAGATTCCTCATCCGGCCAAGGATTG
AAGTCATATGAGGCAATTTGCATAGCCAATCACATTGATTACGAAAAATGGAATAACCA
CCAAAGGAAGTTATCAAACGGCCCAGTGTTCCGAGTTATGGGCCAGTTCTTAGGTTATC
CATCCTTAATCGAGAGAACTCATGAATTTTTTGAGAAAAGTCTTATATACTACAATGGA
AGACCAGACTTGATGCGTGTTCACAACAACACACTGATCAATTCAACCTCCCAACGAGT
TTGTTGGCAAGGACAAGAGGGTGGACTGGAAGGTCTACGGCAAAAAGGATGGAGTATCC
TCAATCTACTGGTTATTCAAAGAGAGGCTAAAATCAGAAACACTGCTGTCAAAGTCTTG
GCACAAGGTGATAATCAAGTTATTTGCACACAGTATAAAACGAAGAAATCGAGAAACGT
TGTAGAATTACAGGGTGCTCTCAATCAAATGGTTTCTAATAATGAGAAAATTATGACTG
CAATCAAAATAGGGACAGGGAAGTTAGGACTTTTGATAAATGACGATGAGACTATGCAA
TCTGCAGATTACTTGAATTATGGAAAAATACCGATTTTCCGTGGAGTGATTAGAGGGTT
AGAGACCAAGAGATGGTCACGAGTGACTTGTGTCACCAATGACCAAATACCCACTTGTG
CTAATATAATGAGCTCAGTTTCCACAAATGCTCTCACCGTAGCTCATTTTGCTGAGAAC
GATGCATGATCCTGCTCTTCGTCAATCATTGTATGAAGTTCAAGATAAGATACCGGGCT
TGCACAGTTCTACTTTCAAATACGCCATGTTGTATTTGGACCCTTCCATTGGAGGAGTG
TCGGGCATGTCTTTGTCCAGGTTTTTGATTAGAGCCTTCCCAGATCCCGTAACAGAAAG
TCTCTCATTCTGGAGATTCATCCATGTACATGCTCGAAGTGAGCATCTGAAGGAGATGA
GTGCAGTATTTGGAAACCCCGAGATAGCCAAGTTTCGAATAACTCACATAGACAAGCTA
GTAGAAGATCCAACCTCTCTGAACATCGCTATGGGAATGAGTCCAGCGAACTTGTTAAA
GACTGAGGTTAAAAAATGCTTAATCGAATCAAGACAAACCATCAGGAACCAGGTGATTA
AGGATGCAACCATATATTTGTATCATGAAGAGGATCGGCTCAGAAGTTTCTTATGGTCA
ATAAATCCTCTGTTCCCTAGATTTTTAAGTGAATTCAAATCAGGCACTTTTTTGGGAGT
CGCAGACGGGCTCATCAGTCTATTTCAAAATTCTCGTACTATTCGGAACTCCTTTAAGA
AAAAGTATCATAGGGAATTGGATGATTTGATTGTGAGGAGTGAGGTATCCTCTTTGACA
CATTTAGGGAAACTTCATTTGAGAAGGGGATCATGTAAAATGTGGACATGTTCAGCTAC
TCATGCTGACACATTAAGATACAAATCCTGGGGCCGTACAGTTATTGGGACAACTGTAC
CCCATCCATTAGAAATGTTGGGTCCACAACATCGAAAAGAGACTCCTTGTGCACCATGT
AACACATCAGGGTTCAATTATGTTTCTGTGCATTGTCCAGACGGGATCCATGACGTCTT
TAGTTCACGGGGACCATTGCCTGCTTATCTAGGGTCTAAAACATCTGAATCTACATCTA
TTTTGCAGCCTTGGGAAAGGGAAAGCAAAGTCCCACTGATTAAAAGAGCTACACGTCTT
AGAGATGCTATCTCTTGGTTTGTTGAACCCGACTCTAAACTAGCAATGACTATACTTTC
TAACATCCACTCTTTAACAGGCGAAGAATGGACCAAAAGGCAGCATGGGTTCAAAAGAA
CAGGGTCTGCCCTTCATAGGTTTTCGACATCTCGGATGAGCCATGGTGGGTTCGCATCT
CAGAGCACTGCAGCATTGACCAGGTTGATGGCAACTACAGACACCATGAGGGATCTGGG
AGATCAGAATTTCGACTTTTTATTCCAAGCAACGTTGCTCTATGCTCAAATTACCACCA
CTGTTGCAAGAGACGGATGGATCACCAGTTGTACAGATCATTATCATATTGCCTGTAAG
TCCTGTTTGAGACCCATAGAAGAGATCACCCTGGACTCAAGTATGGACTACACGCCCCC
AGATGTATCCCATGTGCTGAAGACATGGAGGAATGGGGAAGGTTCGTGGGGACAAGAGA
TAAAACAGATCTATCCTTTAGAAGGGAATTGGAAGAATTTAGCACCTGCTGAGCAATCC
TATCAAGTCGGCAGATGTATAGGTTTTCTATATGGAGACTTGGCGTATAGAAAATCTAC
TCATGCCGAGGACAGTTCTCTATTTCCTCTATCTATACAAGGTCGTATTAGAGGTCGAG
GTTTCTTAAAAGGGTTGCTAGACGGATTAATGAGAGCAAGTTGCTGCCAAGTAATACAC
CGGAGAAGTCTGGCTCATTTGAAGAGGCCGGCCAACGCAGTGTACGGAGGTTTGATTTA
CTTGATTGATAAATTGAGTGTATCACCTCCATTCCTTTCTCTTACTAGATCAGGACCTA
TTAGAGACGAATTAGAAACGATTCCCCACAAGATCCCAACCTCCTATCCGACAAGCAAC
CGTGATATGGGGGTGATTGTCAGAAATTACTTCAAATACCAATGCCGTCTAATTGAAAA
GGGAAAATACAGATCACATTATTCACAATTATGGTTATTCTCAGATGTCTTATCCATAG
ACTTCATTGGACCATTCTCTATTTCCACCACCCTCTTGCAAATCCTATACAAGCCATTT
TTATCTGGGAAAGATAAGAATGAGTTGAGAGAGCTGGCAAATCTTTCTTCATTGCTAAG
ATCAGGAGAGGGGTGGGAAGACATACATGTGAAATTCTTCACCAAGGACATATTATTGT
GTCCAGAGGAAATCAGACATGCTTGCAAGTTCGGGATTGCTAAGGATAATAATAAAGAC
ATGAGCTATCCCCCTTGGGGAAGGGAATCCAGAGGGACAATTACAACAATCCCTGTTTA
TTATACGACCACCCCTTACCCAAAGATGCTAGAGATGCCTCCAAGAATCCAAAATCCCC
TGCTGTCCGGAATCAGGTTGGGCCAATTACCAACTGGCGCTCATTATAAAATTCGGAGT
ATATTACATGGAATGGGAATCCATTACAGGGACTTCTTGAGTTGTGGAGACGGCTCCGG
AGGGATGACTGCTGCATTACTACGAGAAAATGTGCATAGCAGAGGAATATTCAATAGTC
TGTTAGAATTATCAGGGTCAGTCATGCGAGGCGCCTCTCCTGAGCCCCCCAGTGCCCTA
GAAACTTTAGGAGGAGATAAATCGAGATGTGTAAATGGTGAAACATGTTGGGAATATCC
ATCTGACTTATGTGACCCAAGGACTTGGGACTATTTCCTCCGACTCAAAGCAGGCTTGG
GGCTTCAAATTGATTTAATTGTAATGGATATGGAAGTTCGGGATTCTTCTACTAGCCTG
AAAATTGAGACGAATGTTAGAAATTATGTGCACCGGATTTTGGATGAGCAAGGAGTTTT
AATCTACAAGACTTATGGAACATATATTTGTGAGAGCGAAAAGAATGCAGTAACAATCC
TTGGTCCCATGTTCAAGACGGTCGACTTAGTTCAAACAGAATTTAGTAGTTCTCAAACG
TCTGAAGTATATATGGTATGTAAAGGTTTGAAGAAATTAATCGATGAACCCAATCCCGA
TTGGTCTTCCATCAATGAATCCTGGAAAAACCTGTACGCATTCCAGTCATCAGAACAGG
AATTTGCCAGAGCAAAGAAGGTTAGTACATACTTTACCTTGACAGGTATTCCCTCCCAA
TTCATTCCTGATCCTTTTGTAAACATTGAGACTATGCTACAAATATTCGGAGTACCCAC
GGGTGTGTCTCATGCGGCTGCCTTAAAATCATCTGATAGACCTGCAGATTTATTGACCA
TTAGCCTTTTTTATATGGCGATTATATCGTATTATAACATCAATCATATCAGAGTAGGA
CCGATACCTCCGAACCCCCCATCAGATGGAATTGCACAAAATGTGGGGATCGCTATAAC
TGGTATAAGCTTTTGGCTGAGTTTGATGGAGAAAGACATTCCACTATATCAACAGTGTT
TGGCAGTTATCCAGCAATCATTTCCGATTAGGTGGGAGGCTATTTCAGTAAAAGGAGGA
TACAAGCAGAAGTGGAGTACTAGAGGTGATGGGCTCCCAAAAGATACCCGAATTTCAGA
CTCCTTGGCCCCAATCGGGAACTGGATCAGATCTTTGGAATTGGTCCGAAACCAAGTTC
GTCTAAATCCATTCAATAAGATCTTGTTCAATCAGCTATGTCGTACAGTGGATAATCAT
TTGAAGTGGTCAAATTTGCGAAAAAACACAGGAATGATTGAATGGATCAATGGGCGAAT
TTCAAAAGAAGACCGGTCTATACTGATGTTGAAGAGTGACCTACATGAGGAAAACTCTT
GGAGAGATTAAAAAATCAGGAGGAGACTCCAAACTTTAAGTATGAAAAAAACTTTGATC
CTTAAGACCCTCTTGTGGTTTTTATTTTTTATCTGGTTTTGTGGTCTTCGT

The SARS-CoV-2 spike protein was cloned in place of VSV G. An alignment of the cytoplasmic tail of the SARS-CoV-2 spike protein is shown, and the mutations made in the rescued recombinant are shown in red. Amino acid sequence (membrane proximal region, transmembrane domain, cytoplasmic tail).

Amino Acid Sequence of Tail Mutant S_{AA}
(SEQ ID NO: 4)
(K1269A/H1271A)
LNESLIDLQELGKYEQYIKWPWYIWLGFIAGLIAIVMVTIMLCCMTSCCS
CLKGCCSCGSCCKFDEDDSEPVLKGVALAYT
Amino Acid Sequence of Tail Mutant MERS
(SEQ ID NO: 5)
LNESLIDLQELGKYEQYIKWPWYIWLGFIAGLIAIVMVTIML
KLKCNRCCDRYEEYDLEPHAVAVH
Amino Acid Sequence of Tail Mutant VSV G #1
(SEQ ID NO: 6)
LNESLIDLQELGKYEQYIKWPWYIWLGFIAGLIAIVMVTIMLCCMTSCC
SCLRVGTYLCIKLKHTKKRQIYTDIEMNRIGK
Amino Acid Sequence of Tail Mutant VSV G #2
(SEQ ID NO: 7)
LNESLIDLQELGKYEQYIKWPWYIW
LGFIAGLIAIVMVTIML
RVGIYLCIKLKHTKKRQIYTDIEMNRLGK
Amino Acid Sequence of Tail Mutant VSV G
Ecto/TM/tail
(SEQ ID NO: 8)
LNEGWFSSWKSSIASFCFIIGLIIGLFLVLRVGIYLCIKLKHTKK
RQIYTDIEMNRLGK
Amino Acid Sequence of Tail Mutant SΔ21
(21 amino acid truncation)
(SEQ ID NO: 9)
LNESLIDLQELGKYEQYIKWPWYIWLGFIAGLIAIVMVTIMLCC
MTSCCSCLKGCCSCGS*

The Brazil, United Kingdom (UK), and South African (SA) variants or mutation(s) can be included in the rVSV construct. For example, N501Y from UK and SA; 69/70-deletion+N501Y+D614G from UK; and 484K+N501Y+D614G from SA. As another example, the rVSV can include an amino acid sequence of tail mutant S_{Δ21} (21 amino acid truncation) comprising the B.1.1.7 (UK) variant (e.g., SEQ ID NO: 1 with a 21 AA truncation, and N501Y (from UK and SA)), B.1.1.7 (UK+E484K), or N501Y (from UK and SA) and E484K, or B.1.351 (South Africa) (e.g., K417N, E484K and/or N501Y), or P.1 (Brazil) (e.g., E484K). Other substitutions in Spike (SEQ ID NO: 1) can be E488A; E484K; E484D; E484G; S477N; S477G; S477R; K444E; K444N; T345A; T345N; T345S; G446D; G446V; R346G; N450D; N450K; N450Y; F486S; F486Y; L441R; L452R; A352D; T478I; F490S; S494P; P499L; T345A/L517R; S477N/S514F; and/or D614G. See also TABLE 2 for others. As another example, mutation(s) can include one or more of the following: T345A; T345A/L517R; T345A/E484A; T345N; T345S; T345S/F486S; T346G/E484A; R346K/E484K; R346G; R346K; A352D; A372T; A372T/E484K; K378E; K378Q; R408K; K417N; L441R; K444E; K444E/E484A; K444E/E484K; K444N; K444R; V445G; G446D; G446V; N450D; N450K; N450Y; L452R; K458Q; Q474P; G476D; G476S; S4771; S477G; S477N; S477N/S514F; S477R; T478I; T478P; P479L; P479S; V483F; V483G; E484A; E484D; E484K; E484G; F486L; F486S; F486Y; F486V; E488A; F490L; F490S; S494P; P499L; N501Y; G504D; S514F; L517R; K535R; D614G; or combinations thereof or other mutations presently known or identified in the future.

In some embodiments, the recombinant VSV comprises a nucleic acid sequence that is at least 80 (e.g., at least 81, 82, 83, 84, 85, 86, 87, 88, 89, 90, 91, 92, 93, 94, 95, 96, 97, 98, or 99)% identical to the nucleic acid encoding a spike protein, such as SEQ ID NO: 1 (spike portion; S gene of SARS-CoV-2 isolate Wuhan-Hu-1 (GenBank MN908947.3)) or any one of SEQ ID NO: 4-13.

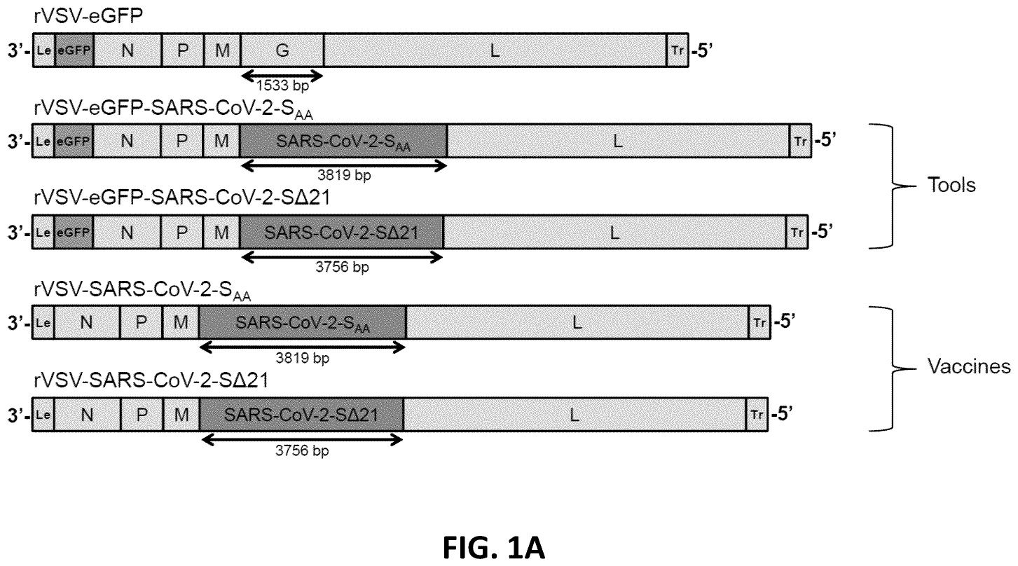

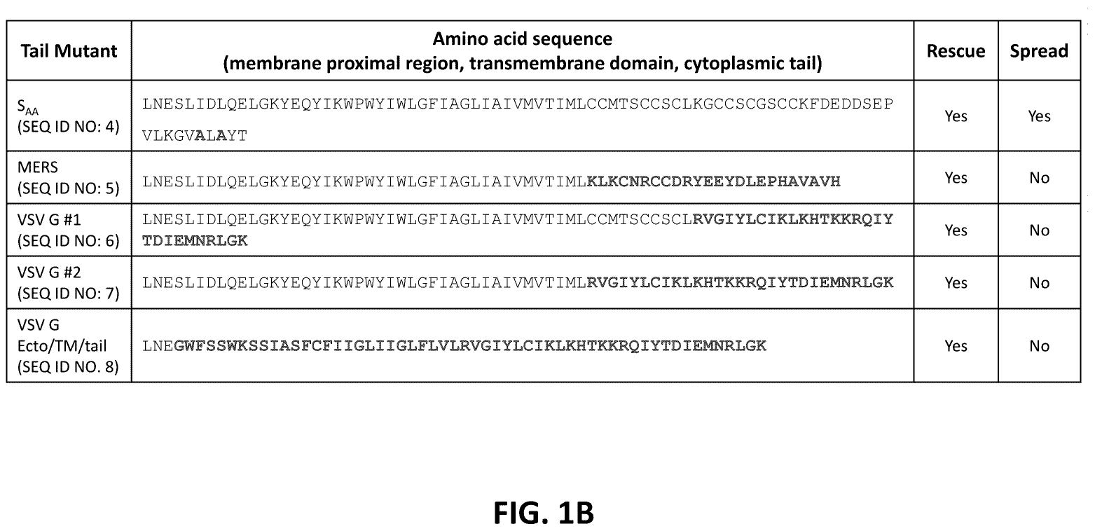

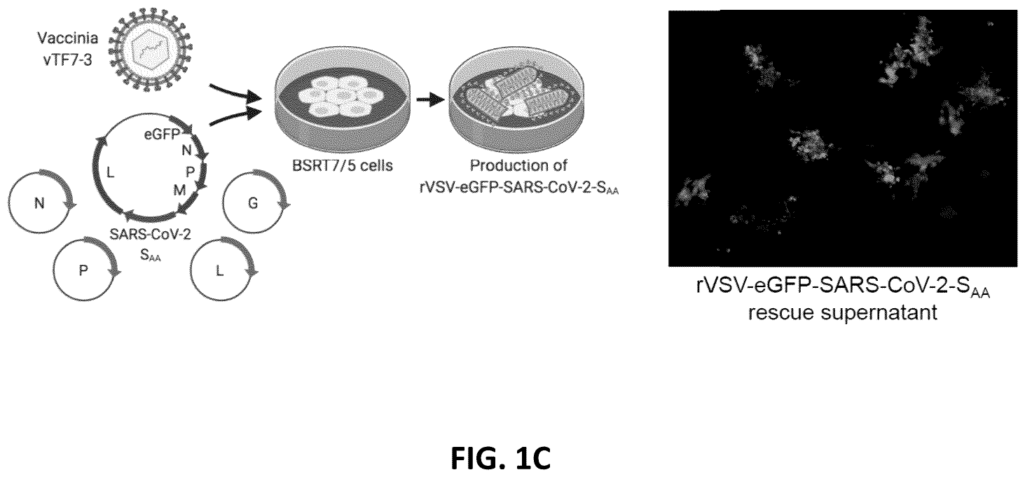

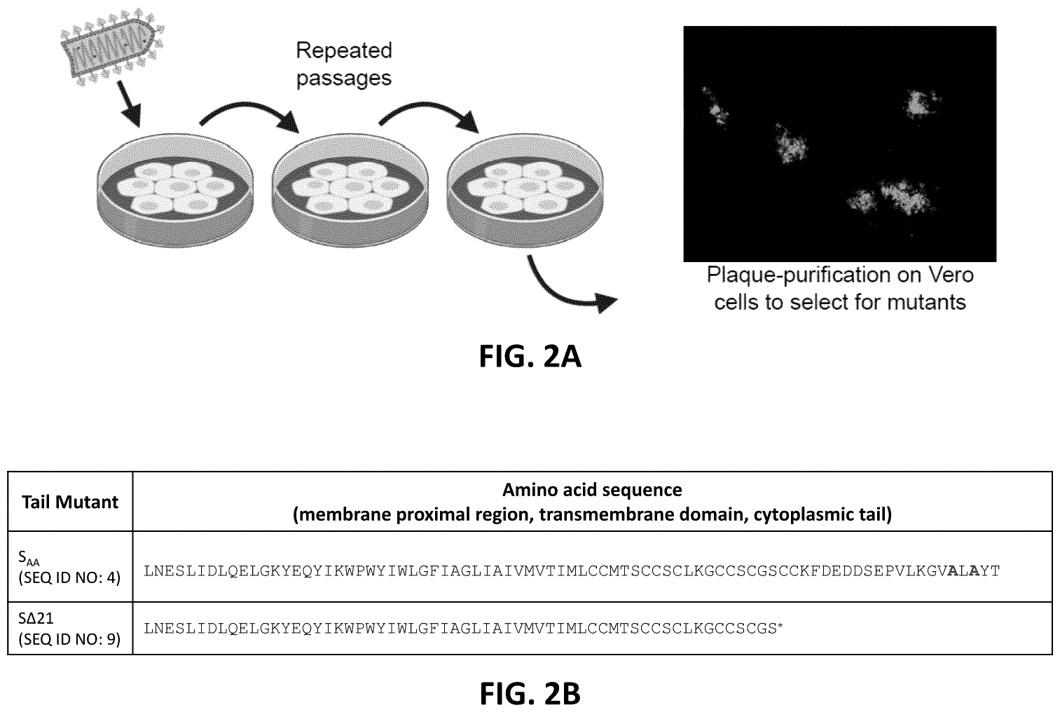

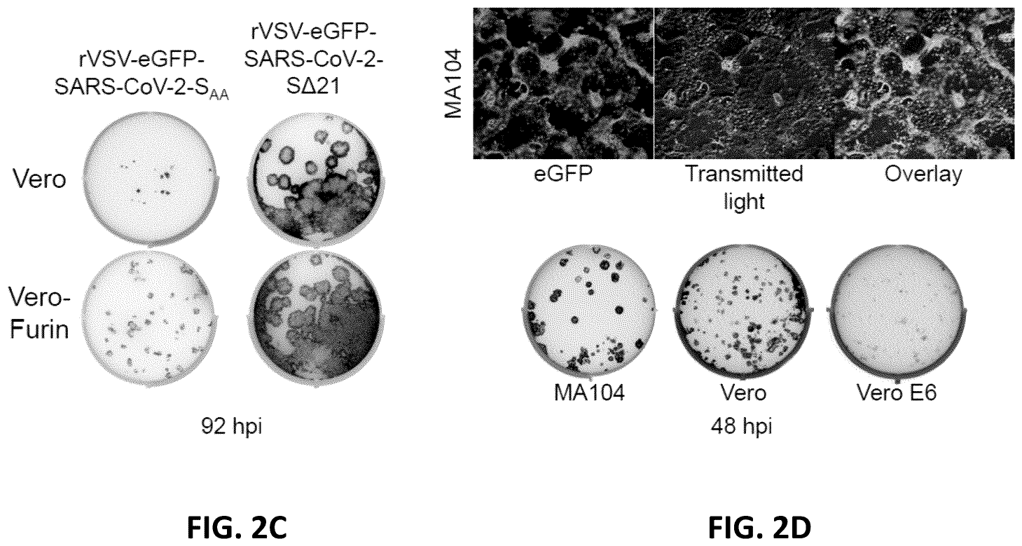

In some embodiments, the recombinant VSV comprises a nucleic acid sequence that is at least 80 (e.g., at least 81, 82, 83, 84, 85, 86, 87, 88, 89, 90, 91, 92, 93, 94, 95, 96, 97, 98, or 99)% identical to the nucleic acid of SEQ ID NO: 2 or SEQ ID NO: 3 (or portions thereof, e.g., N, P, M, SARS-CoV-2 spike or mutant spike, or L portion (see e.g., FIG. 1, FIG. 2B)). In some embodiments, the nucleic acid sequence encoding a SARS-CoV spike protein substantially replaces the endogenous VSV viral glycoprotein (G) in the VSV genome (see e.g., SEQ ID NO: 2, SEQ ID NO: 3) or can be inserted into an intact VSV (i.e., with G).

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process. “Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell. As described herein, codon optimized or codon optimized and wild-type nucleotide sequences of the SARS-CoV-2 spike or variants thereof, including spike lacking the final 63 nucleotides (corresponding to the final 21 amino acids), can be inserted into the VSV genome, optionally in place of the native G gene.

A “codon” is defined as a trinucleotide sequence of DNA or RNA that corresponds to a specific amino acid. The genetic code describes the relationship between the sequence of bases in a gene and the corresponding protein sequence that it encodes. The cell reads the sequence of the gene in groups of three bases.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing a Coronavirus infection in a subject in need of administration of a therapeutically effective amount of a composition comprising an rVSV.

Methods described herein are generally performed on a subject in need thereof. A subject in need of the therapeutic methods described herein can be a subject having, diagnosed with, suspected of having, or at risk for developing a Coronavirus infection. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a composition comprising an rVSV is, for example, an amount that would cause the desired therapeutic effect in a subject while minimizing undesired side effects. In various embodiments, an effective amount of a composition comprising an rVSV described herein can substantially inhibit a Coronavirus infection, slow the progress of a Coronavirus infection, or limit the development of a Coronavirus infection.

According to the methods described herein, administration can be parenteral, pulmonary, oral, topical, intradermal, intramuscular, intraperitoneal, intravenous, intrathecal, intracranial, intracerebroventricular, subcutaneous, intranasal, epidural, ophthalmic, buccal, or rectal administration.

When used in the treatments described herein, a therapeutically effective amount of a composition comprising an rVSV can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit a Coronavirus infection, slow the progress of a Coronavirus infection, or limit the development of a Coronavirus infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a composition comprising an rVSV can occur as a single event or over a time course of treatment. For example, a composition comprising an rVSV can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a Coronavirus infection.

A composition comprising an rVSV can be administered simultaneously or sequentially with another agent, such as an antibiotic, an anti-inflammatory, antiviral, or another therapeutic agent (e.g., chloroquine, hydroxychloroquine, azithromycin). For example, a composition comprising an rVSV can be administered simultaneously with another agent, such as an antibiotic or an anti-inflammatory. Simultaneous administration can occur through administration of separate compositions, each containing one or more of a composition comprising an rVSV, an antibiotic, an anti-inflammatory, or another agent. Simultaneous administration can occur through administration of one composition containing two or more of a composition comprising an rVSV, an antibiotic, an anti-inflammatory, or another agent. A composition comprising an rVSV can be administered sequentially with an antibiotic, an anti-inflammatory, or another agent. For example, a composition comprising an rVSV can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening sera, inhibitors, drug candidates, or experimental therapeutic agents.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to rVSVs, rVSV vectors, plasmids, cells, etc., as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1: SARS CoV-2 Vaccines and High Throughput Screening Assays Based on Vesicular Stomatitis Virus Vectors

This example describes a VSV recombinant in which the native glycoprotein (G) has been replaced by the spike gene of SARS-CoV-2 the causal agent of COVID-19. The native G may stay intact as well. Described herein, is a vaccine candidate (see e.g., Example 2 for more details) and a high throughput screening tool (see e.g., Example 3 for further details) for use under biosafety level 2 conditions to identify inhibitors that work by blocking SARS-CoV-2 spike protein mediated steps in infection.

The VSV Vectored SARS CoV-2 (Aka Wuhan) Vaccine

Shown here is the design of a vesicular stomatitis virus vectored vaccine candidate for Wuhan virus. This vectored vaccine replaces the single surface glycoprotein of VSV with the spike protein (S) of SARS CoV-2 (aka Wuhan-Hu1).

FIG. 1 shows the design strategy and rescue of replication-competent VSV expressing the SARS-CoV-2 spike. Illustration depicting the genomic organization of VSV recombinants. Shown from 3′ to 5′ are the leader RNA, eGFP reporter, nucleocapsid, phosphoprotein, matrix protein, glycoprotein, polymerase (large protein), and a trailer RNA. The SARS-CoV-2 spike protein was cloned in place of VSV G. An alignment of the cytoplasmic tail of the SARS-CoV-2 spike protein is shown, and the mutations made in the rescued recombinant are shown in red (FIG. 1A). A schematic demonstrating the method of virus rescue is shown in FIG. 1C. BSRT7/5 cells were infected with Vaccinia VTF7-3 and transfected with an infectious molecular clone encoding the S_{AA} mutant and helper plasmids N, P, L, and G to rescue recombinant virus (FIG. 1B). Vero cells were infected with rescue supernatant, viral particles of which contain VSV G in trans. Expression of eGFP was imaged 44 hours post-infection (FIG. 1C).

Selection and characterization of a gain-of-function recombinant VSV was demonstrated. rVSV-eGFP-SARS-CoV-2 S_{AA} was plaque-purified and passaged repeatedly on Vero cells. Individual clones were from passaged supernatants were plaque-purified on Vero cells, and RNA from infected cells was deep sequenced (FIG. 2A). Alignment of the cytoplasmic tails of wild-type SARS-CoV-2 S, rVSV-eGFP-SARS-CoV-2-S_{AA}, and the selected mutant. An asterisk indicates where a stop mutation was acquired in the cytoplasmic tail that resulted in a 21 amino acid truncation of the protein (FIG. 2B). A plaque assay was performed to compare rVSV-eGFP-SARS-CoV-2-S_{AA} rescue supernatant and rVSV-eGFP-SARS-CoV-2-S_{Δ21} on Vero and Vero-Furin cells. Expression of eGFP is shown 92 hpi (FIG. 2C).

MA104 cells were infected with rVSV-eGFP-SARS-CoV-2-SΔ21 at an MOI of 0.05 and images showing eGFP expression were acquired 18 hpi. Plaque assays (below), visualized by eGFP expression after 48 hours, were performed to compare viral growth and spread on MA104, Vero, and Vero E6 cells (FIG. 2D).

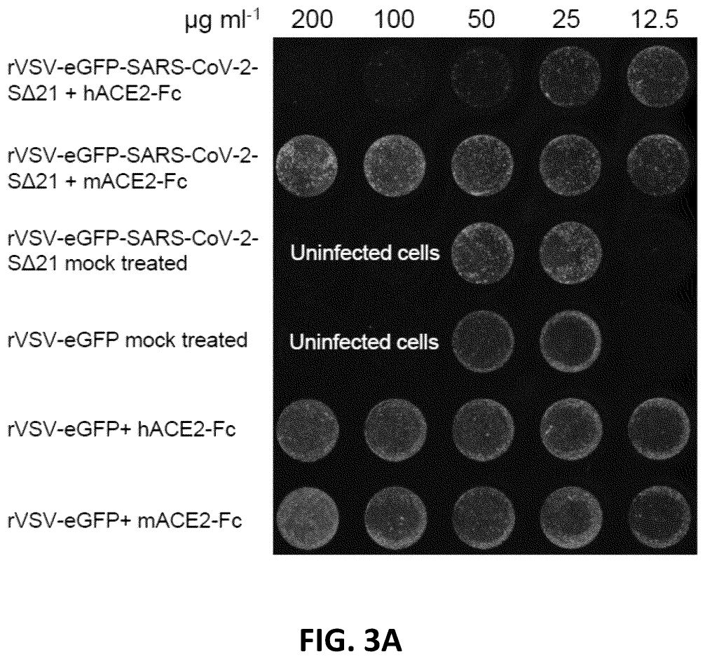

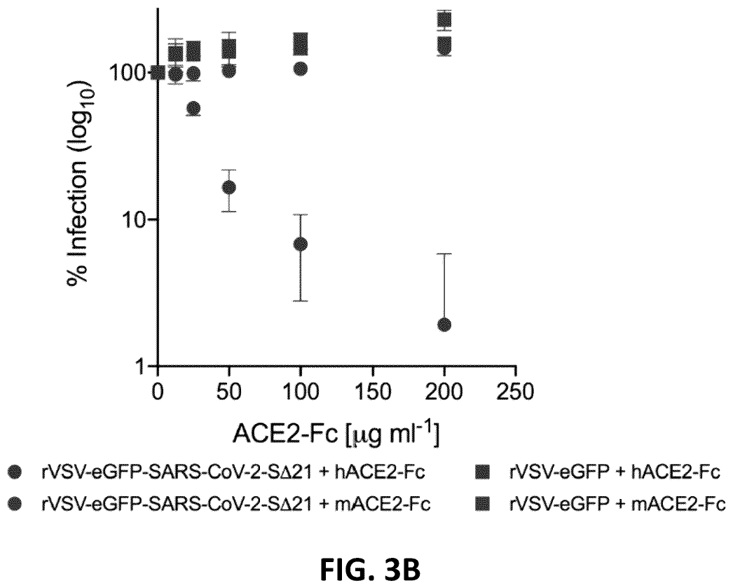

Virus neutralization by soluble receptor was shown. rVSV-SΔ21 or rVSV-eGFP (MOI 1) was incubated with an equal volume of human or murine ACE2-Fc at the indicated concentrations for 1 hr at 37° C., at which time MA104 cells were added to virus-antibody mixture (FIG. 3A). Total eGFP expression was measured 8 hours post-infection using a biomolecular imager and quantified using ImageJ (FIG. 3B). Data represents n=3 experiments with internal duplicates per sample.

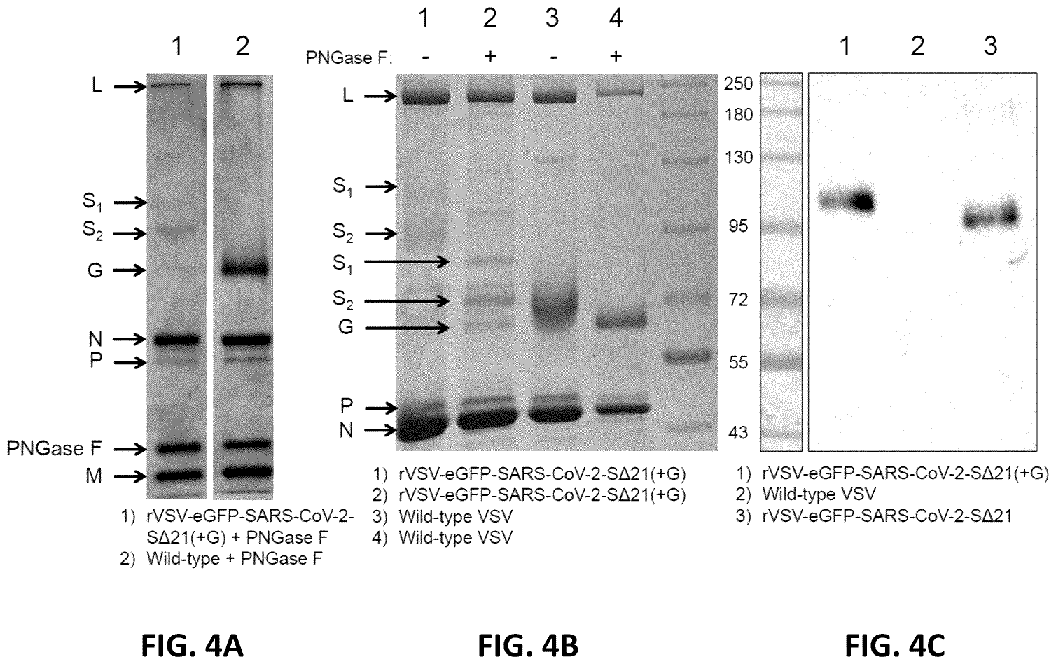

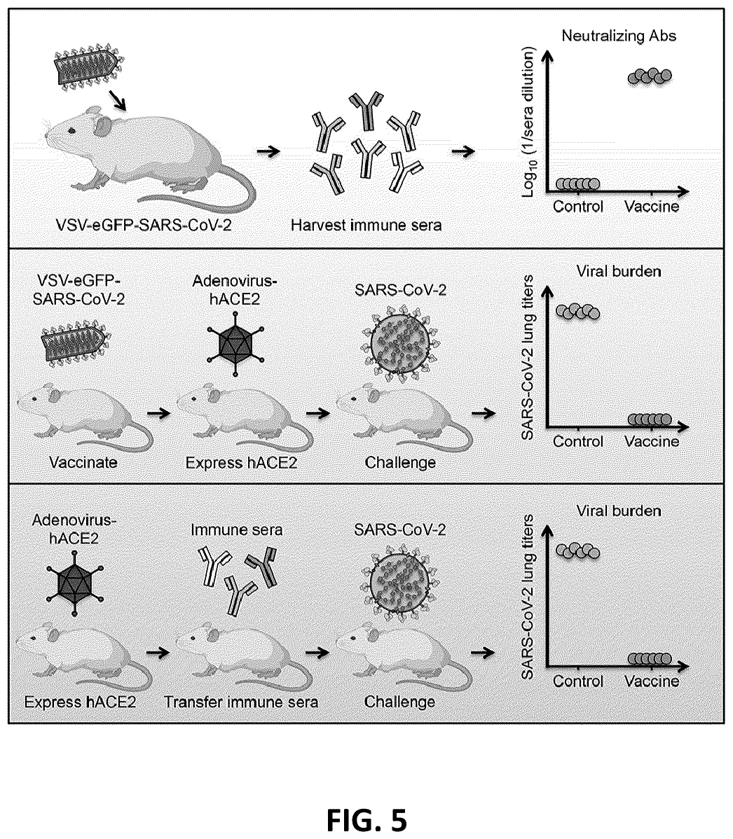

The virus composition is shown in FIG. 4. rVSV-eGFP-SARS-CoV-2-SΔ21 was grown on BSRT7/5 cells in the presence of VSV G. Resulting supernatant, along with wild-type VSV, was sucrose gradient-purified, treated with PNGase F, and run on an 8% high-bis SDS PAGE gel, stained with Coomassie. Viral proteins are labeled. Viruses in (FIG. 4A) were treated with or without PNGase F and run on an 8% SDS-PAGE gel. Viral proteins are labeled. SARS-CoV-2 S1 and S2 are labeled based on expected molecular weight. Bands at the corresponding sizes are absent in VSV samples.

A Western blot (FIG. 4B) was performed on an 8% non-reducing gel using SARS-neutralizing antibody CR3022 on sucrose-gradient purified rVSV-eGFP-SARS-CoV-2-SΔ21 grown on BSRT7/5 cells in the presence of G, rVSV-eGFP-SARS-CoV-2-SΔ21 grown on MA104 cells, and wild-type VSV grown on BSRT7/5 cells. CR3022 was detected with an HRP-fused secondary antibody.

### Example 2: Replication-Competent Vesicular Stomatitis Virus Vaccine Vector Protects Against SARSCoV-2-Mediated Pathogenesis

This example describes the chimeric VSV-SARS-CoV-2 virus as a novel vaccine platform and show immunogenicity and efficacy in mice. This example shows: a replicating VSV-SARS-CoV-2 vaccine induces high-titer neutralizing antibodies; infectious SARS-CoV-2 is undetectable in the lung of vaccinated mice post-challenge; SARS-CoV-2-induced lung inflammation and pathology is decreased in vaccinated mice; and transfer of vaccine-derived immune sera to naive mice protects against SARS-CoV-2. See e.g., Case et al. Volume 28, Issue 3, 9 Sep. 2020, Pages 465-474.e4.

SARS-CoV-2, the etiologic agent of coronavirus induced disease 2019 (COVID-19), has caused a global pandemic with more than 11,000,000 diagnosed infections and a case-fatality rate of ˜5%. An effective and scalable vaccine is of critical importance in mitigating COVID-19, curtailing the pandemic, and restoring social interactions. We developed a replication-competent vesicular stomatitis virus (VSV)-based vaccine by introducing a modified form of the SARS-CoV-2 spike (S) gene in place of the native glycoprotein gene (VSV-eGFP-SARS-CoV-2) (the G protein can also remain intact) (see e.g., FIG. 1). Immunization of mice with VSV-eGFP-SARS-CoV-2 elicits high titers of neutralizing antibodies, including several targeting the receptor binding domain that engages human angiotensin converting enzyme-2 (ACE2). Upon challenge with a human isolate of SARS-CoV-2, mice expressing human ACE2 and immunized with VSV-eGFP-SARS-CoV-2 show profoundly reduced viral infection and inflammation in the lung and are protected against pneumonia. Moreover, passive transfer studies establish immune antibody as a correlate of protection. These data support the development of VSV-eGFP-SARS-CoV-2 as an attenuated, replication-competent vaccine against SARS-CoV-2.

Significance
(a) We immunized BALB/c mice with VSV-eGFP-SARS-CoV-2 and observed robust responses to the viral spike (S) protein and receptor binding domain subunit. These translated into strongly neutralizing antibody responses as determined by focus reduction neutralization tests.
(b) Although single-dose vaccine induced strong immunity, the responses were boosted by a second dose.
(c) One or two doses of VSV-eGFP-SARS-CoV-2, but not the control VSV-eGFP, conferred protection against SARS-CoV-2 challenge in mice expressing human ACE2. This included marked reductions in viral infection in the lung, decreased inflammatory responses, and improvements in tissue histology.
(d) We observed robust immunogenicity and protection despite the fact that VSV-eGFP-SARS-CoV-2 cannot enter cells efficiently and replicate because conventional mice lack the human ACE2 receptor, which we later transduced and expressed for challenge studies. We fully expect greater immunogenicity in animals and humans that natural express ACE2 receptors that support SARS-CoV-2 entry.
(e) We performed passive transfer studies to establish vaccine-derived antibody as a correlate of protection against SARS-CoV-2-induced lung infection and inflammation.
(e) To our knowledge, this is the first evidence for a replication-competent vaccine against SARS-CoV-2.
(f) The disclosed virus replicates to high titer and could provide a cost-efficient and effective alternative for vaccine development, especially in the developing world.

Summary

Severe acute respiratory syndrome coronavirus 2 (SARS-CoV-2) has caused millions of human infections, and an effective vaccine is critical to mitigate coronavirus-induced disease 2019 (COVID-19). Previously, we developed a replication-competent vesicular stomatitis virus (VSV) expressing a modified form of the SARS-CoV-2 spike gene in place of the native glycoprotein gene (VSV-eGFP-SARS-CoV-2). Here, we show that vaccination with VSV-eGFP-SARS-CoV-2 generates neutralizing immune responses and protects mice from SARS-CoV-2. Immunization of mice with VSV-eGFP-SARS-CoV-2 elicits high antibody titers that neutralize SARS-CoV-2 and target the receptor binding domain that engages human angiotensin-converting enzyme-2 (ACE2). Upon challenge with a human isolate of SARS-CoV-2, mice that expressed human ACE2 and were immunized with VSV-eGFP-SARS-CoV-2 show profoundly reduced viral infection and inflammation in the lung, indicating protection against pneumonia. Passive transfer of sera from VSV-eGFP-SARS-CoV-2-immunized animals also protects naive mice from SARS-CoV-2 challenge. These data support development of VSV-SARS-CoV-2 as an attenuated, replication-competent vaccine against SARS-CoV-2.

Introduction

Severe acute respiratory syndrome coronavirus 2 (SARS-CoV-2), a positive-sense, single-stranded, enveloped RNA virus, is the causative agent of coronavirus disease 2019 (COVID-19). Since its outbreak in Wuhan, China in December 2019, SARS-CoV-2 has infected millions of individuals and caused hundreds of thousands of deaths worldwide. Because of its capacity for human-to-human transmission, including from asymptomatic individuals, SARS-CoV-2 has caused a pandemic, leading to significant political, economic, and social disruption (Bai et al., 2020). Currently, social quarantine, physical distancing, and vigilant hand hygiene are the only effective preventative measures against SARS-CoV-2 infections. Thus, effective countermeasures, particularly vaccines, are urgently needed to curtail the virus spread, limit morbidity and mortality, and end the COVID-19 pandemic.

The SARS-CoV-2 spike (S) protein mediates the receptor-binding and membrane fusion steps of viral entry. The S protein also is the primary target of neutralizing antibodies (Baum et al., 2020; Chi et al., 2020; Pinto et al., 2020; Rogers et al., 2020) and can elicit CD4⁺ and CD8⁺ T cell responses (Grifoni et al., 2020). Several SARS-CoV-2 vaccine platforms based on the S protein are being developed, including adenovirus-based vectors, inactivated virus formulations, recombinant subunit vaccines, and DNA- and mRNA-based strategies (Amanat and Krammer, 2020; Lurie et al., 2020). Although several of these vaccines have entered human clinical trials, efficacy data in animals has been published for only a subset of these candidates (Gao et al., 2020; Yu et al., 2020).

We recently reported the generation and characterization of a replication-competent, VSV (designated VSV-eGFP-SARS-CoV-2) that expresses a modified form of the SARS-CoV-2 S protein (Case et al., 2020). We demonstrated that monoclonal antibodies, human sera, and soluble ACE2-Fc potently inhibit VSV-eGFP-SARS-CoV-2 infection in a manner nearly identical to a clinical isolate of SARS-CoV-2. This suggests that chimeric VSV displays the S protein in an antigenic form that resembles native infectious SARS-CoV-2. Because of this data, we hypothesized that a replicating VSV-eGFP-SARS-CoV-2 might serve as an alternative platform for vaccine development. Indeed, an analogous replication-competent recombinant VSV vaccine expressing the Ebola virus (EBOV) glycoprotein protects against lethal EBOV challenge in several animal models (Garbutt et al., 2004; Jones et al., 2005), is safe in immunocompromised nonhuman primates (Geisbert et al., 2008), and was approved for clinical use in humans after successful clinical trials (Henao-Restrepo et al., 2017; Henao-Restrepo et al., 2015). Other live-attenuated recombinant VSV-based vaccines are in pre-clinical development for HIV-1, hantaviruses, filoviruses, arenaviruses, and influenza viruses (Brown et al., 2011; Furuyama et al., 2020; Garbutt et al., 2004; Geisbert et al., 2005; Jones et al., 2005).

Here, we determined the immunogenicity and in vivo efficacy of VSV-eGFP-SARS-CoV-2 as a vaccine in a mouse model of SARS-CoV-2 pathogenesis. We demonstrate that a single dose of VSV-eGFP-SARS-CoV-2 generates a robust neutralizing antibody response that targets both the SARS-CoV-2 S protein and the receptor binding domain (RBD) subunit. Upon challenge with infectious SARS-CoV-2, mice immunized with one or two doses of VSV-eGFP-SARS-CoV-2 showed significant decreases in lung and peripheral organ viral loads, pro-inflammatory cytokine responses, and consequent lung disease. VSV-eGFP-SARS-CoV-2-mediated protection likely is due in part to antibodies, because passive transfer of immune sera to naive mice limits infection after SARS-CoV-2 challenge. This study paves the way for further development of a VSV-vectored SARS CoV-2 vaccine.

Results

Generation of a VSV-eGFP-SARS-CoV-2 as a Vaccine Platform We previously reported a chimeric, replication-competent VSV expressing the SARS-CoV-2 S protein as an effective platform for measuring neutralizing antibodies (Case et al., 2020). Because replication-competent VSVs are in clinical use as vaccines for emerging RNA viruses or in pre-clinical development (Fathi et al., 2019), we tested whether VSV-eGFP-SARS-CoV-2 could protect mice against SARS-CoV-2.

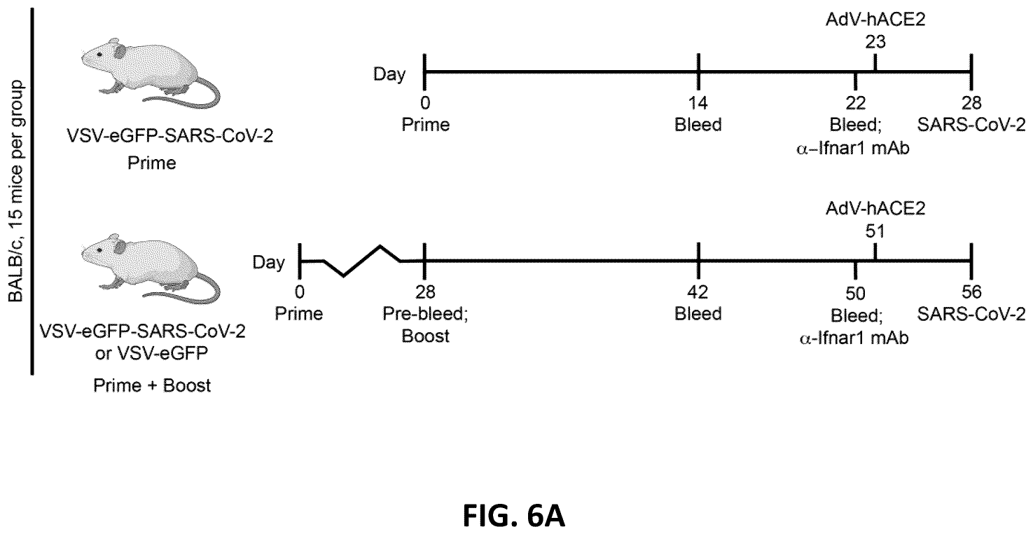

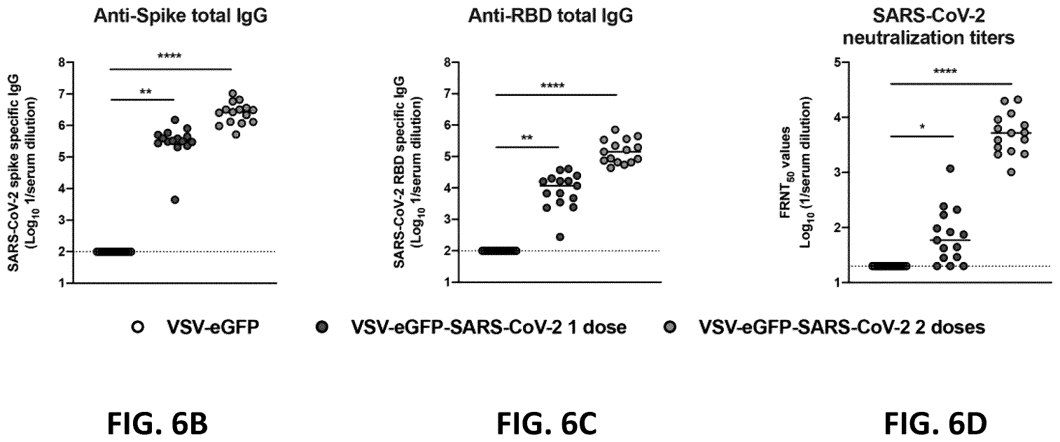

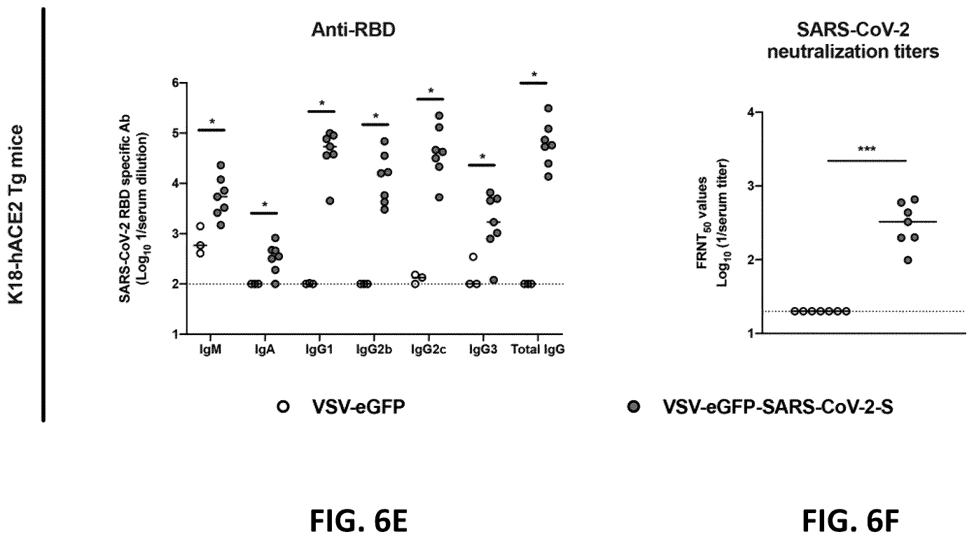

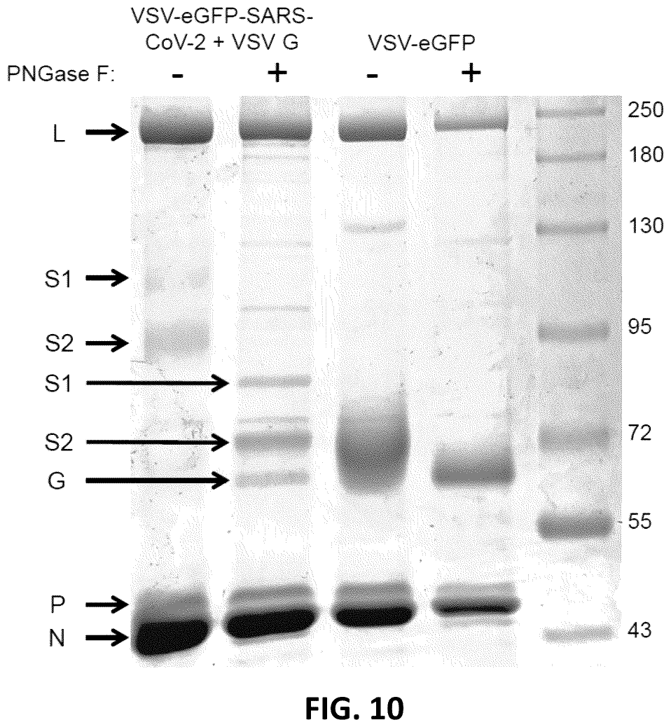

To examine the immune response to VSV-eGFP-SARS-CoV-2, we immunized four-week-old BALB/c mice with 10⁶ plaque-forming units (PFU) of VSV-eGFP-SARS-CoV-2 or a control VSV-eGFP (FIG. 6A). As murine ACE2 does not serve as a receptor for SARS-CoV-2, we spiked our preparation of VSV-eGFP-SARS-CoV-2 with trace amounts of VSV G to permit a single round of infection, an approach used previously for SARS-CoV (Kapadia et al., 2008) (FIG. 10). At 28 days post-priming, one cohort of animals was boosted with the homologous vaccine. Serum was isolated from all animals at three weeks post-priming or boosting, and IgG titers against recombinant SARS-CoV-2 S protein or the RBD were determined by ELISA (FIG. 6B and FIG. 6C). Immunization with VSV-eGFP-SARS-CoV-2 induced high levels of anti-S and anti-RBD-specific IgG compared to control VSV-eGFP with reciprocal median serum endpoint titers of 3.2×10⁵ and 2.7×10⁶ (anti-S) and 1.1×10⁴ and 1.4×10⁵ (anti-RBD) for one and two doses of vaccine, respectively.

We measured neutralizing antibody titers against SARS-CoV-2 after priming or boosting by using a focus-reduction neutralization test (Case et al., 2020). Immunization with a single- or two-dose regimen of VSV-eGFP-SARS-CoV-2 induced neutralizing antibodies (median titers of 1/59 and 1/5,206, respectively) whereas the control VSV-eGFP vaccine did not (FIG. 6D). Boosting was effective and resulted in a 90-fold increase in neutralizing activity after the second dose of VSV-eGFP-SARS-CoV-2. Collectively, these data suggest that VSV-eGFP-SARS-CoV-2 is immunogenic and elicits high titers of antibodies that neutralize infection and target the RBD of the SARS-CoV-2 S protein.

Because VSV-eGFP-SARS-CoV-2 might not enter efficiently into cells of conventional BALB/c mice lacking the human ACE2 (hACE2) receptor, we confirmed immunogenicity in K18-hACE2 transgenic C57BL/6 mice, in which hACE2 expression is driven by an epithelial cell promoter (McCray et al., 2007). We immunized four-week-old K18-hACE2 transgenic mice by intranasal route with 10⁶ PFU of VSV-eGFP-SARS-CoV-2 or VSV-eGFP control. Serum was isolated at three weeks post-priming, and IgG titers against recombinant SARS-CoV-2 RBD were measured by ELISA. We detected robust IgG responses against RBD in VSV-eGFP-SARS-CoV-2 but not VSV-eGFP vaccinated mice (FIG. 6E). Immunoglobulin subclass analysis indicated substantial class-switching occurred, because high levels of IgG2b and IgG2c against RBD were detected (FIG. 6E). Finally, we detected neutralizing antibodies against SARS-CoV-2 (median titer of 1/325) three weeks after immunizing K18-hACE2 transgenic mice with a single dose of VSV-eGFP-SARS-CoV-2 but not VSV-eGFP (FIG. 6F).

VSV-eGFP-SARS-CoV-2 Protects Mice Against SARS-CoV-2 Infection

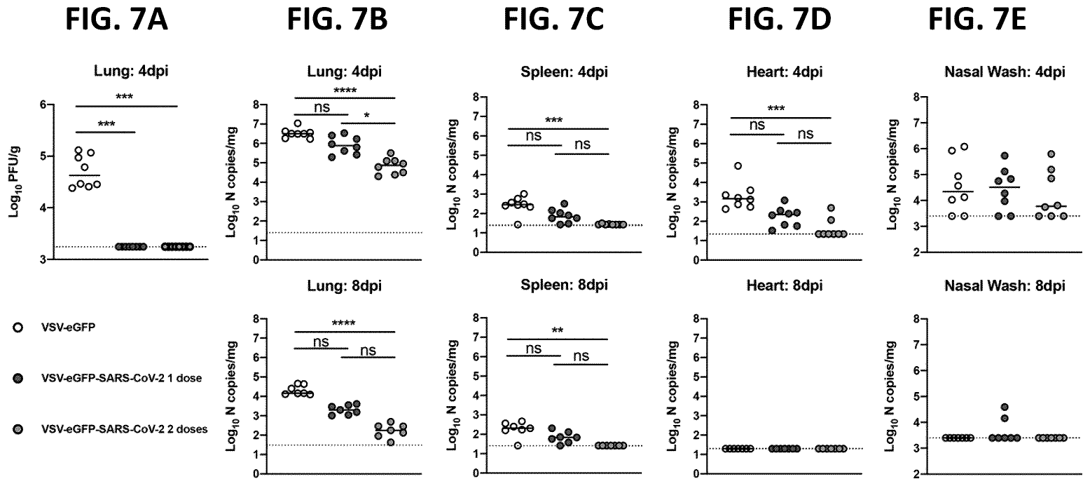

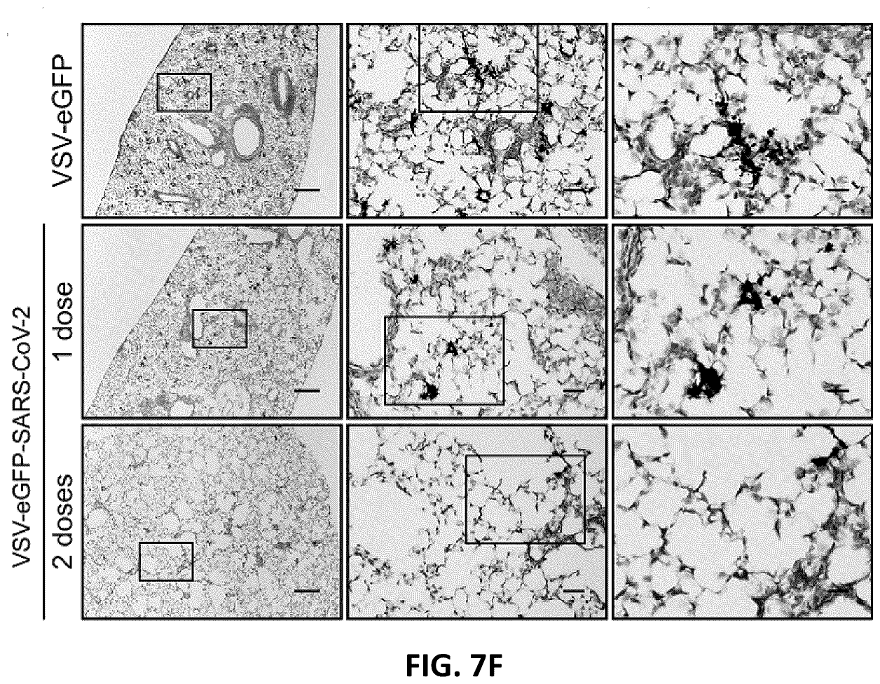

Four weeks after priming or priming and boosting, BALB/c mice were administered 2 mg of anti-Ifnar1 mAb; although not required for infection, this treatment augments pathogenesis of SARS-CoV-2 in the lung and creates a stringent disease model for vaccine protection (Hassan et al., 2020). The following day, mice were inoculated via the intranasal route with a replication-defective adenovirus expressing human ACE2 (AdV-hACE2) that enables receptor expression in the lungs (Hassan et al., 2020). Five days later, mice were challenged via the intranasal route with 3×10⁵ PFU of SARS-CoV-2 (strain 2019 n-CoV/USA_WA1/2020) to evaluate vaccine protection (FIG. 6A). We subsequently measured viral yield both by plaque forming and RT-qPCR assays. At day four post-infection (dpi) infectious virus was not recovered from lungs of mice vaccinated either with one or two doses of VSV-eGFP-SARS-CoV-2 (FIG. 6A). For mice receiving only one dose of VSV-eGFP-SARS-CoV-2 vaccine, we observed a trend toward decreased levels of viral RNA in the lung, spleen, and heart at 4 dpi and in the lung and spleen at 8 dpi in comparison with levels seen in the control VSV-eGFP-vaccinated mice (FIG. 7B-2E). The low levels of SARS-CoV-2 infection in the heart, which were observed previously in this model (Hassan et al., 2020), could be due to spread of the AdV-hACE2 from venous circulation in the lung. Mice that received two doses of VSV-eGFP-SARS-CoV-2 had significantly lower levels of viral RNA in most tissues examined compared to control VSV-eGFP vaccinated mice (FIG. 7B-2E). Consistent with our viral RNA measurements, we observed less SARS-CoV-2 RNA by in situ hybridization in lung tissues of VSV-eGFP-SARS-CoV-2 immunized mice at 4 dpi (FIG. 7F). Collectively, these data establish that immunization with VSV-eGFP-SARS-CoV-2 protects against SARS-CoV-2 infection in mice.

VSV-eGFP-SARS-CoV-2 Limits SARS-CoV-2-Induced Lung Inflammation

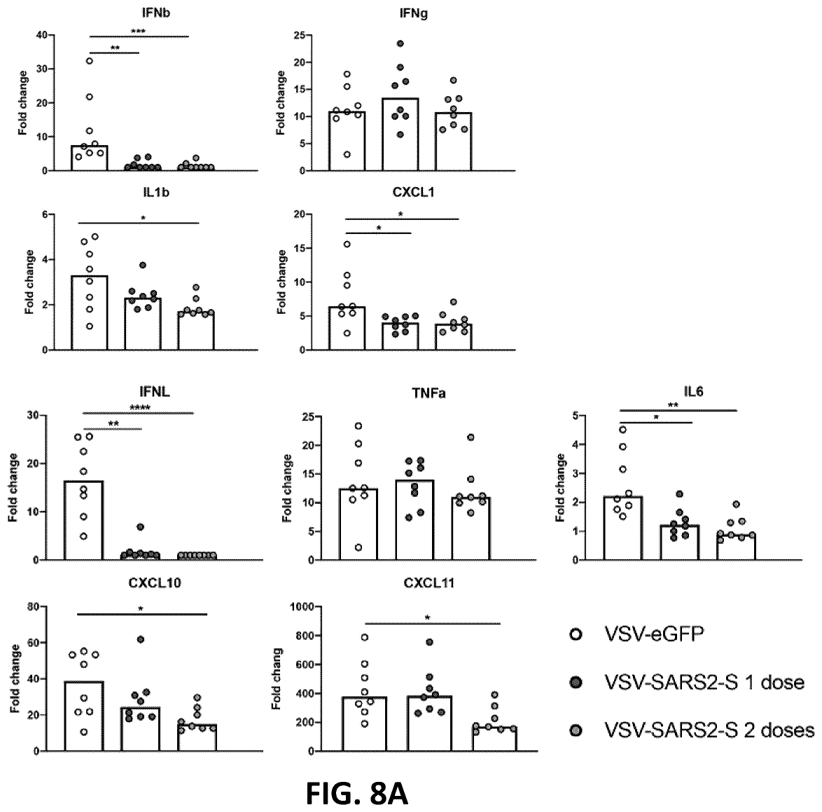

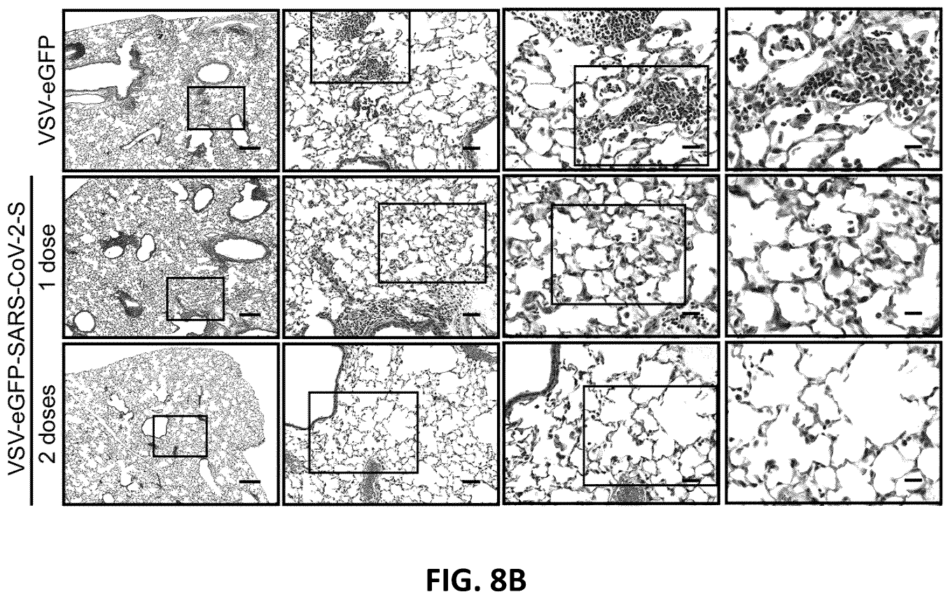

Both SARS-CoV and SARS-CoV-2 typically cause severe lung infection and injury that is associated with high levels of pro-inflammatory cytokines and immune cell infiltrates (Gu and Korteweg, 2007; Huang et al., 2020). The AdV-hACE2-transduced mouse model of SARS-CoV-2 pathogenesis recapitulates several aspects of lung inflammation and coronavirus disease (Hassan et al., 2020). To assess whether VSV-eGFP-SARS-CoV-2 limits virus-induced inflammation, we measured pro-inflammatory cytokine and chemokine mRNA in lung homogenates from vaccinated animals at 4 dpi by RT-qPCR assays (FIG. 8A). Animals immunized with one or two doses of VSV-eGFP-SARS-CoV-2 had significantly lower levels of pro-inflammatory cytokine and chemokine mRNA than did VSV-eGFP vaccinated mice. Specifically, type I and Ill interferons (IFN-β and IFN-λ) were decreased early during infection in both one-dose and two-dose groups of mice immunized with VSV-eGFP-SARS-CoV-2. Although there were no detectable differences in IFN-γ or TNF-α levels between groups, IL-6 and IL-1β were lower at 4 dpi after VSV-eGFP-SARS-CoV-2 vaccination. Similarly, levels of mRNAs encoding chemokines CXCL1, CXCL10, and CXCL11, which recruit immune cells to the lung, were decreased at 4 dpi in VSV-eGFP-SARS-CoV-2 in comparison with VSV-eGFP immunized mice.

To determine the extent of lung pathology in SARS-CoV-2 challenged mice, at 8 dpi, we stained lung sections with hematoxylin and eosin (FIG. 8B). Lung sections from VSV-eGFP-immunized mice showed immune cell (including neutrophils, arrows) infiltration into perivascular, peribronchial, and alveolar locations consistent with viral pneumonia. Lung sections from mice immunized with one dose of VSV-eGFP-SARS-CoV-2 also showed some signs of inflammation. However, mice immunized with two doses of VSV-eGFP-SARS-CoV-2 showed substantially less accumulation of inflammatory cells at the same time point after SARS-CoV-2 infection. These data suggest that immunization with VSV-eGFP-SARS-CoV-2 generates a protective immune response, which limits SARS-CoV-2-induced lung disease in mice. In this model, two sequential immunizations show greater efficacy than a single one.

Vaccine-Induced Sera Limits SARS-CoV-2 Infection

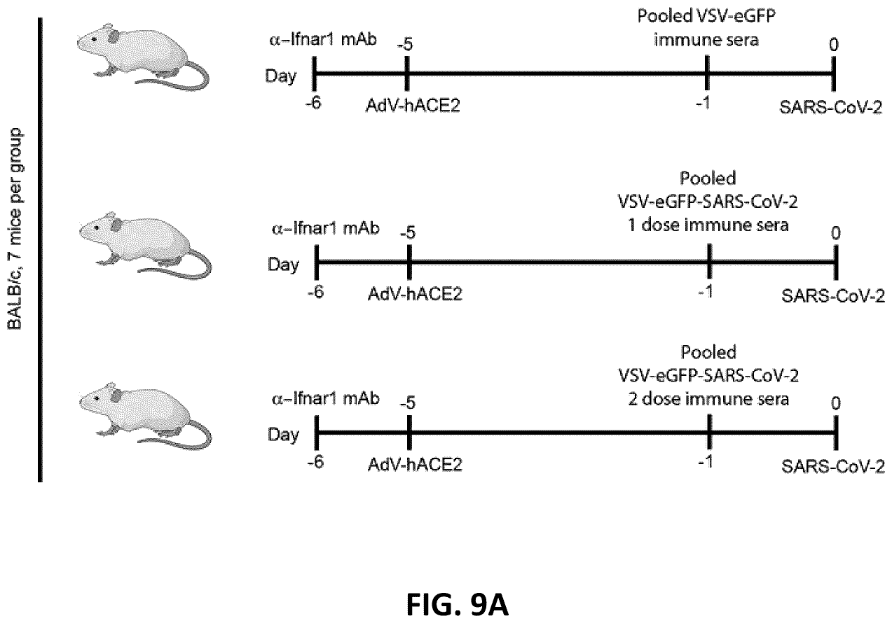

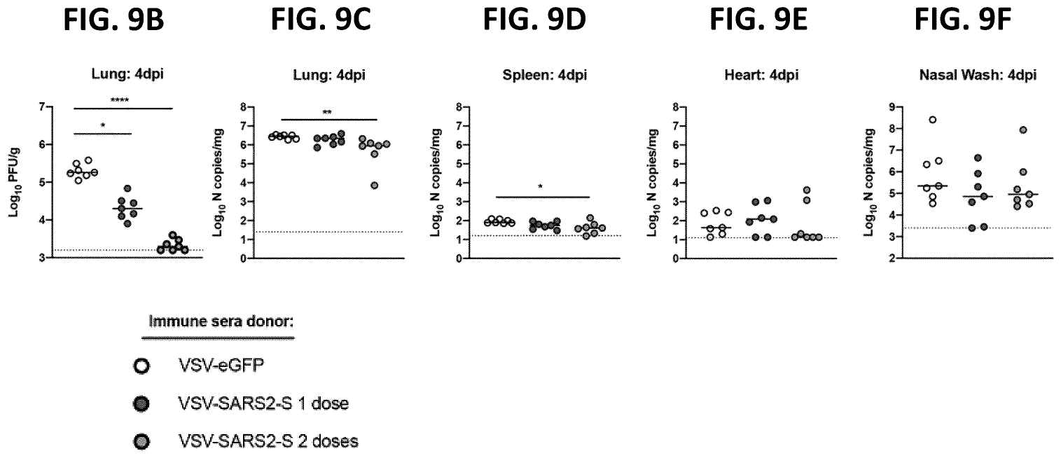

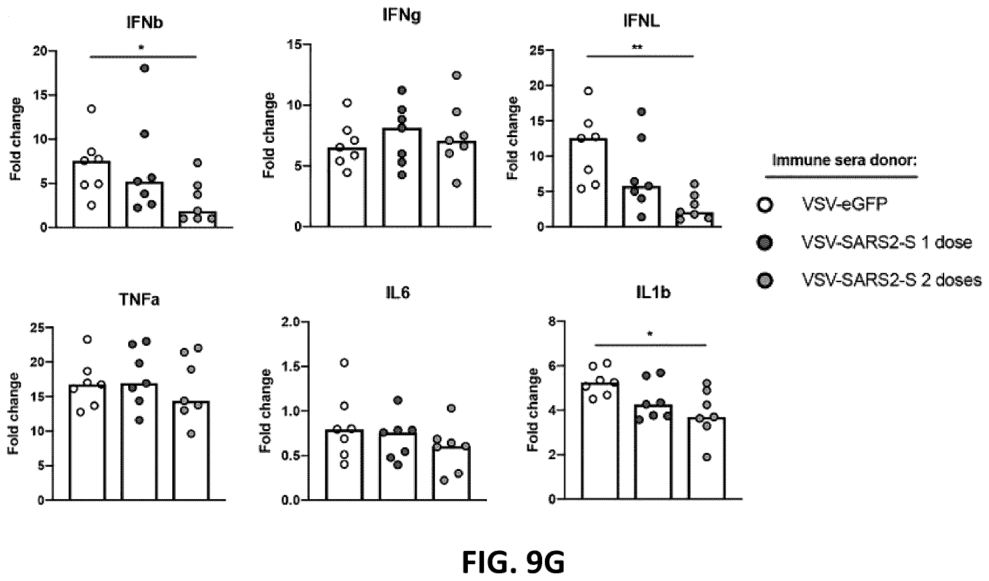

To investigate the contribution of antibodies in vaccine-mediated protection, we performed passive transfer studies. Serum was collected from VSV-eGFP and VSV-eGFP-SARS-CoV-2 vaccinated mice after one or two immunizations. Ten-week-old female BALB/c mice were administered anti-Ifnar1 mAb and AdV-hACE2 as described above to render animals susceptible to SARS-CoV-2. Five days later, 100 μL of pooled immune or control sera was administered by intraperitoneal injection. One day later, mice were inoculated with 3×10⁵ PFU of SARS-CoV-2 via the intranasal route (FIG. 9A). Passive transfer of sera from animals vaccinated with VSV-eGFP-SARS-CoV-2 protected against SARS-CoV-2 infection in comparison with sera from the VSV-eGFP-immunized mice. At 4 dpi, lungs from animals treated with VSV-eGFP-SARS-CoV-2 immune sera from prime-only and boosted animals showed substantially reduced infectious virus burden (FIG. 9B). Although not as striking, significant decreases in viral RNA levels also were observed in the lung and spleen of animals receiving VSV-eGFP-SARS-CoV-2 boosted sera in comparison with the VSV-eGFP sera (FIG. 9C and FIG. 9D). Possibly, some of the viral RNA in lung tissue homogenates after passive transfer could represent neutralized virus within cells that has not yet been cleared. Viral RNA levels in the heart of animals given sera from VSV-eGFP-SARS-CoV-2 boosted mice trended toward, but did not reach, statistical significance (FIG. 9E). No effect was observed in the nasal washes of any treated group (FIG. 9F), consistent with the results from our vaccinated and challenged animals (FIG. 7E).

To determine the effect of the passive transfer of sera on SARS-CoV-2-mediated inflammation, we assessed the induction of several cytokines in the lung at 4 dpi (FIG. 9G). Treatment with sera from animals immunized with two doses of VSV-eGFP-SARS-CoV-2 limited induction of some (IFN-β, IFN-λ, and IL-1 P) pro-inflammatory cytokines after SARS-CoV-2 challenge. Together, these data suggest that antibodies are a major correlate of VSV-eGFP-SARS-CoV-2-mediated protection against SARS-CoV-2.

Discussion

The emergence of SARS-CoV-2 into the human population has caused a global pandemic, resulting in millions of infected individuals and hundreds of thousands of deaths. Despite initial indications that the pandemic had peaked, reopening of countries and renewed human-to-human contact has resulted in a recent surge in case numbers, suggesting that SARS-CoV-2 vaccines will be critical for curtailing the pandemic and resuming normal social interactions. In this study, we tested the efficacy of a replication-competent VSV-eGFP-SARS-CoV-2 vaccine. A single dose of VSV-eGFP-SARS-CoV-2 was sufficient to induce antibodies in BALB/c mice that neutralize SARS-CoV-2 infection and target the RBD and S protein, and a second dose substantially boosted this response. We then challenged mice with SARS-CoV-2 via the intranasal route and observed a complete loss of recovery of infectious virus in the lung in animals immunized with either one or two doses of VSV-eGFP-SARS-CoV-2. In comparison with a single dose, administration of two doses of VSV-eGFP-SARS-CoV-2 elicited greater protection with further diminished viral loads. Immunization with VSV-eGFP-SARS-CoV-2 decreased the induction of several key pro-inflammatory cytokines and protected mice from alveolar inflammation, lung consolidation, and viral pneumonia. We also established an important role for protective antibodies, as passive transfer of immune sera from VSV-eGFP-SARS-CoV-2-immunized animals decreased viral burden and inflammation in the lung.

Recombinant VSV-based vaccines that encode viral glycoproteins have several advantages as a platform. Whereas DNA plasmid and mRNA-based vaccines have not yet been approved in the United States or elsewhere, Merck's ERVEBO, a replication-competent VSV expressing the EBOV glycoprotein, is currently in use in humans (Huttner et al., 2015). As a replicating RNA virus, VSV-based vaccines often can be used as single-dose administration and effectively stimulate both humoral and cellular immunity. Recombinant VSV grows efficiently in mammalian cell culture, enabling simple, large-scale production. Advantages of VSV as a vaccine vector also include the lack of homologous recombination and its non-segmented genome structure, which precludes genetic reassortment and enhances its safety profile (Lichty et al., 2004; Roberts et al., 1999). Other viral-based (e.g., adenoviral, Adv) vaccine vectors are limited to varying degrees (HuAdv5>HuAdv26>ChAdV23) by some level of preexisting immunity to the vector itself (Barouch et al., 2004; Casimiro et al., 2003; Santra et al., 2007). There is almost no preexisting human immunity to VSV, because human infections are rare (Roberts et al., 1999) with the exception of some regions of Central America (Cline, 1976) or a limited number of at-risk laboratory workers (Johnson et al., 1966).

Several vaccine candidates for SARS-CoV-2 have been tested for immunogenicity. Our VSV-eGFP-SARS-CoV-2 vaccine elicited high levels of inhibitory antibodies with median and mean serum neutralizing titers of greater than 1/5,000. Two doses of VSV-eGFP-SARS-CoV-2 induced higher neutralizing titers with more rapid onset than similar dosing of an inactivated SARS-CoV-2 vaccine in the same strain of mice (Gao et al., 2020). Consistent with these results, serum anti-S endpoint titers were higher from mice immunized with two doses of VSV-eGFP-SARS-CoV-2 (1/2,700,000) than the highest two-dose regimen of the inactivated virion vaccine (1/820,000). Two doses of DNA plasmid vaccines encoding variants of the SARS-CoV-2 S protein induced relatively modest neutralizing antibody responses (serum titer of 1/170) in rhesus macaques. Related to this, anti-S titers were approximately 1,000-fold lower after two doses of the optimal DNA vaccine (Yu et al., 2020) when compared to two doses of VSV-eGFP-SARS-CoV-2. In a pre-print study, a single-dose of a chimpanzee Adv vaccine encoding SARS-CoV-2 S protein, ChAdOx1 nCoV-19, also produced relatively low levels of serum neutralizing antibodies in mice and non-human primates (NHPs) (1/40 to 1/80 in BALB/c and CD1 mice and <1/20 in rhesus macaques). This data corresponded with anti-S1 and anti-S2 mean serum titers of between 1/100 and 1/1,000 in BALB/c mice and anti-S titers of <1/1,000 in NHPs (DOI: 10.1101/2020.05.13.093195). Two doses of a recombinant Adv5 vectored SARS-CoV-2 vaccine in humans also produced relatively low RBD binding (1/1,445 at day 28 post-boost) and neutralizing antibody (1/34 at day 28 post-boost) (Zhu et al., 2020). Finally, based on pre-print data (DOI: 10.1101/2020.06.11.145920), BALB/c mice immunized with two 1 μg doses of an mRNA vaccine candidate, mRNA-1273, elicited serum anti-S endpoint titers of 1/100,000. These mice produced mean neutralizing antibodies titers of approximately 1/1,000 and did not show evidence of infectious virus in the lung or nares after SARS-CoV-2 challenge.

Even though VSV-eGFP-SARS-CoV-2 is replication competent and capable of spread, it likely did not do so efficiently in our BALB/c mice because the SARS-CoV-2 S protein cannot efficiently utilize murine ACE2 for viral entry (Letko et al., 2020). This likely explains our need for boosting, because the response we observed likely was enabled by the residual small amount of trans-complementing VSV G to pseudotype the virions expressing the S protein in a manner similar to VSV-SARS (Kapadia et al., 2008), which effectively limited vaccine virus replication to a single cycle. Indeed, in transgenic animals expressing hACE2 receptors competent for S binding, a single dose of VSV-eGFP-SARS-CoV-2 was associated with greater immunogenicity, although a quantitative comparison requires more study because the backgrounds of the mice are different (H-2^{d} BALB/c versus H-2^{b} C57BL/6). Immunization and challenge studies are planned in hACE2 transgenic mice (Bao et al., 2020; Jiang et al., 2020; McCray et al., 2007; Sun et al., 2020) and in NHPs, as they become widely available. Alternatively, hamster models of SARS-CoV-2 infection have been developed with varying degrees of lung pathogenesis (Imai et al., 2020; Sia et al., 2020) and could be used to corroborate vaccine-mediated protection.

Vaccine safety is a key requirement of any platform. Pathogenicity and immunogenicity of VSV are associated with its native glycoprotein G, which, in turn, determines its pan-tropism (Martinez et al., 2003). Replacing the glycoprotein of VSV with a foreign glycoprotein often results in virus attenuation in vivo. Indeed, the vast majority of cases where VSV recombinants express a heterologous viral glycoprotein (e.g., chikungunya virus, H5N1 influenza virus, Lassa virus, lymphocytic choriomeningitis virus, or Ebola virus) and were injected via intracranial route into mice or NHPs, no disease was observed (Mire et al., 2012; Muik et al., 2014; van den Pol et al., 2017; Wollmann et al., 2015). One exception is when VSV expressing the glycoproteins of the highly neurotropic Nipah virus was injected via an intracranial route into adult mice (van den Pol et al., 2017). Should substantial reactogenicity or neuronal infection be observed with VSV-eGFP-SARS-CoV-2, the vaccine could be attenuated further by introducing mutations into the matrix protein (Rabinowitz et al., 1981) or methyltransferase (Li et al., 2006; Ma et al., 2014), rearranging the order of genes (Ball et al., 1999; Wertz et al., 1998), or recoding of the L gene (Wang et al., 2015). The presence of the additional eGFP gene inserted between the leader and N genes also attenuates virus replication in cell culture (Whelan et al., 2000). Further development of a VSV vectored vaccine for SARS-CoV-2 can require the deletion of eGFP from the genome.

Future studies are planned to evaluate the durability of VSV-SARS-CoV-2-induced immunity. Other replication-competent VSV-based vaccines such as the rVSVΔG-ZEBOV-GP have been shown to generate long-lasting immune responses and protection (Kennedy et al., 2017). In addition, we plan to investigate in greater detail the contributions of additional arms of immunity in mediating protection. The robust induction of neutralizing antibodies elicited by one and two doses of VSV-eGFP-SARS-CoV-2 was a correlate of protection, as passive transfer of immune sera reduced viral infection and inflammation in the lung upon SARS-CoV-2 challenge. Nonetheless, it will be important to determine whether additional immune responses, particularly CD8⁺ T cells, have an important protective role. Recently, SARS-CoV-2 specific CD4⁺ and CD8⁺ T cells were shown to be present in 100% and 70% of COVID-19 convalescent patients, respectively, with many of the T cells recognizing peptides derived from the S protein (Grifoni et al., 2020). Indeed, passive transfer of immune sera from vaccinated mice did not completely protect naive mice from SARS-CoV-2 infection, suggesting that T cell responses also might contribute to protection. Although VSV-eGFP-SARS-CoV-2-vaccinated mice were protected against lung infection and inflammation, nasal washes still contained high levels of SARS-CoV-2 RNA. Immunization of VSV-eGFP-SARS-CoV-2 via the intraperitoneal route, while generating systemic immunity that protects against pneumonia, likely did not generate adequate mucosal immunity to neutralize virus at the site of inoculation. This could be overcome by intranasal delivery of the vaccine, as described in studies with influenza A virus (Dutta et al., 2016). Finally, additional experiments are planned in aged animals (hACE2-expressing mice, hamsters, and NHPs) to address immunogenicity and protection in this key target population at greater risk for severe COVID-19. Overall, our data show that VSV-eGFP-SARS-CoV-2 can protect against severe SARS-CoV-2 infection and lung disease, supporting its further development as a vaccine.

Our study, which used an eGFP-expressing variant of VSV-SARS-CoV-2 for tracking purposes, serves as a proof of principle for developing a replication-competent VSV vaccine platform. Another phase of studies will use VSV-SARS-CoV-2 viruses lacking the eGFP reporter gene. AdV-hACE2 transduction could affect the anamnestic response in unpredictable ways because it occurs close to the time of SARS-CoV-2 challenge. It is noted that viral yields from organs of VSV-eGFP-control-vaccinated animals were nearly identical to those observed in SARS-CoV-2-infected unvaccinated animals (Hassan et al., 2020). More importantly, we showed robust immunogenicity of VSV-eGFP-SARS-CoV-2 in K18-hACE2 transgenic mice and that passive transfer of immune sera from VSV-eGFP-SARS-CoV-2 vaccinated to naive mice contributes to protection against SARS-CoV-2 challenge. These results indicate that humoral immunity generated by the VSV-eGFP-SARS-CoV-2 vaccine is not affected substantively by the AdV-hACE2 transduction process itself.

| | | |
|---|---|---|
| REAGENT or RESOURCE | SOURCE | IDENTIFIER |

Antibodies

| | | |
|---|---|---|
| Anti-human IgG peroxidase | Sigma-Aldrich | Cat# A6029-1ML; RRID: |
| | | AB_258272 |
| CR3022 | ter Meulen et al., 2006; | N/A |
| | Yuan et al., 2020 | |
| MAR1-5A3, anti-Ifnar1 mAb | Leinco | I-401; RRID: AB_2491621 |
| Goat Anti-Mouse IgM, Human ads-HRP | SouthernBiotech | 1020-05; RRID: |
| | | AB_2794201 |
| Goat Anti-Mouse IgA-HRP | SouthernBiotech | 1040-05; RRID: |
| | | AB_2714213 |
| Goat Anti-Mouse IgG, Human ads-HRP | SouthernBiotech | 1030-05; RRID: |
| | | AB_2619742 |
| Goat Anti-Mouse IgG1, Human ads-BIOT | SouthernBiotech | 1070-08; RRID: |
| | | AB_2794413 |
| Goat Anti-Mouse IgG2b, Human ads-BIOT | SouthernBiotech | 1090-08; RRID: |
| | | AB_2794523 |
| Goat Anti-Mouse IgG3, Human ads-BIOT | SouthernBiotech | 1100-08; RRID: |
| | | AB_2794575 |
| Goat Anti-Mouse IgG2c, Human ads-BIOT | SouthernBiotech | 1079-08; RRID: |
| | | AB_2794467 |
| Streptavidin-HRP | Invitrogen | 434323; RRID: |
| | | AB_2619743 |

Bacterial and Virus Strains

| | | |
|---|---|---|
| 2019 n-CoV/USA_WA1/2020 | CDC (gift from Natalie | N/A |
| | Thornburg) | |
| AdV-hACE2 | Jia et al., 2005 | N/A |
| Vaccinia virus vTF7-3 | Fuerst et al., 1986 | N/A |
| VSV-eGFP | Chandran et al., 2005 | N/A |

Chemicals, Peptides, and Recombinant Proteins

| | | |
|---|---|---|
| Recombinant SARS-CoV-2 spike protein | Alsoussi et al. | N/A |
| Recombinant SARS-CoV-2 RBD protein | Alsoussi et al. | N/A |
| Blasticidin S HCl | GIBCO/Thermo Fisher | Cat# A1113903 |
| Bromophenol Blue | Millipore Sigma | Cat# BX1410-7 |
| PNGase F | New England Biolabs | Cat# P0704S |
| Cytosine arabinoside | Sigma-Aldrich | Cat# C1768 |
| Formaldehyde Solution | Millipore Sigma | Cat# FX0410-5 |
| HEPES, free acid | Millipore Sigma | Cat #5310-OP |
| Lipofectamine 2000 Transfection Reagent | Invitrogen/Thermo | Cat# 11668019 |
| | Fisher | |
| 1-Step ™ Ultra TMB-ELISA Substrate | ThermoFisher | 34028 |
| Solution | Scientific | |
| TrueBlue peroxidase substrate | KPL/SeraCare | Cat# 5510-0050 |

Experimental Models: Cell Lines

| | | |
|---|---|---|
| BSRT7/5 | Buchholz et al., 1999 | N/A |
| MA-104 | ATCC | Cat# CRL-2378.1: RRID: |
| | | CVCL_3846 |
| Vero CCL81 | ATCC | Cat# CCL-81: RRID: |
| | | CVCL_0059 |
| Vero E6 | ATCC | Cat# CRL-1586: RRID: |
| | | CVCL_0574 |
| Vero-furin | Mukherjee et al., 2016 | N/A |

Experimental Models: Organisms/Strains

| | | |
|---|---|---|
| Mouse: BALB/c | Jackson Laboratory | Cat#000651; RRID: |
| | | IMSR_JAX: 000651 |
| Mouse: B6.Cg-Tg(K18-ACE2)2Prlmn/J | Jackson Laboratory | Cat#034860; |
| | | IMSR_JAX: 034860 |

Oligonucleotides

| | | |
|---|---|---|
| SARS-CoV-2 N F: 5′- | Hassan et al., 2020 | N/A |
| ATGCTGCAATCGTGCTACAA-3′ | | |
| SARS-CoV-2 N R: 5′- | Hassan et al., 2020 | N/A |
| GACTGCCGCCTCTGCTC-3′ | | |
| SARS-CoV-2 N Probe: 5′-/56- | Hassan et al., 2020 | N/A |
| FAM/TCAAGGAAC/ZEN/ | | |
| AACATTGCCAA/3IABKFQ/-3′ | | |
| SARS-CoV-2 RNA ISH probe (S gene) | Advanced Cell | Cat# 4848561 |
| | Diagnostics | |
| Gapdh TaqMan Primer/Probe set | IDT | Mm.PT.39a.1 |
| Ifng TaqMan Primer/Probe set | IDT | Mm.PT.58.41769240 |
| Il6 TaqMan Primer/Probe set | IDT | Mm.PT.58.10005566 |
| Il1b TaqMan Primer/Probe set | IDT | Mm.PT.58.41616450 |
| Tnfa TaqMan Primer/Probe set | IDT | Mm.PT.58.12575861 |
| Cxcl10 TaqMan Primer/Probe set | IDT | Mm.PT.58.43575827 |
| Cxcl11 TaqMan Primer/Probe set | IDT | Mm.PT.58.10773148.g |
| Ifnb TaqMan Primer/Probe set | IDT | Mm.PT.58.30132453.g |
| Ifnl(2/3) TaqMan Primer/Probe set | Thermo Fisher | Mm04204156_gH |
| CXCL1 | IDT | Mm.PT.58.42076891 |

Recombinant DNA

| | | |
|---|---|---|
| pCAGGS-VSV-G | Stanifer et al., 2011 | N/A |
| PGEM3-VSV L | Whelan et al., 1995 | N/A |
| pGEM3-VSV G | Stanifer et al., 2011 | N/A |
| PGEM3-VSV N | Whelan et al., 1995 | N/A |
| pGEM3-VSV P | Whelan et al., 1995 | N/A |
| pVSV-eGFP | Chandran et al., 2005 | N/A |
| pVSV-eGFP-SARS-CoV-2-S_{AA} | Case et al., 2020 | Spike mutated from |
| | | MN908947.3 |

Software and Algorithms

| | | |
|---|---|---|
| BioRender | Biorender.com | N/A |
| Statistics: Prism 8.0 | GraphPad | N/A |

Experimental Model and Subject Details

Cells

BSRT7/5, Vero CCL81, Vero E6, Vero E6-TMPRSS2 (Case et al., 2020), and Vero-furin (Mukherjee et al., 2016) cells were maintained in humidified incubators at 34 or 37° C. and 5% CO₂ in DMEM (Corning) supplemented with glucose, L-glutamine, sodium pyruvate, and 10% fetal bovine serum (FBS). MA104 cells were maintained similarly but in Medium 199 (GIBCO).

Plasmids

The S gene of SARS-CoV-2 isolate Wuhan-Hu-1 (GenBank MN908947.3) was cloned into the backbone of the infectious molecular clone of VSV containing eGFP (pVSV-eGFP) as described (Case et al., 2020). pVSV-eGFP was used as previously described, but contains a mutation K535R, the phenotype of which will be described elsewhere. Expression plasmids of VSV N, P, L, and G were previously described (Stanifer et al., 2011; Whelan et al., 1995).

Recombinant VSV (rVSV)

VSV-eGFP-SARS-CoV-2 and VSV-eGFP were generated and rescued as described previously (Case et al., 2020; Whelan et al., 1995). Briefly, BSRT7/5 cells (Buchholz et al., 1999) were infected with vaccinia virus encoding the bacteriophage T7 RNA polymerase (vTF7-3) (Fuerst et al., 1986) and subsequently transfected with plasmids encoding VSV N, P, L, G, and an antigenome copy of the viral genome under control of the T7 promoter. Rescue supernatants were collected 56 to 72 h post-transfection, clarified by centrifugation (5 min at 1,000×g), and filtered through a 0.22 μm filter. Virus clones were plaque-purified on Vero CCL81 cells containing 25 μg/mL of cytosine arabinoside (Sigma-Aldrich) in the agarose overlay, and plaques were amplified on Vero CCL81 cells. All infections for generating stocks were performed at 37° C. for 1 h and at 34° C. thereafter. Viral supernatants were harvested upon extensive cytopathic effect and clarified of cell debris by centrifugation at 1,000×g for 5 min. Aliquots were maintained at −80° C.

Mouse Experiments

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anesthesia that was induced and maintained with ketamine hydrochloride and xylazine, and all efforts were made to minimize animal suffering.

At four weeks of age, female BALB/c mice (Jackson Laboratory, 000651) were immunized with 10⁶ PFU of VSV-eGFP-SARS-CoV-2 or VSV-eGFP in a total volume of 200 μL per mouse via the intraperitoneal route. In some immunogenicity experiments, heterozygous K18-hACE2 C57BL/6J mice (strain: 2B6.Cg-Tg(K18-ACE2)2Prlmn/J) were obtained (Jackson Laboratory, 034860) and immunized with 10⁶ PFU of VSV-eGFP-SARS-CoV-2 or VSV-eGFP via an intranasal route. Where indicated, mice were boosted with homologous virus at 4 weeks post-priming. Three weeks post-priming or boosting mice were administered 2 mg of anti-Ifnar1 mAb (MAR1-5A3 (Sheehan et al., 2006), Leinco) via intraperitoneal injection. One day later, mice were administered 2.5×10⁶ PFU of mouse codon-optimized AdV-hACE2 (Hassan et al., 2020) in a total volume of 50 μL per mouse via intranasal administration. Five days later, vaccinated mice were challenged with 3×10⁵ PFU of SARS-CoV-2 in a total volume of 75 μL per mouse via intranasal administration. Passive transfer experiments were conducted as described above but using ten-week-old female BALB/c mice. One-hundred microliters of pooled immune sera were administered to mice in each respective group 24 h prior to SARS-CoV-2 challenge. For each immunization (prime or boost), blood from individual mice was collected twice (at days 14 and 22) from the submandibular vein. After clotting of blood at room temperature, serum was obtained and pooled.

Method Details

Gradient Purification of Recombinant Viruses

To generate high titer stocks of VSV-eGFP, virus was grown on BSRT7/5 cells at an MOI of 3. VSV-eGFP-SARS-CoV-2 was grown on BSRT7/5 cells in the presence of VSV G, or on MA104 cells in the absence of VSV G, at MOIs of 1. To generate VSV-eGFP-SARS-CoV-2+VSV G, BSRT7/5 cells were transfected with pCAGGS-VSV-G in Opt-MEM (GIBCO) using Lipofectamine 2000 (Invitrogen) and subsequently infected 8 to 12 h later with VSV-eGFP-SARS-CoV-2 at an MOI of 0.01 in DMEM containing 2% FBS and 20 mM HEPES pH 7.7. This VSV G decorated VSV-eGFP-SARS-CoV-2 was titrated by plaque assay and used for a larger scale infection as described above. Cell supernatants were collected after 48 h and clarified by centrifugation at 1,000×g for 7.5 min. Supernatants were concentrated using a Beckman Optima L-100 XP ultracentrifuge (22,800 RPM×90 min in a 70Ti fixed-angle rotor). Pellets were resuspended in 100 mM NaCl, 10 mM Tris pH 7.4, 1 mM EDTA (NTE) at 4° C. overnight, and virus was banded on a 15%-45% sucrose-NTE gradient (35,000 rpm×3 h in a SW-41Ti swinging-bucket rotor). Virus was extracted by side puncture of tubes, recovered by ultracentrifugation (22,800 RPM×90 min in a 70Ti fixed-angle rotor) and resuspended in NTE at 4° C. overnight. To determine the protein content of purified virions, samples were treated with PNGase F (New England Biolabs) according to the manufacturer's protocol to remove N-linked glycans. Samples were processed by SDS-PAGE under denaturing (100° C., 5 min) and reducing conditions (4×SDS-loading buffer containing 200 mM Tris-HCl pH 6.8, 400 mM dithiothreitol, 8% SDS, 0.4% Bromophenol Blue (Millipore Sigma), and 40% glycerol), and visualized by Coomassie staining.

Measurement of Viral Burden

Mouse tissues were weighed and homogenized with sterile zirconia beads in a MagNA Lyser instrument (Roche Life Science) in 1 mL of DMEM media supplemented to contain 2% heat-inactivated FBS. Tissue homogenates were clarified by centrifugation at 10,000 rpm for 5 min and stored at −80° C. RNA was extracted using MagMax mirVana Total RNA isolation kit (Thermo Scientific) and a Kingfisher Flex extraction machine (Thermo Scientific). Infectious viral titers in lung homogenates were determined by plaque assays on Vero-furin cells. Viral RNA levels were determined by RT-qPCR as described (Hassan et al., 2020) and normalized to tissue weight.

Cytokine Analysis

Total RNA was isolated from lung homogenates as described above and DNAase treated. cDNA was generated using the HighCapacity cDNA Reverse Transcription kit (Thermo Scientific) with the addition of RNase inhibitor according to the manufacturer's instructions. Cytokine and chemokine expression were determined using TaqMan Fast Universal PCR master mix (Thermo Scientific) with commercially available primer/probe sets specific for IFN-γ (IDT: Mm.PT.58.41769240), IL-6 (Mm.PT.58.10005566), IL-1β (Mm.PT.58.41616450), TNF-α (Mm.PT.58.12575861), CXCL10 (Mm.PT.58.43575827), CCL2 (Mm.PT.58.42151692), CCL5 (Mm.PT.58.43548565), CXCL11 (Mm.PT.58.10773148.g), IFN-β (Mm.PT.58.30132453.g), and IFNλ-2/3 (Thermo Scientific Mm04204156_gH). All results were normalized to GAPDH (Mm.PT.39a.1) levels and the fold-change for each was determined using the 2^{-ΔΔCt} method comparing SARS-CoV-2 infected mice to naive controls.

Histology and In Situ Hybridization

Mice were euthanized, and tissues were harvested prior to lung inflation and fixation. The right lung was inflated with approximately 1.2 mL of 10% neutral buffered formalin using a 3-mL syringe and catheter inserted into the trachea. To ensure fixation of virus, inflated lungs were kept in a 40-mL suspension of neutral buffered formalin for 7 days before further processing. Tissues were paraffin-embedded and 5 μm sections were subsequently stained with hematoxylin and eosin. RNA in situ hybridization was performed using the RNAscope 2.5 HD Assay (Brown Kit) according to the manufacturer's instructions (Advanced Cell Diagnostics). Briefly, sections were deparaffinized and treated with H₂O₂ and Protease Plus prior to RNA probe hybridization. Probes specifically targeting SARS-CoV-2 S sequence (cat no 848561) were hybridized followed by signal amplification and detection with 3,3′-Diaminobenzidine. Tissues were counterstained with Gill's hematoxylin and an uninfected mouse was stained in parallel and used as a negative control. Pathology was evaluated from 3 lungs per group, and representative photomicrographs of 10 fields per slide were taken under investigator-blinded conditions. Tissue sections were visualized using a Nikon Eclipse microscope equipped with an Olympus DP71 color camera or a Leica DM6B microscope equipped with a Leica DFC7000T camera using 40×, 200×, or 400× magnification.

Neutralization Assay

Serial dilutions of mouse sera were incubated with 10² focus-forming units (FFU) of SARS-CoV-2 for 1 h at 37° C. Antibody-virus complexes were added to Vero E6 cell monolayers in 96-well plates and incubated at 37° C. for 1 h. Subsequently, cells were overlaid with 1% (w/v) methylcellulose in MEM supplemented with 2% FBS. Plates were harvested 30 h later by removing overlays and fixed with 4% PFA in PBS for 20 min at room temperature. Plates were washed and sequentially incubated with 1 mg/mL of CR3022 (Yuan et al., 2020) 32245784anti-S antibody and HRP-conjugated goat anti-human IgG in PBS supplemented with 0.1% saponin and 0.1% bovine serum albumin. SARS-CoV-2-infected cell foci were visualized using TrueBlue peroxidase substrate (KPL) and quantitated on an ImmunoSpot microanalyzer (Cellular Technologies). Data were processed using Prism software (GraphPad Prism 8.0).

Protein Expression and Purification

Purified RNA from the 2019-nCoV/USA-WA1/2020 SARS-CoV-2 strain was reverse transcribed into cDNA and used as the template for recombinant gene cloning. SARS-CoV-2 RBD and S ectodomain (the S1/S2 furin cleavage site was disrupted, double proline mutations were introduced into the S2 subunit, and foldon trimerization motif was incorporated) were cloned into pFM1.2 with a C-terminal hexahistidine or octahistidine tag, transiently transfected into Expi293F cells, and purified by cobalt-charged resin chromatography (G-Biosciences) as described (Alsoussi et al., 2020).

ELISA

Six-well Maxisorp plates were coated with 2 ug/mL of either SARS-CoV-2 S or RBD protein in 50 mM Na₂CO₃ (70 μL) overnight at 4° C. Plates were then washed with PBS+0.05% Tween-20 and blocked with 200 μL of 1×PBS+0.05% Tween-20+1% BSA+0.02% NaN₃ for 2 h at room temperature (RT). Serum samples were serially diluted (1:3) starting at either 1:100 dilution (day 22 samples; BALB/c) or 1:100 dilution (day 21 samples; K18-hACE2) in blocking buffer. Diluted samples were added to washed plates (50 μL/well) and incubated for 1 h at RT. Bound IgG was detected using HRP-conjugated goat anti-mouse IgG (at 1:2000); bound IgM was detected using biotin-conjugated anti-mouse IgM (at 1:10000); bound IgA was detected using HRP-conjugated goat anti-mouse IgA (at 1:2000); bound IgG1 was detected using biotin-conjugated anti-mouse IgG1 (at 1:10000); bound IgG2b was detected using biotin-conjugated anti-mouse IgG2b (at 1:10000); bound IgG2c was detected using biotin-conjugated anti-mouse IgG2c (at 1:10000); and bound IgG3 was detected using biotin-conjugated anti-mouse IgG3 (at 1:10000). After washing, all plates were incubated with streptavidin-HRP (at 1:5000). Following a 1 h incubation, washed plates were developed with 50 μL of 1-Step Ultra TMB-ELISA, quenched with 2 M sulfuric acid, and the absorbance was read at 450 nm.

Quantification and Statistical Analysis

Statistical significance was assigned when P values were <0.05 using Prism Version 8.2 software (GraphPad). Tests, number of animals (n), median values, and statistical comparison groups are indicated in the Figure legends. Analysis of anti-S, anti-RBD, and neutralization titers in mice after vaccination was performed using a one-way ANOVA with Dunnett's post-test. Differences in IgG subclasses were evaluated using the Mann-Whitney test. Differences in viral titers or chemokine and cytokine levels after SARS-CoV-2 infection of immunized mice were determined using a Kruskal-Wallis test with Dunn's post-test.

### REFERENCES

W. B. Alsoussi, J. S. Turner, J. B. Case, H. Zhao, A. J. Schmitz, J. Q. Zhou, R. E. Chen, T. Lei, A. A. Rizk, K. M. McIntire, et al. A Potently Neutralizing Antibody Protects Mice against SARS-CoV-2 Infection J. Immunol., 205 (2020), pp. 915-922
F. Amanat, F. Krammer SARS-CoV-2 Vaccines: Status Report Immunity, 52 (2020), pp. 583-589
Y. Bai, L. Yao, T. Wei, F. Tian, D. Y. Jin, L. Chen, M. Wang Presumed Asymptomatic Carrier Transmission of COVID-19 JAMA (2020)
L. A. Ball, C. R. Pringle, B. Flanagan, V. P. Perepelitsa, G. W. Wertz Phenotypic consequences of rearranging the P, M, and G genes of vesicular stomatitis virus J. Virol., 73 (1999), pp. 4705-4712
L. Bao, W. Deng, B. Huang, H. Gao, J. Liu, L. Ren, Q. Wei, P. Yu, Y. Xu, F. Qi, et al. The pathogenicity of SARS-CoV-2 in hACE2 transgenic mice Nature, 583 (2020), pp. 830-833
D. H. Barouch, M. G. Pau, J. H. Custers, W. Koudstaal, S. Kostense, M. J. Havenga, D. M. Truitt, S. M. Sumida, M. G. Kishko, J. C. Arthur, et al. Immunogenicity of recombinant adenovirus serotype 35 vaccine in the presence of pre-existing anti-Ad5 immunity J. Immunol., 172 (2004), pp. 6290-6297
A. Baum, B. O. Fulton, E. Wloga, R. Copin, K. E. Pascal, V. Russo, S. Giordano, K. Lanza, N. Negron, M. Ni, et al. Antibody cocktail to SARS-CoV-2 spike protein prevents rapid mutational escape seen with individual antibodies Science (2020), p. eabd0831
K. S. Brown, D. Safronetz, A. Marzi, H. Ebihara, H. Feldmann Vesicular stomatitis virus-based vaccine protects hamsters against lethal challenge with Andes virus J. Virol., 85 (2011), pp. 12781-12791
U. J. Buchholz, S. Finke, K. K. Conzelmann Generation of bovine respiratory syncytial virus (BRSV) from cDNA: BRSV NS2 is not essential for virus replication in tissue culture, and the human RSV leader region acts as a functional BRSV genome promoter J. Virol., 73 (1999), pp. 251-259
J. B. Case, P. W. Rothlauf, R. E. Chen, Z. Liu, H. Zhao, A. S. Kim, L. M. Bloyet, Q. Zeng, S. Tahan, L. Droit, et al. Neutralizing antibody and soluble ACE2 inhibition of a replication-competent VSV-SARS-CoV-2 and a clinical isolate of SARS-CoV-2 Cell Host Microbe (2020) S1931-3128(20)30362-0, in press
D. R. Casimiro, L. Chen, T. M. Fu, R. K. Evans, M. J. Caulfield, M. E. Davies, A. Tang, M. Chen, L. Huang, V. Harris, et al. Comparative immunogenicity in rhesus monkeys of DNA plasmid, recombinant vaccinia virus, and replication-defective adenovirus vectors expressing a human immunodeficiency virus type 1 gag gene J. Virol., 77 (2003), pp. 6305-6313
K. Chandran, N. J. Sullivan, U. Felbor, S. P. Whelan, J. M. Cunningham Endosomal proteolysis of the Ebola virus glycoprotein is necessary for infection Science, 308 (2005), pp. 1643-1645
X. Chi, R. Yan, J. Zhang, G. Zhang, Y. Zhang, M. Hao, Z. Zhang, P. Fan, Y. Dong, Y. Yang, et al. A neutralizing human antibody binds to the N-terminal domain of the Spike protein of SARS-CoV-2 Science, 369 (2020), pp. 650-655
B. L. Cline Ecological associations of vesicular stomatitis virus in rural Central America and Panama Am. J. Trop. Med. Hyg., 25 (1976), pp. 875-883
A. Dutta, C. T. Huang, C. Y. Lin, T. C. Chen, Y. C. Lin, C. S. Chang, Y. C. He Sterilizing immunity to influenza virus infection requires local antigen-specific T cell response in the lungs Sci. Rep., 6 (2016), p. 32973
A. Fathi, C. Dahlke, M. M. Addo Recombinant vesicular stomatitis virus vector vaccines for WHO blueprint priority pathogens Hum. Vaccin. Immunother., 15 (2019), pp. 2269-2285
T. R. Fuerst, E. G. Niles, F. W. Studier, B. Moss Eukaryotic transient-expression system based on recombinant vaccinia virus that synthesizes bacteriophage T7 RNA polymerase Proc. Natl. Acad. Sci. USA, 83 (1986), pp. 8122-8126
W. Furuyama, P. Reynolds, E. Haddock, K. Meade-White, M. Quynh Le, Y. Kawaoka, H. Feldmann, A. Marzi A single dose of a vesicular stomatitis virus-based influenza vaccine confers rapid protection against H5 viruses from different clades NPJ Vaccines, 5 (2020), p. 4
Q. Gao, L. Bao, H. Mao, L. Wang, K. Xu, M. Yang, Y. Li, L. Zhu, N. Wang, Z. Lv, et al. Rapid development of an inactivated vaccine candidate for SARS-CoV-2 Science (2020)
M. Garbutt, R. Liebscher, V. Wahl-Jensen, S. Jones, P. Möller, R. Wagner, V. Volchkov, H. D. Klenk, H. Feldmann, U. Stroher Properties of replication-competent vesicular stomatitis virus vectors expressing glycoproteins of filoviruses and arenaviruses J. Virol., 78 (2004), pp. 5458-5465
T. W. Geisbert, S. Jones, E. A. Fritz, A. C. Shurtleff, J. B. Geisbert, R. Liebscher, A. Grolla, U. Stroher, L. Fernando, K. M. Daddario, et al. Development of a new vaccine for the prevention of Lassa fever PLoS Med., 2 (2005), p. e183
T. W. Geisbert, K. M. Daddario-Dicaprio, M. G. Lewis, J. B. Geisbert, A. Grolla, A. Leung, J. Paragas, L. Matthias, M. A. Smith, S. M. Jones, et al. Vesicular stomatitis virus-based ebola vaccine is well-tolerated and protects immunocompromised nonhuman primates PLoS Pathog., 4 (2008), p. e1000225
A. Grifoni, D. Weiskopf, S. I. Ramirez, J. Mateus, J. M. Dan, C. R. Moderbacher, S. A. Rawlings, A. Sutherland, L. Premkumar, R. S. Jadi, et al. Targets of T Cell Responses to SARS-CoV-2 Coronavirus in Humans with COVID-19 Disease and Unexposed Individuals Cell, 181 (2020), pp. 1489-1501.e15
J. Gu, C. Korteweg Pathology and pathogenesis of severe acute respiratory syndrome Am. J. Pathol., 170 (2007), pp. 1136-1147
A. O. Hassan, J. B. Case, E. S. Winkler, L. B. Thackray, N. M. Kafai, A. L. Bailey, B. T. McCune, J. M. Fox, R. E. Chen, W. B. Alsoussi, et al. A SARS-CoV-2 Infection Model in Mice Demonstrates Protection by Neutralizing Antibodies Cell, 182 (2020), pp. 744-753.e4
A. M. Henao-Restrepo, I. M. Longini, M. Egger, N. E. Dean, W. J. Edmunds, A. Camacho, M. W. Carroll, M. Doumbia, B. Draguez, S. Duraffour, et al. Efficacy and effectiveness of an rVSV-vectored vaccine expressing Ebola surface glycoprotein: interim results from the Guinea ring vaccination cluster-randomised trial Lancet, 386 (2015), pp. 857-866
A. M. Henao-Restrepo, A. Camacho, I. M. Longini, C. H. Watson, W. J. Edmunds, M. Egger, M. W. Carroll, N. E. Dean, I. Diatta, M. Doumbia, et al. Efficacy and effectiveness of an rVSV-vectored vaccine in preventing Ebola virus disease: final results from the Guinea ring vaccination, open-label, cluster-randomised trial (Ebola ça Suffit!) Lancet, 389 (2017), pp. 505-518
C. Huang, Y. Wang, X. Li, L. Ren, J. Zhao, Y. Hu, L. Zhang, G. Fan, J. Xu, X. Gu, et al. Clinical features of patients infected with 2019 novel coronavirus in Wuhan, China Lancet, 395 (2020), pp. 497-506
A. Huttner, J. A. Dayer, S. Yerly, C. Combescure, F. Auderset, J. Desmeules, M. Eickmann, A. Finckh, A. R. Goncalves, J. W. Hooper, et al., VSV-Ebola Consortium The effect of dose on the safety and immunogenicity of the VSV Ebola candidate vaccine: a randomised double-blind, placebo-controlled phase 1/2 trial Lancet Infect. Dis., 15 (2015), pp. 1156-1166
M. Imai, K. Iwatsuki-Horimoto, M. Hatta, S. Loeber, P. J. Halfmann, N. Nakajima, T. Watanabe, M. Ujie, K. Takahashi, M. Ito, et al. Syrian hamsters as a small animal model for SARS-CoV-2 infection and countermeasure development Proc. Natl. Acad. Sci. USA, 117 (2020), pp. 16587-16595
H. P. Jia, D. C. Look, L. Shi, M. Hickey, L. Pewe, J. Netland, M. Farzan, C. Wohlford-Lenane, S. Perlman, McCray P. B. ACE2 receptor expression and severe acute respiratory syndrome coronavirus infection depend on differentiation of human airway epithelia J Virol., 79 (2005), pp. 14614-14621
R. D. Jiang, M. Q. Liu, Y. Chen, C. Shan, Y. W. Zhou, X. R. Shen, Q. Li, L. Zhang, Y. Zhu, H. R. Si, et al. Pathogenesis of SARS-CoV-2 in Transgenic Mice Expressing Human Angiotensin-Converting Enzyme 2 Cell, 182 (2020), pp. 50-58.e8
K. M. Johnson, J. E. Vogel, P. H. Peralta Clinical and serological response to laboratory-acquired human infection by Indiana type vesicular stomatitis virus (VSV) Am. J. Trop. Med. Hyg., 15 (1966), pp. 244-246
S. M. Jones, H. Feldmann, U. Stroher, J. B. Geisbert, L. Fernando, A. Grolla, H. D. Klenk, N. J. Sullivan, V. E. Volchkov, E. A. Fritz, et al. Live attenuated recombinant vaccine protects nonhuman primates against Ebola and Marburg viruses Nat. Med., 11 (2005), pp. 786-790
S. U. Kapadia, I. D. Simon, J. K. Rose SARS vaccine based on a replication-defective recombinant vesicular stomatitis virus is more potent than one based on a replication-competent vector Virology, 376 (2008), pp. 165-172
S. B. Kennedy, F. Bolay, M. Kieh, G. Grandits, M. Badio, R. Ballou, R. Eckes, M. Feinberg, D. Follmann, B. Grund, et al., PREVAIL I Study Group Phase 2 Placebo-Controlled Trial of Two Vaccines to Prevent Ebola in Liberia N. Engl. J. Med., 377 (2017), pp. 1438-1447
M. Letko, A. Marzi, V. Munster Functional assessment of cell entry and receptor usage for SARS-CoV-2 and other lineage B betacoronaviruses Nat. Microbiol., 5 (2020), pp. 562-569
J. Li, J. T. Wang, S. P. Whelan A unique strategy for mRNA cap methylation used by vesicular stomatitis virus Proc. Natl. Acad. Sci. USA, 103 (2006), pp. 8493-8498
B. D. Lichty, A. T. Power, D. F. Stojdl, J. C. Bell Vesicular stomatitis virus: re-inventing the bullet Trends Mol. Med., 10 (2004), pp. 210-216
N. Lurie, M. Saville, R. Hatchett, J. Halton Developing Covid-19 Vaccines at Pandemic Speed N. Engl. J. Med., 382 (2020), pp. 1969-1973
Y. Ma, Y. Wei, X. Zhang, Y. Zhang, H. Cai, Y. Zhu, K. Shilo, M. Oglesbee, S. Krakowka, S. P. Whelan, J. Li mRNA cap methylation influences pathogenesis of vesicular stomatitis virus in vivo J. Virol., 88 (2014), pp. 2913-2926
I. Martinez, L. L. Rodriguez, C. Jimenez, S. J. Pauszek, G. W. Wertz Vesicular stomatitis virus glycoprotein is a determinant of pathogenesis in swine, a natural host J. Virol., 77 (2003), pp. 8039-8047
P. B. McCray Jr., L. Pewe, C. Wohlford-Lenane, M. Hickey, L. Manzel, L. Shi, J. Netland, H. P. Jia, C. Halabi, C. D. Sigmund, et al. Lethal infection of K18-hACE2 mice infected with severe acute respiratory syndrome coronavirus J. Virol., 81 (2007), pp. 813-821
C. E. Mire, A. D. Miller, A. Carville, S. V. Westmoreland, J. B. Geisbert, K. G. Mansfield, H. Feldmann, L. E. Hensley, T. W. Geisbert Recombinant vesicular stomatitis virus vaccine vectors expressing filovirus glycoproteins lack neurovirulence in nonhuman primates PLoS Negl. Trop. Dis., 6 (2012), p. e1567
A. Muik, L. J. Stubbert, R. Z. Jahedi, Y. Geip, J. Kimpel, C. Dold, R. Tober, A. Volk, S. Klein, U. Dietrich, et al. Re-engineering vesicular stomatitis virus to abrogate neurotoxicity, circumvent humoral immunity, and enhance oncolytic potency Cancer Res., 74 (2014), pp. 3567-3578
S. Mukherjee, D. Sirohi, K. A. Dowd, Z. Chen, M. S. Diamond, R. J. Kuhn, T. C. Pierson Enhancing dengue virus maturation using a stable furin over-expressing cell line Virology, 497 (2016), pp. 33-40
D. Pinto, Y. J. Park, M. Beltramello, A. C. Walls, M. A. Tortorici, S. Bianchi, S. Jaconi, K. Culap, F. Zatta, A. De Marco, et al. Cross-neutralization of SARS-CoV-2 by a human monoclonal SARS-CoV antibody Nature, 583 (2020), pp. 290-295
S. G. Rabinowitz, J. Huprikar, M. C. Dal Canto Comparative neurovirulence of selected vesicular stomatitis virus temperature-sensitive mutants of complementation groups II and Ill Infect. Immun., 33 (1981), pp. 120-125
A. Roberts, L. Buonocore, R. Price, J. Forman, J. K. Rose Attenuated vesicular stomatitis viruses as vaccine vectors J. Virol., 73 (1999), pp. 3723-3732
T. F. Rogers, F. Zhao, D. Huang, N. Beutler, A. Burns, W. T. He, O. Limbo, C. Smith, G. Song, J. Woehl, et al. Isolation of potent SARS-CoV-2 neutralizing antibodies and protection from disease in a small animal model Science (2020), p. eabc7520
S. Santra, Y. Sun, J. G. Parvani, V. Philippon, M. S. Wyand, K. Manson, A. Gomez-Yafal, G. Mazzara, D. Panicali, P. D. Markham, et al. Heterologous prime/boost immunization of rhesus monkeys by using diverse poxvirus vectors J. Virol., 81 (2007), pp. 8563-8570
K. C. Sheehan, K. S. Lai, G. P. Dunn, A. T. Bruce, M. S. Diamond, J. D. Heutel, C. Dungo-Arthur, J. A. Carrero, J. M. White, P. J. Hertzog, R. D. Schreiber Blocking monoclonal antibodies specific for mouse IFN-alpha/beta receptor subunit 1 (IFNAR-1) from mice immunized by in vivo hydrodynamic transfection J. Interferon Cytokine Res., 26 (2006), pp. 804-819
S. F. Sia, L. M. Yan, A. W. H. Chin, K. Fung, K. T. Choy, A. Y. L. Wong, P. Kaewpreedee, R. A. P. M. Perera, L. L. M. Poon, J. M. Nicholls, et al. Pathogenesis and transmission of SARS-CoV-2 in golden hamsters Nature, 583 (2020), pp. 834-838
M. L. Stanifer, D. K. Cureton, S. P. Whelan A recombinant vesicular stomatitis virus bearing a lethal mutation in the glycoprotein gene uncovers a second site suppressor that restores fusion J. Virol., 85 (2011), pp. 8105-8115
S. H. Sun, Q. Chen, H. J. Gu, G. Yang, Y. X. Wang, X. Y. Huang, S. S. Liu, N. N. Zhang, X. F. Li, R. Xiong, et al. A Mouse Model of SARS-CoV-2 Infection and Pathogenesis Cell Host Microbe, 28 (2020), pp. 124-133.e4
J. ter Meulen, E. N. van den Brink, L. L. Poon, W. E. Marissen, C. S. Leung, F. Cox, C. Y. Cheung, A. Q. Bakker, J. A. Bogaards, E. van Deventer, et al. Human monoclonal antibody combination against SARScoronavirus: synergy and coverage of escape mutants PLoS Med, 3 (2006), Article e237, 10.1371/journal.pmed.0030237
A. N. van den Pol, G. Mao, A. Chattopadhyay, J. K. Rose, J. N. Davis Chikungunya, Influenza, Nipah, and Semliki Forest Chimeric Viruses with Vesicular Stomatitis Virus: Actions in the Brain J. Virol., 91 (2017), p. 91
B. Wang, C. Yang, G. Tekes, S. Mueller, A. Paul, S. P. Whelan, E. Wimmer Recoding of the vesicular stomatitis virus L gene by computer-aided design provides a live, attenuated vaccine candidate MBio, 6 (2015), p. 6
G. W. Wertz, V. P. Perepelitsa, L. A. Ball Gene rearrangement attenuates expression and lethality of a nonsegmented negative strand RNA virus Proc. Natl. Acad. Sci. USA, 95 (1998), pp. 3501-3506
S. P. Whelan, L. A. Ball, J. N. Barr, G. T. Wertz Efficient recovery of infectious vesicular stomatitis virus entirely from cDNA clones Proc. Natl. Acad. Sci. USA, 92 (1995), pp. 8388-8392
S. P. Whelan, J. N. Barr, G. W. Wertz Identification of a minimal size requirement for termination of vesicular stomatitis virus mRNA: implications for the mechanism of transcription J. Virol., 74 (2000), pp. 8268-8276
G. Wollmann, E. Drokhlyansky, J. N. Davis, C. Cepko, A. N. van den Pol Lassa-vesicular stomatitis chimeric virus safely destroys brain tumors J. Virol., 89(2015), pp. 6711-6724
J. Yu, L. H. Tostanoski, L. Peter, N. B. Mercado, K. McMahan, S. H. Mahrokhian, J. P. Nkolola, J. Liu, Z. Li, A. Chandrashekar, et al. DNA vaccine protection against SARS-CoV-2 in rhesus macaques Science (2020), p. eabc6284
M. Yuan, N. C. Wu, X. Zhu, C. D. Lee, R. T. Y. So, H. Lv, C. K. P. Mok, I. A. Wilson A highly conserved cryptic epitope in the receptor binding domains of SARS-CoV-2 and SARS-CoV Science, 368 (2020), pp. 630-633
F. C. Zhu, Y. H. Li, X. H. Guan, L. H. Hou, W. J. Wang, J. X. Li, S. P. Wu, B. S. Wang, Z. Wang, L. Wang, et al. Safety, tolerability, and immunogenicity of a recombinant adenovirus type-5 vectored COVID-19 vaccine: a dose-escalation, open-label, non-randomised, first-in-human trial Lancet, 395 (2020), pp. 1845-1854

### Example 3: Vaccine Constructs and Variants

This example describes additional vaccine data for the original vaccine construct as well as several variants that have been generated and are being evaluated as vaccines.

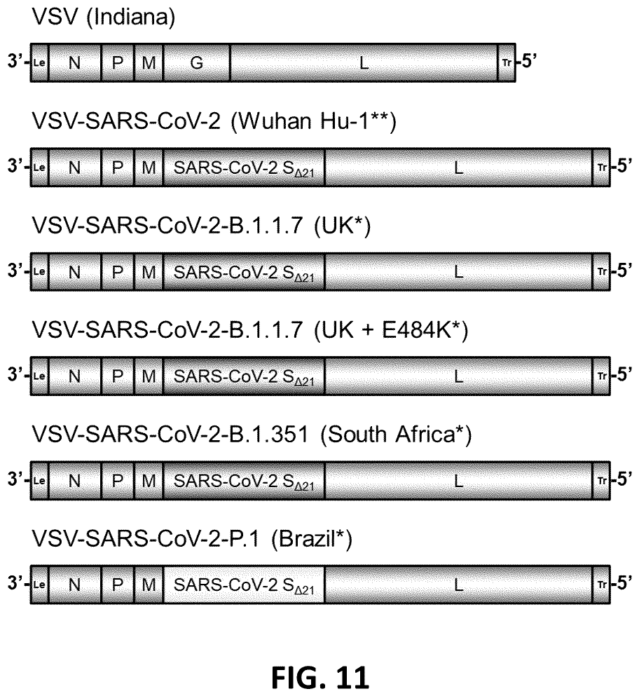

FIG. 11 illustrates a schematic of VSV-SARS-CoV-2 vaccines. Codon optimized (*) or codon optimized and wild-type (**) nucleotide sequences of the SARS-CoV-2 spike lacking the final 63 nucleotides (corresponding to S_{Δ21}) were inserted into the VSV genome in place of the native G gene. Viruses were rescued, plaque-purified, and entire genomes were sequenced to validate the vaccine stocks.

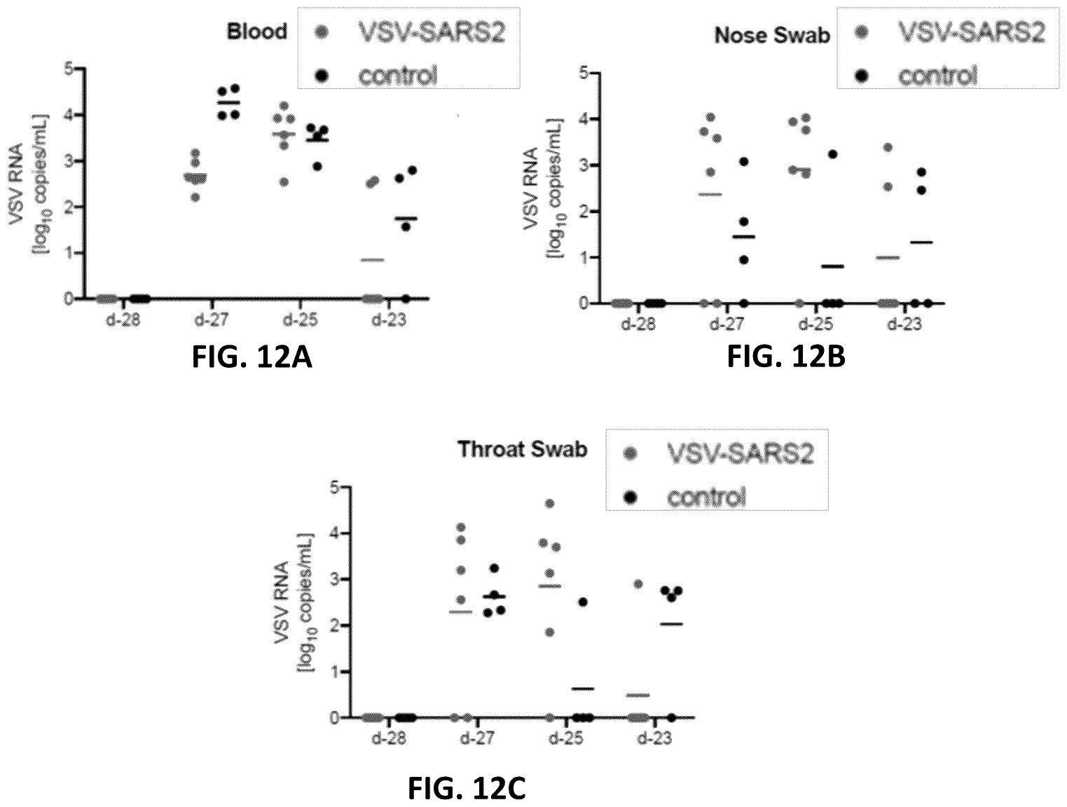

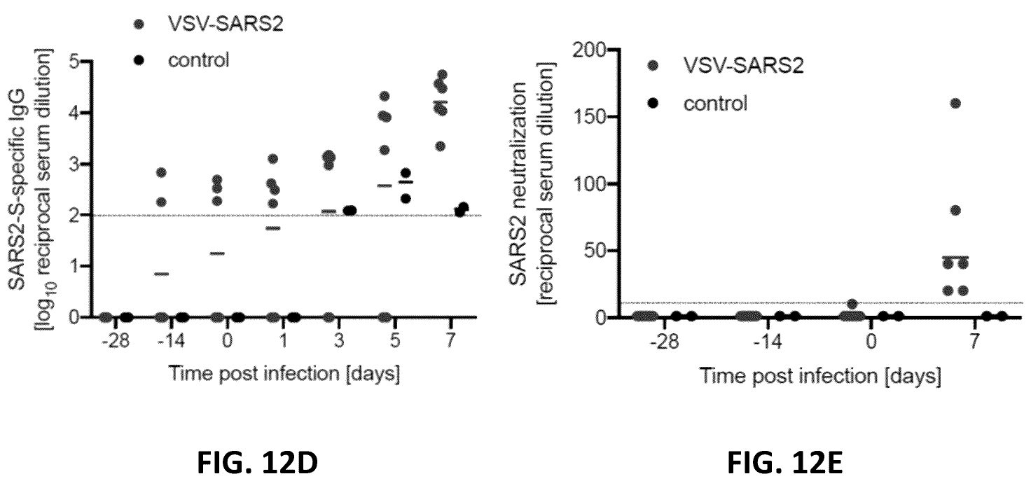

FIG. 12 demonstrates the results of vaccination of rhesus macaques with non-purified VSV-SARS-CoV-2. Rhesus macaques were vaccinated intramuscularly with a single 10⁷ pfu dose of VSV-SARS-CoV-2 (wild-type nucleotide sequence) or VSV-Ebola. A) Blood, nasal swabs, and throat swabs were tested for levels of VSV RNA at the days indicated post-vaccination. B) Spike-specific IgG levels were measured at the indicated days post-vaccination. C) Neutralizing antibody titers were measured at the days indicated post-vaccination (day 0=day challenged with SARS-CoV-2).

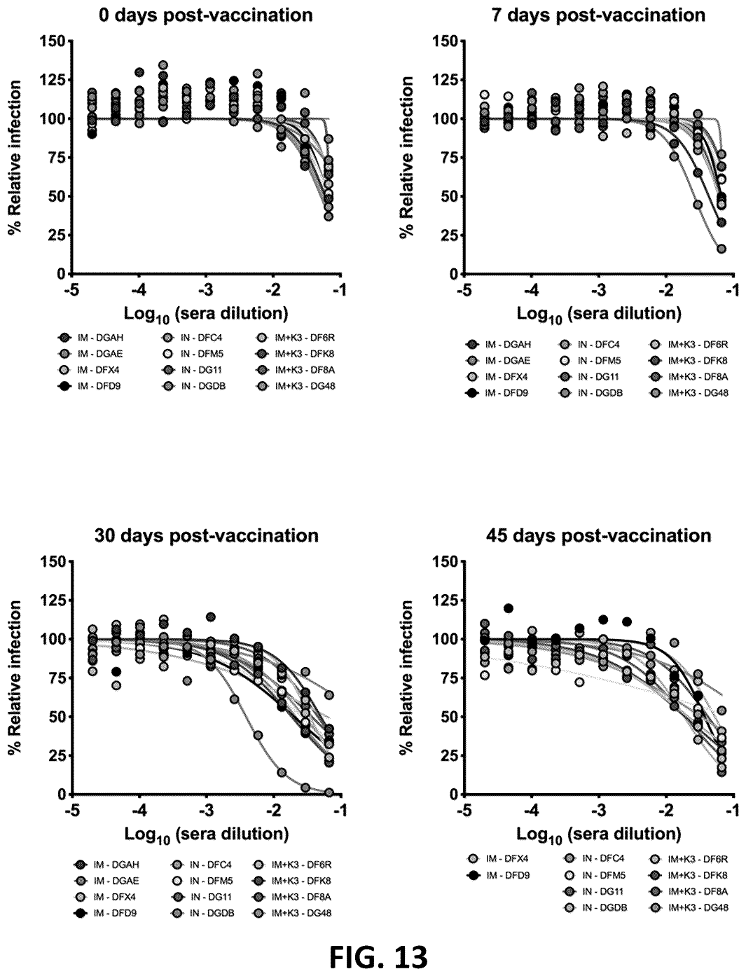

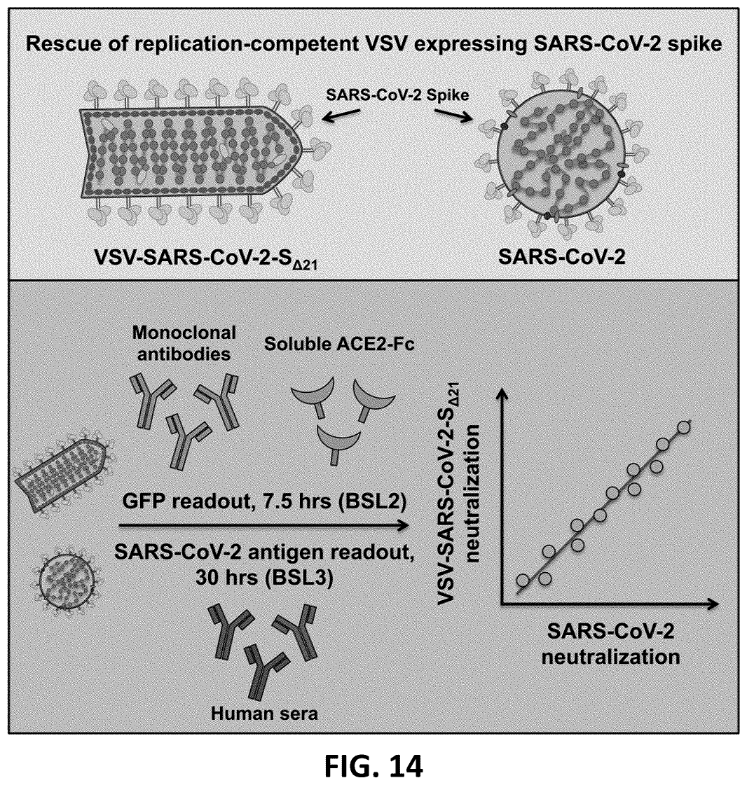

FIG. 13 shows the results of neutralizing antibody titers in rhesus macaques vaccinated with purified VSV-SARS-CoV-2. Rhesus macaques (n=4 per group) were vaccinated with a single 10⁷ pfu dose of VSV-SARS-CoV-2 (wild-type nucleotide sequence) and serum neutralizing antibody titers were measured at the indicated days post-vaccination using an established GFP-reduction neutralization test using VSV-eGFP-SARS-CoV-2. IM=intramuscular, IN=intranasal, IM+adj=intramuscular with K3 CpG adjuvant.

### Example 4: Neutralizing Antibody and Soluble ACE2 Inhibition of a Replication-Competent VSV-SARS-CoV-2 and a Clinical Isolate of SARS-CoV-2

The following example describes: vesicular stomatitis virus (VSV) encoding the SARS-CoV-2 spike replicates to high titers; virus propagation is enhanced by a truncation in the cytoplasmic tail of the spike; neutralization can be assessed by BSL2 and BSL3 high-throughput assays; and SARS-CoV-2- and VSV-SARS-CoV-2-based neutralization assays correlate (see e.g., Case et al. Cell Host & Microbe Volume 28, Issue 3, 9 Sep. 2020, Pages 475-485.e5)

Summary

Antibody-based interventions against SARS-CoV-2 could limit morbidity, mortality, and possibly transmission. An anticipated correlate of such countermeasures is the level of neutralizing antibodies against the SARS-CoV-2 spike protein, which engages with host ACE2 receptor for entry. Using an infectious molecular clone of vesicular stomatitis virus (VSV) expressing eGFP as a marker of infection, we replaced the glycoprotein gene (G) with the spike protein of SARS-CoV-2 (VSV-eGFP-SARS-CoV-2) (both G and S can be coexpressed as well) and developed a high-throughput-imaging-based neutralization assay at biosafety level 2. We also developed a focus-reduction neutralization test with a clinical isolate of SARS-CoV-2 at biosafety level 3. Comparing the neutralizing activities of various antibodies and ACE2-Fc soluble decoy protein in both assays revealed a high degree of concordance. These assays will help define correlates of protection for antibody-based countermeasures and vaccines against SARS-CoV-2. Additionally, replication-competent VSV-eGFP-SARS-CoV-2 provides a tool for testing inhibitors of SARS-CoV-2 mediated entry under reduced biosafety containment.

Introduction

Severe acute respiratory syndrome coronavirus 2 (SARS-CoV-2) is a positive-sense, single-stranded, enveloped RNA virus that was first isolated in Wuhan, China in December, 2019 from a cluster of acute respiratory illness cases (Guan et al., 2020). SARS-CoV-2 is the etiologic agent of coronavirus disease 2019 (COVID-19), which as of Jun. 17, 2020 has more than 8.2 million confirmed cases causing 445,000 deaths. Virtually all countries and territories have been affected, with major epidemics in Central China, Italy, Spain, France, Iran, Russia, Brazil, India, Peru, the United Kingdom, and the United States. SARS-CoV-2 is thought to be of zoonotic origin and is closely related to the original SARS-CoV (Zhang et al., 2020; Zhou et al., 2020). Most cases are spread by direct human-to-human transmission, with community transmission occurring from both symptomatic and asymptomatic individuals (Bai et al., 2020). This has resulted in a global pandemic with severe economic, political, and social consequences. The development, characterization, and deployment of an effective vaccine or antibody prophylaxis or treatment against SARS-CoV-2 could prevent morbidity and mortality and curtail its epidemic spread.

The viral spike protein (S) mediates all steps of coronavirus entry into target cells, including receptor binding and membrane fusion (Tortorici and Veesler, 2019). During viral biogenesis, the S protein undergoes furin-dependent proteolytic processing as it transits through the trans-Golgi network and is cleaved into S1 and S2 subunits that function in receptor binding and membrane fusion, respectively (Walls et al., 2020). Angiotensin-converting enzyme 2 (ACE2) serves as a cell surface receptor (Letko et al., 2020; Wrapp et al., 2020) for SARS-CoV-2, and productive infection is facilitated by additional processing of S2 by the host cell serine protease TMPRSS2 (Hoffmann et al., 2020).

Laboratory studies of SARS-CoV-2 require biosafety level 3 (BSL3) containment with positive-pressure respirators. Single-round pseudotyped viruses complemented by expression of the SARS-CoV-2 S protein in trans serve as biosafety level 2 (BSL2) surrogates that can facilitate studies of viral entry and the inhibition of infection by neutralizing antibodies and other inhibitors (Hoffmann et al., 2020; Lei et al., 2020; Nie et al., 2020; Ou et al., 2020). Such pseudotyping approaches are used routinely by many laboratories for other highly pathogenic coronaviruses, including SARS-CoV and MERS-CoV (Fukushi et al., 2005, 2006; Giroglou et al., 2004; Kobinger et al., 2007). Viral pseudotyping assays are limited by the need to express the glycoprotein in trans and preclude forward genetic studies of the viral envelope protein. Expression of the glycoprotein is often accomplished by plasmid transfection, which requires optimization to minimize batch variation. Assays performed with such pseudotyped viruses rely on relative levels of infectivity as measured by a reporter assay without correlation to an infectious titer. It also is unknown how the display of S proteins on a heterologous virus impacts viral entry, antibody recognition, and antibody neutralization compared to infectious coronavirus. This question is important because neutralization assays are used to establish correlates of protection for vaccine and antibody-based countermeasures, and most manufacturers lack access to high-containment laboratories to test antibody responses against highly pathogenic coronaviruses such as SARS-CoV-2.

Here, we developed a simple and robust BSL2 assay for evaluating SARS-CoV-2 entry and its inhibition by antibodies. We engineered an infectious molecular clone of vesicular stomatitis virus (VSV) to encode the SARS-CoV-2 S protein in place of the native envelope glycoprotein (G) and rescued an autonomously replication-competent virus bearing the spike. Through passage of VSV-eGFP-SARS-CoV-2, we selected a gain-of-function mutation in S that allowed more efficient viral propagation yielding titers of >1×10⁸ plaque-forming units (PFU)/mL. We characterized this variant with respect to inhibition by soluble human ACE2-Fc and monoclonal and polyclonal antibodies from humans and compared those results to neutralization tests with a clinical isolate of SARS-CoV-2. These studies demonstrate that a recombinant VSV expressing SARS-CoV-2 S behaves analogously to a clinical isolate of SARS-CoV-2, providing a useful high-throughput BSL2 assay for studying antibody neutralization or inhibition of viral spike-mediated entry.

Results

A Replication-Competent, Infectious VSV Chimera with SARS-CoV-2 S Protein

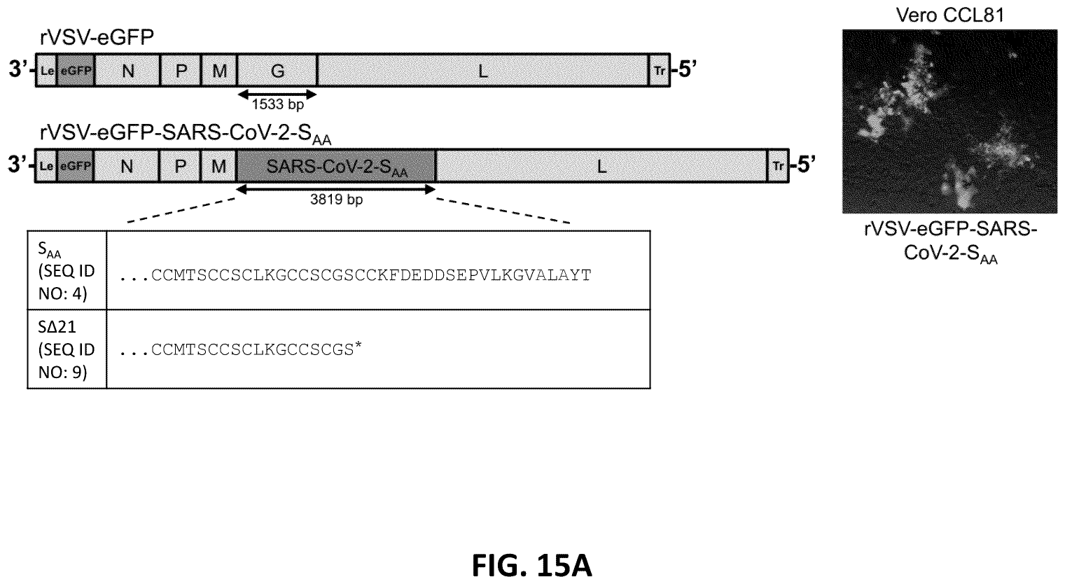

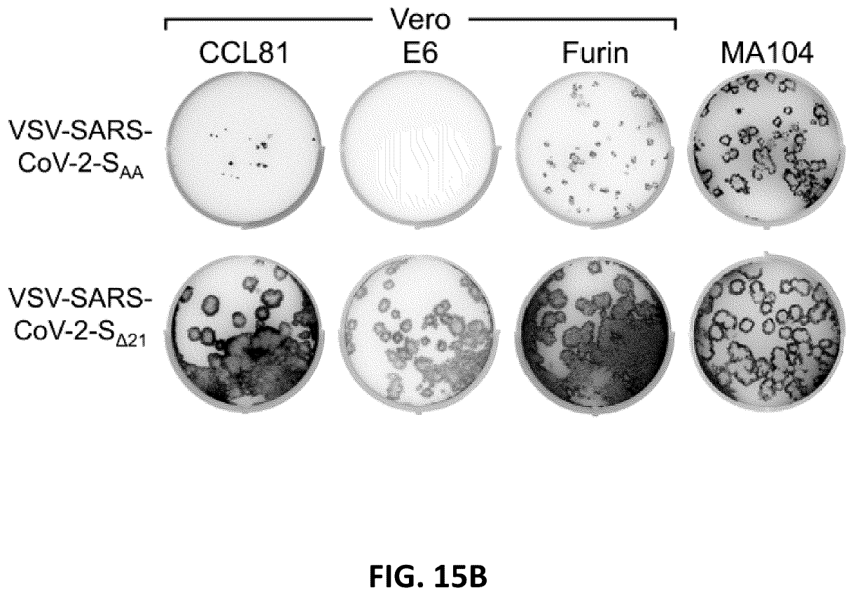

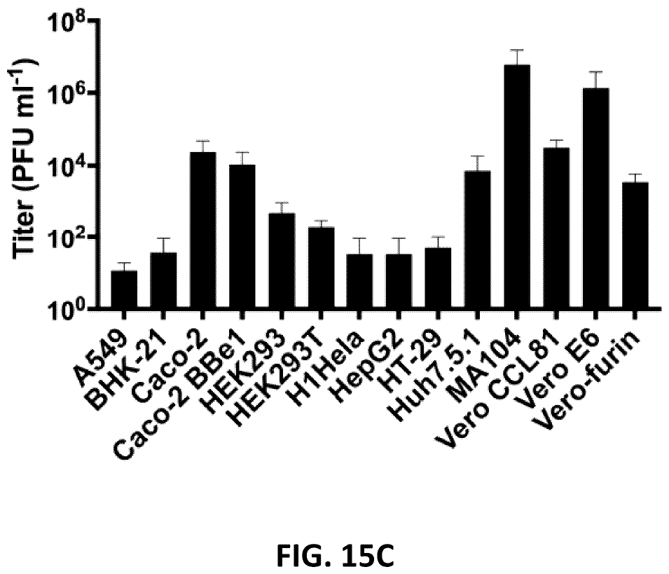

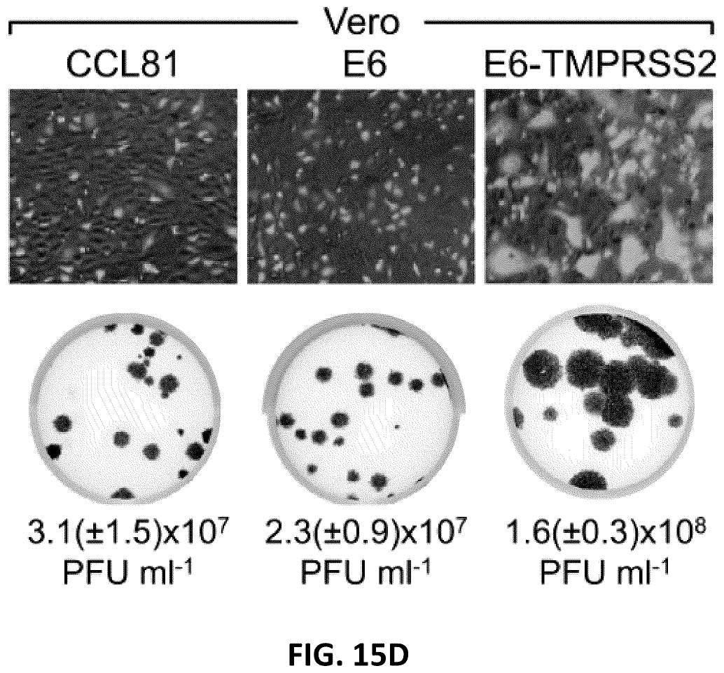

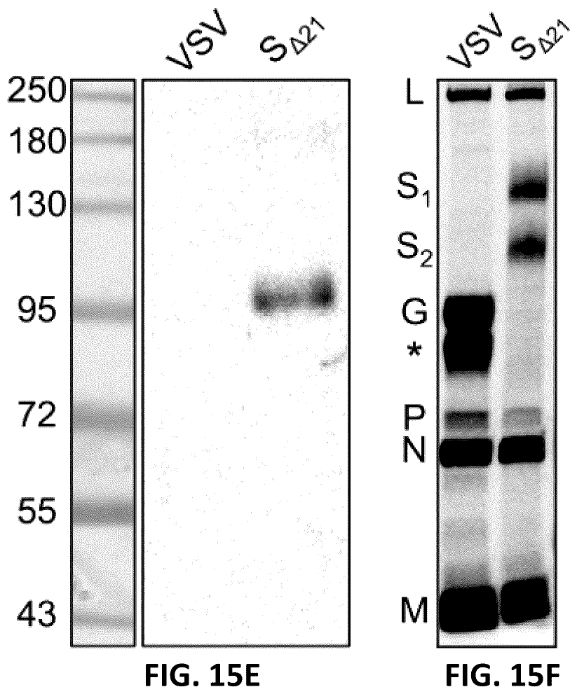

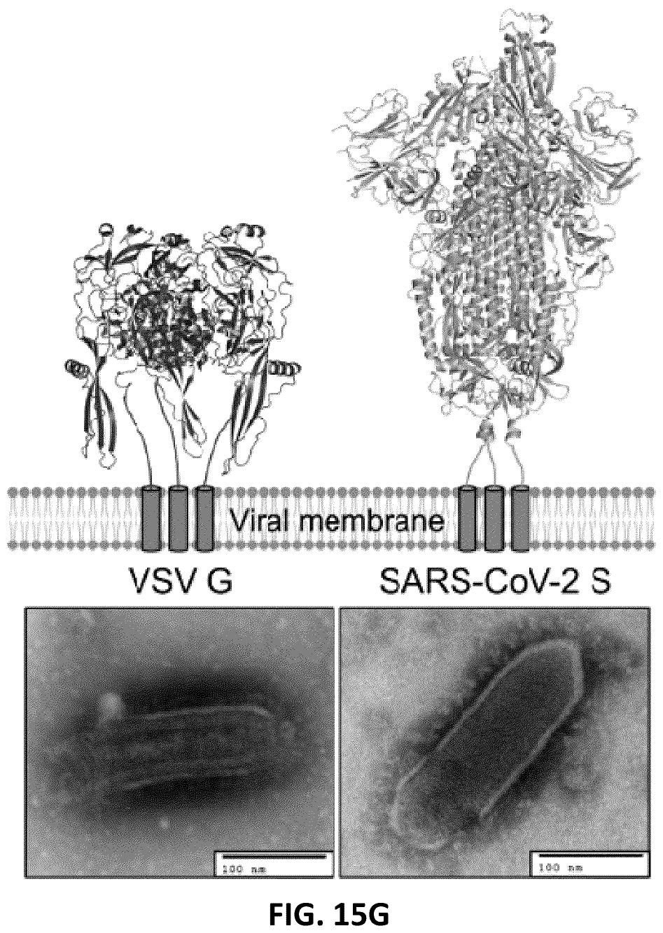

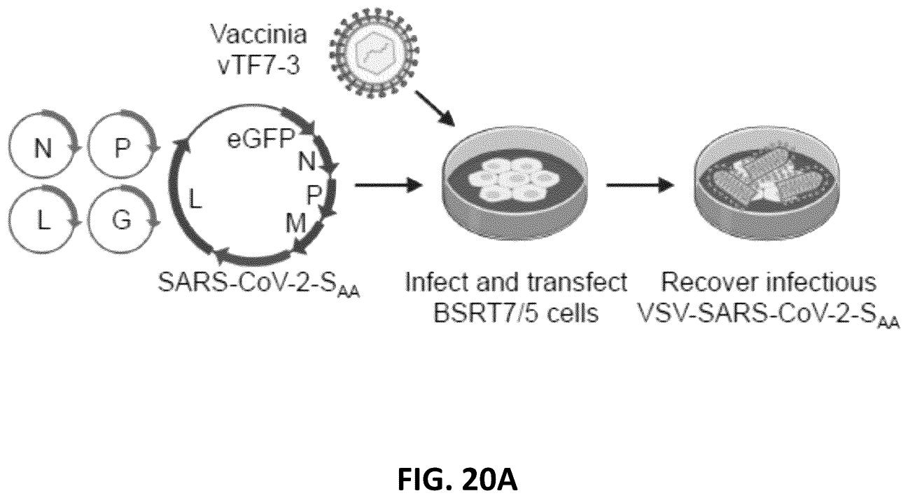

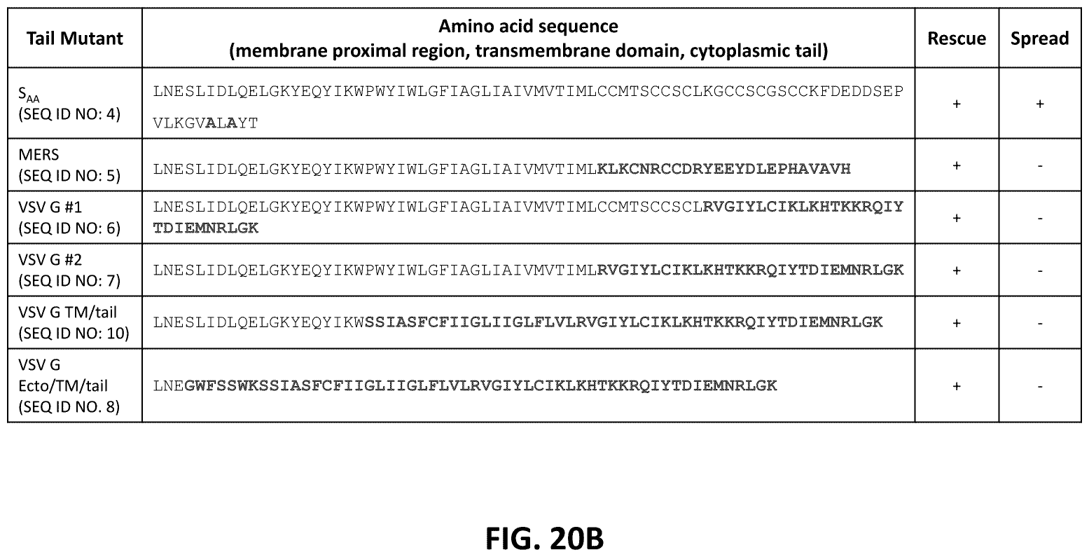

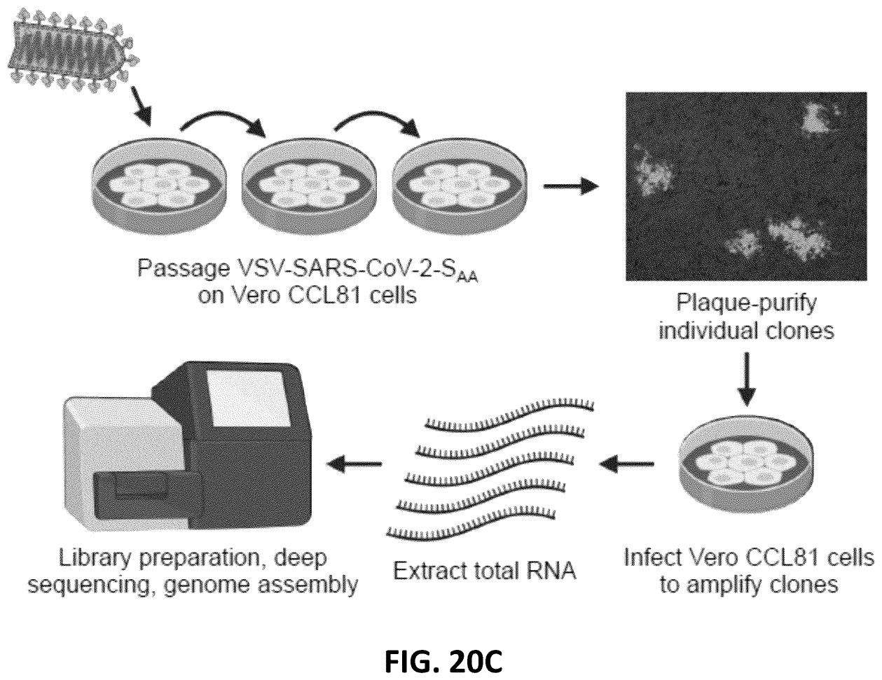

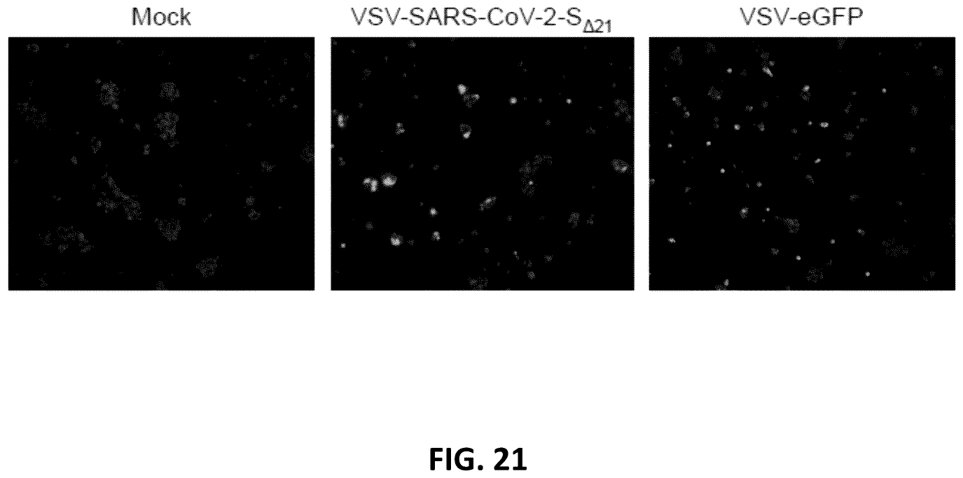

To generate a replication-competent virus to study entry and neutralization of SARS-CoV-2 at BSL2, we engineered an infectious molecular clone of VSV by replacing the endogenous glycoprotein (G) with SARS-CoV-2 S (FIG. 15A; see also FIG. 1A). SARS-CoV-2 S protein contains an endoplasmic reticulum (ER) retention sequence in the cytoplasmic tail (KxHxx-COOH) because virion assembly occurs in ER-Golgi intermediate compartments (Lontok et al., 2004; McBride et al., 2007; Ruch and Machamer, 2012). We pre-emptively altered that sequence to AxAxx to facilitate retargeting of S to the plasma membrane, the site of VSV assembly. Using established approaches (FIG. 20A) (Whelan et al., 1995), we recovered infectious VSV-eGFP-SARS-CoV-2-S_{AA} as determined by expression of the virus-encoded eGFP reporter (FIG. 15A, right). However, VSV-eGFP-SARS-CoV-2-S_{AA} propagation was inefficient on Vero CCL81 cells. This result prompted us to test additional modifications of the cytoplasmic tail of S, which were also defective in autonomous amplification (FIG. 20B). To overcome this, we used a forward genetic approach to isolate two adaptive variants of VSV-eGFP-SARS-CoV-2-S_{AA} (FIG. 20C). Virus was plaque-purified from the transfected cell supernatants, and one variant was passaged twice on Vero CCL81 cells. Following subsequent plaque isolation and serial amplification, we sequenced the viral RNA in infected cells at the seventh passage. A second, independent plaque from transfected cell supernatants was passaged an additional five times on a rhesus monkey MA104 cell line. Both approaches led to the emergence of a virus that contained a single mutation, a cysteine to stop mutation at residue 1253 (TGC to TGA at nucleotide 3759), which truncates the cytoplasmic tail of SARS-CoV-2 S by 21 residues (FIG. 15A). This virus, hereafter referred to as VSV-SARS-CoV-2-SΔ21, was passed 12 times in total to assess genetic stability by next generation sequencing, which revealed no additional mutations in the spike (SRA: SRR11878607; BioProject: PRJNA635934). Comparison of plaque morphology of VSV-SARS-CoV-2-S_{Δ21} and VSV-eGFP-SARS-CoV-2-S_{AA} on three Vero cell subtypes and MA104 cells demonstrates that the selected variant spreads more efficiently (FIG. 15B). Screening of a larger panel of cell types (FIG. 15C) identified MA104 and Vero E6 cells as supporting the highest levels of virus production. Ectopic expression of TMPRSS2 led to a further ˜10-fold increase in viral titer and larger plaque size (FIG. 15D). VSV-SARS-CoV-2-S_{Δ21} also was capable of infecting Calu-3 cells, a human epithelial lung adenocarcinoma cell line (FIG. 21).

SARS-CoV-2-S_{Δ21} is Incorporated into Infectious VSV Particles

To confirm incorporation of SARS-CoV-2 S into particles, we first amplified the virus in the presence of VSV G to allow infection of cell types independently of the S protein. The VSV G trans-complemented VSV-SARS-CoV-2-S_{Δ21} efficiently infects HEK293T cells, which then serve as a source of production of virus particles containing SARS-CoV-2 S protein. Western blotting of supernatants with CR3022, a cross-reactive anti-S monoclonal antibody (mAb) (ter Meulen et al., 2006; Yuan et al., 2020), established the presence of S_{Δ21} in VSV-SARS-CoV-2-S_{Δ21} particles, but not in the parental VSV (FIG. 15E). The protein detected migrated at ˜100 kilodaltons, a band that corresponds to the cleaved S1 subunit of the glycoprotein (Watanabe et al., 2020). To examine whether the S_{Δ21} incorporated into VSV particles is processed to S1 and S2, we performed [³⁵S] cysteine-methionine metabolic labeling in BSRT7 cells, which support robust VSV replication, and analyzed released particles by SDS-PAGE and phosphorimaging. In addition to the VSV structural proteins (N, P, M, and L), two additional bands that correspond in size to glycosylated S1 (107 kDa) and S2_{Δ21} (85 kDa) were observed for VSV-SARS-CoV-2-S_{Δ21} (FIG. 15F). Negative-stain electron microscopy of sucrose-gradient purified virus particles revealed that the membrane protein projecting from VSV-SARS-CoV-2-S_{Δ21} is larger than observed on wild-type VSV particles (FIG. 15G), which reflects the larger size of the coronavirus spike.

A High-Throughput Focus-Forming Assay with a Clinical Isolate of SARS-CoV-2

VSV-SARS-CoV-2-S_{Δ21} has several advantages for detection and measuring of neutralizing antibodies, including lower biosafety containment level, ease of production and use, and rapid reporter gene readout. Nonetheless, the difference in virus morphology (spherical CoV versus bullet-shaped VSV) and possible effects on the conformational display of S on the virion surface raise questions of whether the accessibility of epitopes and stoichiometry of antibody neutralization is similar to authentic SARS-CoV-2. A direct comparison with a clinical isolate of SARS-CoV-2 is necessary to establish the utility of VSV-SARS-CoV-2-S_{Δ21} for assays of viral entry and antibody neutralization.

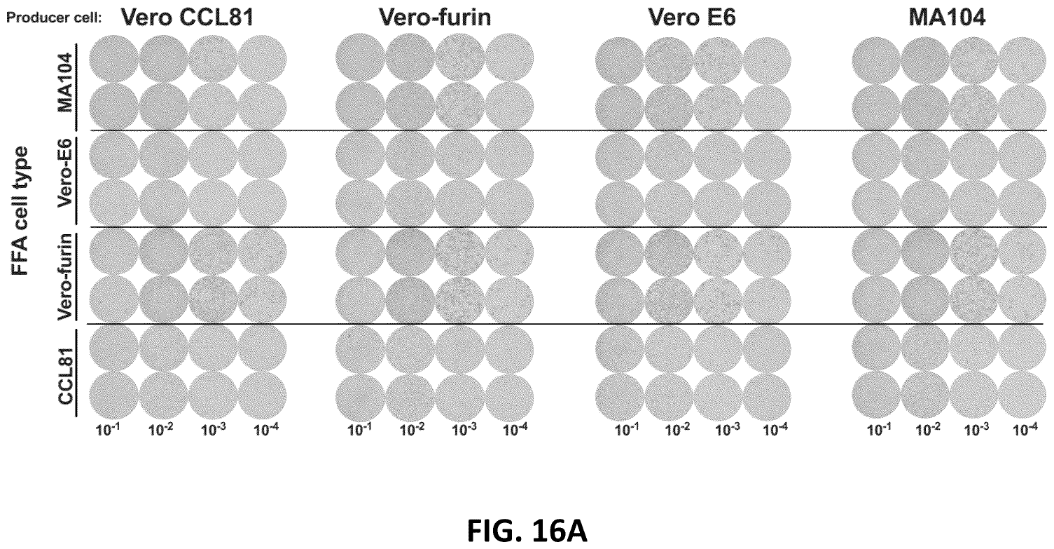

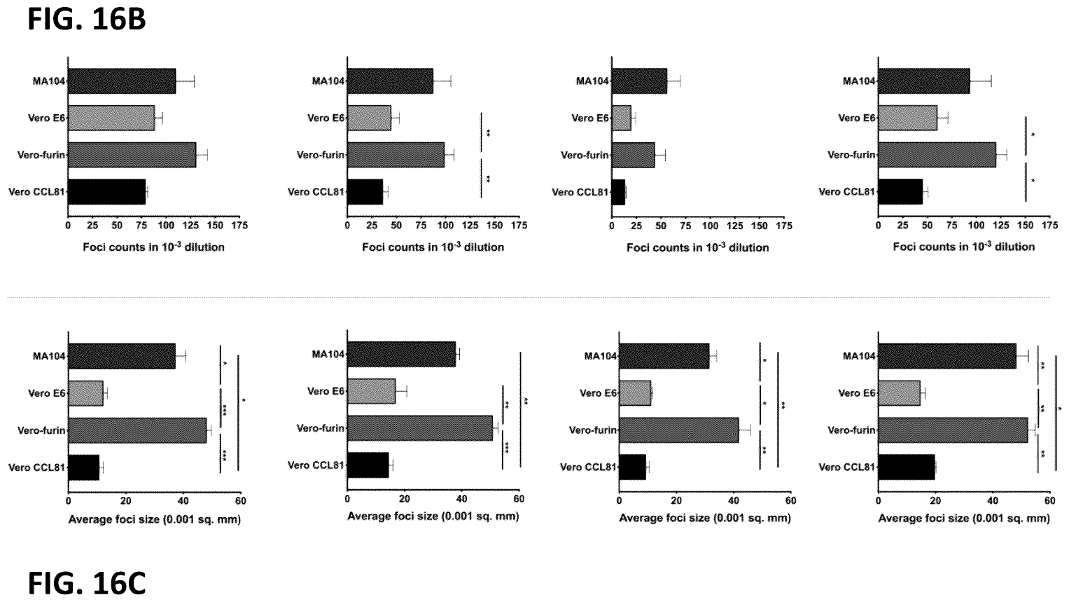

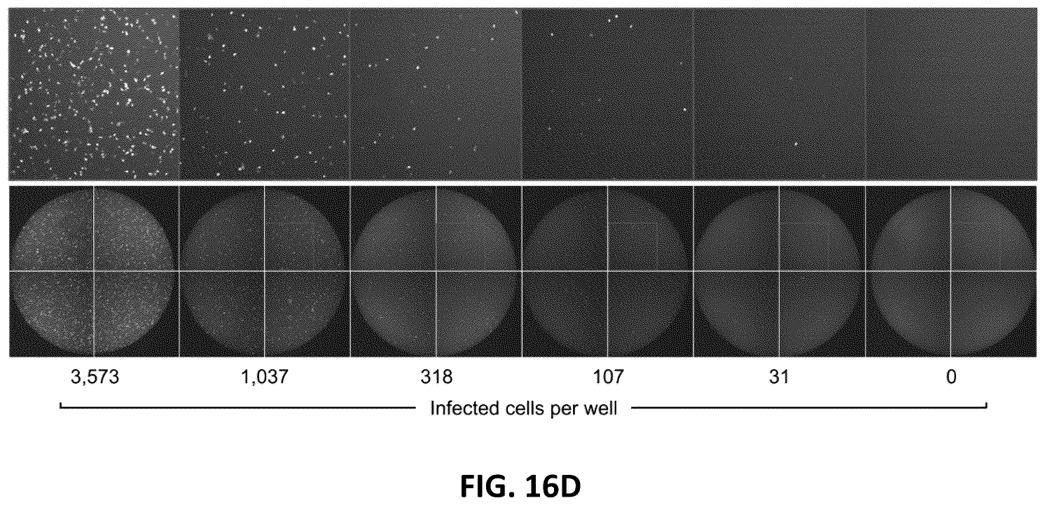

We designed a high-throughput assay for titrating SARS-CoV-2 that could be applied to multiple cell substrates. Instead of using a plaque assay—which relies on the capacity for a virus to cause cell death, which can vary across cell types-we developed a focus-forming assay (FFA) and viral antigen detection as a measure of infectivity. We propagated SARS-CoV-2 in four different producer cell types (Vero CCL81, Vero E6, Vero-furin, and MA104 cells) and then measured the number and size of foci after staining recipient cells with an anti-S mAb. With SARS-CoV-2 stocks generated from each producer cell type, we observed distinct foci across recipient cell substrates at approximately 30 h post-inoculation (FIG. 16A). We consistently observed the highest viral titers and largest foci sizes with Vero-furin and MA104 cells (FIG. 16B and FIG. 16C). However, the larger foci were more difficult to enumerate on an automated Immunospot reader and required additional manual quality control analysis. Because of this, we used Vero E6 cells for our rapid focus-reduction neutralization tests (FRNT) in subsequent experiments.

A High-Throughput, eGFP-Based Neutralization Assay for VSV-SARS-CoV-2-S_{Δ21}

In parallel, we developed a high-throughput method to measure neutralization of VSV-SARS-CoV-2-S_{Δ21}. As VSV-SARS-CoV-2-S_{Δ21} encodes an eGFP reporter and viral gene expression is robust, eGFP-positive cells can be quantified 7.5 h post-infection using a fluorescence microscope with automated counting analysis software. This approach enabled the development of an eGFP-reduction neutralization test (GRNT) (FIG. 16D).

Neutralization of VSV-SARS-CoV-2-SA₂, and SARS-CoV-2 by Human Antibodies

Members of our group recently identified human mAbs from memory B cells of a SARS-CoV survivor that bind to SARS-CoV-2 S (Pinto et al., 2020).

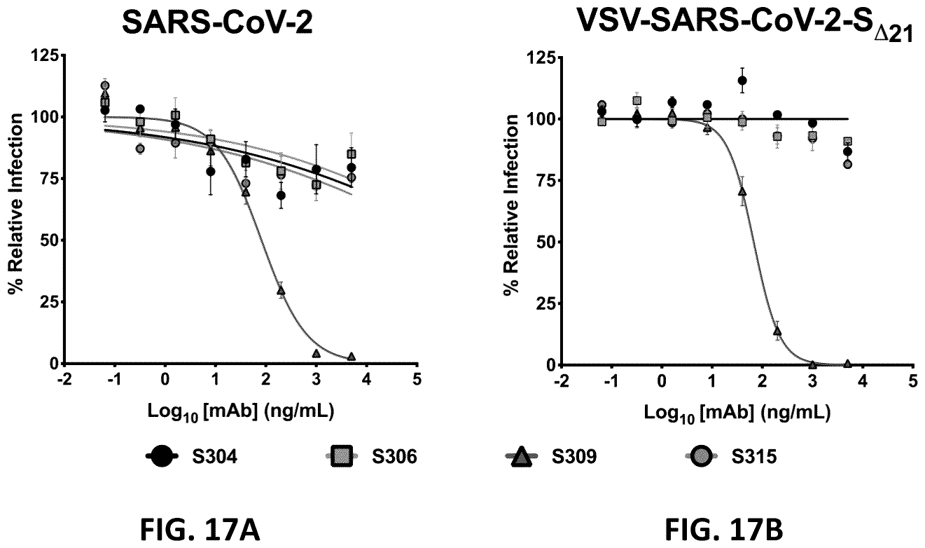

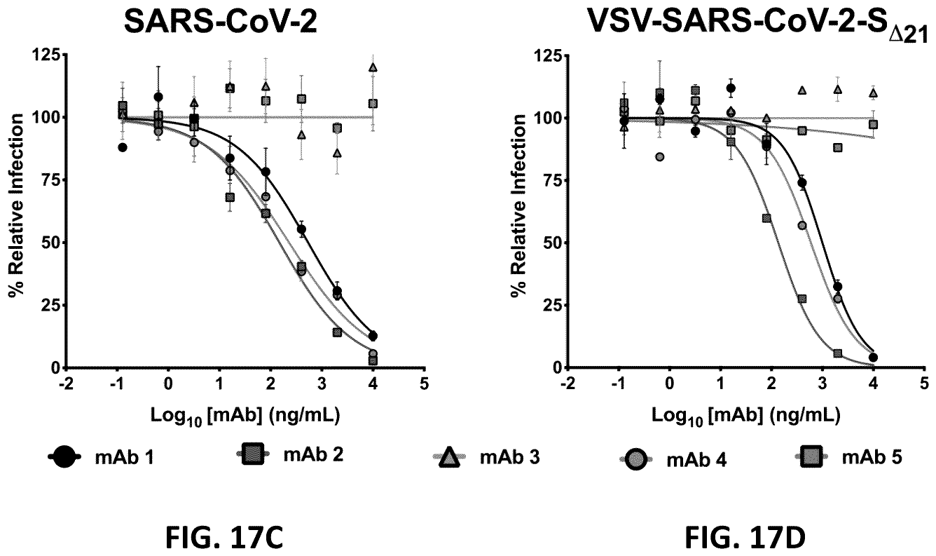

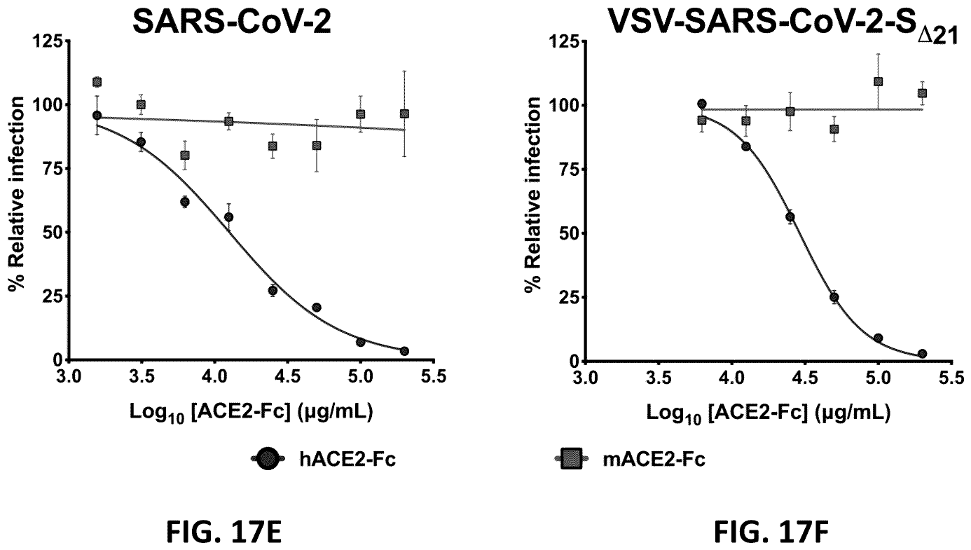

We tested a subset of these (mAbs 304, 306, 309, and 315) for their ability to inhibit VSV-SARS-CoV-2-S_{Δ21} and SARS-CoV-2 infections on Vero E6 cells. While three of these mAbs showed poor inhibitory activity, mAb 309 potently neutralized both SARS-CoV-2 and VSV-SARS-CoV-2-S_{Δ21} (FIG. 17A and FIG. 17B) with similar EC₅₀ values between the two assays (81 and 67 ng/mL for SARS-CoV-2 and VSV-SARS-CoV-2-S_{Δ21}, respectively). To broaden the test panel, we evaluated the activity of a panel of mAbs generated as part of a phage display library (Miersch et al., 2020) by both FRNT and GRNT. Many of these mAbs exhibited moderate neutralization activities in the EC₅₀ range of 100 to 500 ng/mL (FIG. 17C and FIG. 17D). Nonetheless, we observed the same neutralization trend between VSV-SARS-CoV-2-S_{Δ21} and SARS-CoV-2 with highly correlated EC₅₀ values (<2-fold differences).

Neutralization by Human ACE2-Fc Receptor Decoy Proteins

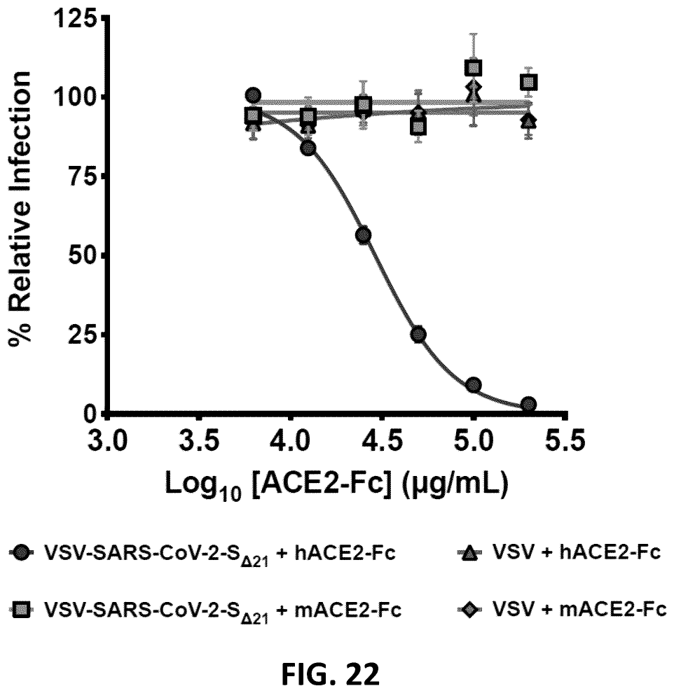

Human ACE2 (hACE2) is an entry receptor for both SARS-CoV and SARS-CoV-2 (Letko et al., 2020; Li et al., 2003, 2005; Wrapp et al., 2020). As a soluble hACE2-Fc decoy protein has been proposed as a therapeutic for SARS-CoV-2 (Kruse, 2020), in part based on its ability to inhibit SARS-CoV infection in cell culture (Moore et al., 2004), we tested whether hACE2-Fc could inhibit infection of VSV-SARS-CoV-2-S_{Δ21} and SARS-CoV-2 using our FRNT and GRNT assays. When pre-mixed with VSV-SARS-CoV-2-S_{Δ21} or SARS-CoV-2, hACE2-Fc, but not murine ACE2-Fc (mACE2-Fc), it dose dependently and equivalently inhibited infection of recipient Vero E6 cells (FIG. 17E and FIG. 17F). As expected, hACE2-Fc did not inhibit infection of wild-type VSV, confirming that neutralization was specific to the SARS-CoV-2 S protein (FIG. 22). We noted that a relatively high concentration of hACE2-Fc was required for inhibition, with EC₅₀ values of 29 and 12.6 μg/mL for VSV-SARS-CoV-2-S_{Δ21} and SARS-CoV-2, respectively. Thus, although soluble hACE2-Fc decoy proteins similarly inhibit infection of VSV-SARS-CoV-2-S_{Δ21} and SARS-CoV-2, the potency is less than anticipated, which suggests that the receptor-binding domain (RBD) on the S protein on the surface of both viruses may not be fully accessible in solution.

Neutralization of VSV-SARS-CoV-2-S_{Δ21} and SARS-CoV-2 by Human Immune Serum

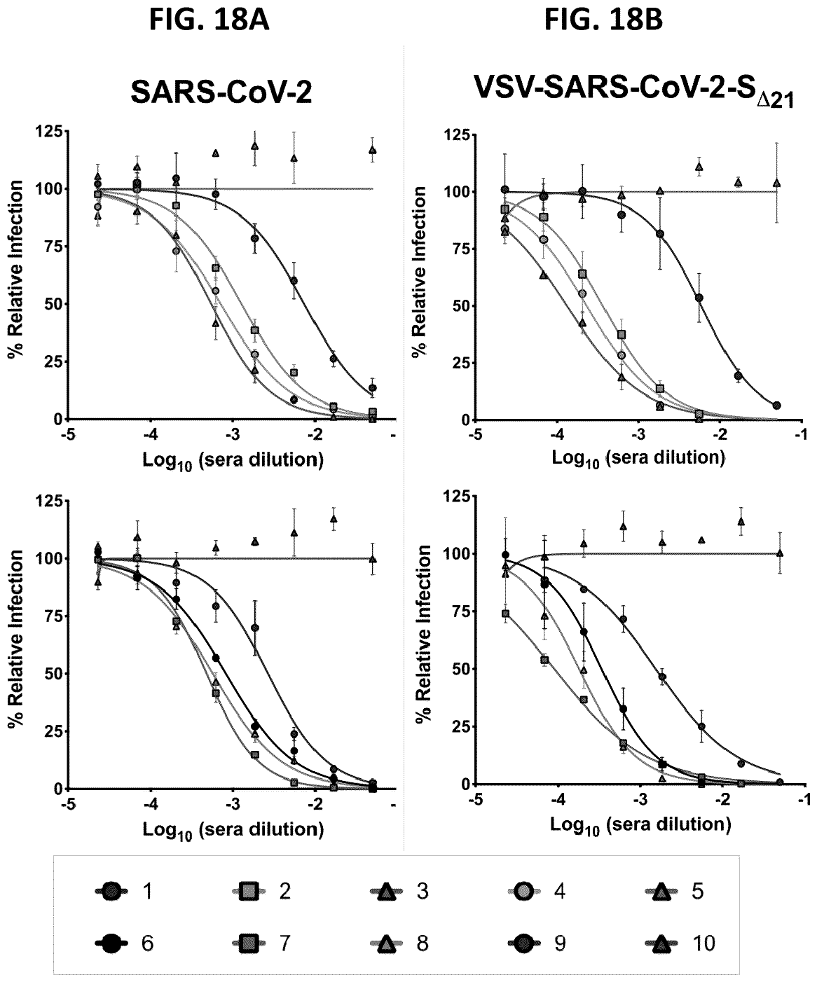

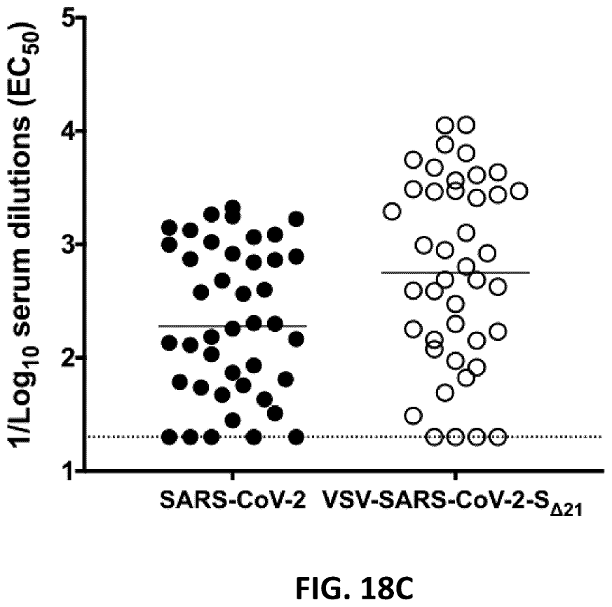

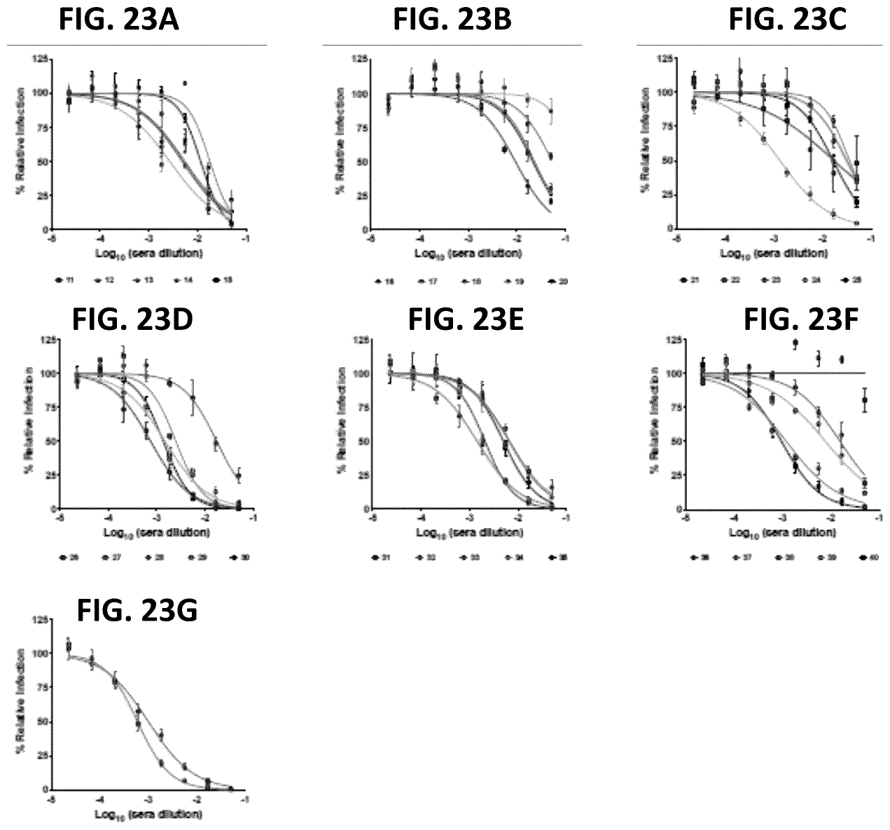

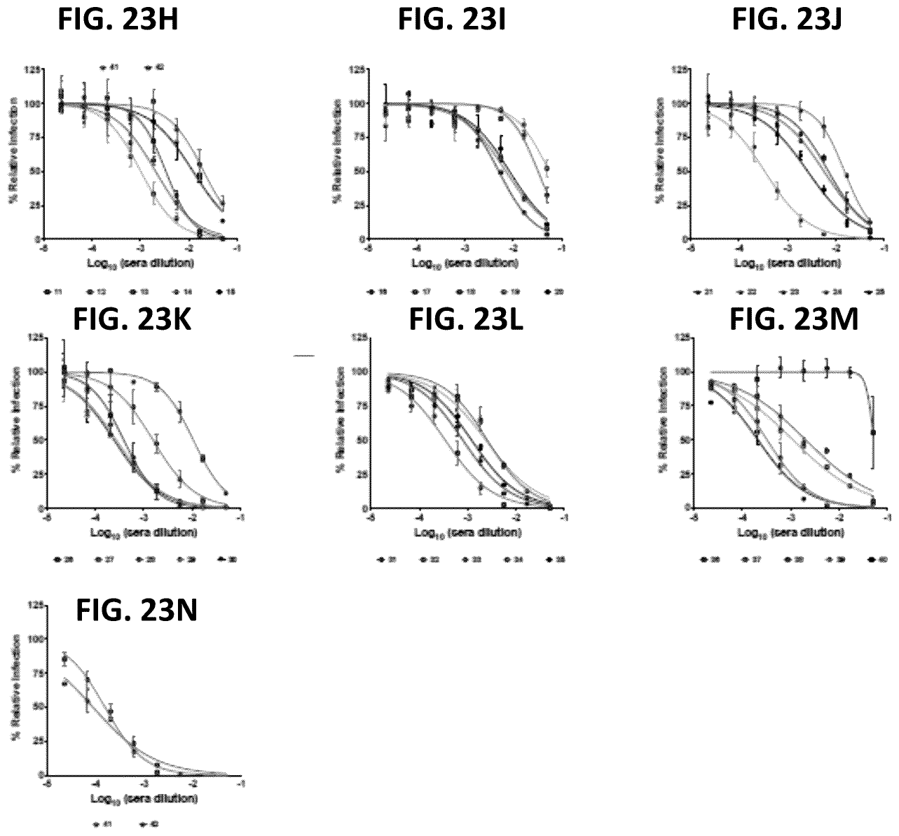

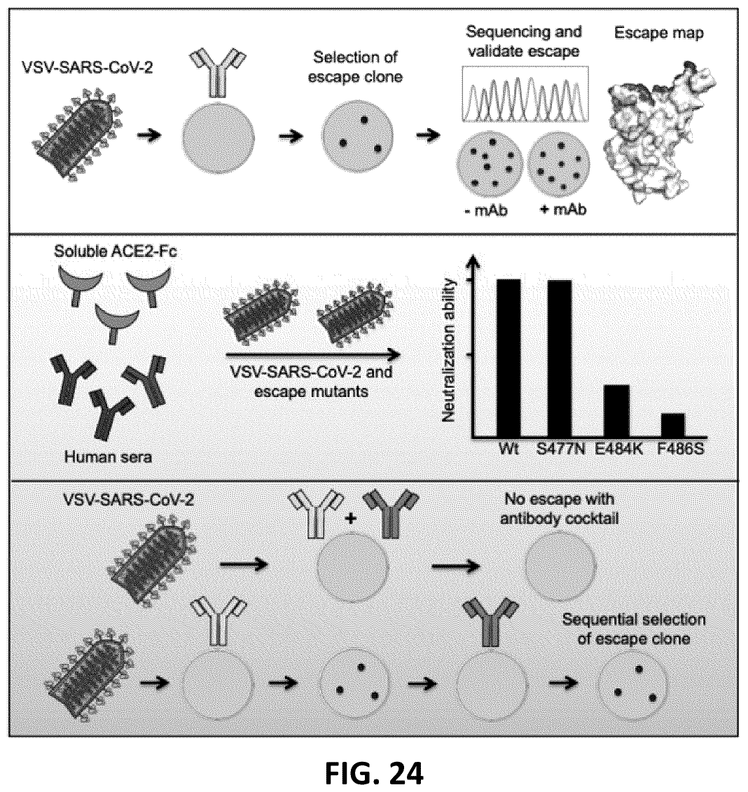

As part of studies to evaluate immune convalescent plasma as a possible therapy for SARS-CoV-2-infected patients (Bloch et al., 2020), we obtained 42 serum samples from 20 individuals at different time points after the onset of COVID-19 symptoms (TABLE 1). These samples were pre-screened using a commercially available IgG ELISA. We tested each sample for neutralization of VSV-SARS-CoV-2-S_{Δ21} and SARS-CoV-2 on Vero E6 cells. We observed that sera with ELISA-negative or indeterminate results generally showed low inhibitory titers (EC₅₀<1/100), whereas ELISA-positive sera generated a broad range of neutralizing antibody activity (EC₅₀>1/200 to >1/1,900) (FIG. 18A and FIG. 23). Remarkably, neutralization of VSV-SARS-CoV-2-S_{Δ21} and SARS-CoV-2 was similar across the entire panel of samples (FIG. 18B, FIG. 18C, and FIG. 23).

TABLE 1
Human Serum ELISA IgG. Serum samples from 20 individuals were
coIlected at different time points post onset of CoVID-19
symptoms and screened using two ELISA assays (Euroimmun or
Epitope). The serum numbers correspond to those of FIG. 18
and FIG. 22. IgG index values were calculated by dividing
the O.D. of the serum sample by a reference O.D. control,
and ratios were interpreted using the following criteria as
recommended by the manufacturer: Negative (−) <0.8,
Indeterminate (+/−) 0.8-1.1, Positive (+) ≥1.1.

| | Days Post | Euroimmun IgG | | Epitope IgG | |
|---|---|---|---|---|---|
| Serum | Symptom Onset | Index | Reactive | Index | Reactive |
| 1 | 14 | 1.3 | + | 2.6 | + |
| 2 | 12 | 6.0 | + | 3.5 | + |
| 3 | 17 | 10.2 | + | 3.9 | + |
| 4 | 16 | 14.9 | + | 4.5 | + |
| 5 | 5 | 0.2 | − | 1.2 | + |
| 6 | 19 | 8.6 | + | 4.4 | + |
| 7 | 17 | 7.1 | + | 4.1 | + |
| 8 | 10 | 5.1 | + | 2.0 | + |
| 9 | 14 | 6.8 | + | 3.1 | + |
| 10 | 6 | 0.2 | − | 1.1 | + |
| 11 | — | 5.6 | + | 0.9 | +/− |
| 12 | — | <0.8 | − | 1.3 | + |
| 13 | 9 | 0.7 | − | 1.1 | + |
| 14 | 20 | 3.6 | + | 2.9 | + |
| 15 | 13 | 0.4 | − | 2.0 | + |
| 16 | 13 | 0.5 | − | 0.9 | − |
| 17 | 11 | 0.3 | − | 0.8 | − |
| 18 | 10 | 0.2 | − | 0.7 | − |
| 19 | 14 | 0.9 | +/− | 1.2 | + |
| 20 | 10 | 0.4 | − | 1.5 | + |
| 21 | 11 | 0.5 | − | 2.0 | + |
| 22 | 10 | 0.3 | − | 0.6 | − |
| 23 | 17 | 7.6 | + | 4.2 | + |
| 24 | 14 | 3.5 | + | 3.3 | + |
| 25 | 13 | 1.5 | + | 2.9 | + |
| 26 | 17 | 14.2 | + | 4.4 | + |
| 27 | 13 | 0.5 | − | 1.9 | + |
| 28 | 14 | 9.2 | + | 4.6 | + |
| 29 | 13 | 3.9 | + | 2.8 | + |
| 30 | 16 | 3.4 | + | 4.6 | + |
| 31 | 15 | 10.7 | + | 3.6 | + |
| 32 | 6 | 0.5 | − | 1.1 | + |
| 33 | 11 | 0.4 | − | 1.8 | + |
| 34 | 12 | 0.6 | − | 2.5 | + |
| 35 | 14 | 3.7 | + | 4.1 | + |
| 36 | 20 | 11.6 | + | 4.2 | + |
| 37 | 7 | 0.7 | − | 1.2 | + |
| 38 | 8 | 1.7 | + | 1.9 | + |
| 39 | 7 | 0.3 | − | 0.7 | − |
| 40 | 9 | 3.4 | + | 2.2 | + |
| 41 | 18 | 10.3 | + | 4.1 | + |
| 42 | 17 | 10.3 | + | 4.6 | + |

VSV-SARS-CoV-2-S_{Δ21} and SARS-CoV-2 Neutralization Assays are Highly Correlative

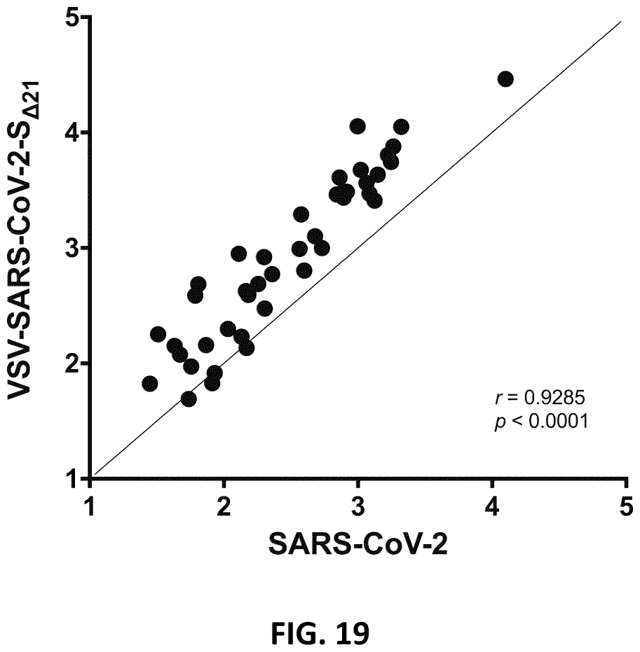

We determined the extent to which the VSV-SARS-CoV-2-S_{Δ21} and SARS-CoV-2 neutralization tests correlated with each other. We compared the GRNT and FRNT EC₅₀ values obtained in assays with mAbs, polyclonal sera, and soluble ACE2 protein (FIG. 19). For the samples with neutralizing activity, we observed a remarkably strong correlation between the two assays (r=0.9285; p<0.001). Moreover, all 11 of the samples that were deemed non-neutralizing in one assay had the same result in the second assay. Together, these results establish the utility of using VSV-SARS-CoV-2-S_{Δ21} as a surrogate for authentic SARS-CoV-2 infection in entry inhibition and neutralization studies.

Discussion

Emerging viral pathogens have caused numerous epidemics and several pandemics over the last century. The most recent example, SARS-CoV-2, has spread to nearly every country in the world in just a few months, causing millions of infections and hundreds of thousands of deaths (https://www.worldometers.info/coronavirus/). Rapid responses to viral outbreaks and generation of countermeasures require readily accessible tools to facilitate study and evaluate antiviral activity. Here, we generated a high-titer, replication-competent chimeric VSV expressing the SARS-CoV-2 S protein that performs similarly to a SARS-CoV-2 clinical isolate across multiple neutralization tests. As access to BSL3 facilities is limited, the finding that VSV-SARS-CoV-2-S_{Δ21} is neutralized similarly by decoy receptors, mAbs, and polyclonal antibodies in comparison to authentic SARS-CoV-2 is important. This tool will enable academic, government, and industry investigators to rapidly perform assays that interrogate SARS-CoV-2 entry, neutralization, and inhibition at a BSL2 level, which should simplify and expedite the discovery of therapeutic interventions and analysis of functional humoral immune responses.

Upon recovery of VSV-SARS-CoV-2-S_{AA}, we selected for a mutant, which contained a 21-amino acid deletion in the cytoplasmic tail. As truncation of the cytoplasmic tail eliminates the modified KxHxx ER retention signal, we suggest that this mutation facilitates more efficient incorporation of the SARS-CoV-2 S protein into the VSV particles. Although truncation of the cytoplasmic tail of HIV envelope protein resulted in conformational alterations in the ectodomain of the protein (Chen et al., 2015), based on the extensive neutralization data presented here, including correlation to neutralization of a clinical isolate of SARS CoV-2, a 21-amino acid truncation does not appear to substantively alter the structure of the S protein ectodomain. It remains to be determined whether fully wild-type S protein can incorporate efficiently into VSV. Indeed, similar mutations were generated in the SARS-CoV S protein cytoplasmic tail to boost incorporation into retroviruses and VSV pseudotypes (Fukushi et al., 2005; Giroglou et al., 2004; Moore et al., 2004).

The value of a chimeric virus depends on its capacity to present viral surface antigens in a similar way to its authentic counterpart (Garbutt et al., 2004). Indeed, the morphology of the bullet-shaped rhabdovirus and the spherical coronavirus and the density and geometry of S protein display could differentially impact antibody engagement and neutralization. Despite this concern, our extensive testing of VSV-SARS-CoV-2-S_{Δ21} with antibodies and soluble ACE2-Fc proteins showed similar neutralization profiles compared to authentic, fully infectious SARS-CoV-2. Thus, VSV-SARS-CoV-2-S_{Δ21}, despite the structural differences of the virion, provides a useful tool for screening antibodies, entry-based antiviral agents, and vaccine responses against SARS-CoV-2. Indeed, convalescent plasma is under investigation as a potential COVID-19 therapeutic (Chen et al., 2020). Our studies suggest that, in addition to testing for anti-S or anti-RBD antibodies (Shen et al., 2020), neutralization assays with VSV-SARS-CoV-2-S_{Δ21} may be a convenient and rapid method to obtain functional information about immune plasma preparations to enable prioritization prior to passive transfer to COVID-19 patients.

Coronaviruses possess a roughly 30 kb RNA genome, which requires that they encode a proofreading enzyme (ExoN in nsp14) (Denison et al., 2011) to counteract the error rate of the viral-RNA-dependent RNA polymerase. The lack of such proofreading enzymes in the genomes of rhabdoviruses suggests that selection of escape mutants to inhibitors of the coronavirus S protein will be faster in VSV-SARS-CoV-2, which further increases the utility of this chimeric virus. Our FRNT and GRNT assays can be used to establish evidence of prior SARS-CoV-2 infection or vaccination, as well as determine waning of functional responses over time, as the likelihood of cross-neutralizing responses with other cosmopolitan coronaviruses (e.g., HCoV-229E and HCoV-OC43) is exceedingly low. Overall, VSV-SARS-CoV-2-S_{Δ21} and our FRNT and GRNT assays can facilitate the development and evaluation of antibody- or entry-based countermeasures against SARS-CoV-2 infection. A similar VSV-SARS-CoV-2 chimera as a reporter of antibody mediated neutralization is described in a companion paper (Dieterle et al., 2020).

Our experiments do not directly test the safety of the recombinant VSV-eGFP-SARS-CoV-2. VSV-Indiana and chimeric VSV in which the native envelope protein is replaced with that of the envelope proteins of Ebola (Garbutt et al., 2004; Jones et al., 2005; Takada et al., 2003), Lassa (Geisbert et al., 2005), Andes (Brown et al., 2011), highly pathogenic avian influenza (Furuyama et al., 2020), and many other viruses are handled at BSL2. VSV-Ebola is a licensed human vaccine, ERVEBO, distributed by Merck. We are actively evaluating VSV-SARS-CoV-2 as a vaccine candidate, and mice inoculated with this virus do not develop disease (J.B.C., P.W.R., S.P.J.W., and M.S.D., unpublished data). Notwithstanding these data, a chimeric VSV containing both the F and G genes of Nipah virus remains pathogenic in mice (van den Pol et al., 2017), suggesting that appropriate caution should be used in handling VSV-SARS-CoV-2 at BSL2.

| | | |
|---|---|---|
| REAGENT or | | |
| RESOURCE | SOURCE | IDENTIFIER |

Antibodies

| | | |
|---|---|---|
| Anti-human IgG | Sigma-Aldrich | Cat# A6029-1ML; RRID: |
| peroxidase | | AB 258272 |
| CR3022 | ter Meulen et al., 2006; Yuan et al., | N/A |
| | 2020 | |
| Goat anti-human | Abcam | Cat# ab6858-1MG; RRID: |
| IgG - H&L (HRP) | | AB_955433 |
| Phage display | Miersch et al., Bioraxiv | N/A |
| antibody set | https://doi.org/10.1101/2020.06.05.137349 | |
| VIR antibody set | Pinto et al., 2020 | S309 PDB: 6WS6 |

Bacterial and Virus Strains

| | | |
|---|---|---|
| 2019 n- | CDC (gift from Natalie Thornburg) | N/A |
| CoV/USA_WA1/2020 | | |
| Vaccinia virus | Fuerst et al., 1986 | N/A |
| vTF7-3 | | |
| VSV-eGFP | Chandran et al., 2005 | N/A |

Biological Samples

| | | |
|---|---|---|
| Patient serum set | This study | N/A |

Chemicals, Peptides, and Recombinant Proteins

| | | |
|---|---|---|
| Blasticidin S HCl | GIBCO/Thermo Fisher | Cat# A1113903 |
| Cytosine | Sigma-Aldrich | Cat# C1768 |
| arabinoside | | |
| Formaldehyde | Millipore Sigma | Cat# FX0410-5 |
| Solution | | |
| HEPES, free acid | Millipore Sigma | Cat #5310-OP |
| Hoechst 33342 | Invitrogen/Thermo Fisher | Cat# H3570 |
| Human ACE2-Fc | This study | GenBank: BAB40370.1 & |
| | | AAC82527.1 |
| L-[³⁵S]-cysteine | Perkin Elmer | Cat# NEG022T |
| L-[³⁵S]-methionine | Perkin Elmer | Cat# NEG009T |
| Lipofectamine 2000 | Invitrogen/Thermo Fisher | Cat# 11668019 |
| Transfection | | |
| Reagent | | |
| Mouse ACE2-Fc | This study | NCBI Reference Sequence: |
| | | NP_001123985.1 |
| Phosphotungstic | Sigma-Aldrich | Cat# 79690-25G |
| acid hydrate | | |
| Pierce ECL | Thermo Scientific/Thermo Fisher | Cat# 32106 |
| Western Blotting | | |
| Substrate | | |
| Protein A Agarose | GoldBio | Cat# P-400-100 |
| Resin | | |
| TRIzol Reagent | Invitrogen/Thermo Fisher | Cat# 15596018 |
| TrueBlue | KPL/SeraCare | Cat# 5510-0050 |
| peroxidase | | |
| substrate | | |

Critical Commercial Assays

| | | |
|---|---|---|
| Anti-SARS-CoV-2 | Euroimmun US, Inc., 1 Bloomfield Ave, | N/A |
| ELISA (IgG) | Mountain Lakes, NJ 07046 | |
| serology test | | |

Deposited Data

| | | |
|---|---|---|
| Nucleotide | This study | BioProject: PRJNA635934; SRA: |
| sequence of VSV- | | SRR11878607 |
| eGFP-SARS-CoV- | | |
| 2-S_{Δ21} | | |

Experimental Models: Cell Lines

| | | |
|---|---|---|
| A549 | Gift from Jan Carette | N/A |
| BHK-21 | ATCC | Cat# CCL-10 |
| BSRT7/5 | Buchholz et al., 1999 | N/A |
| Caco-2 | ATCC | Cat# HTB-37 |
| Caco-2 BBe1 | ATCC | Cat# CRL-2102 |
| Calu-3 | Gift from Stanley Perlman | N/A |
| Expi293F | Thermo Fisher | Cat# A14527 |
| ExpiCHO | GIBCO/Thermo Fisher | Cat# A29127 |
| HEK293 | ATCC | Cat# CRL-1573 |
| HEK293T | ATCC | Cat# CRL-3216 |
| H1Hela | Gift from Jan Carette | N/A |
| HepG2 | ATCC | Cat# HB-8065 |
| HT-29 | ATCC | Cat# HTB-38 |
| Huh7.5.1 | Gift from Harry Greenberg | |
| MA104 | Gift from Harry Greenberg | N/A |
| Vero CCL81 | ATCC | Cat# CCL-81 |
| Vero E6 | ATCC | Cat# CRL-1586 |
| Vero E6-TMPRSS2 | This paper | N/A |
| Vero-furin | Mukherjee et al., 2016 | N/A |

Experimental Models: Organisms/Strains

| | | |
|---|---|---|
| N/A | N/A | N/A |

Oligonucleotides

| | | |
|---|---|---|
| TruSeq RNA Single | Illumina | Cat# 20020492 |
| Indexes Set A | | |
| TruSeq RNA Single | Illumina | Cat# 20020493 |
| Indexes Set B | | |

Recombinant DNA

| | | |
|---|---|---|
| pCAGGS-VSV-G | Stanifer et al., 2011 | N/A |
| pFM1.2-hACE2-Fc | This paper | GenBank: AB046569.1 |
| pFM1.2-mACE2-Fc | This paper | NCBI Reference Sequence: |
| | | NM_001130513.1 |
| pGEM3-VSV L | Whelan et al., 1995 | N/A |
| pGEM3-VSV G | Stanifer et al., 2011 | N/A |
| pGEM3-VSV N | Whelan et al., 1995 | N/A |
| pGEM3-VSV P | Whelan et al., 1995 | N/A |
| pLX304-TMPRSS2 | Zang et al., 2020 | N/A |
| psPAX2 | Gift from Didier Trono (unpublished | Addgene Cat# 12260 |
| | data) | |
| pVSV-eGFP | Chandran et al., 2005 | N/A |
| pVSV-eGFP-SARS- | This study | Spike mutated from MN908947.3 |
| CoV-2-S_{AA} | | |
| pVSV-eGFP-SARS- | This paper | Spike mutated from a codon- |
| CoV-2-S MERS S_{AA} | | optimized version of AHI48572.1 |
| cytoplasmic tail | | |
| switch | | |
| pVSV-eGFP-SARS- | This paper | VSV Indiana G (strain Orsay) |
| CoV-2-S VSV G #1 | | |
| cytoplasmic tail | | |
| switch | | |
| pVSV-eGFP-SARS- | This paper | VSV Indiana G (strain Orsay) |
| CoV-2-S VSV G #2 | | |
| cytoplasmic tail | | |
| switch | | |
| pVSV-eGFP-SARS- | This paper | VSV Indiana G (strain Orsay) |
| CoV-2-S VSV G | | |
| Ecto/TM/tail switch | | |
| pVSV-eGFP-SARS- | This paper | VSV Indiana G (strain Orsay) |
| CoV-2-S VSV G | | |
| TM/tail switch | | |

Software and Algorithms

| | | |
|---|---|---|
| BBMap 38.79 | Bushnell et al., 2017 | https://jgi.doe.gov/data-and-tools/bbtools/ |
| Fastp 0.20.0 | Chen et al., 2018 | https://github.com/OpenGene/fastp |
| Image Analyses: | GE Life Sciences (now Cytiva) | N/A |
| GE InCell Analyzer | | |
| 1000 Workstation | | |
| NUCmer 3.1 | Delcher et al., 2002 | http://mummer.sourceforge.net/ |
| SAMtools 1.9 | Li et al., 2009 | http://www.htslib.org/ |
| SPAdes 3.13.0 | Bankevich et al., 2012 | http://cab.spbu.ru/software/spades/ |
| Statistics: Prism 8.0 | GraphPad | N/A |

Other

| | | |
|---|---|---|
| TruSeq Stranded | Illumina | Cat #20020596 |
| Total RNA Library | | |
| Prep Kit with Ribo- | | |
| Zero | | |
| Human/Mouse/Rat | | |

Data and Code Availability

The authors declare that all data supporting the findings of this study are available within the paper. The sequence of VSV-eGFP-SARS-CoV-2-S_{Δ21} (passage 12) generated during this study is available through NCBI (SRA: SRR11878607, https://www.ncbi.nlm.nih.gov/sra/?term=SRR11878607; BioProject: PRJNA635934).

Experimental Model and Subject Details

Cells

Cells were maintained in humidified incubators at 34 or 37° C. and 5% CO₂ in the indicated media. BSRT7/5, Vero CCL81, Vero E6, Vero E6-TMPRSS2, A549, Caco-2, Caco-2 BBe1, Calu-3, Huh7.5.1, HepG2, H1Hela, BHK-21, HEK293, and HEK293T were maintained in DMEM (Corning or VWR) supplemented with glucose, L-glutamine, sodium pyruvate, and 10% fetal bovine serum (FBS). Vero-furin cells (Mukherjee et al., 2016) also were supplemented with 5 μg/mL of Blasticidin S HCl (GIBCO). MA104 cells were maintained in Medium 199 (GIBCO) containing 10% FBS. HT-29 cells were cultured in complete DMEM/F12 (Thermo Fisher) supplemented with sodium pyruvate, non-essential amino acids, and HEPES (Millipore Sigma). Vero E6-TMPRSS2 cells were generated using a lentivirus vector. Briefly, HEK293T producer cells were transfected with pLX304-TMPRSS2, pCAGGS-VSV-G, and psPAX2, and cell culture supernatants were collected at 48 h and clarified by centrifugation at 1,000×g for 5 min. The resulting lentivirus was used to infect Vero E6 cells for 24 h, and cells were selected with 40 μg/mL Blasticidin S HCl for 7 days.

Recombinant VSV

Recovery of recombinant VSV was performed as described (Whelan et al., 1995). Briefly, BSRT7/5 cells (Buchholz et al., 1999) were inoculated with vaccinia virus vTF7-3 (Fuerst et al., 1986) and subsequently transfected with T7-expression plasmids encoding VSV N, P, L, and G, and an antigenomic copy of the viral genome. Cell culture supernatants were collected at 56-72 h, clarified by centrifugation (5 min at 1,000×g), and filtered through a 0.22 μm filter. Virus was plaque-purified on Vero CCL81 cells in the presence of 25 μg/mL of cytosine arabinoside (Sigma-Aldrich), and plaques in agarose plugs were amplified on Vero CCL81 cells. Viral stocks were amplified on MA104 cells at an MOI of 0.01 in Medium 199 containing 2% FBS and 20 mM HEPES pH 7.7 at 34° C. Viral supernatants were harvested upon extensive cytopathic effect and clarified of cell debris by centrifugation at 1,000×g for 5 min. Aliquots were maintained at −80° C. Construction and use of VSV-SARS-CoV-2 was approved by the Washington University School of Medicine Institutional Biosafety Committee at Biosafety level 2.

SARS-CoV-2

SARS-CoV-2 strain 2019 n-CoV/USA_WA1/2020 was obtained from the Centers for Disease Control and Prevention (gift of Natalie Thornburg). Virus was passaged in the indicated producer cells (FIG. 16A-FIG. 16C). Work with SARS-CoV-2 was approved by the Washington University School of Medicine Institutional Biosafety Committee at Biosafety level 3 with positive pressure respirators.

Method Details

Plasmids

The S gene of SARS-CoV-2 isolate Wuhan-Hu-1 (GenBank MN908947.3) was synthesized in two fragments (Integrated DNA Technologies) and inserted into an infectious molecular clone of VSV (Chandran et al., 2005; Whelan et al., 1995) as previously (Carette et al., 2011; Jae et al., 2014). Modifications to the cytoplasmic tail were assembled identically, and accession numbers or references to the amino acid sequences used for the MERS and VSV G tail mutants can be found in the Key Resources Table. Other plasmids were previously described: VSV N, P, L and G expression plasmids (Stanifer et al., 2011; Whelan et al., 1995), psPAX2 (Addgene), and pLX304-TMPRSS2 (Zang et al., 2020).

Next Generation Sequencing

Total RNA was extracted from Vero CCL81 cells infected with VSV-SARS-CoV-2-S_{Δ21} using TRIzol (Invitrogen) according to the manufacturer's protocol. RNA was used to generate next generation sequencing libraries using TruSeq Stranded Total RNA library kit with Ribo Zero ribosomal subtraction (Illumina). The libraries were quantified using a bioanalyzer (Agilent) and pooled at an equal molar concentration and used to generate paired end 250 bp reads on a MiSeq (Illumina). Raw sequencing data was processed using fastp v0.20.0 (Chen et al., 2018) to cut adaptor sequences and filter out sequences with a Phred quality score <30. Processed reads were aligned to the reference plasmid sequence using BBMap v38.79 (Bushnell et al., 2017). Reads that mapped to the reference were extracted using SAMtools 1.9 (Li et al., 2009). The extracted mapped reads were used as input for de novo assembly with SPAdes v3.13.0 (Bankevich et al., 2012). Assembled contigs produced a 14.2 kb consensus sequence, which was subsequently aligned to the reference plasmid using NUCmer v3.1 (Delcher et al., 2002). Consensus sequences for each RNA sample were generated by aligning contigs to the reference plasmid sequence pVSV(1+)-eGFP-SARS-CoV-2-S with SnapGene v5.0.

Western Blotting

Purified VSV virions were incubated in non-reducing denaturation buffer (55 mM Tris-HCl pH 6.8, 1.67% (w/v) SDS, 7.5% (w/v) glycerol) at 100° C. for 5 min. Viral proteins were separated on a 8% acrylamide gel, transferred onto a nitrocellulose membrane, and incubated with human anti-SARS antibody CR3022 diluted in Tris-buffered saline containing 1% Tween-20 (TBS-T) and 5% milk, followed by incubation with HRP-conjugated goat anti-human antibody (Abcam) diluted in TBS-T containing 1% milk. HRP activity was visualized using the Pierce ECL western blotting kit (Thermo Scientific) and imaged with a ChemiDoc™ MP Imager (Bio-Rad).

Metabolic Radiolabeling of Virions

To generate high titer stocks of VSV-SARS-CoV-2, BSRT7/5 cells were transfected with pCAGGS-VSV-G in Opti-MEM (GIBCO) using Lipofectamine 2000 (Invitrogen) and infected 7 h later with VSV-SARS-CoV-2-S_{Δ21} at an MOI of 0.1 in DMEM containing 2% FBS and 20 mM HEPES pH 7.7. Viral stocks were collected at 48 hpi, and used to infect fresh cells (MOI of 10) for labeling of viral proteins. At 4 hpi, cells were starved in serum free, methionine/cysteine free DMEM (Corning), and exposed to 15 μCi/mL [³⁵S]-methionine and [³⁵S]-cysteine (Perkin Elmer) from 5-24 h in the presence of actinomycin D. Cell culture supernatants were collected, clarified by centrifugation (1,500×g, 5 min), and analyzed by SDS-PAGE and detected by phosphoimage analysis (Li et al., 2006).

Transmission Electron Microscopy

Purified viruses were adhered to glow-discharged, carbon-coated copper grids. Samples were stained with 2% (w/v) phosphotungstic acid (Sigma-Aldrich), pH 7.1, in H₂O and viewed on a JEOL 1200 EX transmission electron microscope (JEOL USA Inc.) equipped with an AMT 8-megapixel digital camera and AMT Image Capture Engine V602 software (Advanced Microscopy Techniques).

Monoclonal Antibodies

Phage-displayed Fab libraries were panned against immobilized SARS-CoV-2 spike RBD in multiple rounds using established methods (Persson et al., 2013). Following four rounds of selection, phage ELISAs were used to screen 384 clones to identify those that bound specifically to RBD. The complementarity determining regions of Fab-phage clones were decoded by sequencing the variable regions and cloning them into mammalian expression vectors for expression and purification of human IgG1 proteins, as described (Tao et al., 2019). A subset of the panel of mAbs was tested for neutralization as a part of this study.

Another set of mAbs (S304, S306, S309, S310, and S315) were isolated from EBV-immortalized memory B cells from a SARS-CoV survivor (Traggiai et al., 2004) and are cross-reactive to SARS-CoV-2 (Pinto et al., 2020). Recombinant antibodies were expressed in ExpiCHO cells transiently co-transfected with plasmids expressing the heavy and light chain as previously described (Stettler et al., 2016).

Human Sera

Human samples were collected from PCR-confirmed COVID-19 patients.

Serum samples were obtained by routine phlebotomy at different days post symptom onset (range: day 5-20). Samples were prescreened by the Euroimmun anti-SARS-CoV-2 IgG ELISA (Lubeck, Germany), a qualitative assay with the Food and Drug Administration Emergency Use Authorization that detects antibodies to the SARS-CoV-2 S protein. This study was approved by the Mayo Clinic Institutional Review Board.

Protein Expression and Purification

DNA fragments encoding human ACE2 (hACE2 residues 1-615) and mouse ACE2 (mACE2, residues 1-615) were synthesized and cloned into pFM1.2 with a C-terminal HRV-3C protease cleavage site (LEVLFQGP) and a human IgG1 Fc region as previously described (Raj et al., 2013). We transiently transfected plasmids into Expi293F cells and harvested cell supernatants 4 days post transfection. Secreted hACE2-Fc and mACE2-Fc proteins were purified by protein A chromatography (GoldBio).

GFP-Reduction Neutralization Test

Patient samples were heat-inactivated at 56° C. for 30 min. Indicated dilutions of samples were incubated with 10² PFU of VSV-SARS-CoV-2-S_{Δ21} for 1 h at 37° C. Antibody-virus complexes were added to Vero E6 cells in 96-well plates and incubated at 37° C. for 7.5 h. Cells subsequently were fixed in 2% formaldehyde (Millipore Sigma) containing 10 μg/mL Hoechst 33342 nuclear stain (Invitrogen) for 45 min at room temperature, when fixative was replaced with PBS. Images were acquired with the InCell 2000 Analyzer (GE Healthcare) automated microscope in both the DAPI and FITC channels to visualize nuclei and infected cells (i.e., eGFP-positive cells), respectively (4× objective, 4 fields per well, covering the entire well). Images were analyzed using the Multi Target Analysis Module of the InCell Analyzer 1000 Workstation Software (GE Healthcare). GFP-positive cells were identified in the FITC channel using the top-hat segmentation method and subsequently counted within the InCell Workstation software. The sensitivity and accuracy of GFP-positive cell number determinations were validated using a serial dilution of virus. A background number of GFP+ cells was subtracted from each well using an average value determined from at least 4 uninfected wells. Data were processed using Prism software (GraphPad Prism 8.0). ACE2 neutralization assays using VSV-SARS-CoV-2-S_{Δ21} were conducted similarly but using an MOI of 1. Infection and imaging of Calu-3 cells was performed using similar methods, though cells were infected at an MA104-calculated MOI of 20. Cells were imaged using a 10× objective, with 9 fields per well. FITC and DAPI fields were overlaid using ImageJ and contrast-adjusted identically.

Focus-Reduction Neutralization Test

Indicated dilutions of mAbs, sera, or protein were incubated with 10² FFU of SARS-CoV-2 for 1 h at 37° C. Antibody-virus complexes were added to indicated cell monolayers in 96-well plates and incubated at 37° C. for 1 h. Subsequently, cells were overlaid with 1% (w/v) methylcellulose in MEM supplemented with 2% FBS. Plates were harvested 30 h later by removing overlays and fixed with 4% paraformaldehyde in PBS for 20 min at room temperature. Plates were washed and sequentially incubated with 1 μg/mL of CR3022 anti-S antibody (ter Meulen et al., 2006; Yuan et al., 2020) and HRP-conjugated goat anti-human IgG in PBS supplemented with 0.1% saponin and 0.1% BSA. SARS-CoV-2-infected cell foci were visualized using TrueBlue peroxidase substrate (KPL) and quantitated on an ImmunoSpot microanalyzer (Cellular Technologies). Data were processed using Prism software (GraphPad Prism 8.0).

Quantification and Statistical Analysis

All statistical tests were performed using GraphPad Prism 8.0 software as described in the indicated figure legends. Statistical significance was assigned when P values were <0.05. The number of independent experiments and the statistical tests used are indicated in the relevant Figure legends. Error bars indicate standard error of the mean unless stated otherwise.

### REFERENCES

Y. Bai, L. Yao, T. Wei, F. Tian, D. Y. Jin, L. Chen, M. Wang Presumed Asymptomatic Carrier Transmission of COVID-19 JAMA (2020), 10.1001/jama.2020.2565
A. Bankevich, S. Nurk, D. Antipov, A. A. Gurevich, M. Dvorkin, A. S. Kulikov, V. M. Lesin, S. I. Nikolenko, S. Pham, A. D. Prjibelski, et al. SPAdes: a new genome assembly algorithm and its applications to single-cell sequencing J Comp. Biol., 19 (2012), pp. 455-477
E. M. Bloch, S. Shoham, A. Casadevall, B. S. Sachais, B. Shaz, J. L. Winters, C. van Buskirk, B. J. Grossman, M. Joyner, J. P. Henderson, et al. Deployment of convalescent plasma for the prevention and treatment of COVID-19 J Clin Invest., 130 (2020), pp. 2757-2765
K. S. Brown, D. Safronetz, A. Marzi, H. Ebihara, H. Feldmann Vesicular stomatitis virus-based vaccine protects hamsters against lethal challenge with Andes virus J. Virol., 85 (2011), pp. 12781-12791
U. J. Buchholz, S. Finke, K. K. Conzelmann Generation of bovine respiratory syncytial virus (BRSV) from cDNA: BRSV NS2 is not essential for virus replication in tissue culture, and the human RSV leader region acts as a functional BRSV genome promoter J. Virol., 73 (1999), pp. 251-259
B. Bushnell, J. Rood, E. Singer BBMerge—Accurate paired shotgun read merging via overlap PLoS ONE, 12 (2017), p. e0185056
J. E. Carette, M. Raaben, A. C. Wong, A. S. Herbert, G. Obernosterer, N. Mulherkar, A.l. Kuehne, P. J. Kranzusch, A. M. Griffin, G. Ruthel, et al. Ebola virus entry requires the cholesterol transporter Niemann-Pick C1 Nature, 477 (2011), pp. 340-343
K. Chandran, N. J. Sullivan, U. Felbor, S. P. Whelan, J. M. Cunningham Endosomal proteolysis of the Ebola virus glycoprotein is necessary for infection Science, 308 (2005), pp. 1643-1645
J. Chen, J. M. Kovacs, H. Peng, S. Rits-Volloch, J. Lu, D. Park, E. Zablowsky, M. S. Seaman, B. Chen HIV-1 ENVELOPE. Effect of the cytoplasmic domain on antigenic characteristics of HIV-1 envelope glycoprotein Science, 349 (2015), pp. 191-195
S. Chen, Y. Zhou, Y. Chen, J. Gu fastp: an ultra-fast all-in-one FASTQ preprocessor Bioinformatics, 34 (2018), pp. i884-i890
L. Chen, J. Xiong, L. Bao, Y. Shi Convalescent plasma as a potential therapy for COVID-19 Lancet Infect. Dis., 20 (2020), pp. 398-400
A. L. Delcher, A. Phillippy, J. Carlton, S. L. Salzberg Fast algorithms for large-scale genome alignment and comparison Nucleic Acids Res., 30 (2002), pp. 2478-2483
M. R. Denison, R. L. Graham, E. F. Donaldson, L. D. Eckerle, R. S. Baric Coronaviruses: an RNA proofreading machine regulates replication fidelity and diversity RNA Biol., 8 (2011), pp. 270-279
M. E. Dieterle, D. Haslwanter, R. H. Bortz, A. S. Wirchnianski, G. Lasso, O. Vergnolle, S. A. Abbasi, J. M. Fels, E. Laudermilch, C. Florez, et al. A replication-competent vesicular stomatitis virus for studies of SARS-CoV-2 spike-mediated cell entry and its inhibition Cell Host & Microbe, 28 (2020), 10.1016/j.chom.2020.06.020
T. R. Fuerst, E. G. Niles, F. W. Studier, B. Moss Eukaryotic transient-expression system based on recombinant vaccinia virus that synthesizes bacteriophage T7 RNA polymerase Proc. Natl. Acad. Sci. USA, 83 (1986), pp. 8122-8126
S. Fukushi, T. Mizutani, M. Saijo, S. Matsuyama, N. Miyajima, F. Taguchi, S. Itamura, I. Kurane, S. Morikawa Vesicular stomatitis virus pseudotyped with severe acute respiratory syndrome coronavirus spike protein J. Gen. Virol., 86 (2005), pp. 2269-2274
S. Fukushi, T. Mizutani, M. Saijo, I. Kurane, F. Taguchi, M. Tashiro, S. Morikawa Evaluation of a novel vesicular stomatitis virus pseudotype-based assay for detection of neutralizing antibody responses to SARS-CoV J. Med. Virol., 78 (2006), pp. 1509-1512
W. Furuyama, P. Reynolds, E. Haddock, K. Meade-White, M. Quynh Le, Y. Kawaoka, H. Feldmann, A. Marzi A single dose of a vesicular stomatitis virus-based influenza vaccine confers rapid protection against H5 viruses from different clades NPJ Vaccines, 5 (2020), p. 4
M. Garbutt, R. Liebscher, V. Wahl-Jensen, S. Jones, P. Möller, R. Wagner, V. Volchkov, H. D. Klenk, H. Feldmann, U. Stroher Properties of replication-competent vesicular stomatitis virus vectors expressing glycoproteins of filoviruses and arenaviruses J. Virol., 78 (2004), pp. 5458-5465
T. W. Geisbert, S. Jones, E. A. Fritz, A. C. Shurtleff, J. B. Geisbert, R. Liebscher, A. Grolla, U. Stroher, L. Fernando, K. M. Daddario, et al. Development of a new vaccine for the prevention of Lassa fever PLoS Med., 2 (2005), p. e183
T. Giroglou, J. Cinatl Jr., H. Rabenau, C. Drosten, H. Schwalbe, H. W. Doerr, D. von Laer Retroviral vectors pseudotyped with severe acute respiratory syndrome coronavirus S protein J. Virol., 78 (2004), pp. 9007-9015
W. J. Guan, Z. Y. Ni, Y. Hu, W. H. Liang, C. Q. Ou, J. X. He, L. Liu, H. Shan, C. L. Lei, D. S. C. Hui, et al., China Medical Treatment Expert Group for Covid-19 Clinical Characteristics of Coronavirus Disease 2019 in China N. Engl. J. Med., 382 (2020), pp. 1708-1720
M. Hoffmann, H. Kleine-Weber, S. Schroeder, N. Kruger, T. Herrler, S. Erichsen, T. S. Schiergens, G. Herrler, N. H. Wu, A. Nitsche, et al. SARS-CoV-2 Cell Entry Depends on ACE2 and TMPRSS2 and Is Blocked by a Clinically Proven Protease Inhibitor Cell, 181 (2020), pp. 271-280.e8
L. T. Jae, M. Raaben, A. S. Herbert, A. I. Kuehne, A. S. Wirchnianski, T. K. Soh, S. H. Stubbs, H. Janssen, M. Damme, P. Saftig, et al. Virus entry. Lassa virus entry requires a trigger-induced receptor switch Science, 344 (2014), pp. 1506-1510
S. M. Jones, H. Feldmann, U. Stroher, J. B. Geisbert, L. Fernando, A. Grolla, H. D. Klenk, N. J. Sullivan, V. E. Volchkov, E. A. Fritz, et al. Live attenuated recombinant vaccine protects nonhuman primates against Ebola and Marburg viruses Nat. Med., 11 (2005), pp. 786-790
G. P. Kobinger, M. P. Limberis, S. Somanathan, G. Schumer, P. Bell, J. M. Wilson Human immunodeficiency viral vector pseudotyped with the spike envelope of severe acute respiratory syndrome coronavirus transduces human airway epithelial cells and dendritic cells Hum. Gene Ther., 18 (2007), pp. 413-422
R. L. Kruse Therapeutic strategies in an outbreak scenario to treat the novel coronavirus originating in Wuhan, China F1000Res., 9 (2020), p. 72
C. Lei, K. Qian, T. Li, S. Zhang, W. Fu, M. Ding, S. Hu Neutralization of SARS-CoV-2 spike pseudotyped virus by recombinant ACE2-Ig Nat. Commun., 11 (2020), p. 2070
M. Letko, A. Marzi, V. Munster Functional assessment of cell entry and receptor usage for SARS-CoV-2 and other lineage B betacoronaviruses Nat. Microbiol., 5 (2020), pp. 562-569
W. Li, M. J. Moore, N. Vasilieva, J. Sui, S. K. Wong, M. A. Berne, M. Somasundaran, J. L. Sullivan, K. Luzuriaga, T. C. Greenough, et al. Angiotensin-converting enzyme 2 is a functional receptor for the SARS coronavirus Nature, 426 (2003), pp. 450-454
F. Li, W. Li, M. Farzan, S. C. Harrison Structure of SARS coronavirus spike receptor-binding domain complexed with receptor Science, 309 (2005), pp. 1864-1868
J. Li, J. T. Wang, S. P. Whelan A unique strategy for mRNA cap methylation used by vesicular stomatitis virus Proc. Natl. Acad. Sci. USA, 103 (2006), pp. 8493-8498
H. Li, B. Handsaker, A. Wysoker, T. Fennell, J. Ruan, N. Homer, G. Marth, G. Abecasis, R. Durbin, 1000 Genome Project Data Processing Subgroup The Sequence Alignment/Map format and SAMtools Bioinformatics, 25 (2009), pp. 2078-2079
E. Lontok, E. Corse, C. E. Machamer Intracellular targeting signals contribute to localization of coronavirus spike proteins near the virus assembly site J. Virol., 78 (2004), pp. 5913-5922
C. E. McBride, J. Li, C. E. Machamer The cytoplasmic tail of the severe acute respiratory syndrome coronavirus spike protein contains a novel endoplasmic reticulum retrieval signal that binds COPI and promotes interaction with membrane protein J. Virol., 81 (2007), pp. 2418-2428
S. Miersch, M. J. Ustav, Z. Li, J. B. Case, S. Ganaie, G. Matusali, F. Colavita, D. Lapa, M. R. Capobianchi, G. Novelli, et al. Synthetic antibodies neutralize SARS-CoV-2 infection of mammalian cells bioRxiv (2020), 10.1101/2020.06.05.137349
M. J. Moore, T. Dorfman, W. Li, S. K. Wong, Y. Li, J. H. Kuhn, J. Coderre, N. Vasilieva, Z. Han, T. C. Greenough, et al. Retroviruses pseudotyped with the severe acute respiratory syndrome coronavirus spike protein efficiently infect cells expressing angiotensin-converting enzyme 2 J. Virol., 78 (2004), pp. 10628-10635
S. Mukherjee, D. Sirohi, K. A. Dowd, Z. Chen, M. S. Diamond, R. J. Kuhn, T. C. Pierson Enhancing dengue virus maturation using a stable furin over-expressing cell line Virology, 497 (2016), pp. 33-40
J. Nie, Q. Li, J. Wu, C. Zhao, H. Hao, H. Liu, L. Zhang, L. Nie, H. Qin, M. Wang, et al. Establishment and validation of a pseudovirus neutralization assay for SARS-CoV-2 Emerg. Microbes Infect., 9 (2020), pp. 680-686
X. Ou, Y. Liu, X. Lei, P. Li, D. Mi, L. Ren, L. Guo, R. Guo, T. Chen, J. Hu, et al. Characterization of spike glycoprotein of SARS-CoV-2 on virus entry and its immune cross-reactivity with SARS-CoV Nat. Commun., 11 (2020), p. 1620
H. Persson, W. Ye, A. Wernimont, J. J. Adams, A. Koide, S. Koide, R. Lam, S. S. Sidhu CDR-H3 diversity is not required for antigen recognition by synthetic antibodies J. Mol. Biol., 425 (2013), pp. 803-811
D. Pinto, Y. J. Park, M. Beltramello, A. C. Walls, M. A. Tortorici, S. Bianchi, S. Jaconi, K. Culap, F. Zatta, A. De Marco, et al. Cross-neutralization of SARS-CoV-2 by a human monoclonal SARS-CoV antibody Nature, 583 (2020), pp. 290-295
V. S. Raj, H. Mou, S. L. Smits, D. H. Dekkers, M. A. Müller, R. Dijkman, D. Muth, J. A. Demmers, A. Zaki, R. A. Fouchier, et al. Dipeptidyl peptidase 4 is a functional receptor for the emerging human coronavirus-EMC Nature, 495 (2013), pp. 251-254
T. R. Ruch, C. E. Machamer The coronavirus E protein: assembly and beyond Viruses, 4 (2012), pp. 363-382
C. Shen, Z. Wang, F. Zhao, Y. Yang, J. Li, J. Yuan, F. Wang, D. Li, M. Yang, L. Xing, et al. Treatment of 5 Critically Ill Patients With COVID-19 With Convalescent Plasma JAMA, 323 (2020), pp. 1582-1589
M. L. Stanifer, D. K. Cureton, S. P. Whelan A recombinant vesicular stomatitis virus bearing a lethal mutation in the glycoprotein gene uncovers a second site suppressor that restores fusion J. Virol., 85 (2011), pp. 8105-8115
K. Stettler, M. Beltramello, D. A. Espinosa, V. Graham, A. Cassotta, S. Bianchi, F. Vanzetta, A. Minola, S. Jaconi, F. Mele, et al. Specificity, cross-reactivity, and function of antibodies elicited by Zika virus infection Science, 353 (2016), pp. 823-826
A. Takada, H. Feldmann, U. Stroeher, M. Bray, S. Watanabe, H. Ito, M. McGregor, Y. Kawaoka Identification of protective epitopes on ebola virus glycoprotein at the single amino acid level by using recombinant vesicular stomatitis viruses J. Virol., 77 (2003), pp. 1069-1074
Y. Tao, M. Mis, L. Blazer, M. Ustav Jnr, Z. Steinhart, R. Chidiac, E. Kubarakos, S. O'Brien, X. Wang, N. Jarvik, et al. Tailored tetravalent antibodies potently and specifically activate Wnt/Frizzled pathways in cells, organoids and mice eLife, 8 (2019), p. e46134
J. ter Meulen, E. N. van den Brink, L. L. Poon, W. E. Marissen, C. S. Leung, F. Cox, C. Y. Cheung, A. Q. Bakker, J. A. Bogaards, E. van Deventer, et al. Human monoclonal antibody combination against SARS coronavirus: synergy and coverage of escape mutants PLoS Med., 3 (2006), p. e237
M. A. Tortorici, D. Veesler Structural insights into coronavirus entry Adv. Virus Res., 105 (2019), pp. 93-116
E. Traggiai, S. Becker, K. Subbarao, L. Kolesnikova, Y. Uematsu, M. R. Gismondo, B. R. Murphy, R. Rappuoli, A. Lanzavecchia An efficient method to make human monoclonal antibodies from memory B cells: potent neutralization of SARS coronavirus Nat. Med., 10 (2004), pp. 871-875
A. N. van den Pol, G. Mao, A. Chattopadhyay, J. K. Rose, J. N. Davis Chikungunya, Influenza, Nipah, and Semliki Forest Chimeric Viruses with Vesicular Stomatitis Virus: Actions in the Brain J. Virol., 91 (2017) e02154-16
A. C. Walls, Y. J. Park, M. A. Tortorici, A. Wall, A. T. McGuire, D. Veesler Structure, Function, and Antigenicity of the SARS-CoV-2 Spike Glycoprotein Cell, 181 (2020), pp. 281-292.e6
Y. Watanabe, J. D. Allen, D. Wrapp, J. S. McLellan, M. Crispin Site-specific glycan analysis of the SARS-CoV-2 spike Science (2020), p. eabb9983
S. P. Whelan, L. A. Ball, J. N. Barr, G. T. Wertz Efficient recovery of infectious vesicular stomatitis virus entirely from cDNA clones Proc. Natl. Acad. Sci. USA, 92 (1995), pp. 8388-8392
D. Wrapp, N. Wang, K. S. Corbett, J. A. Goldsmith, C. L. Hsieh, O. Abiona, B. S. Graham, J. S. McLellan Cryo-EM structure of the 2019-nCoV spike in the prefusion conformation Science (2020)
M. Yuan, N. C. Wu, X. Zhu, C. D. Lee, R. T. Y. So, H. Lv, C. K. P. Mok, I. A. Wilson A highly conserved cryptic epitope in the receptor binding domains of SARS-CoV-2 and SARS-CoV Science, 368 (2020), pp. 630-633
R. Zang, M. F. Gomez Castro, B. T. McCune, Q. Zeng, P. W. Rothlauf, N. M. Sonnek, Z. Liu, K. F. Brulois, X. Wang, H. B. Greenberg, et al. TMPRSS2 and TMPRSS4 promote SARS-CoV-2 infection of human small intestinal enterocytes Sci. Immunol., 5 (2020), p. eabc3582
T. Zhang, Q. Wu, Z. Zhang Probable Pangolin Origin of SARS-CoV-2 Associated with the COVID-19 Outbreak Curr Biol., 30 (2020), pp. 1346-1351.e2
P. Zhou, X. L. Yang, X. G. Wang, B. Hu, L. Zhang, W. Zhang, H. R. Si, Y. Zhu, B. Li, C. L. Huang, et al. A pneumonia outbreak associated with a new coronavirus of probable bat origin Nature, 579 (2020), pp. 270-273

### Example 5: Identification of SARS-CoV-2 Spike Mutations that Attenuate Monoclonal and Serum Antibody Neutralization

The following example describes VSV-SARS2-mutants to characterize antibody panels.

We have extended the above disclosure to generate a large panel of variants in the SARS-CoV-2 spike protein incorporated into the VSV-vector (see e.g., Liu et al. Cell Host Microbe 2021 Mar. 10; 29(3):477-488.e4. doi: 10.1016/j.chom.2021.01.014. Epub 2021 Jan. 27).

Those substitutions were largely selected as escape mutants to neutralizing antibodies or were designed to probe the function of circulating viral variants.

The substitutions in Spike (numbering is SARS CoV2 S) having amino acid mutation (reference sequence is SEQ ID NO: 1; S gene of SARS-CoV-2 isolate Wuhan-Hu-1 (GenBank MN908947.3)): E488A; E484K; E484D; E484G; S477N; S477G; S477R; K444E; K444N; T345A; T345N; T345S; G446D; G446V; R346G; N450D; N450K; N450Y; F486S; F486Y; L441R; L452R; A352D; T478I; F490S; S494P; P499L; T345A/L517R; S477N/S514F; and/or D614G. Others possible mutations to include can be any one or more of those listed in TABLE 2, such as T345A; T345A/L517R; T345A/E484A; T345N; T345S; T345S/F486S; T346G/E484A; R346K/E484K; R346G; R346K; A352D; A372T; A372T/E484K; K378E; K378Q; R408K; K417N; L441R; K444E; K444E/E484A; K444E/E484K; K444N; K444R; V445G; G446D; G446V; N450D; N450K; N450Y; L452R; K458Q; Q474P; G476D; G476S; S4771; S477G; S477N; S477N/S514F; S477R; T478I; T478P; P479L; P479S; V483F; V483G; E484A; E484D; E484K; E484G; F486L; F486S; F486Y; F486V; E488A; F490L; F490S; S494P; P499L; N501Y; G504D; S514F; L517R; K535R; and/or D614G; or combinations thereof.

This is a facile way of screening antibodies and soluble receptors and other inhibitors for their ability to neutralize VSV-SARS CoV-2 spike and for the currently dominant D614G variant. This panel will become increasingly relevant as the common substitutions in spike become more dominant.

Abstract

Neutralizing antibodies against the SARS-CoV-2 spike (S) protein are a goal of COVID-19 vaccines and have received emergency use authorization as therapeutics. However, viral escape mutants could compromise efficacy. To define immune-selected mutations in the S protein, we exposed a VSV-eGFP-SARS-CoV-2-S chimeric virus, in which the VSV glycoprotein is replaced with the S protein, to 19 neutralizing monoclonal antibodies (mAbs) against the receptor-binding domain (RBD) and generated 50 different escape mutants.

Each mAb had a unique resistance profile, although many shared residues within an epitope of the RBD. Some variants (e.g., S477N) were resistant to neutralization by multiple mAbs, whereas others (e.g., E484K) escaped neutralization by convalescent sera. Additionally, sequential selection identified mutants that escape neutralization by antibody cocktails. Comparing these antibody-mediated mutations with sequence variation in circulating SARS-CoV-2 revealed substitutions that may attenuate neutralizing immune responses in some humans and thus warrant further investigation.

Introduction

Control of the ongoing severe acute respiratory syndrome coronavirus 2 (SARS-CoV-2) pandemic will require deployment of multiple countermeasures, including therapeutics and vaccines. Therapeutic candidates that have received emergency use authorization (EUA) or are in development include several monoclonal antibodies (mAbs) (ACTIV-3/TICO LY-CoV555 Study Group, 2020; Chen et al., 2021; Weinreich et al., 2021) that recognize the SARS-CoV-2 spike (S) protein, which decorates the virion surface (Ke et al., 2020). The S protein is comprised of an N-terminal subunit (S1) that mediates receptor binding and a C-terminal subunit (S2) responsible for virus-cell membrane fusion (Wrapp et al., 2020). During viral entry into cells, the receptor-binding domain (RBD) of S1 engages the primary receptor, human angiotensin converting enzyme 2 (hACE2) (Letko et al., 2020). Processing of S by host cell proteases, typically TMPRSS2, TMPRSS4, or endosomal cathepsins, facilitates the S2-dependent fusion of viral and host-cell membranes (Hoffmann et al., 2020; Zang et al., 2020). Potently neutralizing antibodies against SARS-CoV-2 target the RBD (ACTIV-3/TICO LY-CoV555 Study Group, 2020; Brouwer et al., 2020; Rogers et al., 2020; Wu et al., 2020b; Zost et al., 2020), with many inhibiting infection by blocking receptor engagement (Alsoussi et al., 2020; Wu et al., 2020b). Understanding the epitopes recognized by protective antibodies and whether natural variation in the S protein is associated with resistance to neutralization may predict the utility of antibody-based countermeasures.

RNA viruses exist as a swarm or “quasispecies” of genome sequences around a core consensus sequence (Dolan et al., 2018). Under conditions of selection, such as those imposed by neutralizing antibodies or drugs, variants of the swarm can escape genetically and become resistant. The relative fitness of escape mutants determines whether they are lost rapidly from the swarm or provide a competitive advantage. The intrinsically high error rates of viral RNA-dependent RNA polymerases (RdRp) result in the stochastic introduction of mutations during viral genome replication with substitutions approaching a nucleotide change per genome for each round of replication (Sanjuen et al., 2010). Coronaviruses, because of their large genome size, encode a proofreading 3′ to 5′ exoribonuclease (ExoN, nsp14) that helps to correct errors made by the RdRp during replication (Smith and Denison, 2013). As a result of ExoN activity, the frequency of escape from antibody neutralization by coronaviruses is less than for other RNA viruses lacking such an enzyme (Smith et al., 2013).

To date, 4,150 mutations have been identified in the S gene of SARS-CoV-2 isolated from humans (CoV-GLUE, 2021; GISAID, 2021). These mutations give rise to 1,246 amino acid changes, including 187 substitutions in the RBD. The abundance of many variants in the human population suggests that they are not accompanied by a fitness loss. Multiple mechanisms likely account for the emergence of such substitutions including host adaptation, immune selection during natural infection, and possibly reinfection of individuals with incomplete or waning immunity. Convalescent plasma therapy, vaccination, and administration of therapeutic antibodies each could select for additional variants, and their effectiveness as countermeasures might be compromised by preexisting resistant mutants. Thus, as therapeutic antibodies and vaccines are deployed, it is increasingly important to define the patterns of antibody resistance that arise. The impact of SARS-CoV-2 adaptation for infection of other hosts including mice (Dinnon et al., 2020; Gu et al., 2020), mink (Oude Munnink et al., 2021), and domesticated animals (Halfmann et al., 2020; Shi et al., 2020) could also contribute to selection of new variants.

Here, we used a panel of antibodies including the previously reported 2B04, 1B07, and 2H04 mAbs (Alsoussi et al., 2020) and newly generated neutralizing mAbs against SARS-CoV-2 RBD to select for escape variants and define the mutational landscape of resistance. To facilitate selection, we used a chimeric, infectious vesicular stomatitis virus (VSV) in which the endogenous glycoprotein was replaced with the S protein of SARS-CoV-2 (Case et al., 2020). VSV-eGFP-SARS-CoV-2-S_{A}21 (herein, VSV-SARS-CoV-2) replicates to high titer (10⁷-10⁸ plaque forming units ml⁻¹ within 48 h), mimics the SARS-CoV-2 requirement for human ACE2 as a receptor, and is neutralized by SARS-CoV-2 S-specific mAbs (Case et al., 2020). In three selection campaigns using 19 different mAbs, we isolated 50 different escape mutants within the RBD. Many escape mutations arose proximal to or within the ACE2 binding footprint, suggesting that multiple neutralizing mAbs inhibit infection by interfering with receptor engagement. Cross-neutralization studies involving 29 of the escape mutants and 10 mAbs identified mutants that were resistant to multiple antibodies and also those with unique resistance profiles. Remarkably, substitutions at residue E484 of S protein were associated with resistance to neutralization by polyclonal human immune sera, suggesting that some individuals generate neutralizing antibodies recognizing a focused target on the RBD. Resistance to inhibition by soluble recombinant human ACE2, a candidate decoy molecule drug (Chan et al., 2020; Monteil et al., 2020) currently in clinical trials (NCT04375046 and NCT04287686), was observed with an F486S substitution. Cross-referencing of our 50 resistant mutants with sequences of clinical isolates of SARS-CoV-2 demonstrates that some already circulating variants will be resistant to monoclonal and polyclonal antibodies. This data and functional approach may be useful for monitoring and evaluating the emergence of escape from antibody-based therapeutic and vaccine countermeasures as they are deployed.

Results

Selection of mAb Escape Mutants in SARS-CoV-2 S

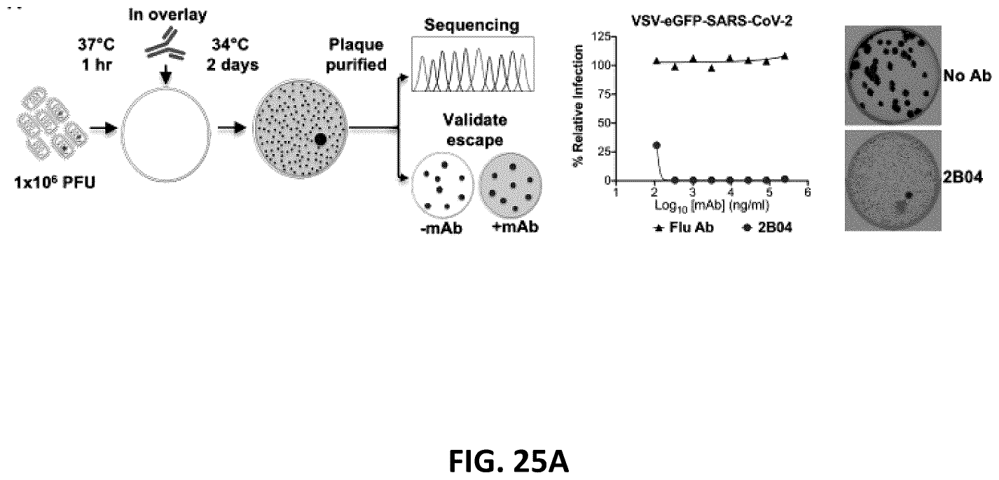

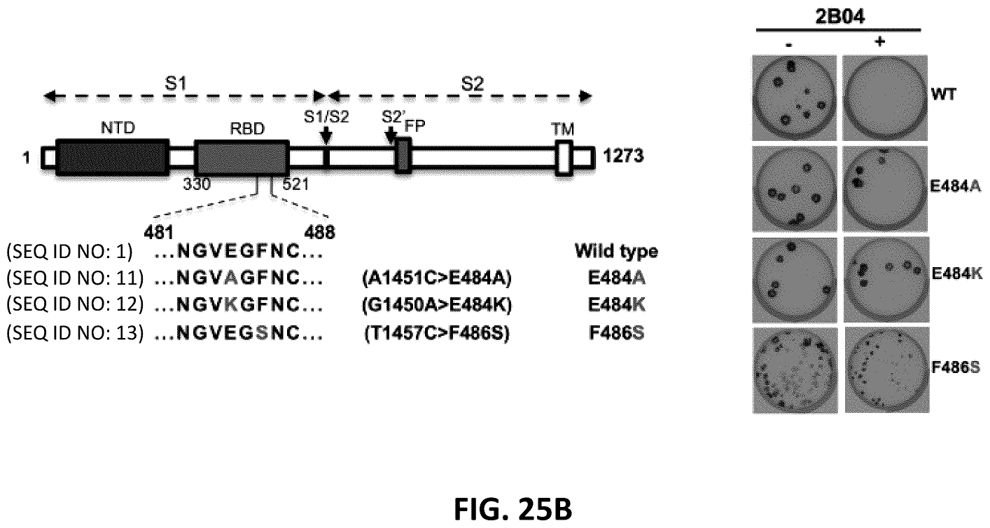

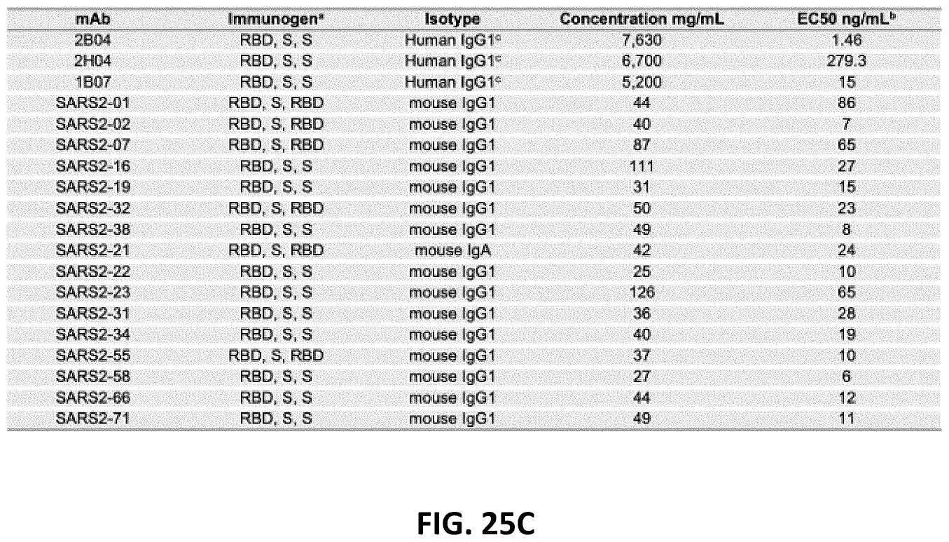

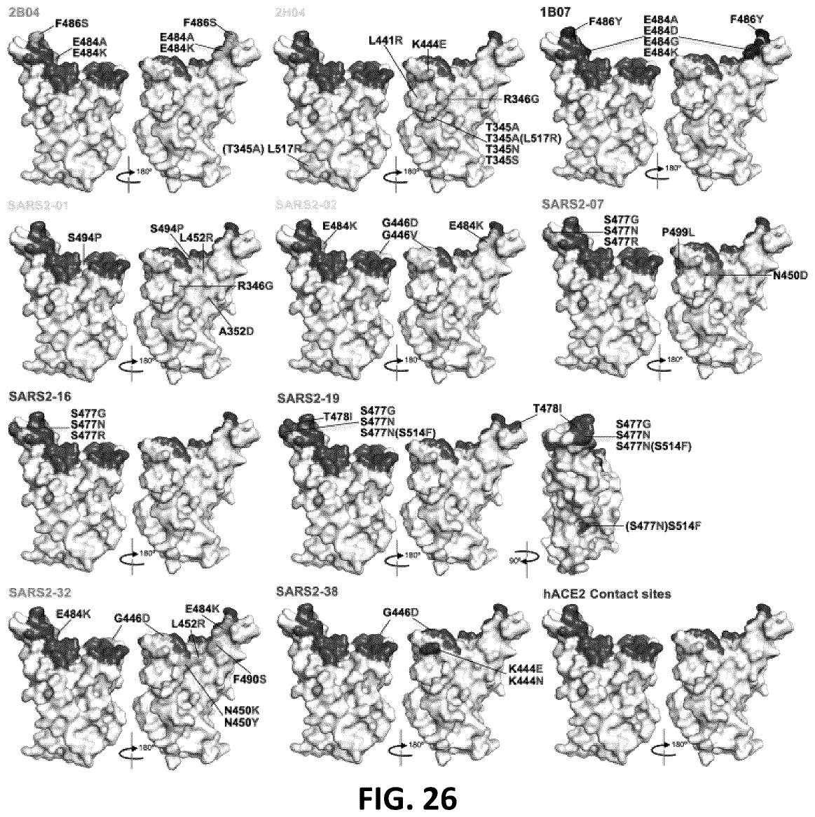

To select for SARS-CoV-2 S variants that escape neutralization, we used VSV-SARS-CoV-2 (Case et al., 2020) and mAb 2B04, which was generated from cloned murine B cells following immunization of C57BL/6 mice with recombinant RBD and boosted with recombinant S. Antibody neutralization resistant mutants were recovered by plaque isolation (FIG. 25A), and their resistance was verified by subsequent virus infection in the presence or absence of antibody. Antibody 2B04 failed to inhibit VSV-SARS-CoV-2-resistant variants as judged by plaque number and size (FIG. 25B). Sequence analysis identified the mutations E484A, E484K, and F486S (FIG. 25B), each of which falls within the RBD and maps to residues involved in ACE2 binding (FIG. 26) (Lan et al., 2020).

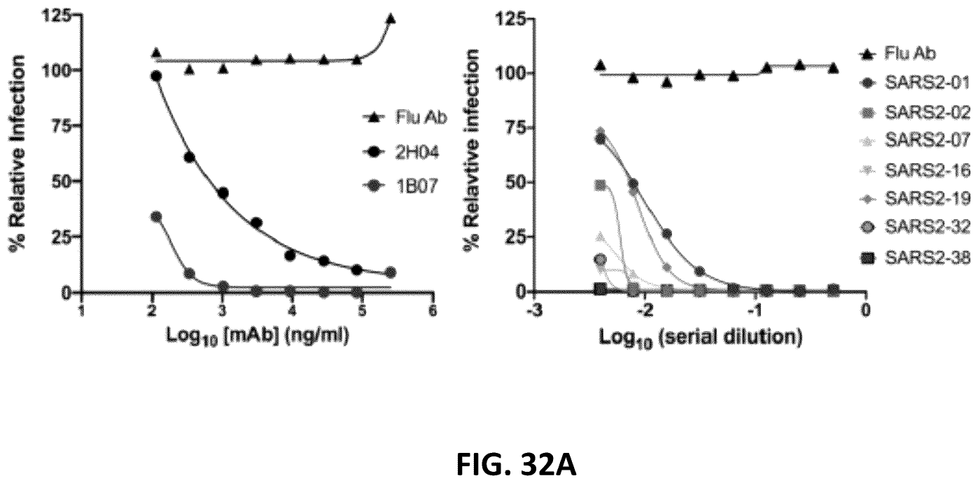

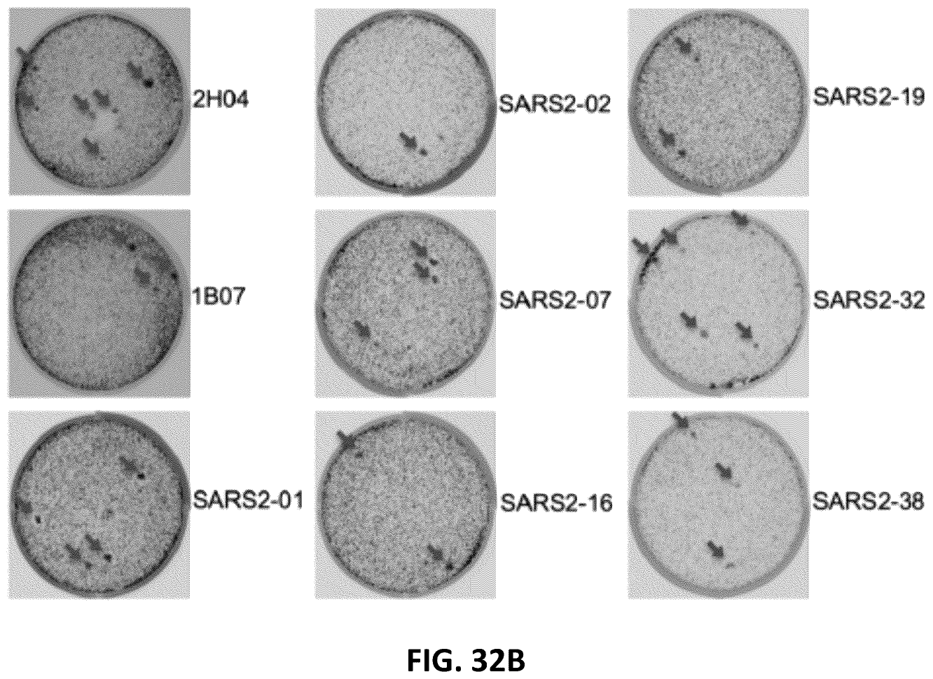

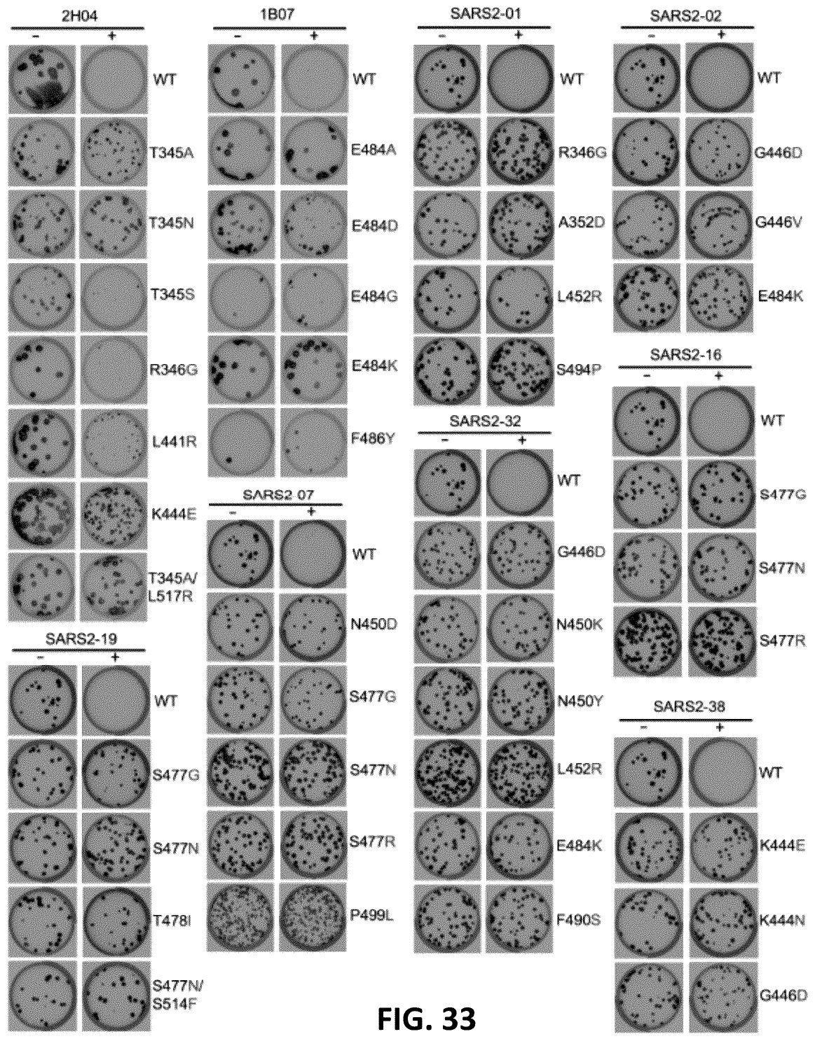

We extended this neutralization escape approach to nine additional inhibitory mAbs (FIG. 25C, FIG. 32, and FIG. 33). Sequence analysis of each isolated plaque identified multiple mutations within the RBD (TABLE 2), which we positioned on the reported crystal structure (PDB: 6M0J) (FIG. 26): 2B04 (green), 2H04 (lime), 1B07 (blue), SARS2-01 (yellow), SARS2-02 (teal), SARS2-07 (tangerine), SARS2-16 (violet), SARS2-19 (red), SARS2-32 (fuschia), and SARS2-38 (magenta). Substitutions that led to resistance of mAbs 2B04, 1B07, SARS2-02, SARS2-07, SARS2-16, SARS2-32, and SARS2-38 cluster within and proximal to the ACE2 binding site. Resistance to antibodies SARS2-01 and SARS2-19 mapped to substitutions at sites on the side of the RBD (FIG. 26). mAb 2H04 gave rise to resistance mutations that map exclusively on the side and base of the RBD (FIG. 26). The identification of resistance mutations at the side of the RBD suggest that the mechanism of virus neutralization may be allosteric or possibly through blocking interactions with alternative attachment factors. The presence of resistance mutations at the base of RBD, which lie outside the 2H04 binding footprints, suggests an allosteric mechanism of resistance, perhaps related to the ability of the RBD to adopt the “up” conformation requisite for ACE2 binding.

TABLE 2
List of mutants. Sanger sequencing of isolated escape variants
selected for by each mAb. The mutated nucleotides and residues
in the RBD region of S are highlighted in red.

| | | |
|---|---|---|
| mAb | Nucleotide | Amino Acid |

Campaign 1

| | | |
|---|---|---|
| 2B04 | A1451C | E484A |
| | G1450A | E484K |
| | T1457C | F4 [symbol] |
| 2H04 | A1033G | T345A |
| | C1034A | T345N |
| | A1033T | T345 [symbol] |
| | A1036 [symbol] | R346G |
| | T1322G | L441R |
| | A1330 [symbol] | K444E |
| | A1033G/ | T345A/ |
| | T1550G | L517R |
| 1B07 | A1451C | E484A |
| | A1452C | E484D |
| | A1451 [symbol] | E484G |
| | G1450A | E484K |
| | T1457A | F48 [symbol] |
| SARS2-01 | A1036 [symbol] | R346G |
| | C1055A | A352D |
| | T1355 [symbol] | L452R |
| | T1480C | S4 [symbol] 4P |
| SARS2-02 | G1337A | G446C |
| | G1337T | G446V |
| | G1450A | E484K |
| SARS2-07 | A1348G | N450 [symbol] |
| | A1429G | S477G |
| | G1430A | S477N |
| | C1431G | S477R |
| | C1496T | P499L |
| SARS2-16 | A1429G | S477G |
| | G1430A | S477N |
| | C1431A | S477R |
| SAR82-19 | A1429G | S477G |
| | G1430A | S477N |
| | C1433T | T478I |
| | G1430A/ | S477N/ |
| | C1541T | S514F |
| SARS2-32 | G1337A | G446D |
| | T1350G | N450K |
| | A1348T | N450V |
| | T1355G | L452R |
| | G1450A | E484K |
| | T1469C | F490B |
| SARS2-38 | A1330G | K444E |
| | G1332T | K444M |
| | G1337A | G446D |

Campaign 2

| | | |
|---|---|---|
| SARS2-21 | G1427A | G478 [symbol] |
| | G1428A | G476 [symbol] |
| | C1433T | T478I |
| | C1435T | P479 [symbol] |
| | T1456C | F486L |
| | T1467C | F486 [symbol] |
| SARS2-22 | A1330 [symbol] | K444E |
| | G1332T | K444N |
| | A1331G | K444R |
| | T1334G | V445G |
| | G1337A | G44 [symbol] D |
| SARS2-23 | C1431G | S477R |
| | G1447T | V483F |
| | T1448G | V483G |
| | A1452C | E484D |
| SARS2-31 | A1132G | K378E |
| | A1132C | K378Q |
| | G1223A | R408 [symbol] |
| | G1551A | G504D |
| SARS2-34 | A1421C | Q474P |
| | G1827A | G476D |
| | A1429G | S477G |
| | G1430A | S477N |
| | A1429C | S477R |
| | C1438T | P479L |
| SARS2-55 | G1450A | E484K |
| SARS2-58 | A1429G | S477G |
| | G1430T | S477I |
| | G1430A | 3477N |
| | T1456 [symbol] | F4 [symbol] |
| SARS2-88 | A1372C | K458Q |
| | A1452C | E484D |
| | T1468C | F490L |
| SARS2-71 | G1427A | G476D |
| | A1427G | S477G |
| | C1433T | T478I |
| | A1432C | T478P |
| | C1436T | P479L |
| | T1456G | F486V |

Campaign 3

| Virus | mAb | Nucleotide | Amino Acid |
|---|---|---|---|
| VEV-SARS-CoV-S E484A | 2H04 | A1033G/A1451C | T345A/E484A |
| | | A1036G/A1451C | T346G/E484A |
| | | A [symbol] /A1451C | K444E/E454A |
| VSV-SARS-CoV-S E404K | 2H04 | G1037A/G1450A | R346K/E484K |
| | | G1114A/G1450A | A372T/E484K |
| | | A1330G/G1450A | K444E/E454K |
| VSV-SARS-CoV-S F [symbol] | 2H04 | A10 [symbol] /T1457C | T [symbol] |

[symbol]

indicates data missing or illegible when filed

From this panel of mAbs, we observed resistance substitutions at shared positions. Four mAbs yielded substitutions at E484 (2B04, 1B07, SARS2-02, and SARS2-32), three resulted in changes to residues G446 (SARS2-02, SARS2-32, and SARS2-38) and S477 (SARS2-07, SARS2-16, and SARS2-19), and two promoted escape substitutions at F486 (2B04 and 1B07), K444 (2H04 and SARS2-38), L452 (SARS2-01 and SARS2-32), N450 (SARS2-07 and SARS2-32), and R346 (2H04 and SARS2-01). The overlapping nature of these epitopes suggests that they represent major antigenic sites within the RBD. Although amino acid changes were selected at the same position, many of the substitutions were distinct, consistent with a unique mode of binding for each antibody.

Two mAbs gave rise to variants containing linked amino acid substitutions: 2H04 (T345A and L517R) and SARS2-19 (S477N and S514F). For 2H04, substitution T345A likely arose first, as we isolated this mutation alone, and acquisition of the L517R substitution appeared to enhance infectivity as judged by plaque morphology (FIG. 33). For SARS2-19, S477N was isolated as a single variant, suggesting that this substitution arose first, however, acquisition of the S514F did not alter plaque morphology (FIG. 33). As the L517R or S514F substitutions were not identified in isolation, it remains unclear whether they cause resistance to 2H04 or SARS2-19, respectively. Collectively, these results show that escape mutational profiling can identify key epitopes and dominant antigenic sites.

Escape Mutants Confer Cross-Resistance to Multiple mAbs

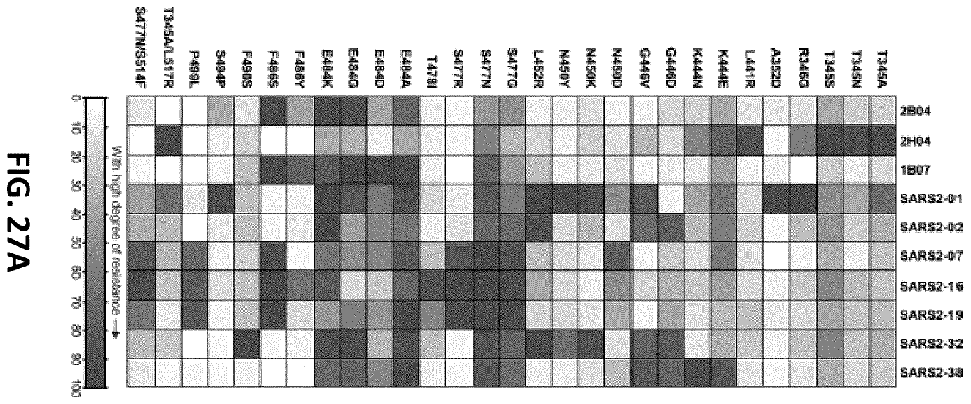

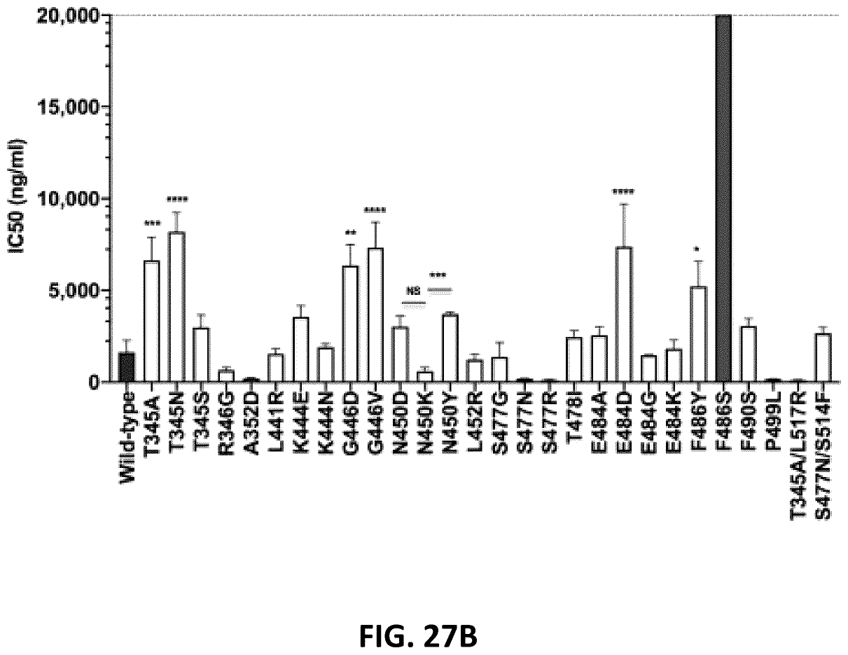

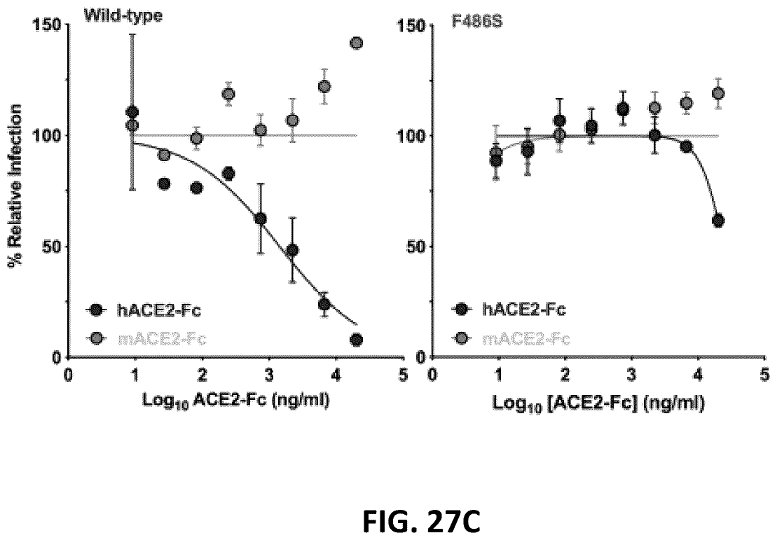

We next evaluated whether individual mutants could escape neutralization by the other inhibitory mAbs in the panel. We tested the 29 identified escape mutants for neutralization by ten different mAbs. We defined the degree of resistance as a percentage by expressing the number of plaques formed by each mutant in the presence or absence of antibody. We plotted the degree of resistance to neutralization as a heatmap and arbitrarily set 50% as the cutoff value for defining resistance (FIG. 27A). Substitutions at residues T345, R346, K444, G446, N450, L452, S477, T478, E484, F486, and P499 were each associated with resistance to more than one mAb, with substitutions at S477 and E484 exhibiting broad resistance (FIG. 27A). For residues at which multiple alternate amino acids with different side chains were selected, each particular substitution was associated with a unique resistance profile. For example, K444E was resistant to SARS2-38 and 2H04 with some resistance to SARS2-1, SARS2-2, and SARS2-7, whereas K444N conferred complete resistance to SARS2-38, partial resistance to 2H04, and only weak resistance to SARS2-1 and SARS2-2. G446D was resistant to SARS2-2, SARS2-32, and SARS2-38, but G446V acquired resistance to SARS2-01. Substitutions N450K and N450Y were resistant to SARS2-01 and SARS2-32, whereas N450D facilitated resistance to SARS2-07. Substitution L452R conferred resistance to SARS2-01, SARS2-02, and SARS2-32; S477N, S477G, and S477R were each highly resistant to SARS2-07, SARS2-16, and SARS2-19, and S477N and S477G result in a degree of resistance across the entire panel of antibodies; and T478I yielded resistance to SARS2-16 and SARS2-19.

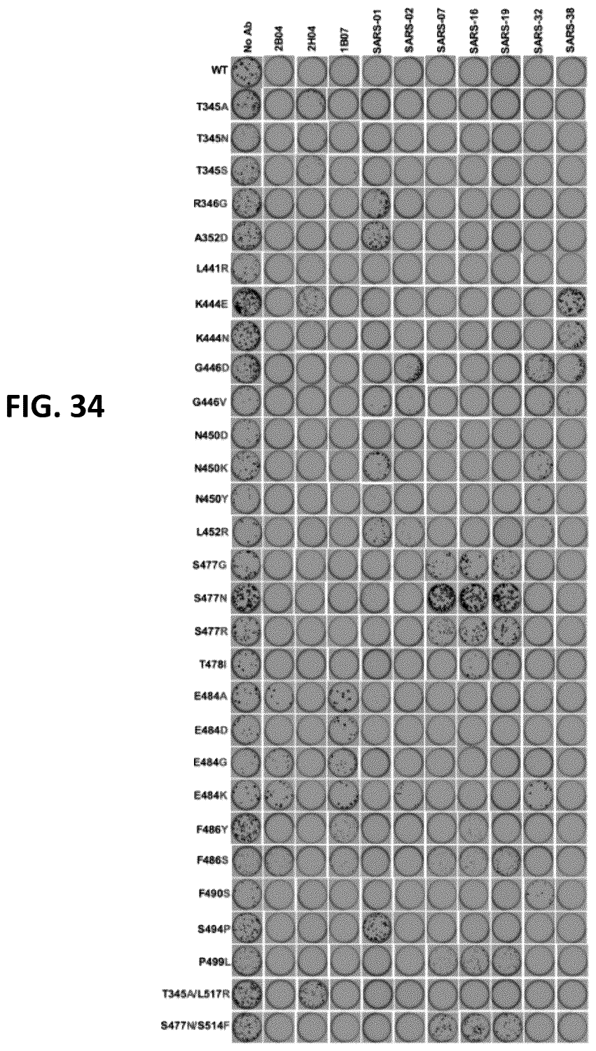

Escape variants at residue E484 were isolated using 2B04, 1B07, SARS2-02, and SARS2-32, and specific substitutions at this residue led to varying degrees of resistance across the entire panel of antibodies. E484A exhibited a high degree of resistance to 2B04, 1B07, SARS2-01, SARS2-07, SARS2-19, SARS2-32, and SARS2-38; E484G exhibited resistance to 2B04, 1B07, SARS2-01, SARS2-32, and SARS2-38; E484K was resistant to 2B04, 1B07, SARS2-01, SARS2-02, SARS2-16, and SARS2-32; and E484D was resistant only to 1B07 (FIG. 27A and FIG. 34). Substitution F486S was resistant to 2B04, 1B07, SARS2-07, SARS2-16, and SARS2-19, whereas F486Y exhibited resistance only to 1B07 and SARS2-16. Finally, substitution P499L was resistant to SARS2-07, SARS2-16, and SARS2-19. In addition to demonstrating that some mutations confer resistance to multiple neutralizing mAbs, these data suggest that each mAb recognizes distinct yet partially overlapping epitopes.

Soluble Human ACE2-Fc Receptor Decoy Inhibition of Escape Mutants

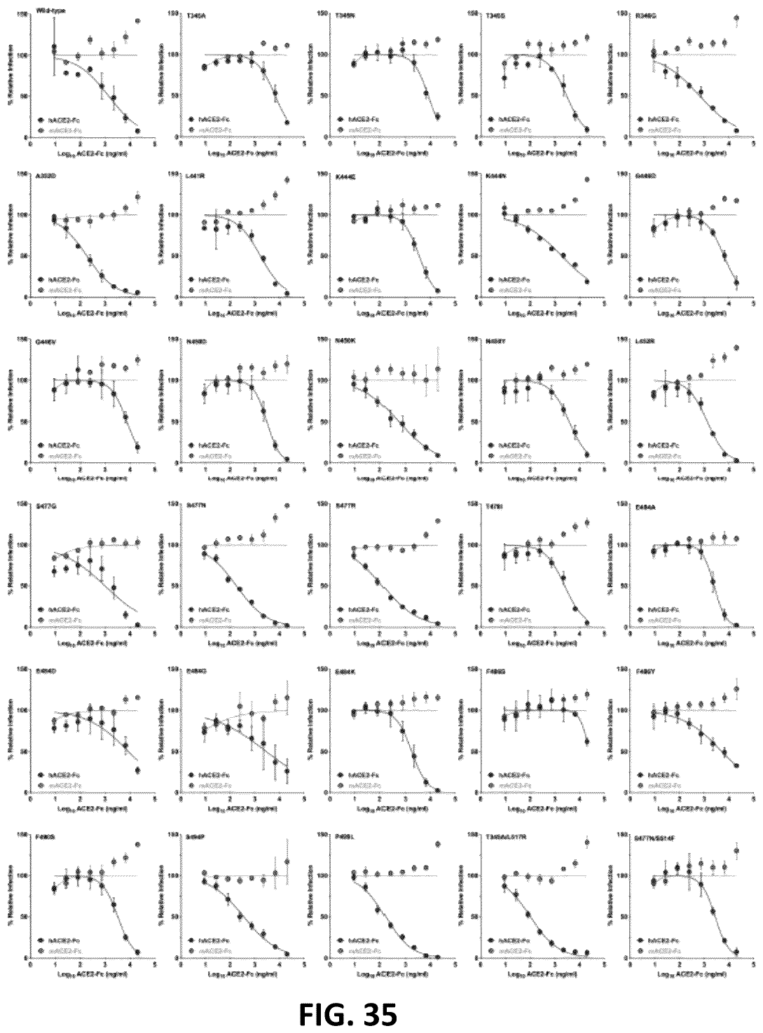

Soluble human ACE2 decoy receptors are under evaluation in clinical trials for treatment of COVID-19 (NCT04375046 and NCT04287686). As several of the escape mutants contain substitutions within or proximal to the ACE2 binding site, we evaluated the ability of soluble recombinant ACE2 to inhibit infection of each variant. We incubated each VSV-SARS-CoV-2 mutant with increasing concentrations of soluble human (h) or murine (m) ACE2-Fc for 1 h at 37° C. and measured residual infectivity on Vero E6 cells (FIG. 27B and FIG. 35). As observed with chimeric viruses expressing the wild-type S protein, the escape mutants were inhibited by hACE2-Fc but not mACE2-Fc. However, the extent of neutralization by hACE2-Fc varied substantially (FIG. 27B), with some mutants more sensitive to receptor inhibition and others exhibiting relative resistance. Substitutions at residues R346, A352, N450, S477, S494, and P499 were more sensitive to inhibition by soluble hACE2 than the wild-type S, as evidenced by reduced IC₅₀ values (FIG. 27B) and leftward shifts of the inhibition curves (FIG. 35). This effect was substitution dependent, as N450K was 6-fold more sensitive to hACE2 than N450Y (p<0.001). Several mutants required higher (3-5-fold) concentrations of hACE2 to block infection, including substitutions at T345A, T345N, G446D, G446V, E484D, and F486Y. Again, the specific substitution of a given residue impacted the effect, as T345A and T345N required higher concentrations of hACE2 to inhibit infection, whereas T345S was similar to wild type. Of the four substitutions observed at position E484, only E484D was less sensitive (4.6-fold, p<0.0001) to hACE2 inhibition. The most striking effect was observed for F486S, where we achieved only 38% inhibition at the highest concentration (20 μg/mL) of hACE2-Fc tested (FIG. 27B and FIG. 27C). Residue F486 is located on the top of the hACE2 contact loop of RBD, and the presence of a large hydrophobic residue facilitates efficient receptor engagement (Lan et al., 2020; Shang et al., 2020). Although this substitution alters sensitivity to soluble ACE2 inhibition of infection, its impact on cell surface ACE2 engagement by virus was not examined.

Escape Mutants Exhibit Resistance to Neutralization by Polyclonal Human Immune Sera

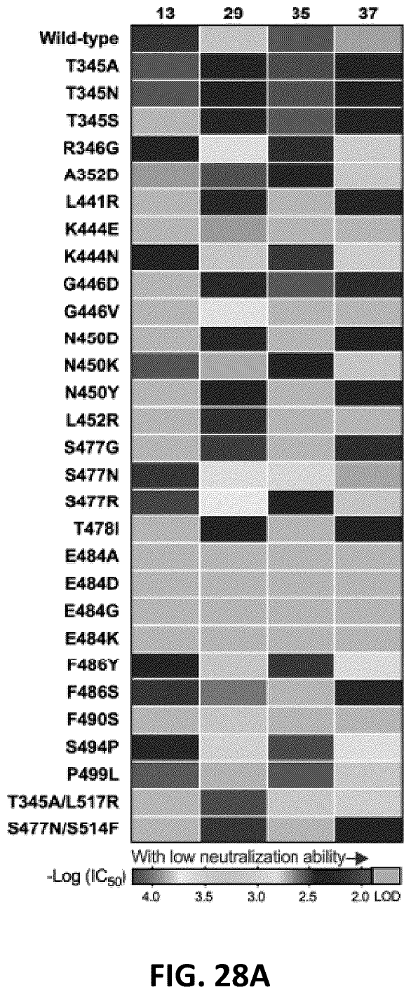

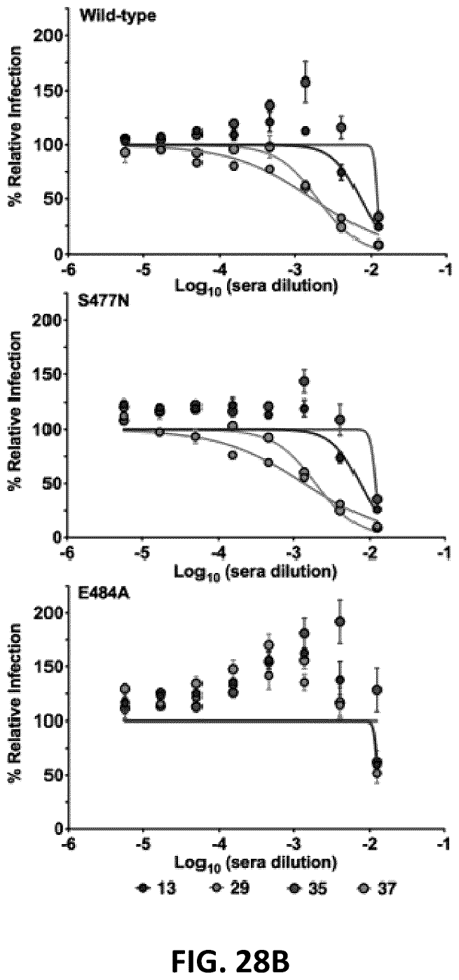

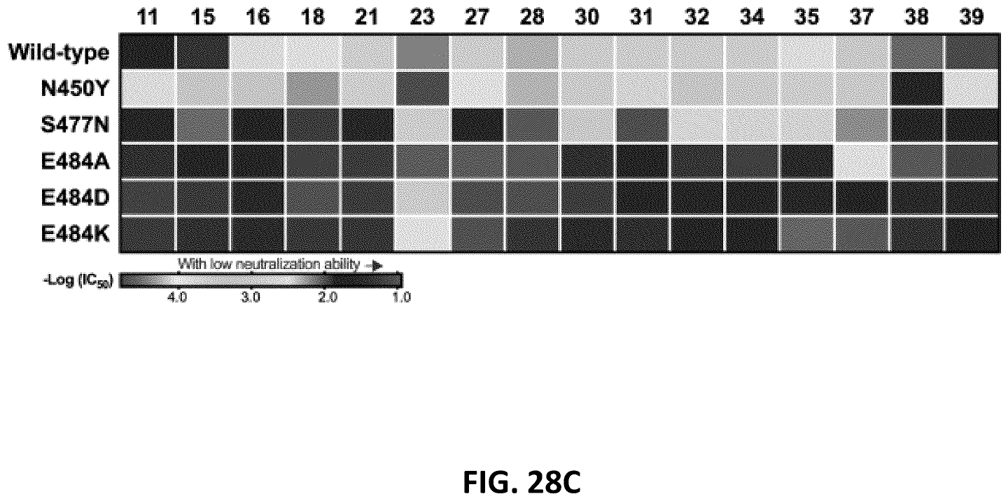

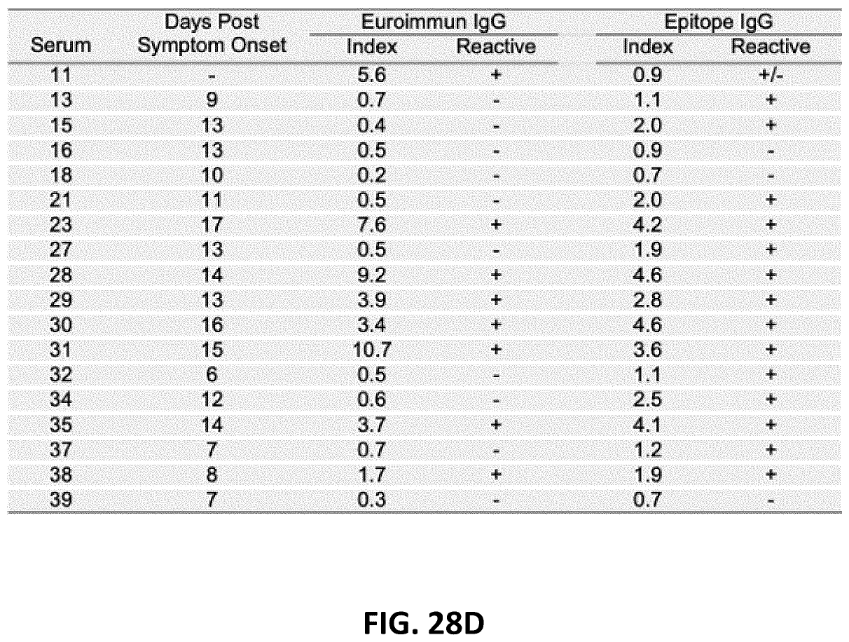

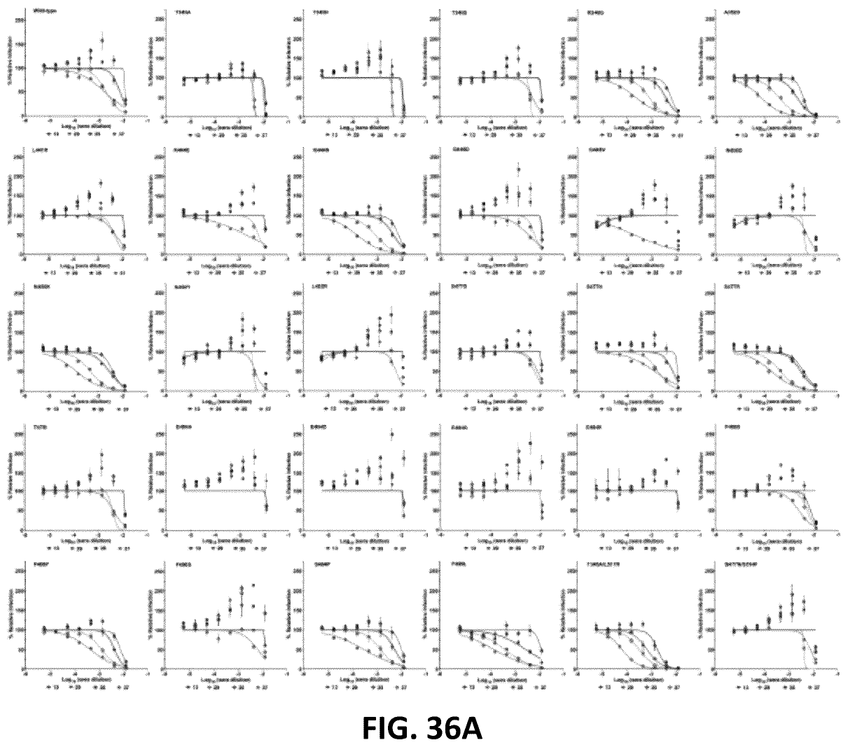

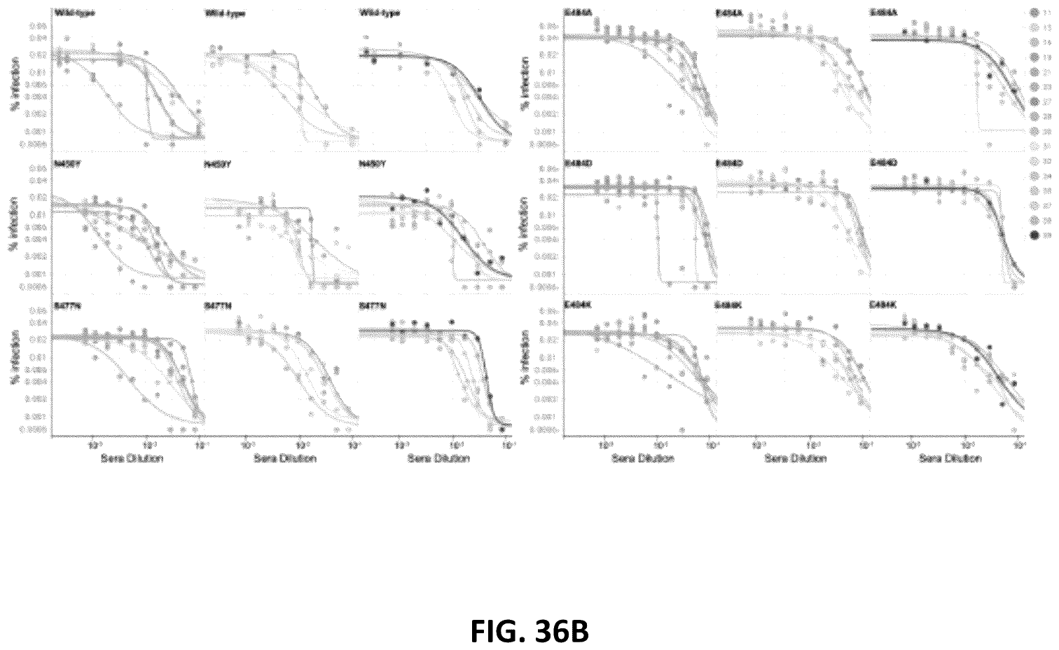

We previously evaluated the ability of convalescent sera from SARS-CoV-2-infected humans to neutralize VSV-SARS-CoV-2 and defined a strong correlation with inhibition of a clinical isolate of SARS-CoV-2 using a focus reduction neutralization test (FRNT) (Case et al., 2020). We tested four of the serum samples (13, 29, 35, and 37) from patients who had recovered from COVID-19 against our panel of escape mutants (FIG. 28D). All four sera neutralized infection of VSV-SARS-CoV-2-S displaying the wild-type S protein, as we previously demonstrated. Remarkably, several of the escape mutants were resistant to neutralization at the highest concentration (1:80 initial dilution) of sera tested. All four of the substitutions at residue E484 were resistant to each of the four sera, suggesting that this is part of a dominant neutralizing epitope. Indeed, change at E484 was the only position that led to escape from sera 29 (FIG. 28A, FIG. 28B, and FIG. 36A). Four other substitutions (K444E, G446V, L452R, and F490S) resulted in resistance to neutralization of sera 13, 35, and 37 (FIG. 28A and FIG. 36A). Substitutions N450D and N450Y, but not N450K, were resistant to sera 13 and 35. Sera 13 and 35 also did not efficiently neutralize S477G, L441R, and T478I. All four sera neutralized the single substitution S477N as well as wild-type virus (FIG. 28A and FIG. 28B). Substitution S477N was sensitive to neutralization by sera 13 and 35 except in the presence of a second S514F substitution (FIG. 28A and FIG. 36A). Additional amino acid substitutions that conferred resistance to serum 13 include T345S and G446D. Substitution F486S, which altered sensitivity to soluble ACE2, escaped neutralization by serum 35 but not 13, 29, or 37. We consistently noticed that some sera also led to an increase in infectivity of specific escape mutants (e.g., E484A) at some concentrations (FIG. 28B). The significance of this increase is unclear but was observed consistently across sera for several mutants (FIG. 36A).

To extend these findings, we employed a higher throughput screening assay to test 16 additional human sera (11, 15, 16, 18, 21, 23, 27, 28, 30, 31, 32, 34, 35, 37, 38, and 39) for their ability to neutralize the VSV-SARS-CoV-2 mutants N450Y, S477N, E484A, E484D, and E484K (FIG. 28D). Although we observed neutralization of the various mutants at the highest concentrations of human sera tested (1:10), VSV-SARS-CoV-2 variants with substitutions at residue 484 were consistently less sensitive to neutralization by all sera (FIG. 28C and FIG. 36B). Thus, individual escape mutants can exhibit resistance to neutralization by polyclonal human convalescent sera. This observation suggests that the repertoire of antigenic sites on RBD that bind neutralizing antibodies is limited in some humans. We again observed the increase in infectivity of substitutions at residue E484 in the presence of multiple human sera (FIG. 36B).

Comparison of Escape Mutants with S Sequence Variants Isolated in Humans

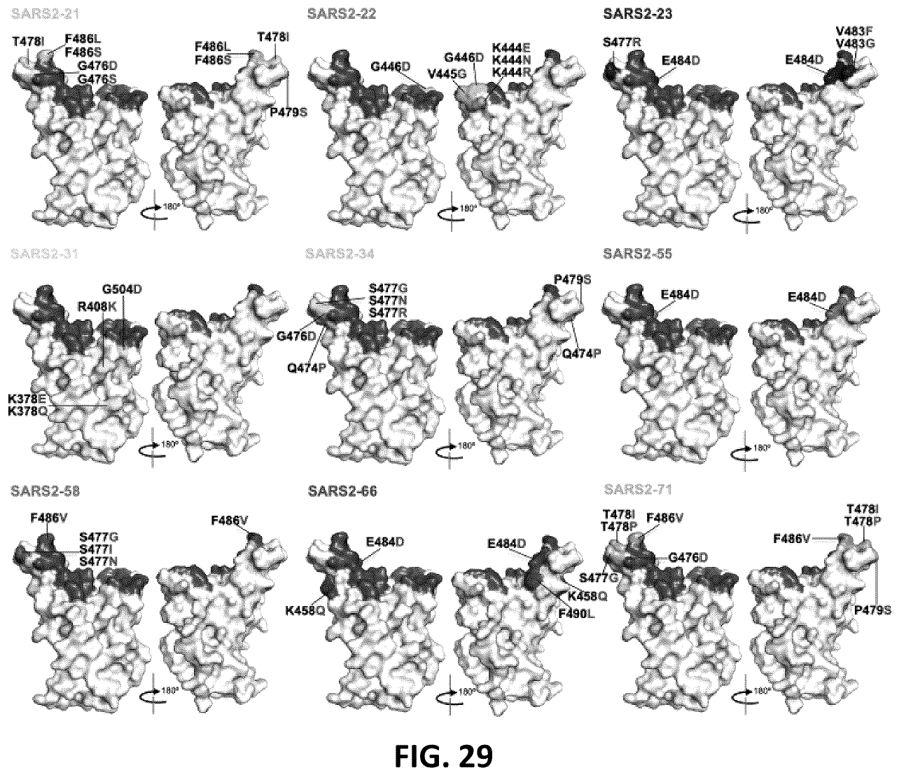

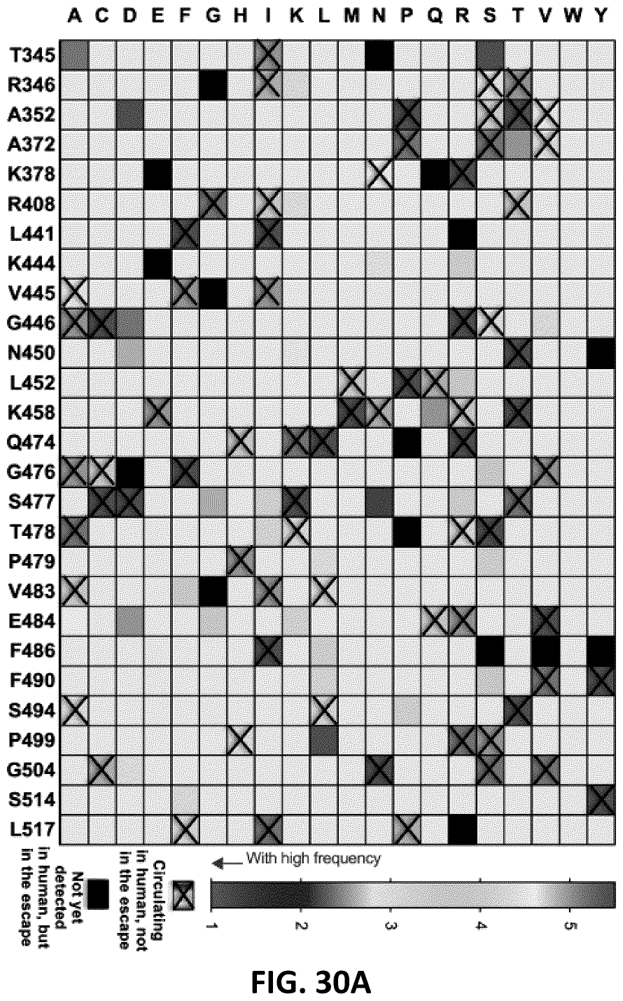

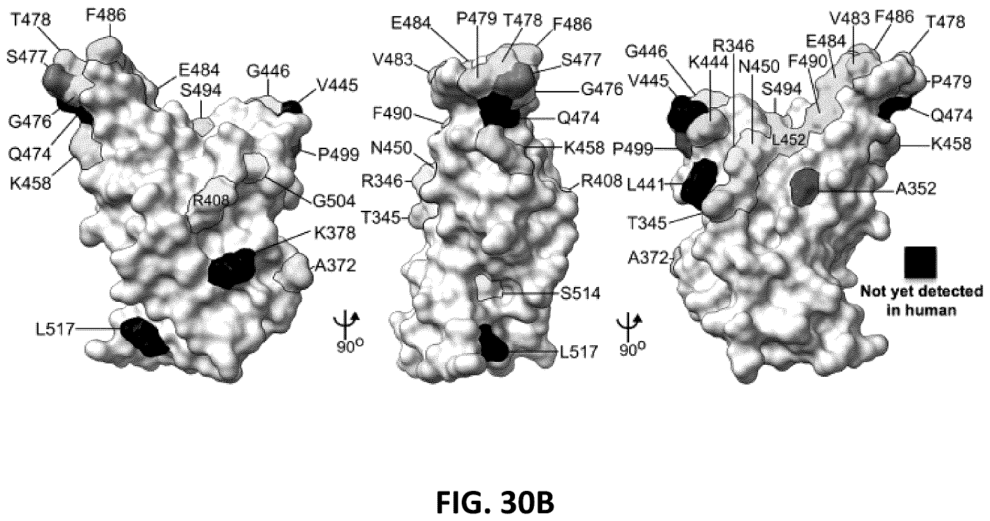

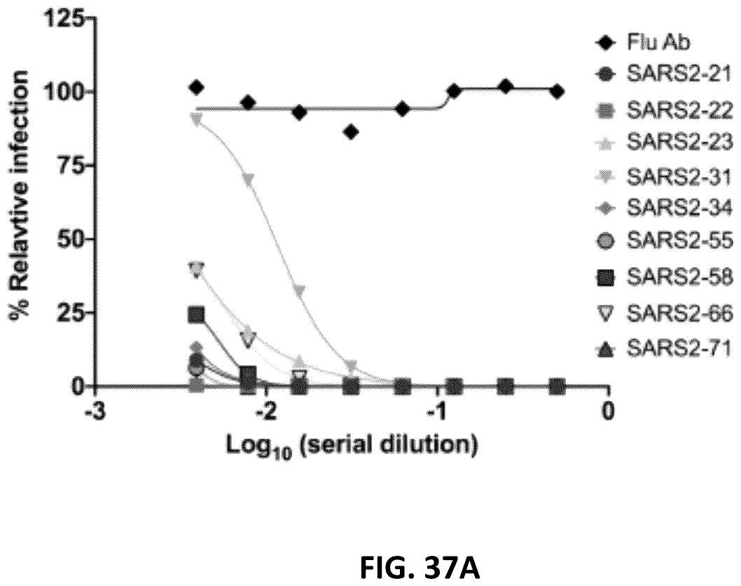

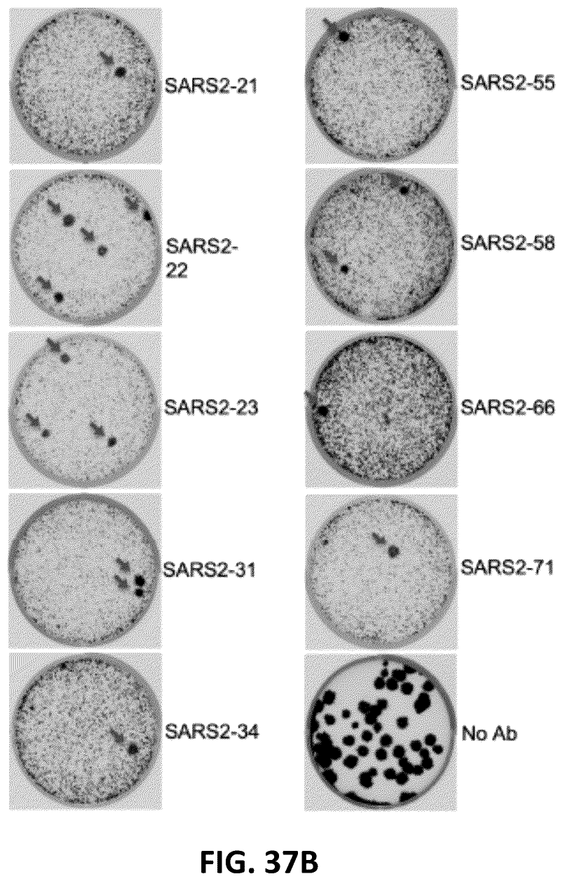

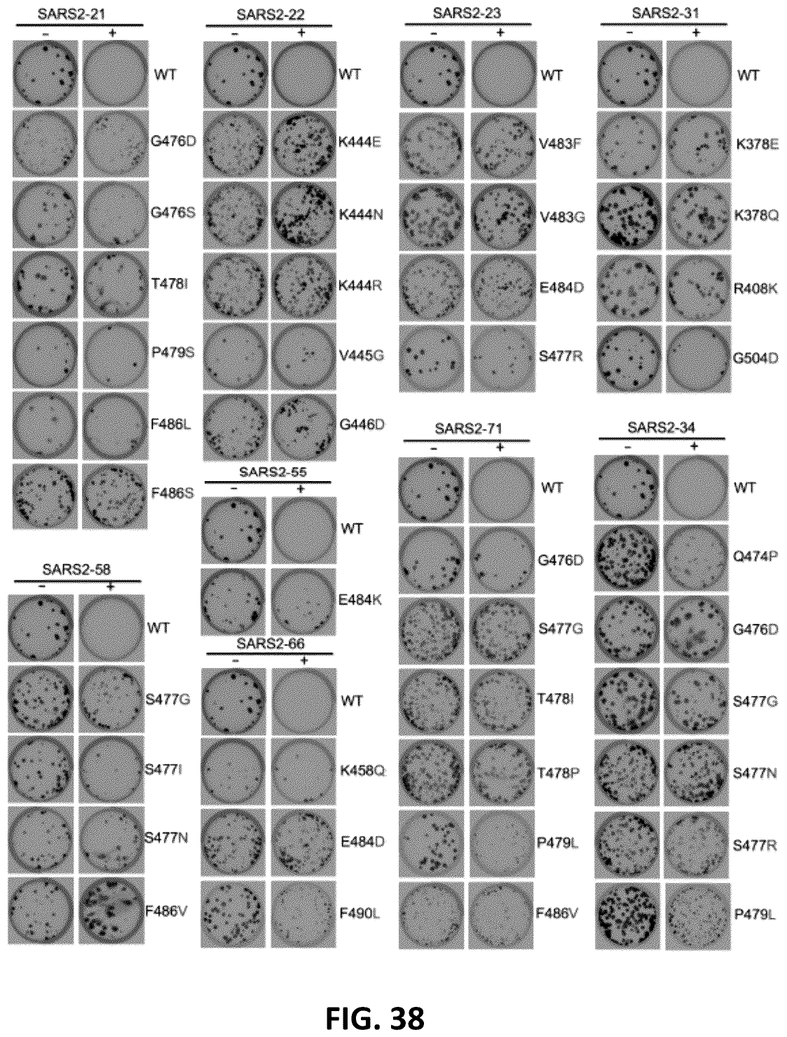

To broaden our analysis, we performed a second campaign of escape mutant selection using nine additional neutralizing mAbs generated against the RBD (FIG. 29, FIG. 37, and FIG. 38; TABLE 2). This effort generated 19 additional escape mutants, bringing the total to 48. To determine whether any of the 48 escape mutants we isolated represent S protein variants circulating in humans, we compiled all publicly available genome sequences of SARS-CoV-2. Using 323,183 genomes from GISAID, we calculated the substitution frequencies throughout RBD protein (FIG. 30A) and mapped the identified residues onto RBD structure (FIG. 30B). Of the 48 escape variants we selected, 32 are present in circulating human isolates of SARS-CoV-2 (FIG. 30A). The most frequent S sequence variant seen in clinical isolates is D614G, which is present in 69% of sequenced isolates. The fourth most frequent substitution is S477N, which is present in 4.6% of sequenced isolates and the dominant virus in Oceana. The penetrance of the remaining substitutions among clinical isolates is relatively low, with G446V, T478I, E484K, S4771, and S494P ranking 79, 102, 123, 135, and 146 of the top 150 variants in S or roughly 0.05% of sequenced variants. Collectively, this analysis highlights that neutralizing mAbs against RBD can select for variants or changes at positions that already exist within the human population and establishes that some substitutions are present at high frequency.

Sequential Selection of 2B04 and 2H04 Escape Mutants

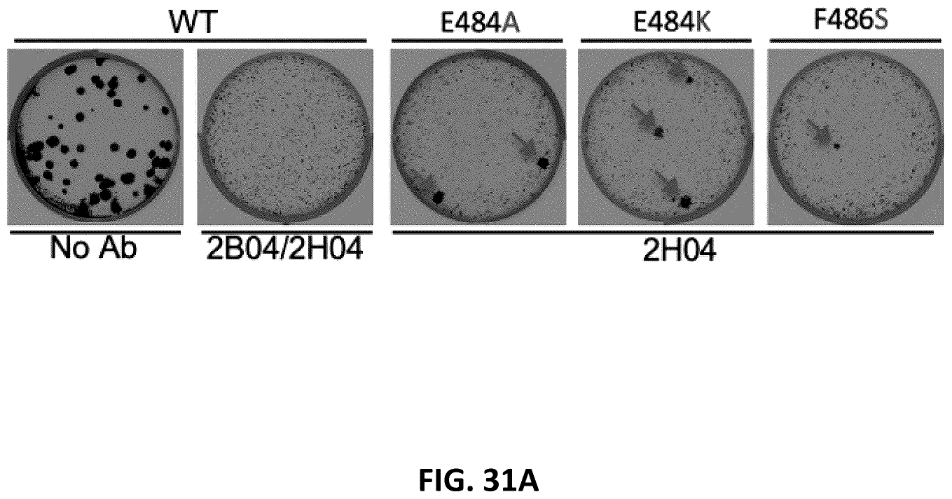

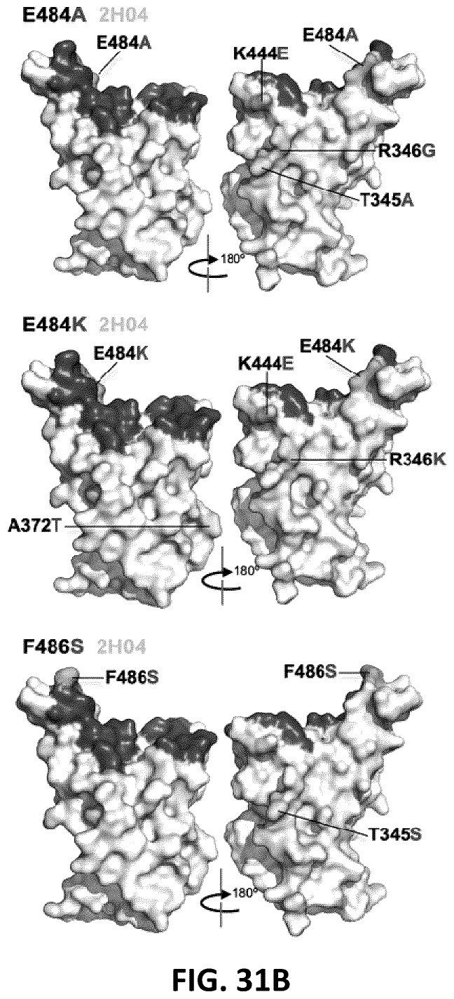

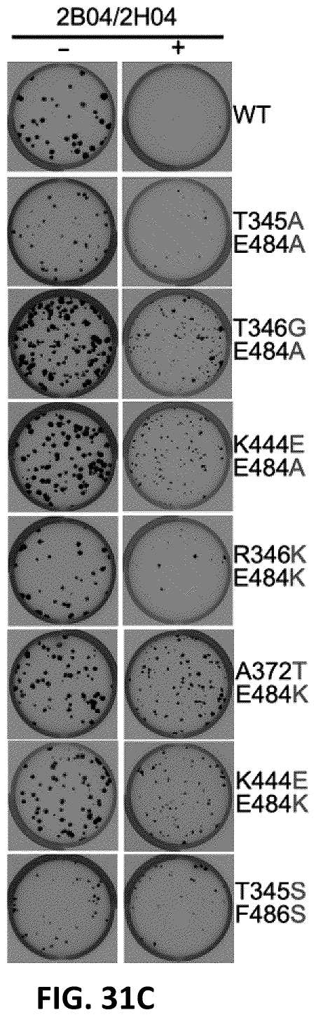

To examine whether mutations resistant to antibody combinations could be isolated, we undertook a third selection campaign using a combination of 2B04 and 2H04. We were unable to isolate mutants resistant to the two antibodies when added concurrently. However, using the 2B04 resistant viruses E484A, E484K, and F486S, we selected additional mutations by growth in the presence of 2H04 (FIG. 31; TABLE 2). These selected variants were resistant to both antibodies. For the 2B04-resistant mutant E484A, we selected T345A, R346G, and K444E; for mutant E484K, we isolated R346K, A372T, and K444E; and for mutant F486S, we selected T345S. Two of the mutants (R346K and A372T) were not seen in our prior selection campaigns with 2H04 alone, although both variants exist in human isolates (FIG. 30). Taken together, this analysis suggests that cocktails of mAbs binding distinct epitopes on SARS-CoV-2 S protein pose an increased but not complete barrier to resistance, especially if circulating strains already encode substitutions that compromise effectiveness of one of the two mAbs.

Discussion

Therapeutic mAbs, convalescent plasma, and vaccines are in clinical development as countermeasures against SARS-CoV-2. The efficacy of these strategies will be impacted by viral mutants that escape antibody binding and neutralization. To define the landscape of mutations in the RBD associated with resistance, we selected escape mutants to 19 neutralizing mAbs, including some in clinical development. Characterization of escape mutants identified several that exhibit resistance to multiple antibodies, convalescent human sera, and soluble receptor decoys. Resistance to neutralization by serum from naturally infected humans suggests that the neutralizing response to SARS-CoV-2 in some individuals may be dominated by antibodies that recognize relatively few epitopes. Many of the escape mutants we identified contain substitutions in residues at which variation is observed in circulating human isolates of SARS-CoV-2. If a similar limited polyclonal response occurs following S protein-based vaccination, escape variants could emerge in the human population and compromise the efficacy of such vaccines.

From 19 different mAbs that neutralize SARS-CoV-2, we isolated 50 viral mutants that escape neutralization. Selection of escape mutants was facilitated by the use of VSV-SARS-CoV-2, which we previously validated as an effective mimic of SARS-CoV-2 S protein-mediated infection (Case et al., 2020). The mAbs were obtained following immunization with soluble RBD, and although some mice received a boost with stabilized S ectodomain protein, all escape substitutions map within the RBD. Multiple different mAbs led to resistance substitutions at K444, G446, N450, L452, S477, T478, P479, E484, F486, and P499, suggesting that they comprise major antigenic sites within the RBD. In earlier work, substitutions at residues K444, N450, E484, and F486 were identified using two antibodies in clinical development (ACTIV-3/TICO LY-CoV555 Study Group, 2020), and a separate study using three different antibodies defined resistance substitutions at R346, N440, E484, F490, and Q493 (Greaney et al., 2021; Weisblum et al., 2020).

The mutations we selected also inform the mechanism by which the different antibodies function. All of the resistance mutations we identified map within or proximal to the ACE2 binding site. Likely, the majority of the antibodies we tested neutralize infection by interfering with receptor engagement. Antibodies from human survivors also interfere with receptor engagement (Wu et al., 2020b; Zost et al., 2020), suggesting a common mechanism of neutralization. Some of the resistance mutations from 2H04, SARS2-01, and SARS2-31 we identified map outside the ACE2 binding site, including at the side and the base of the RBD. Direct competition with ACE2 binding is consistent with the escape mutants we selected with 2B04, whereas an indirect mechanism of action fits with the escape mutants we identified to 2H04. Our finding of an escape mutant to 2H04 located at the base of the RBD, outside the footprint of the antibody, suggests a possible allosteric mechanism of resistance. This mutation might affect the ability of the RBD to adopt the up conformation necessary for engagement of the cellular receptors, perhaps by shielding the epitope or stabilizing the RBD in the down conformation. Further structural and functional work is required to define how different mutations promote antibody resistance and determine the mechanisms by which specific antibodies inhibit SARS-CoV-2 infection.

The relatively low genetic barrier to resistance combined with knowledge of the presence of relevant substitutions in clinical isolates suggests that effective mAb therapy will likely require a combination of at least two neutralizing antibodies (ACTIV-3/TICO LY-CoV555 Study Group, 2020; Baum et al., 2020; Du et al., 2020; Greaney et al., 2021; Li et al., 2020; Weisblum et al., 2020). Profiling whether different residues are associated with resistance to specific antibodies could facilitate the selection of combinations based on their non-overlapping resistance mutations. Although we isolated several escape mutants that exhibit cross-resistance to multiple antibodies, other antibodies are associated with unique and non-overlapping resistance. Resistance to such combinations could still arise through sequential escape whereby a resistant variant to one antibody acquires resistance to a second. Sequential escape could be favored in vivo for two antibodies with different half-lives, or when a pre-existing resistant variant to one antibody already is circulating. Indeed, while we could not generate escape mutants to the antibody cocktail of 2B04 and 2H04, we readily isolated escape mutants to both mAbs through sequential selection.

Substitution of S477N, the fourth most abundant S protein sequence variant in circulating human isolates of SARS-CoV-2, led to a degree of resistance to all of the mAbs we profiled, including 2B04 and 2H04. How S477N could confer such broad resistance is of interest, given its penetrance among clinical isolates (6.5%). One possible explanation may relate to changes in glycosylation at this position. Additional analysis is required to determine how broad the resistance associated with S477N is, and to probe the mechanism by which it occurs. The broad mAb resistance observed here for S477N was not accompanied by resistance to neutralization by human convalescent sera, suggesting that other epitopes-such as those centered around E484—are more dominant in humans. Among our panel of mutants, we isolated a total of 14 substitutions at sites of glycosylation, including eight N-linked glycans sites: T345N, K444N, S477N, L441R, L517R, L452R, S477R, and K444R; and six O-linked glycans: F486S, T345S, F490S, P479S, F486Y, and N450Y.

Substitutions at position E484 were associated with relative resistance to neutralization by several convalescent human sera. Four variants at this position (E484A, E484D, E484G, and E484K) exhibited resistance to each of the human convalescent sera we tested. This suggests that in some humans, neutralizing antibodies may be directed toward a narrow repertoire of epitopes following natural infection. Substitution at position E484 has become increasingly common among clinical isolates. As of October 2020, just 0.03% of sequenced isolates exhibited variation at E484, which led us to suspect that variation at this position may come with an apparent fitness cost for viral replication. However, by January 2021, the prevalence of substitutions at this position had increased to 0.09%. Substitution E484K is likely to increase in penetrance further as it linked together with N501Y and K417N changes that are present in variant 501.V2, which is believed to be more transmissible (Tegally et al., 2020). The relative resistance of the substitutions at E484 to the human sera tested highlight how variants at even a single position can affect neutralization. Given the apparent limited breadth of the human neutralizing antibody response to natural infection, it will be important to define the epitope repertoire following vaccination and develop strategies that broaden neutralizing antibody responses. In this regard, the 50 viral mutants described here, combined with additional mutants reported in related studies (ACTIV-3/TICO LY-CoV555 Study Group, 2020; Greaney et al., 2021; Li et al., 2020; Weisblum et al., 2020), provide a compendium of functionally relevant S protein variants that could be used to profile sera from vaccine recipients in existing clinical trials.

Among the escape variants we selected, there were several that altered susceptibility to neutralization by soluble ACE2. Substitution F486S was particularly notable, as we were unable to attain 50% neutralization at the highest concentrations of soluble hACE2 tested (>20 μg/mL). The finding of an antibody escape mutant mapping to a critical residue within the ACE2 binding site raises questions regarding possible receptor usage by viruses containing S proteins with F486S. Future studies that introduce F486S into an infectious cDNA clone of SARS-CoV-2 are needed to determine the significance of this change to hACE2 interactions in vivo. The escape variants we selected were also examined for their sensitivity to neutralization by soluble mACE2. For the wild-type S sequence and some escape mutants (e.g., L441R, K444N, L452R, and S477N), we observed a modest increase in infectivity at increasing concentration of soluble mACE2. Further studies using the infectious molecular clone of SARS-CoV-2 will be required to discern the significance of this observation.

We did not directly address the fitness of the mutants generated in this study, and any studies of the fitness of the VSV-SARS-CoV-2 variants would pertain to their relative replicative fitness measured in cell culture (Domingo et al., 2012). We can, however, make several inferences about fitness of specific viral mutants based on the prevalence of the corresponding mutants among circulating human isolates of SARS-CoV-2. Over 60% of the mutants we isolated in this study already circulate as natural viral variants. The escape mutants we isolated were based on single nucleotide changes starting from the sequence of the S protein of the SARS-CoV-2 Wuhan-Hu-1 strain (Wu et al., 2020a). In the context of VSV-SARS-CoV-2, the fitness of the variants in cell culture relates to their ability to resist neutralization by the indicated mAb and infect Vero cells presumably through interactions with ACE2. Our functional screens complement other systematic mutational analyses of the amino acid residues of the RBD of the SARS-CoV-2 S, such as those based on yeast display (Starr et al., 2020).

Use of chimeric VSV that depends on SARS-CoV-2 S protein for entry into cells enabled the selection of 50 escape mutants. Although chimeric VSV serves as an effective mimic of SARS-CoV-2 S protein-mediated entry and viral neutralization, sequence analysis of circulating human isolates revealed that 34 of those escape mutants are present in the context of infectious SARS-CoV-2. The remaining 16 variants may represent S sequences with compromised fitness in the background of SARS-CoV-2. Here, a number of polyclonal human sera that we profiled against the panel of escape mutants. Additional human sera samples at lower dilutions may help determine the extent to which serum-based neutralization of virus is affected by individual or combinations of escape mutants.

| | | |
|---|---|---|
| REAGENT or RESOURCE | SOURCE | IDENTIFIER |

Antibodies

| | | |
|---|---|---|
| 2B04 | Alsoussi | PDB: 7K9H |
| | et al., 2020 | |
| 2H04 | Alsoussi | PDB: 7K9J |
| | et al., 2020 | |
| 1B07 | Alsoussi | N/A |
| | et al., 2020 | |
| Anti-influenza | Stadlbauer | N/A |
| NA antibody | et al., 2019 | |
| SARS2 antibody | This study | N/A |
| set | | |

Bacterial and virus strains

| | | |
|---|---|---|
| VSV-eGFP-SARS- | Case | N/A |
| COV-2 | et al., 2020 | |
| 50 Escape | This study | N/A |
| mutants set | | |
| Patient serum | Case | N/A |
| set | et al., 2020 | |

Chemicals, peptides, and recombinant proteins

| | | |
|---|---|---|
| Formaldehyde | Millipore | Cat# |
| Solution | Sigma | FX0410-5 |
| HEPES, free | Millipore | Cat# |
| acid | Sigma | 5310-OP |
| Hoechst 33342 | Invitrogen/ | Cat# |
| | Thermo | H3570 |
| | Fisher | |
| NucRed Live | Invitrogen/ | Cat# |
| 647 | Thermo | R37106 |
| | Fisher | |
| RNeasy ® | QIAGEN | Cat# |
| Mini Kit | | 74106 |
| OneStep | QIAGEN | Cat# |
| RT-PCR Kit | | 210212 |
| Gel | QIAGEN | Cat# |
| Extraction | | 28706 |
| Kit | | |
| Minimum | GIBCO | Cat# |
| Essential | | 11700-077 |
| Medium | | |
| L-Glutamine | Sigma | Cat# |
| solution | | RNBG9716 |
| Agarose \|™ | VWR Life | Cat# |
| | Science | 0710-500G |
| Human | Case | GenBank: |
| ACE2-Fc | et al., 2020 | BAB40370.1 & |
| | | AAC82527.1 |
| Mouse | Case | NCBI |
| ACE2-Fc | et al., 2020 | Reference |
| | | Sequence: |
| | | NP_ |
| | | 001123985.1 |

Critical commercial assays

| | | |
|---|---|---|
| Sanger | Genewiz | N/A |
| sequencing | | |

Deposited data

| | | |
|---|---|---|
| Nucleotide | Case | BioProject: |
| sequence of | et al., 2020 | PRJNA635934; |
| VSV-eGFP- | | SRA: |
| SARS-CoV-2- | | SRR11878607 |
| S_{Δ21} | | |

Experimental models: cell lines

| | | |
|---|---|---|
| MA104 | Gift from | N/A |
| | Harry | |
| | Greenberg | |
| Vero | ATCC | Cat# |
| CCL81 | | CCL-81; |
| | | RRID: |
| | | CVCL_0059 |
| Vero | ATCC | Cat# |
| E6 | | CRL-1586; |
| | | RPID: |
| | | CVCL_0574 |
| Vero | Case | N/A |
| E6- | et al., 2020 | |
| TMPRSS2 | | |

Experimental models: organisms/strains

| | | |
|---|---|---|
| Mouse: | Jackson | Cat# |
| BALB/c | Laboratory | 000651; |
| | | RRID: |
| | | IMSR_ |
| | | JAX: |
| | | 000651 |

Oligonucleotides

| | | |
|---|---|---|
| Reverse | This study | N/A |
| Primer 1 | | |
| GTCTACAGCATCTG | | |
| TAATGG | | |
| Forward | This study | N/A |
| Primer 1 | | |
| GATTCTTCTTCAG | | |
| GTTGGACAG | | |
| Reverse | This study | N/A |
| Primer 2 | | |
| GAACAGCAACCTG | | |
| GTTAGAAG | | |
| Forward | This study | N/A |
| Primer 2 | | |
| CAGAGACATTGC | | |
| TGACACTAC | | |
| Reverse | This study | N/A |
| Primer 3 | | |
| CACTATTAAATT | | |
| GGTTGGCAATC | | |
| Forward | This study | N/A |
| Primer 3 | | |
| GTACAATCACTT | | |
| CTGGTTGG | | |
| Forward | This study | N/A |
| Primer 4 | | |
| CACACTTTCC | | |
| TCGTGAAGG | | |

Recombinant DNA

| | | |
|---|---|---|
| pFM1.2- | Case | GenBank: |
| hACE2-Fc | et al., 2020 | AB046569.1 |
| pFM1.2- | Case | NCBI |
| mACE2-Fc | et al., 2020 | Reference |
| | | Sequence: |
| | | NM_ |
| | | 001130513.1 |

Software and algorithms

| | | |
|---|---|---|
| Image | GE Life | N/A |
| Analyses: | Sciences | |
| GE InCell | (now | |
| Analyzer | Cytiva) | |
| 1000 | | |
| Workstation | | |
| InCell 6500 | Cytiva | N/A |
| confocal | | |
| imager | | |
| IN Carta | Cytiva | N/A |
| image | | |
| analysis | | |
| software | | |
| Statistics: | GraphPad | N/A; RRID: |
| Prism 8.0 | | SCR_005375 |
| Snapgene | GSL | N/A; RRID: |
| | Biotech | SCR_015052 |
| Spotfire | Tibco | N/A; PRID: |
| | | SCR_008858 |

Experimental Model and Subject Details

Cells

Cells were cultured in humidified incubators at 34° or 37° C. and 5% CO₂ in the indicated media. Vero CCL81, Vero E6 and Vero E6-TMPRSS2 were maintained in DMEM (Corning or VWR) supplemented with glucose, L-glutamine, sodium pyruvate, and 10% fetal bovine serum (FBS). MA104 cells were propagated in Medium 199 (GIBCO) containing 10% FBS. Vero E6-TMPRSS2 cells were generated using a lentivirus vector described as previously (Case et al., 2020).

VSV-SARS-CoV-2 Mutants

Plaque assays were performed to isolate the VSV-SARS-CoV-2 escape mutant on Vero E6-TMPRSS2 cells with the indicated mAb in the overlay. The concentration of mAb in the overlay was determined by neutralization assays at a multiplicity of infection (MOI) of 100. Escape clones were plaque-purified on Vero-E6 TMPRSS2 cells in the presence of mAb, and plaques in agarose plugs were amplified on MA104 cells with the mAb present in the medium. Viral stocks were amplified on MA104 cells at an MOI of 0.01 in Medium 199 containing 2% FBS and 20 mM HEPES pH 7.7 (Millipore Sigma) at 34° C. Viral supernatants were harvested upon extensive cytopathic effect and clarified of cell debris by centrifugation at 1,000×g for 5 min. Aliquots were maintained at −80° C.

Mouse Experiments

Animal studies were carried out in accordance with the recommendations in the Guide for the Care and Use of Laboratory Animals of the National Institutes of Health. The protocols were approved by the Institutional Animal Care and Use Committee at the Washington University School of Medicine (Assurance number A3381-01). Virus inoculations were performed under anesthesia that was induced and maintained with ketamine hydrochloride and xylazine, and all efforts were made to minimize animal suffering. At four weeks of age, female BALB/c mice (catalog 000651) were purchased from The Jackson Laboratory.

Method Details

Sequencing of the S gene

Viral RNA was extracted from VSV-SARS-CoV-2 mutant viruses using RNeasy Mini kit (QIAGEN), and S was amplified using OneStep RT-PCR Kit (QIAGEN). The mutations were identified by Sanger sequencing (GENEWIZ).

Plaque Assays

Plaque assays were performed on Vero and Vero E6-TMPRSS2 cells. Briefly, cells were seeded into 6 or 12 well plates for overnight. Virus was serially diluted using DMEM and cells were infected at 37° C. for 1 h. Cells were cultured with an agarose overlay in the presence of Ab or absence of Ab at 34° C. for 2 days. Plates were scanned on a biomolecular imager and expression of eGFP is show at 48 h post-infection.

Protein Expression and Purification

Soluble hACE2-Fc and mACE2-Fc were generated and purified as described as previously (Case et al., 2020).

Monoclonal Antibodies

mAbs 2B04, 1B07 and 2H04 were described previously (Alsoussi et al., 2020). Other mAbs (SARS2-01, SARS2-02, SARS2-07, SARS2-16, SARS2-19, SARS2-21, SARS2-22, SARS2-23, SARS2-31, SARS2-32, SARS2-34, SARS2-38, SARS2-55, SARS2-58, SARS2-66 and SARS2-71) were generated as follows. BALB/c mice were immunized and boosted twice (two and four weeks later) with 5-10 μg of RBD and S protein (twice) sequentially, each adjuvanted with 50% AddaVax and given via an intramuscular route. Mice received a final, non-adjuvanted boost of 25 μg of SARS-CoV-2 S or RBD (25 μg split via intravenous and interperitoneal routes) 3 days prior to fusion of splenocytes with P3X63.Ag.6.5.3 myeloma cells. Hybridomas producing antibodies were screened by ELISA with S protein, flow cytometry using SARS-CoV-2 infected cells, and single endpoint neutralization assays.

Human Immune Sera

The human sera samples 11, 13, 15, 16, 18, 21, 23, 27, 28, 29, 30, 31, 32, 34, 35, 37, 38, 39 used in this study were previously reported (Case et al., 2020), Human donor samples were collected from PCR-confirmed COVID-19 patients. Sera samples were obtained by routine phlebotomy (Case et al., 2020). This study was approved by the Mayo Clinic Institutional Review Board.

Neutralization Assays Using a Recombinant VSV-SARS-CoV-2

Briefly, serial dilutions of sera beginning with a 1:80 initial dilution were three-fold serially diluted in 96-well plate over eight dilutions. Indicated dilutions of human serum were incubated with 10² PFU of VSV-SARS-CoV-2 for 1 h at 37° C. Human serum-virus complexes then were added to Vero E6 cells in 96-well plates and incubated at 37° C. for 7.5 h. Cells were fixed at room temperature in 2% formaldehyde containing 10 μg/mL of Hoechst 33342 nuclear stain for 45 min. Fixative was replaced with PBS prior to imaging. Images were acquired using an In Cell 2000 Analyzer automated microscope (GE Healthcare) in both the DAPI and FITC channels to visualize nuclei and infected cells (×4 objective, 4 fields per well). Images were analyzed using the Multi Target Analysis Module of the In Cell Analyzer 1000 Workstation Software (GE Healthcare). GFP-positive cells were identified using the top hat segmentation method and counted within the InCell Workstation software. ACE2 neutralization assays using VSV-SARS-CoV-2 were conducted similarly. The initial dilution started at 20 μg/mL and was three-fold serially diluted in 96-well plates over eight dilutions. mAb neutralization assays using VSV-SARS-CoV-2 were conducted similarly but using an MOI of 100.

High-Throughput Assay Using a Recombinant VSV-SARS-CoV-2

Serial dilutions of patient sera beginning with a 1:10 initial dilution were performed in 384-well plates and were incubated with 10⁴ PFU of VSV-SARS-CoV-2 for 1 h at 37° C. Vero E6 cells then were added to the human serum-virus complexes in 384-well plates at 3×10³ cells per well and incubated at 37° C. for 16 h. Cells were fixed at room temperature in 4% formaldehyde and then rinsed with PBS. Cells were stained at room temperature with NucRed Live 647 (Invitrogen) for 30 min. Images were acquired using an InCell 6500 confocal imager (Cytiva) to visualize nuclei and infected cells (4× objective, 1 field per well). Images were segmented using InCarta (Cytiva). Virally-infected cells were identified by comparing to the uninfected threshold in Spotfire (Tibco). Cells were also quality-controlled (gated) based on nuclear parameters.

Quantification and Statistical Analysis

All statistical tests were performed as described in the indicated figure legends. Non-linear regression (curve fit) was performed to calculate IC₅₀ values for FIG. 27C, FIG. 28B, FIG. 35, and FIG. 36A using Prism 8.0 (GraphPad). Non-linear regression (curve fit) was performed for FIG. 25A, FIG. 32A, and FIG. 37A using Prism 8.0. Non-linear regression (curve fit) was performed to calculate IC₅₀ values for FIG. 36B using Spotfire (Tibco) after adding additional baseline and plateau points. Statistical significance in data FIG. 27B was calculated by one-way ANOVA with Dunnett's post-test using Prism 8.0. The number of independent experiments used are indicated in the relevant Figure legends.

### REFERENCES

Alsoussi W. B., Turner J. S., Case J. B., Zhao H., Schmitz A. J., Zhou J. Q., Chen R. E., Lei T., Rizk A. A., McIntire K. M. A Potently Neutralizing Antibody Protects Mice against SARS-CoV-2 Infection. J. Immunol. 2020; 205:915-922.
Baum A., Fulton B. O., Wloga E., Copin R., Pascal K. E., Russo V., Giordano S., Lanza K., Negron N., Ni M. Antibody cocktail to SARS-CoV-2 spike protein prevents rapid mutational escape seen with individual antibodies. Science. 2020; 369:1014-1018.
Brouwer P. J. M., Caniels T. G., van der Straten K., Snitselaar J. L., Aldon Y., Bangaru S., Torres J. L., Okba N. M. A., Claireaux M., Kerster G. Potent neutralizing antibodies from COVID-19 patients define multiple targets of vulnerability. Science. 2020; 369:643-650.
Case J. B., Rothlauf P. W., Chen R. E., Liu Z., Zhao H., Kim A. S., Bloyet L. M., Zeng Q., Tahan S., Droit L. Neutralizing Antibody and Soluble ACE2 Inhibition of a Replication-Competent VSV-SARS-CoV-2 and a Clinical Isolate of SARS-CoV-2. Cell Host Microbe. 2020; 28:475-485.e5.
Chan K. K., Dorosky D., Sharma P., Abbasi S. A., Dye J. M., Kranz D. M., Herbert A. S., Procko E. Engineering human ACE2 to optimize binding to the spike protein of SARS coronavirus 2. Science. 2020; 369:1261-1265.
Chen P., Nirula A., Heller B., Gottlieb R. L., Boscia J., Morris J., Huhn G., Cardona J., Mocherla B., Stosor V. SARS-CoV-2 Neutralizing Antibody LY-CoV555 in Outpatients with Covid-19. N Engl J Med. 2021; 384:229-237.
CoV-GLUE 2021. http://cov-glue.cvr.gla.ac.uk/#/home
Dinnon K. H., 3rd, Leist S. R., Schsfer A., Edwards C. E., Martinez D. R., Montgomery S. A., West A., Yount B. L., Jr., Hou Y. J., Adams L. E. A mouse-adapted model of SARS-CoV-2 to test COVID-19 countermeasures. Nature. 2020; 586:560-566.
Dolan P. T., Whitfield Z. J., Andino R. Mapping the Evolutionary Potential of RNA Viruses. Cell Host Microbe. 2018; 23:435-446.
Domingo E., Sheldon J., Perales C. Viral quasispecies evolution. Microbiol. Mol. Biol. Rev. 2012; 76:159-216.
Du S., Cao Y., Zhu Q., Yu P., Qi F., Wang G., Du X., Bao L., Deng W., Zhu H. Structurally Resolved SARS-CoV-2 Antibody Shows High Efficacy in Severely Infected Hamsters and Provides a Potent Cocktail Pairing Strategy. Cell. 2020; 183:1013-1023.e13.
GISAID 2021. https://www.gisaid.org/
Greaney A. J., Starr T. N., Gilchuk P., Zost S. J., Binshtein E., Loes A. N., Hilton S. K., Huddleston J., Eguia R., Crawford K. H. D. Complete Mapping of Mutations to the SARS-CoV-2 Spike Receptor-Binding Domain that Escape Antibody Recognition. Cell Host Microbe. 2021; 29:44-57.e9.
ACTIV-3/TICO LY-CoV555 Study Group A Neutralizing Monoclonal Antibody for Hospitalized Patients with Covid-19. N Engl J Med. 2020 doi: 10.1056/NEJMoa2033130.
Gu H., Chen Q., Yang G., He L., Fan H., Deng Y. Q., Wang Y., Teng Y., Zhao Z., Cui Y. Adaptation of SARS-CoV-2 in BALB/c mice for testing vaccine efficacy. Science. 2020; 369:1603-1607.
Halfmann P. J., Hatta M., Chiba S., Maemura T., Fan S., Takeda M., Kinoshita N., Hattori S. I., Sakai-Tagawa Y., Iwatsuki-Horimoto K. Transmission of SARS-CoV-2 in Domestic Cats. N. Engl. J. Med. 2020; 383:592-594.
Hoffmann M., Kleine-Weber H., Schroeder S., Kruger N., Herrler T., Erichsen S., Schiergens T. S., Herrler G., Wu N. H., Nitsche A. SARS-CoV-2 Cell Entry Depends on ACE2 and TMPRSS2 and Is Blocked by a Clinically Proven Protease Inhibitor. Cell. 2020; 181:271-280.e8.
Ke Z., Oton J., Qu K., Cortese M., Zila V., McKeane L., Nakane T., Zivanov J., Neufeldt C. J., Cerikan B. Structures and distributions of SARS-CoV-2 spike proteins on intact virions. Nature. 2020; 588:498-502.
Lan J., Ge J., Yu J., Shan S., Zhou H., Fan S., Zhang Q., Shi X., Wang Q., Zhang L., Wang X. Structure of the SARS-CoV-2 spike receptor-binding domain bound to the ACE2 receptor. Nature. 2020; 581:215-220.
Letko M., Marzi A., Munster V. Functional assessment of cell entry and receptor usage for SARS-CoV-2 and other lineage B betacoronaviruses. Nat. Microbiol. 2020; 5:562-569.
Li Q., Wu J., Nie J., Zhang L., Hao H., Liu S., Zhao C., Zhang Q., Liu H., Nie L. The Impact of Mutations in SARS-CoV-2 Spike on Viral Infectivity and Antigenicity. Cell. 2020; 182:1284-1294.e9.
Monteil V., Kwon H., Prado P., Hagelkruys A., Wimmer R. A., Stahl M., Leopoldi A., Garreta E., Hurtado Del Pozo C., Prosper F. Inhibition of SARS-CoV-2 Infections in Engineered Human Tissues Using Clinical-Grade Soluble Human ACE2. Cell. 2020; 181:905-913.e7.
Oude Munnink B. B., Sikkema R. S., Nieuwenhuijse D. F., Molenaar R. J., Munger E., Molenkamp R., van der Spek A., Tolsma P., Rietveld A., Brouwer M. Transmission of SARS-CoV-2 on mink farms between humans and mink and back to humans. Science. 2021; 371:172-177.
Rogers T. F., Zhao F., Huang D., Beutler N., Burns A., He W. T., Limbo O., Smith C., Song G., Woehl J. Isolation of potent SARS-CoV-2 neutralizing antibodies and protection from disease in a small animal model. Science. 2020; 369:956-963.
Sanjusn R., Nebot M. R., Chirico N., Mansky L. M., Belshaw R. Viral mutation rates. J. Virol. 2010; 84:9733-9748.
Shang J., Ye G., Shi K., Wan Y., Luo C., Aihara H., Geng Q., Auerbach A., Li F. Structural basis of receptor recognition by SARS-CoV-2. Nature. 2020; 581:221-224.
Shi J., Wen Z., Zhong G., Yang H., Wang C., Huang B., Liu R., He X., Shuai L., Sun Z. Susceptibility of ferrets, cats, dogs, and other domesticated animals to SARS-coronavirus 2. Science. 2020; 368:1016-1020.
Smith E. C., Denison M. R. Coronaviruses as DNA wannabes: a new model for the regulation of RNA virus replication fidelity. PLoS Pathog. 2013; 9:e1003760.
Smith E. C., Blanc H., Surdel M. C., Vignuzzi M., Denison M. R. Coronaviruses lacking exoribonuclease activity are susceptible to lethal mutagenesis: evidence for proofreading and potential therapeutics. PLoS Pathog. 2013; 9:e1003565.
Stadlbauer D., Zhu X., McMahon M., Turner J. S., Wohlbold T. J., Schmitz A. J., Strohmeier S., Yu W., Nachbagauer R., Mudd P. A. Broadly protective human antibodies that target the active site of influenza virus neuraminidase.
Science. 2019; 366:499-504 Starr T. N., Greaney A. J., Hilton S. K., Ellis D., Crawford K. H. D., Dingens A. S., Navarro M. J., Bowen J. E., Tortorici M. A., Walls A. C. Deep Mutational Scanning of SARS-CoV-2 Receptor Binding Domain Reveals Constraints on Folding and ACE2 Binding. Cell. 2020; 182:1295-1310.e20.
Tegally H., Wilkinson E., Giovanetti M., Iranzadeh A., Fonseca V., Giandhari J., Doolabh D., Pillay S., James San E., Msomi N. Emergence and rapid spread of a new severe acute respiratory syndrome-related coronavirus 2 (SARS-CoV-2) lineage with multiple spike mutations in South Africa. medRxiv. 2020 doi: 10.1101/2020.12.21.20248640.
Weinreich D. M., Sivapalasingam S., Norton T., Ali S., Gao H., Bhore R., Musser B. J., Soo Y., Rofail D., Im J. REGN-COV2, a Neutralizing Antibody Cocktail, in Outpatients with Covid-19. N Engl J Med. 2021; 384:238-251.
Weisblum Y., Schmidt F., Zhang F., DaSilva J., Poston D., Lorenzi J. C., Muecksch F., Rutkowska M., Hoffmann H. H., Michailidis E. Escape from neutralizing antibodies by SARS-CoV-2 spike protein variants. eLife. 2020; 9:e61312.
Wrapp D., Wang N., Corbett K. S., Goldsmith J. A., Hsieh C. L., Abiona O., Graham B. S., McLellan J. S. Cryo-EM structure of the 2019-nCoV spike in the prefusion conformation. Science. 2020; 367:1260-1263.
Wu F., Zhao S., Yu B., Chen Y. M., Wang W., Song Z. G., Hu Y., Tao Z. W., Tian J. H., Pei Y. Y. A new coronavirus associated with human respiratory disease in China. Nature. 2020; 579:265-269.

Wu Y., Wang F., Shen C., Peng W., Li D., Zhao C., Li Z., Li S., Bi Y., Yang Y. A noncompeting pair of human neutralizing antibodies block COVID-19 virus binding to its receptorACE2. Science. 2020; 368:1274-1278
Zang R., Gomez Castro M. F., McCune B. T., Zeng Q., Rothlauf P. W., Sonnek N. M., Liu Z., Brulois K. F., Wang X., Greenberg H. B. TMPRSS2 and TMPRSS4 promote SARS-CoV-2 infection of human small intestinal enterocytes. Sci. Immunol. 2020; 5:eabc3582.
Zost S. J., Gilchuk P., Case J. B., Binshtein E., Chen R. E., Nkolola J. P., Schafer A., Reidy J. X., Trivette A., Nargi R. S. Potently neutralizing and protective human antibodies against SARS-CoV-2. Nature. 2020; 584:443-449.

### Example 6: Use VSV-SARS2 to Study and Anticipate the Evolution of the Virus in Nature

As variants of SARS-CoV-2 emerge in the human population, their ability to resist therapeutic antibodies, receptor decoys, other inhibitors of viral entry and infection, convalescent plasma, and possibly vaccine induced immunity is of concern. Disclosed herein is also the use VSV-SARS-CoV-2 and variants thereof to study and anticipate the evolution of the virus in nature. Essentially an inhibitor of the spike function can be used to identify mutants that allow for escape from inhibition in the laboratory. This cannot be easily done with SARS-CoV-2 virus itself because that virus has an enzyme called a “proof reading enzyme” on endonuclease that excises nucleotide errors. Using this approach over 200 variants have been generated in the lab and they can be rapidly characterized.

As an example, we started out by selecting substitutions E484K and E484A—at the time we first isolated those variants they were not yet reported in sequences of human viruses, but are now. We have consistently seen this over time as more human viral sequences are reported they match mutants that we have isolated.

Here is disclosed the use of VSV-SARS-CoV-2 in which the coronavirus S sequence represents the nucleotide sequence of a circulating viral strain, as a tool to anticipate the evolution of that Spike sequence in nature. Using monoclonal antibodies, soluble receptors or other inhibitors of spike function we can rapidly explore the mutational landscape that can result in resistance to any given inhibitor. Comparing those data to sequence data obtained from surveillance of circulating viruses will allow anticipation of resistance mutations. That knowledge will inform whether specific therapeutics are rendered ineffective and can also be used to inform design of a vaccine. This is possible because the VSV-SARS-CoV-2 S chimeras, like SARS-CoV-2 depend upon the functions of S necessary for infection. Those functions are inhibited by antibodies, receptor decoys, and vaccine induced antibodies as well as other molecules that inhibit S function. The rapidity of escape attainable with VSV provides an assay platform in which the S sequence of any natural variant can be rapidly evolved in the laboratory to anticipate sequences that could represent additional new variants of concern.

1. A recombinant vesicular stomatitis virus (rVSV) comprising a nucleic acid sequence encoding at least a portion of a coronavirus spike (S) protein or a functional fragment or a functional variant thereof.

2. A recombinant vesicular stomatitis virus (rVSV) construct or vaccine platform comprising:
at least a portion of a vesicular stomatitis virus (VSV); and
at least a portion of, a functional fragment of, or functional variant of the spike (S) of SARS-CoV-2 (SEQ ID NO: 1) and/or at least about 80% identical to SEQ ID NO: 1.

3. A recombinant vesicular stomatitis virus (rVSV) comprising in its genome a nucleic acid sequence encoding at least a portion of, a functional fragment of, or functional variant of the spike (S) of SARS-CoV-2 (SEQ ID NO: 1) and/or at least about 80% identical to a functional portion or fragment of SEQ ID NO: 1.

4. The rVSV of claim 1, wherein the rVSV is replication-competent.

5. The rVSV of claim 2, wherein the at least a portion of, a functional fragment, or functional variant of the spike (S) of SARS-CoV-2 comprises one or more mutations or attenuations or truncations.

6. The rVSV of claim 1, wherein at least a portion of the rVSV comprises genes encoding a leader region (Le), a reporter gene, nucleocapsid (N), phosphoprotein (P), matrix (M), glycoprotein (G), SARS-CoV-2 (S), large polymerase (L), or trailer region (Tr), or combinations thereof.

7. The rVSV of claim 2, wherein the rVSV construct displays the spike (S) in an antigenic form that resembles native infectious SARS-CoV-2.

8. The rVSV of claim 2, wherein the coding region of the glycoprotein (G) of the VSV is replaced by the coding region of a spike (S) protein of SARS-CoV-2 or a functional variant, portion, or fragment of SEQ ID NO: 1.

9. The rVSV of claim 1, wherein the spike (S) protein is selected from a functional mutation, insertion, deletion, or substitution thereof and 80% identical to SEQ ID NO: 1.

10. The rVSV of claim 1, wherein the spike (S) protein is selected from any one of SEQ ID NO: 1 or 4-9 or a functional portion or fragment thereof.

11. The rVSV of claim 1, comprising a tail mutation capable of allowing for its incorporation into a VSV construct.

12. The rVSV of claim 11, wherein the tail mutation is selected from one or more of the following: S_{Δ21} (tail mutant 21 amino acid truncation or deletion of residues 1253-1273) or S_{AA} (tail mutant K1269A/H1271A).

13. The rVSV of claim 1, comprising a spike mutation selected from one or more of the following:
T345A;
T345A/L517R;
T345A/E484A;
T345N;
T345S;
T345S/F486S;
T346G/E484A;
R346K/E484K;
R346G;
R346K;
A352D;
A372T;
A372T/E484K;
K378E;
K378Q;
R408K;
K417N;
L441R;
K444E;
K444E/E484A;
K444E/E484K;
K444N;
K444R;
V445G;
G446D;
G446V;
N450D;
N450K;
N450Y;
L452R;
K458Q;
Q474P;
G476D;
G476S;
S477I;
S477G;
S477N;
S477N/S514F;
S477R;
T478I;
T478P;
P479L;
P479S;
V483F;
V483G;
E484;
E484A;
E484D;
E484G;
E484K;
F486L;
F486S;
F486Y;
F486V;
E488A;
F490L;
F490S;
S494P;
P499L;
N501Y;
G504D;
S514F;
L517R;
K535R;
D614G; or
combinations thereof.

14. The rVSV of claim 1, wherein the spike (S) protein is selected from S_{AA}, S_{Δ21}, K535R, or a functional variant, mutant, or fragment thereof having at least 80% identity to SEQ ID NO: 1.

15. The rVSV of claim 1, wherein the nucleic acid sequence encoding at least a portion of a Coronavirus spike protein (S) protein, or functional fragment or variant thereof, substantially replaces the endogenous VSV viral glycoprotein (G) in the VSV genome.

16. The rVSV of claim 1, wherein
the nucleic acid encoding the at least a portion or a functional fragment of a Coronavirus spike protein (S) protein or a functional fragment or variant thereof has a sequence 80% identical to a functional portion or fragment of the Coronavirus spike protein portion of SEQ ID NO: 2 or SEQ ID NO: 3; or
the nucleic acid encoding the at least a portion of a Coronavirus spike protein (S) protein or functional fragment or variant thereof encodes a polypeptide having a sequence 80% identical to a functional portion or fragment of SEQ ID NO: 1 or SEQ ID NO: 4-9.

17. The rVSV of claim 1, wherein the at least a portion of a Coronavirus spike (S) protein or functional fragment or variant thereof comprises an amino acid sequence that is identical to the at least a portion of the Coronavirus spike protein sequence of SEQ ID NO: 3 or SEQ ID NO: 8-10.

18. A pharmaceutical composition comprising the rVSV of claim 1, and a pharmaceutically acceptable carrier.

19. The pharmaceutical composition of claim 18, further comprising an adjuvant.

20. A Coronavirus vaccine comprising the rVSV of claim 1.

21. The Coronavirus vaccine of claim 20, further comprising an adjuvant, K3 CpG.

22. The vaccine of claim 21, wherein the vaccine is a replication-competent vaccine against SARS-CoV-2.

23. The vaccine of claim 21, wherein the recombinant VSV is a live virus.

24. A nucleic acid encoding the rVSV according to claim 1.

25. The nucleic acid of claim 24, wherein the nucleic acid has a sequence that is at least 80% identical to the portion encoding the Coronavirus spike protein of a functional portion or fragment of SEQ ID NO: 2 or SEQ ID NO: 3.

26. An expression vector comprising the nucleic acid of claim 1.

27. A cell comprising the nucleic acid according to claim 24.

28. A method for inducing an immune response against Coronavirus in a subject, the method comprising administering to the subject at least one dose of a composition comprising the rVSV of claim 1.

29. The method of claim 28, wherein the administration of the composition generates a robust neutralizing antibody response that targets both a SARS-CoV-2 S protein and a receptor binding domain (RBD) subunit.

30. The method of claim 28, wherein the administration of the composition stimulates both humoral and cellular immunity.

31. The method of claim 28, wherein the administration of the composition results in a response comprising decreases lung or peripheral organ viral loads, pro-inflammatory cytokine responses, and/or consequent lung disease.

32. The method of claim 28, wherein the administration of the composition results in protection from alveolar inflammation, lung consolidation, or viral pneumonia.

33. The method of claim 28, wherein the administration of the composition results in protection against severe SARS-CoV-2 infection and lung disease.

34. The method of claim 28, wherein the subject produces protective antibodies in the sera of the subject.

35. The method of claim 34, wherein passive transfer of immune sera from immunized subject decreases viral burden or inflammation in the lung.

36. The method of claim 28, wherein a second dose substantially boosted the response.

37. The method of claim 28, wherein the route of administration is intramuscular or intranasal.

38. The method of claim 28, wherein the subject is a human.

39. The method of claim 28, wherein the subject has been exposed to Coronavirus.

40. The method of claim 28, wherein the subject does not have, but is at risk of developing a Coronavirus infection.

41. The method of claim 28, wherein the subject is traveling to a region where the Coronavirus is prevalent.

42. A method for protecting a subject from Coronavirus, comprising administering to the subject at least one dose of the rVSV of claim 1, wherein, optionally, the rVSV confers protection against SARS-CoV-2-induced lung infection and/or inflammation, such as pneumonia.

43. The method of claim 42, wherein the subject is a human.

44. The method of claim 42, wherein the subject is exposed to Coronavirus.

45. The method of claim 42, wherein the subject does not have, but is at risk of developing a Coronavirus infection.

46. The method of claim 42, wherein the subject is traveling to a region where the Coronavirus is prevalent.

47. A method of treating a Coronavirus infection in a subject, the method comprising
administering to the subject the sera of a subject that received a composition comprising the rVSV of claim 1; or
administering to the subject the composition comprising the rVSV of claim 1.

48. The method of claim 47, wherein the subject is a human.

49. A method of making virus-like particles (VLP) comprising:
transfecting a cell with the expression vector of claim 26; culturing the cell under conditions such that the cell produces a Coronavirus VLP; and collecting the Coronavirus VLP.

50. The method of claim 49, wherein the cells are BSRT7 cells, Vero, or MA104.

51. The method of claim 49, wherein the cells are infected with Vaccinia VTF7-3 and transfected with an infectious molecular clone encoding S or an S mutant and helper plasmids N, P, L, and G to rescue recombinant virus.

52. The method of claim 49, further comprising infecting the cells with a rescue supernatant, viral particles of which contain VSV G in trans.

53. A method for screening treatments for Coronavirus comprising:
providing a cell infected with the rVSV of claim 1, further comprising a reporter gene;
contacting the cell with an experimental therapeutic agent; and
detecting the reporter gene expression to determine if the experimental therapeutic agent neutralized the rVSV.

54. A method for screening a subject for Coronavirus antibodies:
providing a biological sample from a subject;
contacting a cell with the rVSV of claim 1, further comprising a reporter gene; and
detecting the reporter gene expression to determine if antibodies in the sample neutralized the rVSV, compared to a cell not having Coronavirus antibodies or a biological sample from a subject not having been infected with Coronavirus.

55. The method of claim 54, wherein the vesicular stomatitis virus (VSV) encodes a SARS-CoV-2 spike or mutant, or truncated or attenuated variant thereof at least 80% identical to a functional portion or fragment of SEQ ID NO: 1.

56. A neutralization assay comprising:
a rVSV construct expressing a reporter gene and a SARS-CoV-2 spike protein or mutant, or truncated or attenuated variant thereof at least 80% identical to a functional portion or fragment of SEQ ID NO: 1.

57. The rVSV construct of claim 2, wherein the SARS-CoV-2 spike (S) is S_{Δ21} or S_{AA} or mutant, or truncated or attenuated variant thereof at least 80% identical to a functional portion or fragment of SEQ ID NO: 1.

58. The neutralization assay of claim 56, wherein the assay is a BSL2 assay for evaluating SARS-CoV-2 entry and its inhibition by antibodies.

59. The neutralization assay of claim 56, wherein the assay is a high-throughput-imaging-based neutralization assay at biosafety level 2.

60. The neutralization assay of claim 56, wherein the assay is capable of testing inhibitors of SARS-CoV-2 mediated entry under reduced biosafety containment.

61. The neutralization assay of claim 60, wherein the inhibitors are selected from monoclonal antibodies or an ACE receptor.

62. The neutralization assay of claim 56, wherein the neutralization assay correlates with a focus-reduction neutralization test with a clinical isolate of SARS-CoV-2 at biosafety level 3.

63. The neutralization assay of claim 56, wherein the spike protein is a functional spike protein having ACE2 receptor binding activity.

64. A method of measuring neutralization comprising:
infecting a cell with an rVSV-CoV-2-S comprising a reporter gene construct and imaging the reporter gene after contacting the cell with a test agent or treatment with an inhibiting agent.

65. The method of claim 64, wherein the test agent is analyzed for neutralization activity.

66. The method of claim 64, wherein imaging of the reporter gene is performed using a fluorescence microscope with automated counting analysis software.

67. A method of identifying escape mutants comprising:
selecting a spike protein mutation;
generating a VSV-SARS-CoV-2-S having the spike protein mutation;
contacting the VSV-SARS-CoV-2-S with an inhibitor of spike function; and
identifying if the mutation is an escape mutation based on reporter gene signal.

68. The method of claim 67, wherein the spike protein mutation represents a nucleotide sequence of a circulating viral strain.

69. The method of claim 67, wherein the inhibitor of spike function is a monoclonal antibody, a soluble receptor, or other inhibitors of spike function, such as antibodies, Fc, receptor decoys, vaccine induced antibodies, or molecules.

70. The method of claim 67, comprising comparing escape mutation results to sequence data obtained from surveillance of circulating viruses.

71. The method of claim 70, wherein if escape mutations correlate with circulating viral mutations, a vaccine can be designed to include the mutation.


SEQUENCE LISTING (13 sequences)

SEQ ID NO: 1 - protein, 1273 aa, Homo sapiens
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 17286 bp, Homo sapiens
acgaagacaaacaaaccattattatcattaaaaggctcaggagaaactttaacagtaatcagaattctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatggccatatgaaaaaaactaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctactctcgtctagatcaggcggtaggagagatagatgagatcgaagcacaacgagctgaaaagtccaattatgagttgttccaagaggacggagtggaagagcatactaggccctcttattttcaggcagcagatgattctgacacagaatctgaaccagaaattgaagacaatcaaggcttgtatgtaccagatccggaagctgagcaagttgaaggctttatacaggggcctttagatgactatgcagatgaggacgtggatgttgtattcacttcggactggaaacagcctgagcttgaatccgacgagcatggaaagaccttacggttgacattgccagagggtttaagtggagagcagaaatcccagtggcttttgacgattaaagcagtcgttcaaagtgccaaacactggaatctggcagagtgcacatttgaagcatcgggagaaggggtcatcataaaaaagcgccagataactccggatgtatataaggtcactccagtgatgaacacacatccgtaccaatcagaagccgtatcagatgtttggtctctctcaaagacatccatgactttccaacccaagaaagcaagtcttcagcctctcaccatatccttggatgaattgttctcatctagaggagaattcatctctgtcggaggtaacggacgaatgtctcataaagaggccatcctgctcggtctgaggtacaaaaagttgtacaatcaggcgagagtcaaatattctctgtagactatgaaaaaaagtaacagatatcacaatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctagcttccagcttctgaacaatccccggtttactcagtctctcctaattccagcctttcgaacaactaatatcctgtcttttctatccctatgaaaaaaactaacagagatcgatctgtttccttgacacgcgtaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcgcattagcttacacataagcggccgccctgcacacagattcttcatgtttgaaccaaatcaacttgtgatatcatgctcaaagaggccttaattatattttaatttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttggcagttatccagcaatcatttccgattaggtgggaggctatttcagtaaaaggaggatacaagcagaagtggagtactagaggtgatgggctcccaaaagatacccgaatttcagactccttggccccaatcgggaactggatcagatctttggaattggtccgaaaccaagttcgtctaaatccattcaataagatcttgttcaatcagctatgtcgtacagtggataatcatttgaagtggtcaaatttgcgaaaaaacacaggaatgattgaatggatcaatgggcgaatttcaaaagaagaccggtctatactgatgttgaagagtgacctacatgaggaaaactcttggagagattaaaaaatcaggaggagactccaaactttaagtatgaaaaaaactttgatccttaagaccctcttgtggtttttatttttttatctggttttgtggtcttcgtgggtcggcatggcatctccacctcctcgcggtccgacctgggcatccgaaggaggacgtcgtccactcggatggctaagggagagctcggatccggctgctaacaaagcccgaaaggaagctgagttggctgctgccaccgctgagcaataactagcataaccccttggggcctctaaacgggtcttgaggggttttttgctgaaaggaggaactatatccggatcgagatcctctagagtcgacctgcaggcatgcaagcttgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggggatctcgatcccgcgaaattaatacgactcactatagg

SEQ ID NO: 3 - DNA, 14211 bp, Homo sapiens
acgacaaacaaaccattattatcattaaaaggctcaggagaaactttaacagtaatcagaattctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcttgctggagttcgtgaccgccgccgggatcactctcggcatggacgagctgtacaagtgatggccatatgaaaaaaactaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctactctcgtctagatcaggcggtaggagagatagatgagatcgaagcacaacgagctgaaaagtccaattatgagttgttccaagaggacggagtggaagagcatactaggccctcttattttcaggcagcagatgattctgacacagaatctgaaccagaaattgaagacaatcaaggcttgtatgtaccagatccggaagctgagcaagttgaaggctttatacaggggcctttagatgactatgcagatgaggacgtggatgttgtattcacttcggactggaaacagcctgagcttgaatccgacgagcatggaaagaccttacggttgacattgccagagggtttaagtggagagcagaaatcccagtggcttttgacgattaaagcagtcgttcaaagtgccaaacactggaatctggcagagtgcacatttgaagcatcgggagaaggggtcatcataaaaaagcgccagataactccggatgtatataaggtcactccagtgatgaacacacatccgtaccaatcagaagccgtatcagatgtttggtctctctcaaagacatccatgactttccaacccaagaaagcaagtcttcagcctctcaccatatccttggatgaattgttctcatctagaggagaattcatctctgtcggaggtaacggacgaatgtctcataaagaggccatcctgctcggtctgaggtacaaaaagttgtacaatcaggcgagagtcaaatattctctgtagactatgaaaaaaagtaacagatatcacaatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctagcttccagcttctgaacaatccccggtttactcagtctctcctaattccagcctttcgaacaactaatatcctgtcttttctatccctatgaaaaaaactaacagagatcgatctgtttccttgacacgcgtaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgatgcaaatttgatgaagacgactctgagccagtgctcaaaggagtcgcattagcttacacataagcggccgccctgcacacagattcttcatgtttgaaccaaatcaacttgtgatatcatgctcaaagaggccttaattatattttaatttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagttttttgacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttggcagttatccagcaatcatttccgattaggtgggaggctatttcagtaaaaggaggatacaagcagaagtggagtactagaggtgatgggctcccaaaagatacccgaatttcagactccttggccccaatcgggaactggatcagatctttggaattggtccgaaaccaagttcgtctaaatccattcaataagatcttgttcaatcagctatgtcgtacagtggataatcatttgaagtggtcaaatttgcgaaaaaacacaggaatgattgaatggatcaatgggcgaatttcaaaagaagaccggtctatactgatgttgaagagtgacctacatgaggaaaactcttggagagattaaaaaatcaggaggagactccaaactttaagtatgaaaaaaactttgatccttaagaccctcttgtggtttttattttttatctggttttgtggtcttcgt

SEQ ID NO: 4 - protein, 81 aa, Homo sapiens
LNESLIDLQELGKYEQYIKWPWYIWLGFIAGLIAIVMVTIMLCCMTSCCSCLKGCCSCGSCCKFDEDDSEPVLKGVALAYT

SEQ ID NO: 5 - protein, 66 aa, Homo sapiens
LNESLIDLQELGKYEQYIKWPWYIWLGFIAGLIAIVMVTIMLKLKCNRCCDRYEEYDLEPHAVAVH

SEQ ID NO: 6 - protein, 81 aa, Homo sapiens
LNESLIDLQELGKYEQYIKWPWYIWLGFIAGLIAIVMVTIMLCCMTSCCSCLRVGIYLCIKLKHTKKRQIYTDIEMNRLGK

SEQ ID NO: 7 - protein, 71 aa, Homo sapiens
LNESLIDLQELGKYEQYIKWPWYIWLGFIAGLIAIVMVTIMLRVGIYLCIKLKHTKKRQIYTDIEMNRLGK

SEQ ID NO: 8 - protein, 59 aa, Homo sapiens
LNEGWFSSWKSSIASFCFIIGLIIGLFLVLRVGIYLCIKLKHTKKRQIYTDIEMNRLGK

SEQ ID NO: 9 - protein, 60 aa, Homo sapiens
LNESLIDLQELGKYEQYIKWPWYIWLGFIAGLIAIVMVTIMLCCMTSCCSCLKGCCSCGS

SEQ ID NO: 10 - protein, 69 aa, Homo sapiens
LNESLIDLQELGKYEQYIKWSSIASFCFIIGLIIGLFLVLRVGIYLCIKLKHTKKRQIYTDIEMNRLGK

SEQ ID NO: 11 - protein, 8 aa, Homo sapiens
NGVAGFNC

SEQ ID NO: 12 - protein, 8 aa, Homo sapiens
NGVKGFNC

SEQ ID NO: 13 - protein, 8 aa, Homo sapiens
NGVEGSNC